(19) United States
(12) Patent Application Publication
(10) Pub. No.: US 2023/0203153 A1
(43) Pub. Date: Jun. 29, 2023

(54) ANTIBODIES SPECIFIC TO ABCB5 AND USES THEREOF

(71) Applicants: Children's Medical Center Corporation, Boston, MA (US); TICEBA GmbH, Heidelberg (DE); RHEACELL GmbH & Co., KG, Heidelberg (DE)

(72) Inventors: Markus H. Frank, Cambridge, MA (US); Christoph Ganss, Heidlberg (DE); Christoph Maßlo, Heidelberg (DE); Mark Andreas Kluth, Heidelberg (DE); Jan Reckenbeil, Heidelberg (DE)

(73) Assignees: Children's Medical Center Corporation, Boston, MA (US); TICEBA GmbH, Heidelberg (DE); RHEACELL GmbH & Co., KG, Heidelberg (DE)

(21) Appl. No.: 17/922,286

(22) PCT Filed: Apr. 30, 2021
(86) PCT No.: PCT/US2021/030342

§ 371 Date: Oct. 28, 2022

Related U.S. Application Data
(60) Provisional application No. 63/018,440, filed on Apr. 30, 2020.

(51) Int. Cl. C07K 16/28; C12N 15/63; A61P 35/04

(52) U.S. Cl. CPC C07K 16/28; C12N 15/63; A61P 35/04; C07K 2317/565; C07K 2317/33; C07K 2317/35; C07K 2317/622; C07K 2319/02; C07K 2319/03

(57) ABSTRACT

Disclosed herein are anti-ABCB5 antibodies possessing superior binding and biological activities, for instance, relative to commercially available ABCB5 antibodies, pharmaceutical compositions comprising such. Also provided herein are therapeutic and diagnostic applications of such anti-ABCB5 antibodies.

### RELATED APPLICATIONS

This application claims the benefit under 35 U.S.C. § 119(e) of U.S. provisional application No. 63/018,440, filed Apr. 30, 2020 which is incorporated by reference herein in it's entirety.

### BACKGROUND OF INVENTION

Cancers, one of the leading causes of death, are a large family of diseases characterized by the uncontrolled growth of cells in a body. Numerous therapies have been developed to treat cancer, including for example, surgical removal of the cancer, chemotherapeutic drugs, and radiation therapy. However, many cancers still do not effectively have a cure. In particular, human malignant melanoma is a highly chemorefractory cancer, with very few effective treatment options.

ABCB5 is a multidrug resistance (MDR) mediator expressed in diverse human malignancies, where it is specifically overexpressed on therapy-resistant CD133(+) tumor subpopulations previously found to represent CSC. ABCB5 confers cancer cell drug resistance to chemotherapeutic agents such as 5-fluorouracil (5-FU).

ABCB5⁺ stem cells are also found in normal tissue and have a role in tissue regeneration and aging. Regenerative medicine involves the repair, regeneration, maintenance, and replacement of tissues and organs using exogenous materials such as scaffolds. The scaffolds may be seeded with cells, such as primary cells or stem cells, and various factors to encourage tissue growth.

### SUMMARY

In some aspects an antibody binding to ATP-binding cassette transporter family member B5 (ABCB5), is provided. The antibody comprises a heavy chain variable domain (V_{H}), which comprises (i) a heavy chain complementary determining region 1 (HC CDR1) having at least 90% sequence identity to a sequence set forth as any one of SEQ ID NOs: 49-56, (ii) a heavy chain complementary determining region 2 (HC CDR2) having at least 90% sequence identity to a sequence set forth as any one of SEQ ID NOs: 57-65; and (iii) a heavy chain complementary determining region 3 (HC CDR3) having at least 90% sequence identity to a sequence set forth as any one of SEQ ID NOs: 66-72; and/or wherein the antibody comprises a light chain variable domain (V_{L}), which comprises (i) a light chain complementary determining region 1 (LC CDR1) having at least 90% sequence identity to a sequence set forth as any one of SEQ ID NOs: 51-54, 73-76 and 80; (ii) a light chain complementary determining region 2 (LC CDR2) having at least 90% sequence identity to a sequence set forth as any one of SEQ ID NOs: 60-63 and 77-78; and (iii) a light chain complementary determining region 3 (LC CDR3) having at least 90% sequence identity to a sequence set forth as any one of SEQ ID NOs: 67, 69, 70, 79, 81, and 103. In some embodiments the antibody optionally is not AB100 or Ab101.

In other aspects an antibody binding to ATP-binding cassette transporter family member B5 (ABCB5), is provided. The antibody comprises a heavy chain variable domain (V_{H}), which comprises (i) a heavy chain complementary determining region 1 (HC CDR1) set forth as GFTFSSYX₁MN (SEQ ID NO: 109) or GYTFTX₂YYMH (SEQ NO: 110), in which X₁ is S or D or T and X₂ is S or N, (ii) a heavy chain complementary determining region 2 (HC CDR2) set forth as YISSSX₃X₄TIYYADSVKG (SEQ ID NO: 111) or IINPSGGSTSYAQKFXsG (SEQ ID NO: 112), in which X₃ is S or G, and X₄ is S or N; and X₅ is K or Q and (iii) a heavy chain complementary determining region 3 (HC CDR3) set forth as NYQYGDYGGY (SEQ ID NO: 66) or DX₆AVTGTAYYYYYGMDV (SEQ ID NO: 113), in which X₆ is Q or L; and/or wherein the antibody comprises a light chain variable domain (V_{L}), which comprises (i) a light chain complementary determining region 1 (LC CDR1) set forth as X₇ASHDISNFLN (SEQ ID NO: 114) or RASX₈SVNSX₉YLA (SEQ ID NO: 115), in which X₇ is Q or H; X₈ is L or Q; and X₉ is N or K (ii) a light chain complementary determining region 2 (LC CDR2) set forth as DAYNLQT (SEQ ID NO: 77) or GTSSRAT (SEQ ID NO: 78) and (iii) a light chain complementary determining region 3 (LC CDR3) set forth as QQYDYFLSIT (SEQ ID NO: 79) or QQFGSSPLT (SEQ ID NO: 81), optionally wherein the antibody is not AB100 or Ab101.

In other aspects an antibody binding to ATP-binding cassette transporter family member B5 (ABCB5), wherein the antibody binds the same epitope as Ab100 or Ab101 or competes against Ab100 or Ab101 from binding to the ABCB5 is provided.

In yet other aspects an antibody which recognizes an epitope of human ABCB5 comprising SEQ ID NO. 104 or having at least 80% sequence identity thereto is provided.

In some embodiments the antibody specifically binds human ABCB5. In other embodiments the antibody cross-reacts with human ABCB5 and a non-human ABCB5. In some embodiments the antibody binds ABCB5 expressed on cell surface. In other embodiments the antibody comprises a HC CDR1, a HC CDR2, and a HC CDR3, which collectively contains no more than 10 amino acid variations as compared with the HC CDR1, HC CDR2, and HC CDR3 of Ab100 or Ab101; and/or a LC CDR1, a LC CDR2, and a LC CDR3, which collectively contains no more than 10 amino acid variations as compared with the LC CDR1, LC CDR2, and LC CDR3 of Ab100 or Ab101. The antibody comprises a HC CDR1, a HC CDR2, and a HC CDR3, which collectively contains no more than 8 amino acid variations as compared with the HC CDR1, HC CDR2, and HC CDR3 of Ab100 or Ab101 in other embodiments.

In some embodiments the antibody comprise a HC CDR1, a HC CDR2, and a HC CDR3, which collectively contains no more than 5 amino acid variations as compared with the HC CDR1, HC CDR2, and HC CDR3 of Ab100 or Ab101.

In some embodiments the antibody comprise a LC CDR1, a LC CDR2, and a LC CDR3, which collectively contains no more than 8 amino acid variations as compared with the LC CDR1, LC CDR2, and LC CDR3 of Ab100 or Ab101.

In some embodiments the antibody comprise a LC CDR1, a LC CDR2, and a LC CDR3, which collectively contains no more than 5 amino acid variations as compared with the HC CDR1, HC CDR2, and HC CDR3 of Ab100 or Ab101.

In some embodiments the antibody comprises a HC CDR1, a HC CDR2, and a HC CDR3, at least one of which contains no more than 5 amino acid variations as the counterpart HC CDR of Ab100 or Ab101; and/or a LC CDR1, a LC CDR2, and a LC CDR3, at least one of which contains no more than 5 amino acid variations as the counterpart LC CDR of Ab100 or Ab101.

In some embodiments the antibody comprises a HC CDR1, a HC CDR2, and a HC CDR3, at least one of which contains no more than 2 amino acid variations as the counterpart HC CDR of Ab100 or Ab101. In some embodiments the at least one HC CDR is HC CDR3. In some embodiments the antibody comprises a LC CDR1, a LC CDR2, and a LC CDR3, at least one of which contains no more than 2 amino acid variations as the counterpart LC CDR of Ab100 or Ab101.

In some embodiments the antibody comprises the same heavy chain complementary determining regions (HC CDRs) and/or the same light chain complementary determining regions (LC CDRs) as Ab100 or Ab101. In some embodiments the antibody comprises the same heavy chain variable domain as Ab100 or Ab101 and/or the same light chain variable domain as Ab100 or Ab101.

In some embodiments the antibody comprises a heavy chain variable domain that is at least 85% identical to the heavy chain variable domain of Ab100 or Ab101, and/or a light chain variable domain that is at least 85% identical to the light chain variable domain of Ab100 or Ab101.

In some embodiments the antibody is a human antibody or a humanized antibody.

In some embodiments the antibody is a full-length antibody. In some embodiments the full-length antibody is an IgG molecule. In some embodiments the antibody contains an altered Fc fragment relative to a naturally-occurring counterpart, or wherein the antibody contains an afucosylated Fc fragment, or wherein the antibody's antigen binding site is masked to allow protease mediated activation. In some embodiments the antibody contains an altered IgG1 Fc fragment, which comprises K214R. In some embodiments the antibody comprises a sequence having at least 90% sequence identity to a heavy chain variable sequence set forth as any of SEQ ID NO: 1-8 and 13-17 and a light chain variable sequence set forth as any of SEQ ID NO: 20-23 and 26-29. In some embodiments the antibody comprises a sequence having at least 90% sequence identity to a heavy chain sequence set forth as any of SEQ ID NO: 31-44 and a light chain sequence set forth as any of SEQ ID NO: 45-47.

In some embodiments the antibody a single-chain diabody (scDb), a bi-, tri-, tetra-, penta- or hexa-valent scFv tandem repeat (TaFv), is an antigen-binding fragment. In some embodiments the antigen-binding fragment is Fab, Fab′, F(ab′)2, or Fv.

In some embodiments the antibody is a single-chain antibody, a bispecific antibody or a nanobody.

In some embodiments the antibody is conjugated to a detectable label.

In aspects of the invention an antibody-drug conjugate (ADC), comprising an antibody disclosed herein coupled to a therapeutic agent is provided. In some embodiments the antibody is an scFv or a bi-, tri-, tetra-, penta- or hexa-valent scFv tandem repeat (TaFv) bivalent tandem scFv repeat (TaFv) thereof. In some embodiments the therapeutic agent is an auristatin peptide, auristatin E(AE), monomethylauristatin E(MMAE), or synthetic analog of dolastatin.

An antibody-drug conjugate (ADC), comprising an antibody binding specifically to ATP-binding cassette transporter family member B5 (ABCB5), coupled to a therapeutic agent through a linker is provided in aspects of the invention. In some embodiments the antibody is an scFv or a bi-, tri-, tetra-, penta- or hexa-valent scFv tandem repeat (TaFv) bivalent tandem scFv repeat (TaFv) thereof. In some embodiments the therapeutic agent is an auristatin peptide, auristatin E(AE), monomethylauristatin E(MMAE), or synthetic analog of dolastatin. In some embodiments n the linker is a flexible linker. In some embodiments the linker is selected from the group consisting of a peptide linker, a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain. In some embodiments the linker is a PEG linker comprising 2-24 PEG units. In some embodiments the linker is a sulfamide linker. In some embodiments the linker is a peptide linker comprising GSTSGGGSGGGSGGGGSS (SEQ ID NO. 84) or GGGGSS (SEQ ID NO. 86).

In some aspects the invention is a bispecific antibody, wherein the antibody has a region of antigen binding specificity for ATP-binding cassette transporter family member B5 (ABCB5) and a region of antigen binding specificity for an immune effector cell antigen. In some embodiments the immune effector cell antigen is Cd16 or CD3. In some embodiments region of antigen binding specificity for an immune effector cell antigen is an anti-Cd16 scFv or anti-CD3 scFv. In some embodiments region of antigen binding specificity for ABCB5 is an anti-ABCB5 scFv or an anti-ABCB5 monoclonal antibody. In some embodiments antibody comprises IgG-scFv fusion proteins [IgG-scFv's]. In some embodiments an antibody comprises Single-chain diabodies (scDb's). In some embodiments antibody comprises tandem scFv's (TaFv's).

A nucleic acid or a nucleic acid set, which collectively encode the antibody binding to ABCB5 disclosed herein is provided. In some embodiments the nucleic acid is a vector or a vector set. In some embodiments the vector(s) is an expression vector(s).

A host cell, comprising the vector or vector set as disclosed herein is also provided. In some embodiments the host cell is selected from the group consisting of a bacterial cell, a yeast cell, an insect cell, a plant cell, and a mammalian cell.

In other aspects a genetically engineered immune cell, which expresses a chimeric receptor comprising an extracellular domain and at least one cytoplasmic signaling domain, wherein the extracellular domain is a single chain antibody binding to ATP-binding cassette transporter family member B5 (ABCB5) is provided. In some embodiments the single chain antibody comprises a heavy chain variable domain and/or a light chain variable domain set forth in any one of SEQ ID NOs 1-8, 13-17 and 20-23, 26-29 respectively.

In some aspects a pharmaceutical composition, comprising (a) an antibody binding to ABCB5 as disclosed herein and (b) a pharmaceutically acceptable carrier is provided.

In other aspects a method for treating cancer in a subject is provided. The method comprises administering to a subject in need thereof an effective amount of the pharmaceutical composition disclosed herein. In some embodiments the human patient has a metastatic cancer. In some embodiments the subject has undergone or is undergoing an additional treatment of the disease. In some embodiments the disease is cancer and the treatment of cancer is surgery, a chemotherapy, an immune therapy, a radiotherapy, or a combination thereof.

A method for detecting presence of ABCB5 is provided in other aspects. The method comprises contacting an anti-ABCB5 antibody of claims 1-22, alone or in combination with other anti-ABCB5 antibodies specifically binding to and/or capturing other ABCB5 epitopes, optionally selected from the group consisting of (RFGAYLIQAGRMTPEG (SEQ ID NO. 104), TMFGNNDKTTLKHDAE (SEQ ID NO. 105), VTGMIETAAMTGFANKDKQELKHAGKIATEALENIRTIVSLTREKAFEQMYEEMLQT QHRNTSKKAQI (SEQ ID NO. 106), and QDIKKADEQMESMTYSTERKTNSLPLHSVKSIKSDFIDKAEESTQSKEISLPEVSLLK (SEQ ID NO. 107), with a biological sample suspected of containing ABCB5, and measuring binding of the anti-ABCB5 antibody to ABCB5 in the sample. In some embodiments the treatment comprises administering to the subject an immune checkpoint antagonist.

A method for detecting presence of ABCB5, comprising contacting an anti-ABCB5 antibody as disclosed herein with a biological sample suspected of containing ABCB5, and measuring binding of the anti-ABCB5 antibody to ABCB5 in the sample is provided in aspects of the invention. In some embodiments the biological sample is in vivo and the contacting step is performed by administering the subject an effective of the anti-ABCB5 antibody.

In some aspects a method for treating a tumor in a subject is provided. The method comprises obtaining immune leukocyte cells like T cells, NK cells monocytes and/or macrophages or combinations thereof from a subject having a tumor; transducing the T cells in vitro with a vector that contains a nucleic acid encoding a chimeric antigen receptor (CAR) including a scFv that specifically recognizes ABCB5, whereby the transduced T cells, NK cells, monocytes and/or macrophages immune cells express the CAR; expanding the transduced TCAR immune cells in vitro; and infusing the expanded transduced TCAR immune cells into the subject having a tumor, whereby an anti-tumor immuneT cell response is raised, wherein cells in the tumor express ABCB5. In some embodiments the antibody is conjugated to a detectable label. In some embodiments the biological sample is in vivo and the contacting step is performed by administering the subject an effective of the anti-ABCB5 antibody.

In some aspects a method for treating a tumor in a subject, comprising: obtaining T cells from a subject having a tumor; transducing the T cells in vitro with a vector that contains a nucleic acid encoding a chimeric antigen receptor (CAR) including a scFv that specifically recognizes ABCB5, whereby the transduced T cells express the CAR; expanding the transduced T cells in vitro; and infusing the expanded transduced T cells into the subject having a tumor, whereby an anti-tumor T cell response is raised, wherein cells in the tumor express ABCB5 is provided.

In other aspects an isolated chimeric antigen receptor (CAR) is provided. It includes an ABCB5 binding domain, a transmembrane domain and an intracellular signaling domain wherein the ABCB5 binding domain comprises a human variable heavy chain (V_{H}) domain. In some embodiments the ABCB5 binding domain comprises an antibody as described herein.

In aspects of the invention an isolated cell or cell population comprising one or more CAR as defined in any of claims 69 to 70. In some embodiments said cell is selected from the group consisting of a T cell, a Natural Killer (NK) cell, a cytotoxic T lymphocyte (CTL), and a regulatory T cell is provided.

In aspects of the invention a pharmaceutical composition comprising a cell or a pharmaceutical composition as disclosed herein and a pharmaceutical acceptable carrier, excipient or diluent is provided.

In aspects of the invention a method for treating cancer comprising administering a cell or a pharmaceutical composition as disclosed herein is provided.

In aspects of the invention a cell or a pharmaceutical composition as disclosed herein for use in therapy is provided.

In aspects of the invention a cell or a pharmaceutical composition as disclosed herein for use in the treatment of cancer is provided.

In aspects of the invention a use of a cell or a pharmaceutical composition as disclosed herein in the manufacture of a medicament for the treatment of cancer is provided. In some embodiments, the mammalian subject is a human. In some embodiments, the mammalian subject is a non-human primate. Non-limiting examples of non-human primate subjects include macaques, marmosets, tamarins, monkeys, baboons, gorillas, chimpanzees, and orangutans. Other exemplary subjects include domesticated animals such as dogs and cats; livestock such as horses, cattle, pigs, sheep, goats, and chickens; and other animals such as mice, rats, guinea pigs, and hamsters.

In some embodiments, the composition is administered subcutaneously, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs.

In some aspects compositions for use in the treatment of cancer are provided.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description or illustrated in the drawings. The disclosure is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows a basic assessment of antibody batch purity, stability, degradation etc. by SDS-PAGE under denaturing (96° C., 5 min) and reducing (100 mM DTT) conditions.

FIG. 2 shows the results of a linear peptide ELISA binding study (20 μM peptides). The data shows a comparative assessment of quantitative binding to ABCB5 linear epitope peptide (3rd extracellular loop) and corresponding peptide segments of homologous (ABCB1, 4, 11) and orthologous (murine ABCB5) proteins with increasing specificity/cross-reactivity.

FIG. 3 shows the results of an ABCB5 recombinant protein ELISA binding study. The data shows an evaluation of specific binding to recombinant ABCB5 protein.

FIGS. 4A-4B show results of cyclic (biotinylated) peptide ELISA binding studies (200 nM peptides)—(ABCB5/no Ab) (FIG. 4A) and (ABCB5/BSA) (FIG. 4B). The data shows an evaluation of specific binding and affinity estimation to more native cyclic ABCB5 peptide (cyclization by dilsufide bond between additional cysteines at both ends) with N-terminal biotin modification for immobilization (on streptavidin-precoated plates).

FIGS. 5A-5D show results of flow cytometry (FACS) binding studies on human tumor cell lines with ABCB5+ subpopulations. The data shows an evaluation of specific binding to cellular ABCB5 on human tumor cell lines, indicating detection with APC-labeled 2nd anti-human antibody. The human tumor cell lines include MCC13 Merkel cell carcinoma cells (FIG. 5A), G361 melanoma cells (FIG. 5B), A375 melanoma cells (FIG. 5C), and HT-29 colorectal cancer cells (FIG. 5D).

FIG. 6 shows immunofluorescence (IF) staining of human tumor cell lines with ABCB5+ subpopulation. The data shows an evaluation and visualization of specific binding to cellular ABCB5 in human tumor cell lines, indicating detection with AF594-labeled secondary anti-human antibody.

FIGS. 7A-7B show results of an antibody-dependent cellular cytotoxicity (ADCC) reporter bioassay and indicate the average potency/efficacy of Ab100 (FIG. 7A) and b12 isotype control Ab (FIG. 7B) on tumor cell lines.

FIGS. 8A-8B show results of an antibody-dependent cellular phagocytosis (ADCP) reporter bioassay and indicate the average potency/efficacy of Ab100 (FIG. 8A) and b12 isotype control Ab (FIG. 8B) on tumor cell lines.

FIGS. 9A-9B show results of (cell-based) pAkt/Akt ELISA assays, with specific inhibition of the pAkt1/Akt1 ratio by anti-ABCB5 antibodies as a result of inhibition of the PI3K signaling pathway. The human tumor cell lines with ABCB5+ subpopulation were G361 cells (FIG. 9A) and A375 cells (FIG. 9B).

FIG. 10 shows the results of anti-ABCB5 in vivo anti-tumor efficacy testing in mouse models. The tumor model was A375 (CRL-1619) human CDX melanoma (tumor prevention model). 10⁷ cells were delivered s.c. using antibodies.

FIG. 11 shows an assessment of antibody batch purity, stability, degradation etc. by SDS-PAGE under denaturing (96° C., 5 min) and reducing (100 mM DTT) conditions for anti-ABCB5 antibody Ab101.

FIG. 12 shows an assessment of antibody batch purity, stability, degradation etc. by SDS-PAGE under denaturing (96° C., 5 min) and reducing (100 mM DTT) conditions for anti-ABCB5 antibodies Ab42 (left) and Ab43 (right).

FIG. 13 shows an assessment of antibody batch purity, stability, degradation etc. by SDS-PAGE under denaturing (96° C., 5 min) and reducing (100 mM DTT) conditions for anti-ABCB5 antibodies Ab44 (left) and Ab45 (right).

FIG. 14 left panel shows the results of a linear peptide ELISA binding study (20 μM peptides) of ABCB5 antibody Ab101. The data shows a comparative assessment of quantitative binding to ABCB5 linear epitope peptide (3rd extracellular loop) and corresponding peptide segments of homologous (ABCB1, 4, 11) and orthologous (murine ABCB5) proteins—with increasing specificity/cross-reactivity. The right panel shows the results of an ABCB5 recombinant protein ELISA binding study of ABCB5 antibodies Ab101, Ab42, Ab43, Ab44 or Ab45, with data also shown for BSA negative control antigen. The data shows specific binding to recombinant ABCB5 protein.

FIG. 15 left panel shows the results of a linear peptide ELISA binding study (20 μM peptides) of ABCB5 antibody Ab42 (top left), Ab43 (bottom left), Ab44 (top right) or Ab45 (bottom right). The data shows a comparative assessment of quantitative binding to ABCB5 linear epitope peptide (3rd extracellular loop) and corresponding peptide segments of homologous (ABCB1, 4, 11) and orthologous (murine ABCB5) proteins, with scrambled peptide negative control with increasing specificity/cross-reactivity.

FIG. 16 shows results of cyclic (biotinylated) peptide ELISA binding studies (200 nM peptides) of ABCB5 antibodies Ab101, Ab42, Ab43, Ab44 or Ab45. The data shows an evaluation of specific binding and affinity estimation to more native cyclic ABCB5 peptide (cyclization by dilsufide bond between additional cysteines at both ends) with N-terminal biotin modification for immobilization (on streptavidin-precoated plates), with comparison to scrambled cyclic peptide negative controls.

FIG. 17 shows results of cyclic (biotinylated) peptide ELISA binding studies (200 nM peptides) of ABCB5 antibodies Ab101, Ab42, Ab43, Ab44 or Ab45 in an experiment using a broad range of monoclonal antibody concentrations. The data shows an evaluation of specific binding and affinity estimation to more native cyclic ABCB5 peptide (cyclization by dilsufide bond between additional cysteines at both ends) with N-terminal biotin modification for immobilization (on streptavidin-precoated plates), with comparison to scrambled cyclic peptide negative controls.

FIG. 18 shows results of flow cytometry (FACS) binding studies—human tumor cell lines with ABCB5+ subpopulation. The data shows an evaluation of specific binding of ABCB5 antibodies Ab101, Ab43, Ab45, Ab42 or Ab44 to cellular ABCB5 on human tumor cell lines, indicating detection with APC-labeled 2nd anti-human antibody. Isotype control staining is shown as well. The human tumor cell lines include in the panels from left to right MCC13 Merkel cell carcinoma cells, G361 melanoma cells, A375 melanoma cells, and HT-29 colorectal cancer cells.

FIG. 19 shows immunofluorescence (IF) staining of human tumor cell lines with ABCB5+ subpopulation (top panels: MCC13 Merkel cell carcinoma cells, bottom panels: A375 melanoma cells), using ABCB5 antibodies Ab101, Ab42, Ab43, Ab44 or Ab45, or isotype control. The data shows an evaluation and visualization of specific binding to cellular ABCB5 in these human tumor cell lines, indicating detection with AF594-labeled secondary anti-human antibody.

FIG. 20 shows immunofluorescence (IF) staining of human tumor cell lines with ABCB5+ subpopulation (top panels: HT-29 colorectal cancer cells, bottom panels: A549 lung cancer cells), using ABCB5 antibodies Ab101, Ab42, Ab43, Ab44 or Ab45, or isotype control. The data shows an evaluation and visualization of specific binding to cellular ABCB5 in these human tumor cell lines, indicating detection with AF594-labeled secondary anti-human antibody.

FIG. 21 shows results of (cell-based) pAkt/Akt ELISA assays, with specific inhibition of the pAkt1/Akt1 ratio by anti-ABCB5 antibodies Ab101, Ab42, Ab43, Ab44 or Ab45 as a result of inhibition of the PI3K signaling pathway. Isotype and no antibody negative controls are shown as well. The human tumor cell lines with ABCB5+ subpopulation were A375 melanoma cells (left panel) and HT-29 colorectal cancer cells (right panel).

FIG. 22 shows results of flow cytometrically determined antibody internalization (cellular uptake) assessment of ABCB5 antibody Ab101 conjugated to pHrodo green pH-sensitive dye in G361 human melanoma cells (left panel) or HT-29 human colorectal cancer cells (right panel). The % gated cells are shown in top panels and MFI is shown in bottom panels as a function of time. The experiment was performed at 37 degree C. demonstrating antibody uptake, and, as a negative control, at 4 degree C.

FIG. 23 shows results of Immunofluorescence (IF) visualization-determined antibody internalization (cellular uptake) assessment of ABCB5 antibodies Ab101 or Ab42 conjugated to pHrodo red pH-sensitive dye in MCC13 human Mekel carcinoma cells (left panels) or A549 human lung cancer cells (right panels). The experiment demonstrated specific antibody uptake of the tested ABCB5 antibodies. An isotype control antibody was used as a negative control.

FIG. 24 shows in the left 4 panels the effects of anti-ABCB5 antibody Ab101/ADC MMAE drug conjugate vs. unconjugated Ab101 on apoptosis induction (Caspase 3/7Glo) in G361 melanoma cells (top left panel) or MKL-1 Merkel cell carcinoma cells (top right panel), and on cytotoxicity induction (CellTiterGlo assay) in G361 melanoma cells (bottom left panel) or MKL-1 Merkel cell carcinoma cells (bottom right panel). Right 4 panels: Effects of anti-ABCB5 antibody Ab101/ADC MMAE drug conjugate vs. isotype control/ADC MMAE drug conjugate vs. free MMAE on apoptosis induction (Caspase 3/7Glo) in G361 melanoma cells (top left panel) or MCC13 Merkel cell carcinoma cells (top right panel), and on cytotoxicity induction (CellTiterGlo assay) in G361 melanoma cells (bottom left panel) or MCC13 Merkel cell carcinoma cells (bottom right panel).

### DETAILED DESCRIPTION

Described herein are compositions (including pharmaceutical compositions) and methods for the design, preparation, manufacture and/or formulation of anti-ABCB5 antibodies. Also provided are systems, processes, devices and kits for the selection, design and/or utilization of the antibodies described herein. ATP-binding cassette (ABC) transporters play a pivotal role in physiology and pathology. They are involved in the transport of structurally diverse molecules ranging from small ions, sugars, and peptides to more complex organic molecules. ATP-binding cassette, sub-family B, member 5 (ABCB5) is a multidrug resistance (MDR) mediator expressed in diverse human malignancies. ABCB5 confers cancer cell drug resistance to chemotherapeutic agents such as 5-fluorouracil (5-FU). “ABCB5⁺ stem cells” or “ABCB5⁺ cells,” as used herein, refers to cells having the capacity to self-renew and to differentiate into mature cells of multiple adult cell lineages.

ABCB5⁺ stem cells are also found in normal tissue and have a role in tissue regeneration and aging. Regenerative medicine involves the repair, regeneration, maintenance, and replacement of tissues and organs using exogenous materials such as scaffolds. The scaffolds may be seeded with cells, such as primary cells or stem cells, and various factors to encourage tissue growth.

Antibodies for conducting research on ABCB5 and other uses, such as isolated stem cells and putative therapeutics have been described in the literature. Provided herein, therefore, are antibodies or portions thereof or nucleic acids encoding antibody compositions which have been designed to produce a therapeutic outcome and optionally improve one or more of the stability and/or clearance in tissues, accessibility to circulation, protein half-life and/or modulation of a cell's status, antibody target affinity and/or specificity, reduction of antibody cross reactivity, increase of antibody purity, increase or alteration of antibody effector function and/or antibody activity.

The present disclosure is based, at least in part, on the development of anti-ABCB5 antibodies, which possessed unexpected superior features compared with known anti-ABCB5 antibodies. For instance, the anti-ABCB5 antibodies disclosed herein may possess superior/unexpected features, for example, (a) binding to intact cell surface ABCB5 but not denatured ABCB5, (b) no or insignificant cross-reactivity to other related cell surface proteins such as ABCB1 and ABCB4; (c) capable of inducing significant cancer cell death relative to commercially available ABCB5 antibodies, (d) capable of selectively binding an intracellular epitope; and (e) capable of selectively binding an extracellular epitope.

Antibodies, also known as immunoglobulins, are glycoproteins produced by B cells. Using a unique and highly evolved system of recognition, antibodies can recognize a target and tag a target epitope, foreign entity, cancer cell or invading microbe for attack by the immune system thereby neutralizing its effect. The production of antibodies is the main function of the humoral immune system. Antibodies are secreted by a plasma cell which is a type of white blood cell.

Antibodies occur in two physical forms, a soluble form that is secreted from the cell, and a membrane-bound form that is attached to the surface of a B cell and is referred to as the B cell receptor (BCR). Soluble antibodies are released into the blood and tissue fluids, as well as many secretions to continue to survey for invading microorganisms and other non-self antigens.

Frequently the binding of an antibody to an antigen has no direct biological effect. Rather, the significant biological effects are a consequence of secondary “effector functions” of antibodies. The immunoglobulins mediate a variety of these effector functions. These functions include fixation of complement, binding of phagocytic cells, lymphocytes, platelets, mast cells, and basophils which have immunoglobulin receptors. This binding can activate the cells to perform some function.

Accordingly, provided herein are antibodies capable of binding ABCB5, as well as nucleic acids encoding said antibodies, and uses thereof for therapeutic, research, and diagnostic purposes. Also provided herein are kits for therapeutic and/or diagnostic use of the antibodies, as well as methods for producing anti-ABCB5 antibodies. In addition, the present disclosure provides chimeric antigen receptors comprising extracellular antigen binding domains derived from any of the anti-ABCB5 antibodies described herein.

Several ABCB5 isoforms are involved in cancer and disease. As used herein, an “ABCB5 isoform” is an ABCB5 protein having one variant of ABCB5 structure. In some embodiments, the ABCB5 isoform is ABCB5 isoform 1 (1257 amino acids). ABCB5 isoform 1 comprises two transmembrane domains (TMDs) with 6 transmembrane (TM) helices each, i.e. it comprises altogether 12 transmembrane helices (TMs 1-12). In some embodiments, the ABCB5 isoform is isoform 2 (812 amino acids). ABCB5 isoform 2 comprises one TMD with 6 transmembrane (TM) helices (TMs 1-6). TMs 1-6 of ABCB5 isoform 2 correspond to TMs 7-12 of ABCB5 isoform 1.

Thus, the present disclosure provides antibodies that bind to ABCB5. In some embodiments, the anti-ABCB5 antibody binds cell surface-displayed ABCB5 on an extracellular loop. In other embodiments, the anti-ABCB5 antibody binds an intracellular loop of ABCB5. Alternatively or in addition, the anti-ABCB5 antibody may have low binding affinity to denatured ABCB5 or does not bind to the denatured ABCB5.

An antibody (interchangeably used in plural form) is an immunoglobulin molecule capable of specific binding to a target antigen (e.g., ABCB5 in the present disclosure), through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. Thus, an antibody variable region consists of a “framework” region interrupted by three “antigen binding sites”. The antigen binding sites are defined using various terms: (i) Complementarity Determining Regions (CDRs), three in the VH (HCDR1, HCDR2, HCDR3), and three in the VL (LCDR1, LCDR2, LCDR3), are based on sequence variability; (ii) “Hypervariable regions,” “HVR,” or “HV,” three in the VH (H1, H2, H3) and three in the VL (L1, L2, L3), refer to the regions of an antibody variable domains which are hypervariable in structure as defined by Chothia and Lesk. “Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

In some embodiments, the anti-ABCB5 antibody as described herein can bind and inhibit the activity of the ABCB5 receptor by at least 50% (e.g., 60%, 70%, 80%, 90%, 95% or greater). The apparent inhibition constant (Ki^{app} or K_{i,app}), which provides a measure of inhibitor potency, is related to the concentration of inhibitor required to reduce signaling/transport activity and is not dependent on protein concentrations. The inhibitory activity of an anti-ABCB5 antibody described herein can be determined by routine methods known in the art.

The antibodies described herein can be murine, rat, human, primate, porcine, or any other origin (including chimeric or humanized antibodies). Such antibodies are non-naturally occurring, i.e., would not be produced in an animal without human act (e.g., immunizing such an animal with a desired antigen or fragment thereof).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

Antibody heavy and light chain constant regions are well known in the art, e.g., those provided in the IMGT database (imgt.org) or at vbase2.org/vbstat.php., both of which are incorporated by reference herein.

In some embodiments the antibody is an antigen binding fragment. As used herein, the term “antigen binding fragment” refers to an antibody fragment such as, for example, a diabody, a Fab, a Fab′, a F(ab′)2, an Fv fragment, a disulfide stabilized Fv fragment (dsFv), a (dsFv) 2, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment (i.e., portion of the heavy chain which is included in the Fab fragment). According to other particular embodiments, the antigen-binding fragment comprises Fab and F(ab′)

In yet another example, the antibody described herein can be a single-domain antibody, which interacts with the target antigen via only one single variable domain such as a single heavy chain domain (as opposed to traditional antibodies, which interact with the target antigen via heavy chain and light chain variable domains). A single-domain antibody can be a heavy-chain antibody (VHH) which contains only an antibody heavy chain and is devoid of light chain. In additional to a variable region (for example, a VH), a single-domain antibody may further comprise a constant region, for example, CH₁, CH₂, CH₃, CH₄, or a combination thereof.

In some embodiments, the antibodies and antigen binding fragments thereof comprise a fragment crystallizable (Fc) region. The Fc region is the tail region of an antibodies and antigen binding fragments thereof which contains constant domains (e.g., CH₂ and CH₃); the other region of the antibodies and antigen binding fragments thereof being the Fab region which contains a variable domain (e.g., VH) and a constant domain (e.g., CHO, the former of which defines binding specificity.

As described herein, antibodies can comprise a VH domain. In some embodiments, the VH domain further comprises one or more constant domains (e.g., CH₂ and/or CH₃) of an Fc region and/or one or more constant domains (e.g., CH₁) of a Fab region. In some embodiments, each of the one or more constant domains (e.g., CH₁, CH₂, and/or CH₃) can comprise or consist of portions of a constant domain. For example, in some embodiments, the constant domain comprises 99% or less, 98% or less, 97% or less, 96% or less, 95% or less, 90% or less, 80% or less, 70% or less, 60% or less, 50% or less, 40% or less, 30% or less, 20% or less, or 10% or less of the corresponding full sequence.

Alternatively, an antibody described herein may comprise up to 5 (e.g., 4, 3, 2, or 1) amino acid residue variations in one or more of the CDR regions of one of the antibodies known in the art and/or exemplified herein and binds the same epitope of antigen with substantially similar affinity (e.g., having a KD value in the same order). In one example, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-ABCB5 antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen (ABCB5) or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (e.g., binding not detectable in a conventional assay).

In some embodiments, the antibodies described herein specifically bind to ABCB5 as relative to other related cell surface receptors, for example, ABCB1, 4, and 11. In some embodiments, the antibodies described herein do not bind to one or more of the related cell surface proteins such as those described herein. In some embodiments, the antibodies described herein do not bind to one more of the related proteins expressed on the cell surface of stem cells.

In some embodiments, the antibodies described herein specifically binds to ABCB5 of a specific species (e.g., human ABCB5) as relative to ABCB5 from other species. For example, the antibodies described herein may specifically binds to human ABCB5 as relative to mouse ABCB5. In other embodiments, the antibodies described herein may cross-react with human ABCB5 and one or more ABCB5 from a non-human species (e.g., a non-human primate such as macaque or pig). In some embodiments, the antibodies cross-react with human, macaque, and pig ABCB5 with similar binding affinity but have significantly lower binding affinity to mouse ABCB5.

In some embodiments, an anti-ABCB5 antibody as described herein has a suitable binding affinity for the target antigen (e.g., human ABCB5) or antigenic epitopes thereof. As used herein, “binding affinity” refers to the apparent association constant or KA, which is the ratio of association and dissociation constants, K-on and K-off, respectively. The KA is the reciprocal of the dissociation constant (K_{D}). The anti-ABCB5 antibody described herein may have a binding affinity (K_{D}) of at least 10⁻⁸, 10⁻⁹, 10⁻¹⁰ M, or lower for the target antigen or antigenic epitope. An increased binding affinity corresponds to a decreased value of K_{D}. Higher affinity binding of an antibody for a first antigen relative to a second antigen can be indicated by a higher KA (or a smaller numerical value K_{D}) for binding the first antigen than the KA (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). In some embodiments, the anti-ABCB5 antibodies described herein have a higher binding affinity (a higher KA or smaller K_{D}) to ABCB5 as compared to the binding affinity to another membrane protein (e.g., ABCB1, ABCB4 and ABCB11). Differences in binding affinity (e.g., for specificity or other comparisons) can be at least 1.5, 2, 2.5, 3, 4, 5, 10, 15, 20, 37.5, 50, 70, 80, 91, 100, 500, 1,000, 5,000, 10,000 or 10⁵ fold. In some embodiments, any of the anti-ABCB5 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance (SPR), fluorescent activated cell sorting (FACS) or spectroscopy (e.g., using a fluorescence assay). Exemplary conditions for evaluating binding affinity are: HBS-P buffer (10 mM HEPES pH7.4, 150 mM NaCl, 0.005% (v/v) surfactant P20) and PBS buffer (10 mM PO₄⁻³, 137 mM NaCl, and 2.7 mM KCl). These techniques can be used to measure the concentration of bound proteins as a function of target protein concentration. The concentration of bound protein ([Bound]) is generally related to the concentration of free target protein ([Free]) by the following equation: [Bound]=[Free]/(Kd+[Free]).

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-ABCB5 antibodies disclosed herein exhibit one or more bioactivities, including blocking the intracellular signaling of ABCB5 or molecular transport activity or competing against commercially available ABCB5⁺ antibodies. Thus, in addition to the antibody sequences disclosed herein the antibodies of the invention may share sequence identity with those antibody sequences and similar function but have some amino acid differences.

In some embodiments, the antibodies and antigen binding fragments thereof comprises a heavy chain variable region having an amino acid sequence sharing at least 80%, 81%, 82%, 83%, 84%, 85%, 86%, 87%, 88%, 89%, 90%, 91%, 92%, 93%, 94%, 95%, 96%, 97% 98%, or 99% identity with any of the amino acid sequences provided herein. In some embodiments, the amino acid sequence of the antibody comprises an amino acid sequence provided herein.

Several lead candidate antibodies for therapeutic, diagnostic, and research use were identified, characterized and modified in order to develop a robust set of ABCB5 binding agents. Two of the full length antibodies include Ab-100 and Ab-101.

The antibodies described herein are unique in that they possesses several mode(s) of action/related properties, including: antibody-dependent cellular cytotoxicity (ADCC), antibody-dependent (cellular) phagocytosis (ADCP), likely complement-dependent cytotoxicity (CDC), and inhibition of signal transduction processes. The antibodies can be used alone as a therapeutic or diagnostic agent or together with other therapeutic agents. For instance a synergistic co-treatment, with ‘classic’ cytostatics (e.g. Etoposide, Paclitaxel, Doxorubicin, etc.) provides enhanced therapeutic benefit. The antibodies may also demonstrate chemoresistance reversal via blocking of ABCB5-mediated drug efflux with other therapeutic antibodies (e.g. anti-EGFR Cetuximab, anti-PD-1 Nivolumab, anti-VEGF Bevacizumab or similar) through mechanistically non-related synergistic effects or with small-molecule inhibitors (e.g. BRAF Inhibitors Vemurafenib, Dabrafenib, Trametinib, MEK inhibitors) through mechanistically related synergistic effects.

Thus, Ab-100 has useful properties associated with ADCC, ADCP, CDC, and inhibition of signal transduction processes. Ab-100 has the following amino acid sequence:

Heavy chain (hc) peptide sequence:
EVQLVESGGGLVQPGGSLRLSCAASGFTFSSYSMNWVRQAPGKGLEWVSYISSSSSTIYYADSVKGRFTISRDNA
KNSLYLQMNSLRDEDTAVYYCASNYQYGDYGGYWGRGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALHNHYTQKSLSLSPGK*
Light chain (lc) peptide sequence:
(SEQ ID NO: 116)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC*

The variable domain of heavy chain (V_{H}) region and light chain (V_{L}), region are underlined.

Ab-101 also possesses several mode(s) of action/related properties, including: suitability for modification by means of (chemical) drug conjugation and capability of cellular uptake (internalization) of conjugated antibody upon antigen (ABCB5) binding on cell surface for targeted drug delivery. The antibody can be used alone, combined with or linked to therapeutic agents. Ab-101 has the following amino acid sequence:

Heavy chain (hc) peptide sequence:
(SEQ ID NO: 117)
QVQLVQSGAEVKKPGASVKVSCKASGYTFTSYYMHWVRQAPGQGLEWMGIINPSGGSTSYAQKFQGRVTMTRDTS
TSTVYMELSSLRSEDTAVYYCARDQAVTGTAYYYYYGMDVWGQGTTVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSL
SLSPGK*
Light chain (lc) peptide sequence:
(SEQ ID NO: 47)
EIVLTQSPGTLSLSPGERATLSCRASQSVNSNYLAWYQQKPGQAPRLLIYGTSSRATGIPDSFSGSGSGTDFTLT
ISRLEPEDFAVYYCQQFGSSPLTFGGGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC*

The variable domain of heavy chain (V_{H}) region and light chain (V_{L}), region are underlined.

Each of Ab100 and Ab101 are fully human monoclonal IgG1 kappa (allotypes G1m17, nG1m1 heavy chain and Km3 light chain) and both bind with high specificity and affinity to 3rd extracellular loop (3rd EC loop) epitope (RFGAYLIQAGRMTPEG, SEQ ID NO. 104) of human ABCB5 transcript variant 2 or beta isoform [NCBI accession NM_178559.5] and all other ABCB5+ variants that include this epitope.

Thus, some of the antibodies of the invention may bind to an epitope of human ABCB5 comprising SEQ ID NO. 104, fragments thereof or polypeptides having at least 75%, 80%, or 90% sequence identity to SEQ ID NO. 104. In some embodiments the antibodies of the invention may bind to an epitope of the extracellular loop 3 of human ABCB5 and fragments thereof and optionally various other adjacent and/or exposed transmembrane residues.

The term “epitope” refers to the portion(s) of an antigen (e.g. human ABCB5) that contact an antibody. Epitopes can be linear, i.e., involving binding to a single sequence of amino acids, or conformational, i.e., involving binding to two or more sequences of amino acids in various regions of the antigen that may not necessarily be contiguous. The antibodies provided herein may bind to different (overlapping or non-overlapping) epitopes within the extracellular domain of the human ABCB5 protein. Thus, epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays. Methods of determining spatial conformation of epitopes include techniques in the art, for example, x-ray crystallography, x-ray co-crystallography, antigen mutational analysis, 2-dimensional nuclear magnetic resonance and HDX-MS. The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on ABCB5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments, e.g., BIACORE®. surface plasmon resonance (SPR) analysis. In some embodiments, an antibody competes with, and inhibits binding of another antibody to a target by at least 50%, 60%, 70%, 80%, 90% or 100%. The level of inhibition or competition can be different depending on which antibody is the “blocking antibody” (i.e., the cold antibody that is incubated first with the target). Two antibodies “cross-compete” if antibodies block each other both ways by at least 50%, i.e., regardless of whether one or the other antibody is contacted first with the antigen in the competition experiment. Competitive binding assays for determining whether two antibodies compete or cross-compete for binding include: competition for binding to cells expressing ABCB5, e.g., by flow cytometry. Other methods include: SPR (e.g., BIACORE®), solid phase direct or indirect radioimmunoassay (RIA), solid phase direct or indirect enzyme immunoassay (EIA), sandwich competition assay; solid phase direct biotin-avidin EIA; solid phase direct labeled assay, or solid phase direct labeled sandwich assay.

As used herein, the term “high affinity” for an IgG antibody refers to an antibody having a K_{D} of 10⁻⁸M or less, 10⁻⁹M or less, or 10⁻¹⁰ M or less for a target antigen. However, “high affinity” binding can vary for other antibody isotypes. For example, “high affinity” binding for an IgM isotype refers to an antibody having a K_{D} of 10⁻¹⁰ M or less, or 10⁻⁸M or less.

In addition to Ab100 and Ab101 and antibodies that bind to the same epitope thereof, several anti-ABCB5 antibodies having enhanced properties have been designed.

For instance several antibodies having Framework region (FR) optimized regions have been designed. The sequences and changes relative to Ab100 are highlighted (italicized and underlined) in each of the sequences below and shown throughout the application. CDRs are underlined and bold. The variable heavy chain is included in single brackets [ ] and the variable light chain is in triple brackets [[[ ]]].

Ab1: Heavy chain (hc) variable domain (V_{H}) peptide sequence:
(SEQ ID NO: 1)
EVQLLESGGGLVQPGGSLRLSCAASGFTFSSYSMNWVRQAPGKGLEWVSYISSSSSTIYYADSVKGRFTISRDNS
KNTLYLQMNSLRAEDTAVYYCAKNYQYGDYGGYWGRGTLVTVSS
Light chain (lc) variable domain (V_{L}) peptide sequence:
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK

Other enhanced antibody species, derived from Ab100/Ab101, have increased binding affinity properties by corresponding selection from affinity maturation approaches. Ab100-derived species' sequences with enhanced affinity are given below. CDRs are underlined and bold:

Ab2: Heavy chain (hc) variable domain (V_{H}) peptide sequence:
(SEQ ID NO: 2)
EVQLVESGGGLVQPGGSLRLSCAASGFTFSSY [symbol] MNWVRQAPGKGLEWVSYISSSS [symbol] TIYYADSVKGRFTISRDNA
KNSLYLQMNSLRDEDTAVYYCASNYQYGDYGGYWGRGTLVTVSS
Light chain (lc) variable domain (V_{L}) peptide sequence:
(SEQ ID NO: 21)
AIQLTQSPSSLTGSVGDRVTITC [symbol] ASHDISNFLNWYQQKPGQAPKLLIYDAYNLQTGVPSRFSGTGSGTHFTLTI
NSLQPEDVGTYFCQQYDYFLSITFGQGTRLEIK
Ab3: Heavy chain (hc) variable domain (V_{H}) peptide sequence:
(SEQ ID NO: 3)
EVQLVESGGGLVQPGGSLRLSCAASGFTFSSYSMNWVRQAPGKGPEWVSYISSS [symbol] STIYYADSVKGRFTISRDNA
KNSLYLQMNSLRDEDTAVYYCASNYQYGDYGGYWGRGTLVTVSS
Light chain (lc) variable domain (V_{L}) peptide sequence:
(SEQ ID NO: 22)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIK
Ab4: Heavy chain (hc) variable domain (V_{H}) peptide sequence:
(SEQ ID NO: 4)
EVQLVESGGGLVQPGGSLRLSCAASGFTFSSYSMNWVRQAPGKGPEWVSYISSSSSTIYYADSVKGRFTISRDNA
KNSLYLQMNSLRDEDTAVYYCASNYQYGDYGGYWGRGTLVTVSS
Light chain (lc) variable domain (V_{L}) peptide sequence:
(SEQ ID NO: 22)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIK

Fc-modified versions of Ab100 having full sequence heavy chain include:

Ab5: Heavy chain (hc) full peptide sequence having an increased in vitro
ADCC/ADCP efficacy and in vivo ½ life (light chain same as Ab100) ([heavy
chain variable chain] in brackets):
(SEQ ID NO: 31)

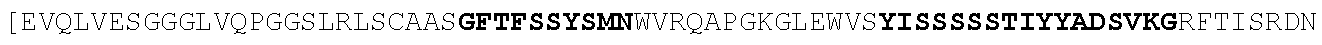

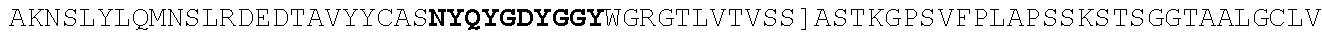

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALKFHYTQKSLSLSPG
K

Combined versions of Ab100 include:

Ab6: Heavy chain (hc) variable domain (VH) peptide sequence AB1xB04:
(SEQ ID NO: 6)
EVQLLESGGGLVQPGGSLRLSCAASGFTFSSY [symbol] MNWVRQAPGKGLEWVSYISSSS [symbol] TIYYADSVKGRFTISRDNS
KNTLYLQMNSLRAEDTAVYYCAKNYQYGDYGGYWGRGTLVTVSS
Light chain (lc) variable domain (V_{L}) peptide sequence AB1xB04:
(SEQ ID NO: 23)
DIQMTQSPSSLSASVGDRVTITC [symbol] ASHDISNFLNWYQQKPGKAPKLLIYDAYNLQTGVPSRFSGSGSGTDFTFTI
SSLQPEDIATYYCQQYDYFLSITFGQGTKVEIK
Ab7: Heavy chain (hc) variable domain (V_{H}) peptide sequence AB1xG02:
(SEQ ID NO: 7)
EVQLLESGGGLVQPGGSLRLSCAASGFTFSSYSMNWVRQAPGKGPEWVSYISSS [symbol] TIYYADSVKGRFTISRDNS
KNTLYLQMNSLRAEDTAVYYCAKNYQYGDYGGYWGRGTLVTVSS
Light chain (lc) variable domain (V_{L}) peptide sequence AB1xG02:
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK
Ab8: Heavy chain (hc) variable domain (V_{H}) peptide sequence AB1xH01:
(SEQ ID NO: 8)
EVQLLESGGGLVQPGGSLRLSCAASGFTFSSYSMNWVRQAPGKGPEWVSYISSSSSTIYYADSVKGRFTISRDNS
KNTLYLQMNSLRAEDTAVYYCAKNYQYGDYGGYWGRGTLVTVSS
Light chain (lc) variable domain (V_{L}) peptide sequence AB1xH01:
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK
Ab9: Heavy chain (hc) full peptide sequence AB1 HCII.d:
(SEQ ID NO: 32)

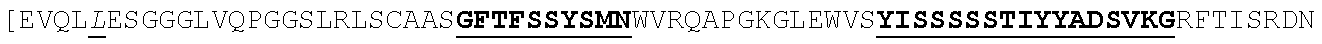

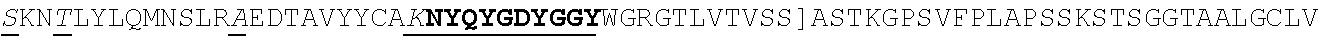

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALKFHYTQKSLSLSPG
K*
Light chain (lc) variable domain (V_{L}) peptide sequence AB1 HCII.d:
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK
Ab10: Heavy chain (hc) full peptide sequence B04 HCII.d:
(SEQ ID NO: 33)

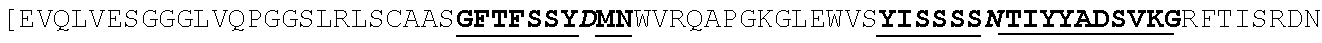

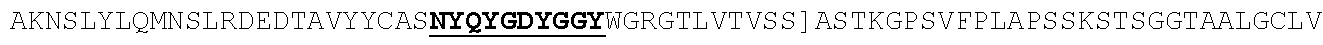

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALKFHYTQKSLSLSPG
K*
Light chain (lc) variable domain (V_{L}) peptide sequence B04 HCII.d:
(SEQ ID NO: 21)
AIQLTQSPSSLTGSVGDRVTITC [symbol] ASHDISNFLNWYQQKPGQAPKLLIYDAYNLQTGVPSRFSGTGSGTHFTLTI
NSLQPEDVGTYFCQQYDYFLSITFGQGTRLEIK
Ab11: Heavy chain (hc) full peptide sequence G02 HCII.d:
(SEQ ID NO: 34)

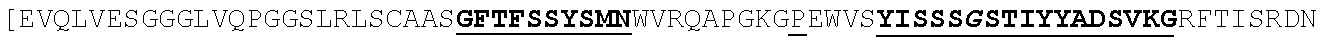

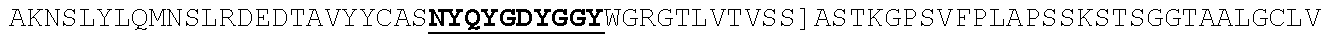

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALKFHYTQKSLSLSPG
K*
Light chain (lc) variable domain (V_{L}) peptide sequence G02 HCII.d:
(SEQ ID NO: 22)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIK
Ab12:
(SEQ ID NO: 35)

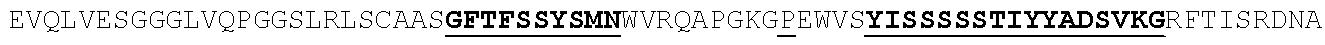

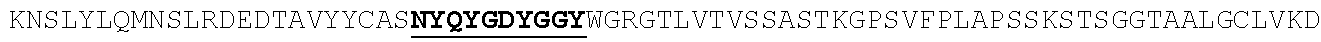

YFPEPVTVSWNSGALTSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALKFHYTQKSLSLSPGK*
Light chain (lc) variable domain (V_{L}) peptide sequence H01 HCII.d:
(SEQ ID NO: 22)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIK
Ab:13 Heavy chain (hc) full peptide sequence AB1xB04 HCII.d:
(SEQ ID NO: 36)

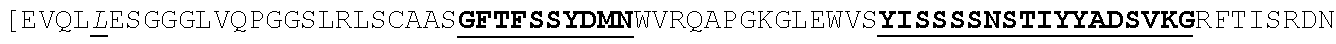

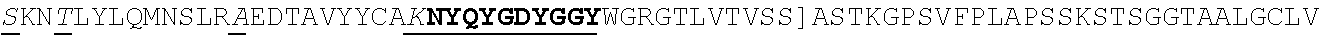

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALKFHYTQKSLSLSPG
K*
Light chain (lc) variable domain (V_{L}) peptide sequence AB1xB04 HCII.d:
(SEQ ID NO: 23)
DIQMTQSPSSLSASVGDRVTITC [symbol] ASHDISNFLNWYQQKPGKAPKLLIYDAYNLQTGVPSRFSGSGSGTDFTFTI
SSLQPEDIATYYCQQYDYFLSITFGQGTKVEIK
Ab14: Heavy chain (hc) full peptide sequence AB1xG02 HCII.d:
(SEQ ID NO: 37)

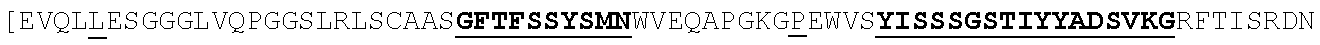

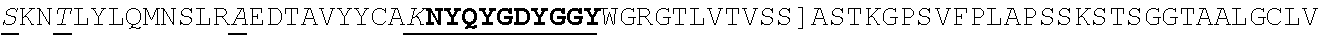

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALKFHYTQKSLSLSPG
K*
Light chain (lc) variable domain (V_{L}) peptide sequence AB1xG02 HCII.d:
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK
Ab15: Heavy chain (hc) full peptide sequence AB1xH01 HCII.d:
(SEQ ID NO: 118)

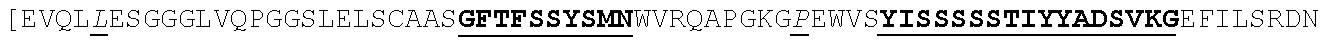

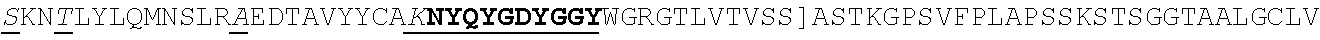

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALKFHYTQKSLSLSPG
K*
Light chain (lc) variable domain (VL) peptide sequence AB1xH01 HCII.d:
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK

Stably aglycosylated versions of Ab100 with multiple Fc modifications include:

Ab 16: Heavy chain (hc) full peptide sequence aglycosylated:
(SEQ ID NO: 38)

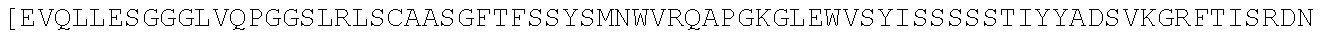

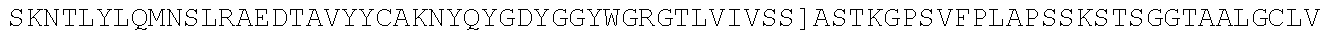

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVEDVSHEDPEVKFNWYVDGVEVHNAKTKPREEQY
NGAYRVVSVLTVLHQDWLNGKEYKCKVSNIYGPAPIEKTISKAKGQPREPQVYALPPSREEMTKNQVSLTCLVKG
FYPSDIAVEWVSNGQPENDYKTTPPVLDSDGSFFLYSKLTVDKSRWQQGNVFSCSVLHEALHNHYTQKSLSLSPG
K*
Light chain (lc) variable domain (V_{L}) peptide sequence AB1 aglycosylated:
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK
Ab17: Heavy chain (hc) full peptide sequence aglycosylated:
(SEQ ID NO: 39)

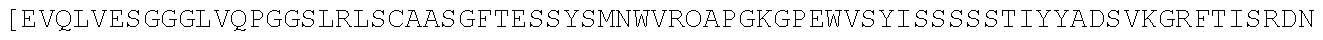

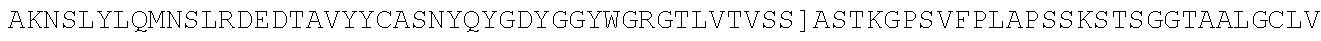

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVEDVSHEDPEVKFNWYVDGVEVHNAKTKPREEQY
NGAYRVVSVLTVLHQDWLNGKEYKCKVSNIYGPAPIEKTISKAKGQPREPQVYALPPSREEMTKNQVSLTCLVKG
FYPSDIAVEWVSNGQPENDYKTTPPVLDSDGSFFLYSKLTVDKSRWQQGNVFSCSVLHEALHNHYTQKSLSLSPG
K*
Light chain (lc) variable domain (V_{L}) peptide sequence H01 aglycosylated:
(SEQ ID NO: 22)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIK

The sequences and changes relative to Ab101 are highlighted in each of the sequences below. CDRs are underlined and bold:

Affinity-maturated versions of Ab101 include:

Ab42: Heavy chain (hc) variable domain (V_{H}) peptide sequence:
(SEQ ID NO: 13)
QVQLVQSGAEVKKPGASVKVSCKTSGYTFTSYYMHWVRQSPGQGLEWMGIINPSGGSTSYAQKFQGRVTMTRDTS
TSTVYMELSSLRSEDTAVYYCARDQAVTGTAYYYYYGMDVWGQGTTVTVSS
Light chain (lc) variable domain (V_{L}) peptide sequence:
(SEQ ID NO: 26)
EIVLTQSPGTLSLSPGERATLSCRAS [symbol] SVNSNYLAWYQQKPGQAPRLLIYGTSSRATGIPDSFSGSGSGTDFTLT
ISRLEPEDFAVYYCQQFGSSPLTFGGGTKVEIK
Ab43: Heavy chain (hc) variable domain (V_{H}) peptide sequence:
(SEQ ID NO: 14)
QVQLVQSGAEVKKPGASVKVSCKASGYTFTSYYMHWVRQSPGQGLEWMGIINPSGGSTSYAQKFQGRVTMTRDTS
TSTVYMELSSLRSEDTAVYYCARDQAVTGTAYYYYYGMDVWGQGTTVTVSS
Light chain (lc) variable domain (V_{L}) peptide sequence:
(SEQ ID NO: 27)
EIVLTQSPGTLSLSPGERATLSCRASQSVNS [symbol] YLAWYQQKPGQAPRLLIYGTSSRATGIPDSFSGSGSGTDFTLT
ISRLEPEDFAVYYCQQFGSSPLTFGGGTKVEIK
Ab44: Heavy chain (hc) variable domain (V_{H}) peptide sequence:
(SEQ ID NO: 15)
QVQLVQSGAEVKKPGASVKVSCKASGYTFTSYYMHWVRQAPGQGLEWMGIINPSGGSTSYAQKF [symbol] GRVTMTRDTS
TSTVYMELSSLRSEDTAVYYCARDQAVTGTAYYYYYGMDVWGQGTTVTVSS
Light chain (lc) variable domain (V_{L}) peptide sequence:
(SEQ ID NO: 28)
EIVLTQSPGTLSLSPGERATLSCRASQSVNSNYLAWYQQKPGQAPRLLIYGTSSRATGIPDSFSGSGSGTDFTLT
ISRLEPEDFAVYYCQQFGSSPLTFGGGTKVEIK
Ab45:Heavy chain (hc) variable domain (V_{H}) peptide sequence:
(SEQ ID NO: 16)
QVQLVQSGAEVKKPGASVKVSCKASGYTFT [symbol] YYMHWVRQAPGQGPEWMGIINPSGGSTSYAQKFQGRVTMTRDTS
TSTVYMELSSLRSEDTAVYYCARD [symbol] AVTGTAYYYYYGMDVWGQGTTVTVSS
Light chain (lc) variable domain (V_{L}) peptide sequence:
(SEQ ID NO: 29)
EFVLTQSPGTLSLSPGERATLSCRASQSVNSNYLAWYQQKPGQAPRLLIYGTSSRATGIPDSFSGSGSGTDFTLT
ISRLEPEDFAVYYCQQFGSSPLTFGGGTKVEIK

Additionally, several anti-ABCB5 antibodies having modified formats have been designed. These include bispecific antibody formats (fully human IgG1 kappa anti-ABCB5 antibodies or scFv in combination with anti-CD16 scFv and anti-CD3 scFv). Amongst many different options to generate recombinant bi-specific antibody derivatives (i.e. derived antibody format binds specifically to two different antigens), three distinct formats were designed, where the first specificity/binding site(s) recognizes ABCB5 (tumor) antigen and the specificity/binding site(s) is for a certain immune effector cell antigen (i.e. CD3 on cytotoxic T cell lymphocytes [CTLs] or against CD16 on natural killer [NK] cells. These constructs may be referred to as bi-specific [CTL/NK] cell engagers.

IgG-scFv Fusion Proteins [IgG-scFv's]

In these examples an existing full length IgG(1) antibody molecule with given specificity is taken and enhanced by recombinantly adding a sufficiently effective binding site (e.g. a full scFv fragment, a diabody, a Fab fragment or similar) for another antigen (->2^{nd} newly introduced specificity). The site of addition could either be the C-terminal end of light chain, of heavy chain or any other potentially suitable site of antibody polypeptide.

Ab18: Heavy chain (hc) full peptide sequence AB1 with C-terminal fused
anti-CD3 scFv:
(SEQ ID NO: 119)

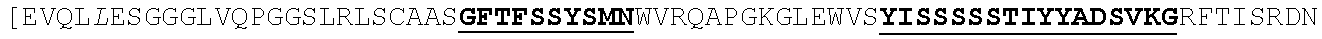

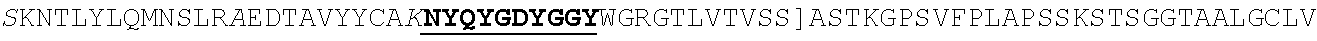

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSPG

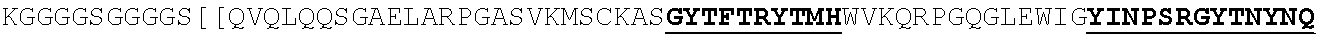

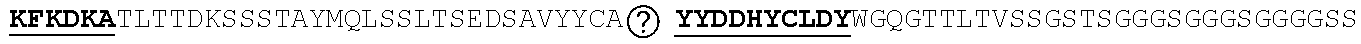

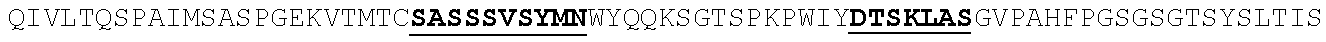

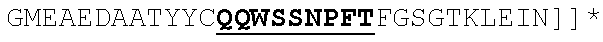

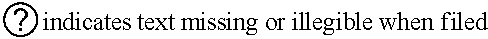

[symbol]

=variable domain of heavy chain (V_{H}) ABCB5 antigen specificity [symbol]
=anti-CD3 scFv (V_{H}-V_{L} or V_{L}-V_{H}) CD3 antigen specificity—double brackets

Light chain (lc) variable domain (V_{L}) peptide sequence AB1 (-> Abi LC) not
subject to changes
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK
Ab19: Heavy chain (hc) full peptide sequence H01 with C-terminal fused
anti-CD3 scFv:
(SEQ ID NO: 120)

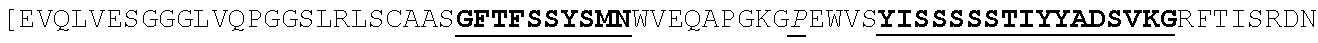

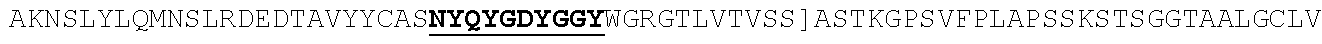

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSPG

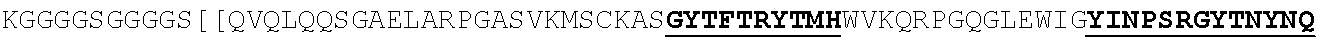

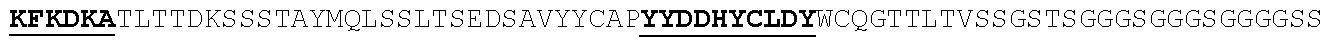

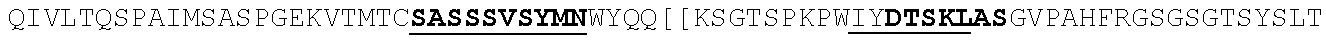

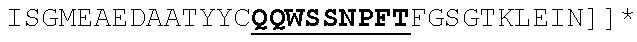

Light chain (lc) variable domain (V_{L}) peptide sequence H01:
(SEQ ID NO: 22)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIK
Ab20: Heavy chain (hc) variable domain (V_{H}) peptide sequence AB1 (not
addressed):
(SEQ ID NO: 1)
EVQLLESGGGLVQPGGSLRLSCAASGFTFSSYSMNWVRQAPGKGLEWVSYISSSSSTIYYADSVKGRFTISRDNS
KNTLYLQMNSLRAEDTAVYYCAKNYQYGDYGGYWGRGTLVTVSS
Light chain (lc) full peptide sequence AB1 with C-terminal fused anti-CD3
scFv:
(SEQ ID NO: 121)

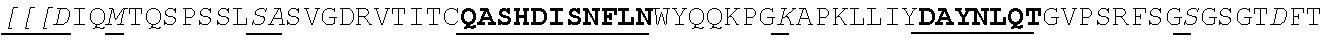

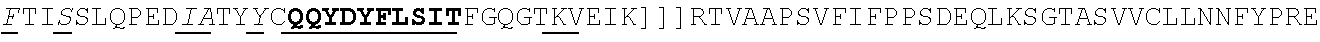

AKVQWKVDNALQSGNSQESVTEQDSKDSTYSLSSTLTLSKADYEKHKVYACEVTHQGLSSPVTKSFNRGECGGGG

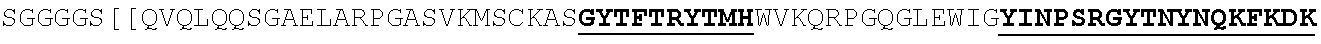

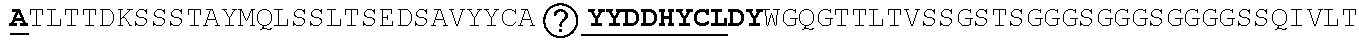

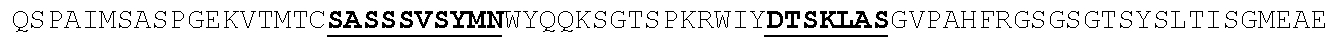

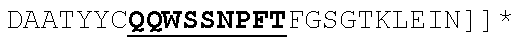

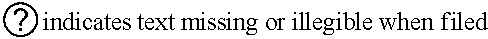

[symbol]

=variable domain of light chain (V_{L}) ABCB5 antigen specificity [symbol]
=anti-CD3 scFv (V_{H}-V_{L} or V_{L}-V_{H}) CD3 antigen specificity

AB21: Heavy chain (hc) variable domain (V_{H}) peptide sequence H01:
(SEQ ID NO: 4)
EVQLVESGGGLVQPGGSLRLSCAASGFTFSSYSMNWVRQAPGKGPEWVSYISSSSSTIYYADSVKGRFTISRDNA
KNSLYLQMNSLRDEDTAVYYCASNYQYGDYGGYWGRGTLVTVSS
Light chain (lc) full peptide sequence H01 with C-terminal fused anti-CD3
scFv:
(SEQ ID NO: 122)

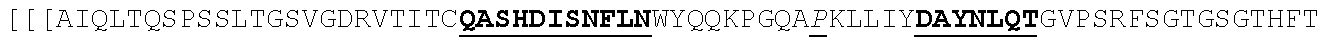

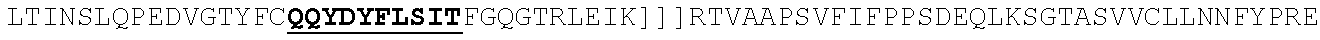

AKVQWKVDNALQSGNSQESVTEQDSKDSTYSLSSTLTLSKADYEKHKVYACEVTHQGLSSPVTKSFNRGECGGGG

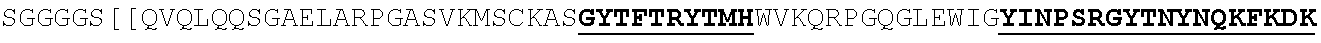

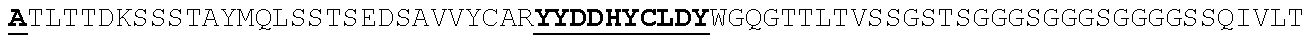

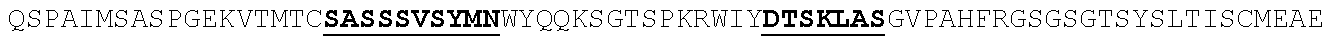

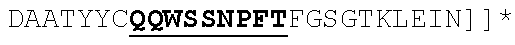

Ab22: Heavy chain (hc) full peptide sequence AB1 with C-terminal fused
anti-Cdl6 scFv:
(SEQ ID NO: 123)

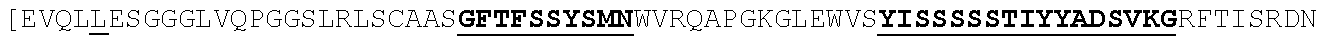

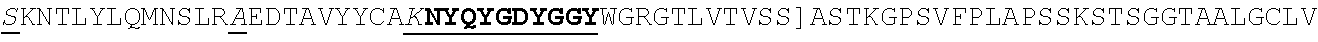

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSPG

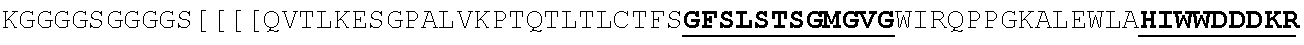

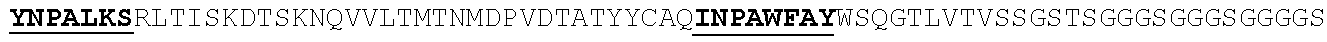

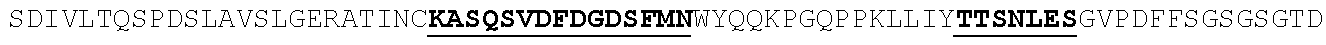

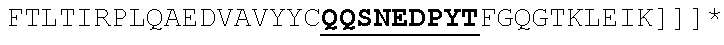

[symbol]

=variable domain of heavy chain (V_{H}) ABCB5 antigen specificity [symbol]
=anti-Cd16 scFv (V_{H}-V_{L} or V_{L}-V_{H})

Light chain (lc) variable domain (V_{L}) peptide sequence AB1 (not addressed):
(SEQ ID NO: 20)
DIQMTQSPSSLSASVGDRVTITCQASHDISNFLNWYQQKPGKAPKLLIYDAYNLQTGVPSRFSGSGSGTDFTFTI
SSLQPEDIATYYCQQYDYFLSITFGQGTKVEIK
Ab23: Heavy chain (hc) full peptide sequence H01 with C-terminal fused
anti-Cdl6 scFv:
(SEQ ID NO: 124)

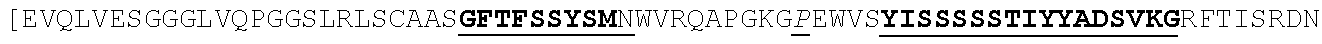

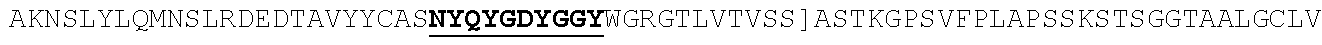

KDYFPEPVTVSWNSGALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKG
FYPSDIAVEWESNGQPENNYKTTPPVLDSDGSFFLYSKLTVDKSRWQQGNVFSCSVMHEALHNHYTQKSLSLSPG

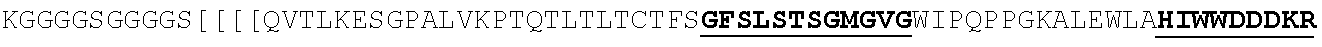

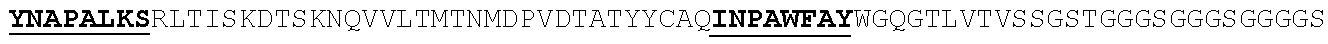

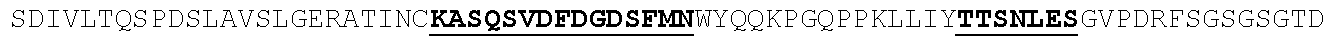

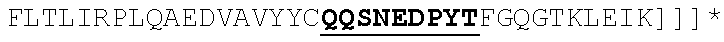

Light chain (lc) variable domain (V_{L}) peptide sequence H01 (not addressed):
(SEQ ID NO: 22)
AIQLTQSPSSLTGSVGDRVTITCQASHDISNFLNWYQQKPGQAPKLLIYDAYNLQTGVPSRFSGTGSGTHFTLTI
NSLQPEDVGTYFCQQYDYFLSITFGQGTRLEIK
Ab24: Heavy chain (hc) variable domain (V_{H}) peptide sequence AB1 (not
addressed):
(SEQ ID NO: 1)
EVQLLESGGGLVQPGGSLRLSCAASGFTFSSYSMNWVRQAPGKGLEWVSYISSSSSTIYYADSVKGRFTISRDNS
KNTLYLQMNSLRAEDTAVYYCAKNYQYGDYGGYWGRGTLVTVSS
Light chain (lc) full peptide sequence AB1 with C-terminal fused anti-Cdl6
scFv:
(SEQ ID NO: 125)

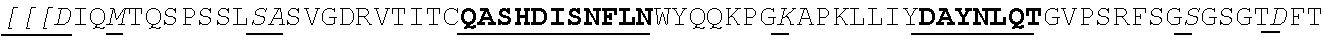

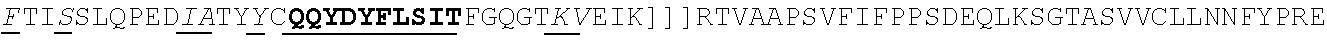

AKVQWKVDNALQSGNSQESVTEQDSKDSTYSLSSTLTLSKADYEKHKVYACEVTHQGLSSPVTKSFNRGECGGGG

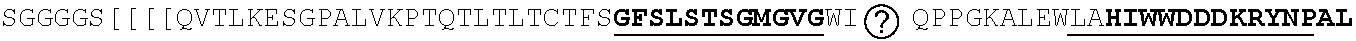

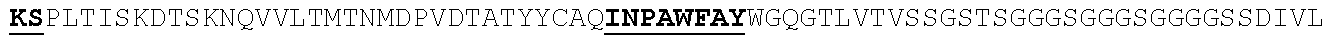

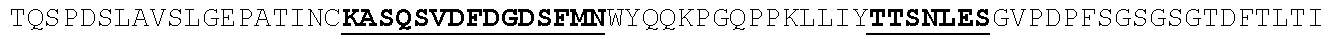

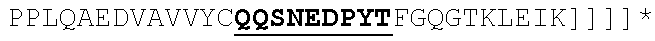

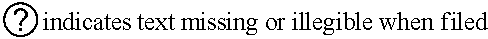

[symbol]

=variable domain of light chain (V_{L}) ABCB5 antigen specificity [symbol]
=anti-Cd16 scFv (V_{H}-V_{L} or V_{L}-V_{H})

Ab25: Heavy chain (hc) variable domain (V_{H}) peptide sequence H01 (not
addressed):
(SEQ ID NO: 4)
EVQLVESGGGLVQPGGSLRLSCAASGFTFSSYSMNWVRQAPGKGPEWVSYISSSSSTIYYADSVKGRFTISRDNA
KNSLYLQMNSLRDEDTAVYYCASNYQYGDYGGYWGRGTLVTVSS
Light chain (lc) full peptide sequence H01 with C-terminal fused anti-Cdl6
scFv:
(SEQ ID NO: 126)

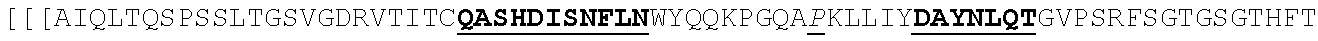

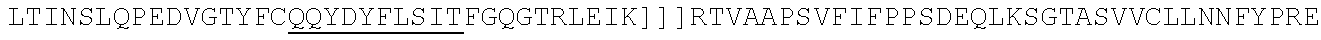

AKVQWKVDNALQSGNSQESVTEQDSKDSTYSLSSTLTLSKADYEKHKVYACEVTHQGLSSPVTKSFNRGECGGGG

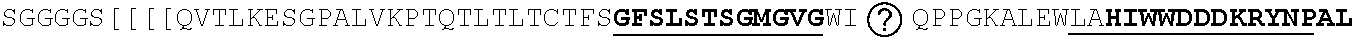

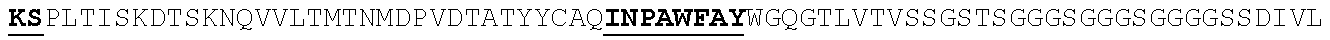

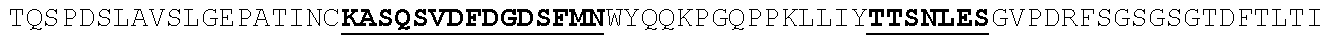

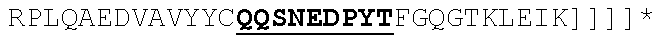

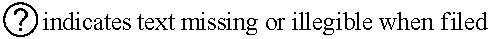

Single-Chain Diabodies (scDb's)

Recombinant antibody fragments in which 2 sets of V_{H} and V_{L} domains (yielding functional scFv fragments) were recombinantly coded/expressed as a continuous single polypeptide chain according to the scheme: e.g. V_{H}A-V_{L}B-V_{H}B-V_{L}A. Since these recombinant antibody fragments were volitionally assembled by employing only variable domains, all the resulting construct combinations did not include a constant domain. Therefore all these constructs e.g. do not possess an Fc region.

Ab26: Full peptide sequence combined variable domains of AB1 and anti-CD3
(V_{H}A-V_{L}B-V_{H}B-V_{L}A) :
(SEQ ID NO: 127)

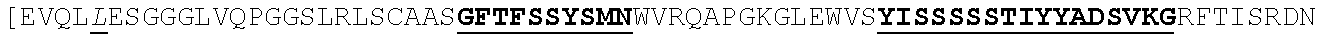

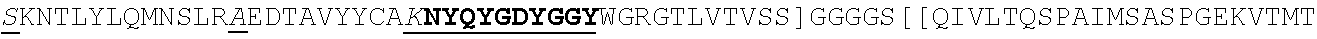

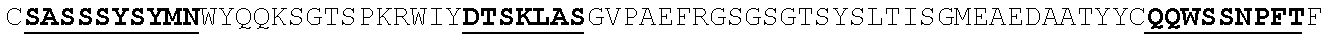

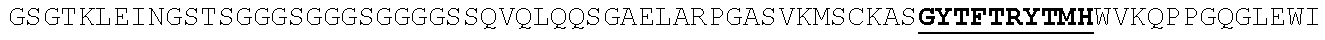

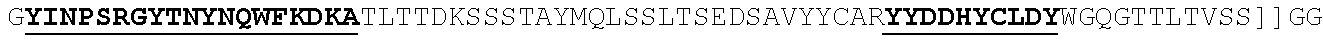

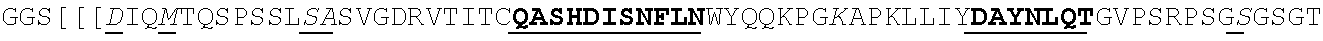

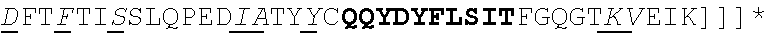

[symbol]

=variable domain of heavy chain (V_{H}) ABCB5 antigen specificity [symbol]
=anti-CD3 scFv (V_{H}-V_{L} or V_{L}-V_{H}) CD3 antigen specificity [symbol]
=variable domain of light chain (V_{L}) ABCB5 antigen specificity

Ab27: Full peptide sequence combined variable domains of AB1 and anti-CD3
(V_{H}B-V_{L}A-V_{H}A-V_{L}B):
(SEQ ID NO: 128)

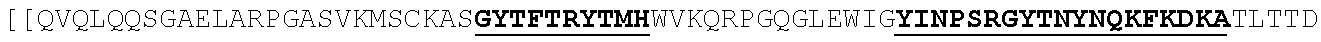

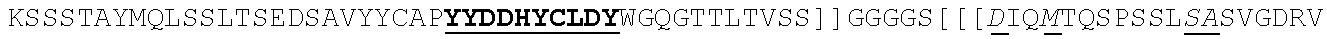

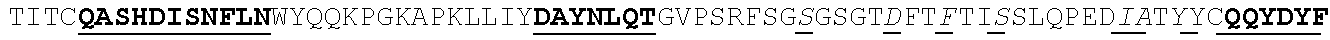

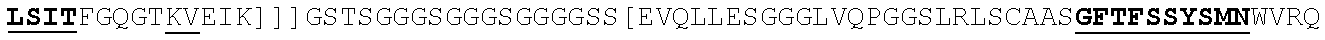

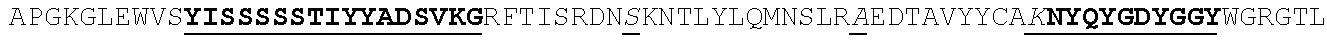

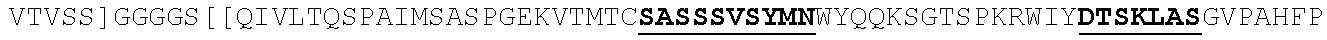

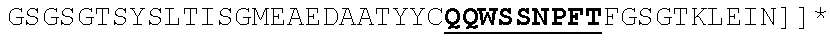

Ab28: Full peptide sequence combined variable domains of H01 and anti-CD3
(V_{H}B-V_{L}A-V_{H}A-V_{L}B):
(SEQ ID NO: 129)

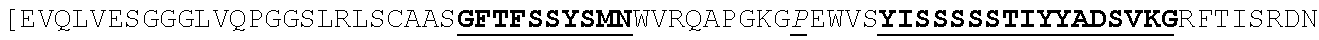

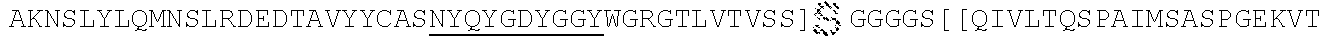

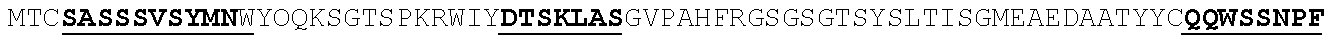

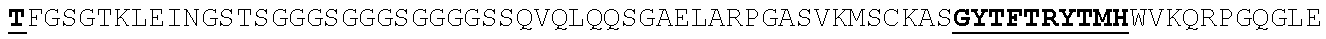

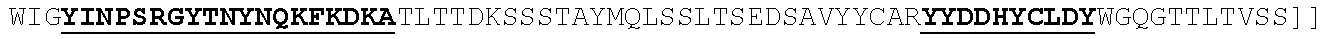

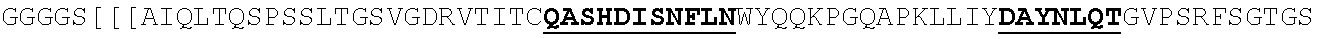

additional Serine amino acid residue (Ser or [symbol] )
Ab29: Full peptide sequence combined variable domains of H01 and anti-CD3
(V_{H}B-V_{L}A-V_{H}A-V_{L}B):
(SEQ ID NO: 130)

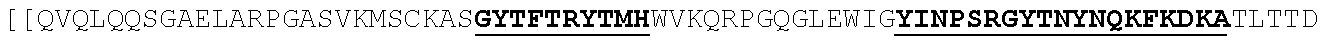

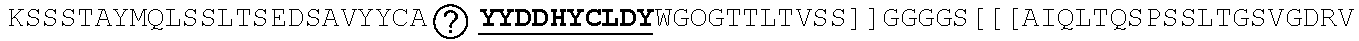

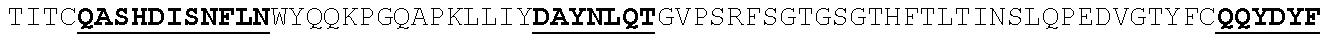

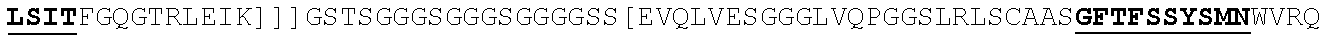

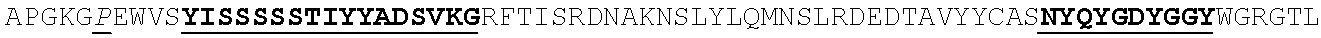

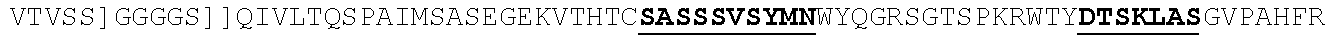

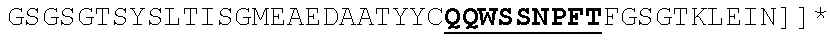

Ab30: Full peptide sequence combined variable domains of AB1 and anti-Cdl6
(V_{H}B-V_{L}A-V_{H}A-V_{L}B):
(SEQ ID NO: 131)

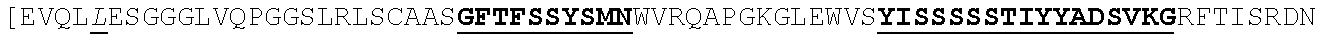

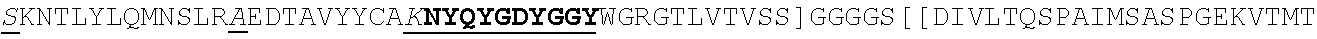

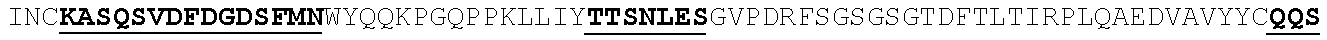

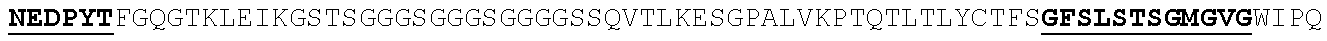

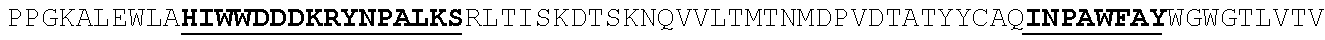

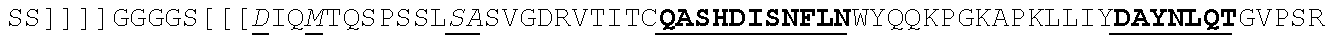

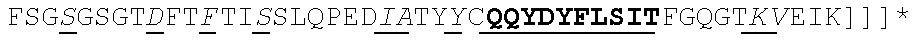

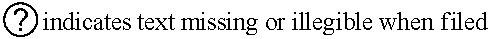

[symbol]

=variable domain of heavy chain (V_{H})->ABCB5 antigen specificity [symbol]
=anti-Cd16 scFv (V_{H}-V_{L} or V_{L}-V_{H}) [symbol]
=variable domain of light chain (V_{L})->ABCB5 antigen specificity

Ab31: Full peptide sequence combined variable domains of AB1 and anti-Cdl6
(V_{H}B-V_{L}A-V_{H}A-V_{L}B):
(SEQ ID NO: 132)

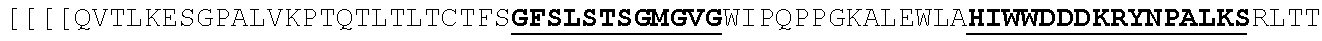

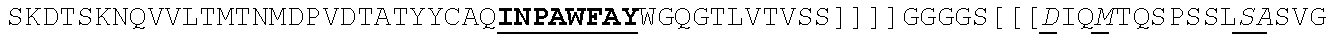

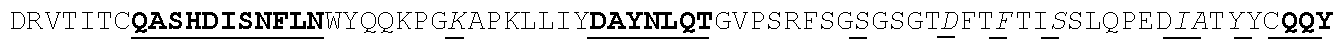

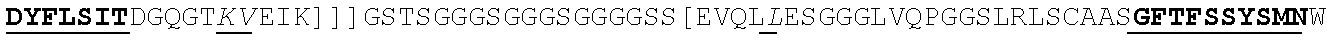

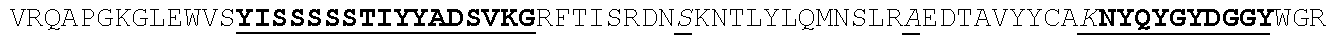

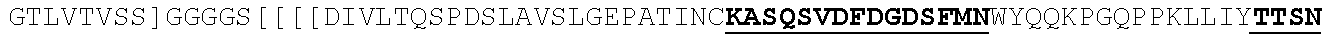

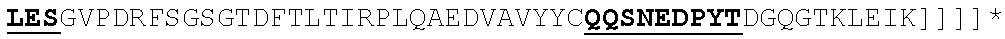

Ab32: Full peptide sequence combined variable domains of H01 and anti-Cdl6
(V_{H}B-V_{L}A-V_{H}A-V_{L}B):
(SEQ ID NO: 133)

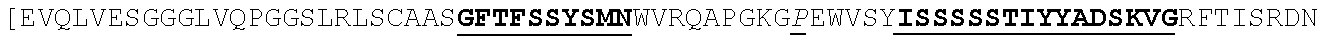

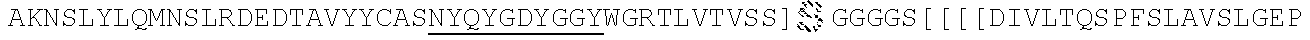

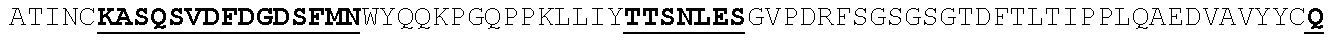

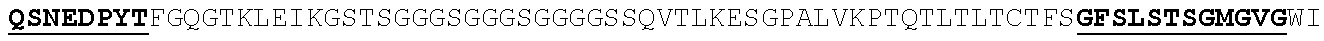

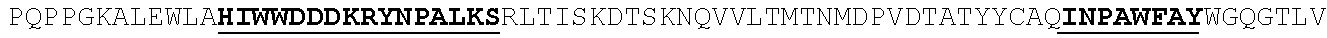

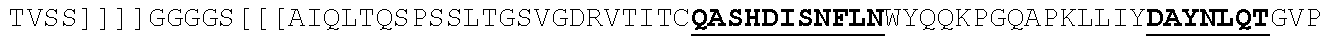

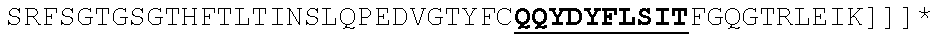

Ab33: Full peptide sequence combined variable domains of H01 and anti-Cdl6
(V_{H}B-V_{L}A-V_{H}A-V_{L}B):
(SEQ ID NO: 134)

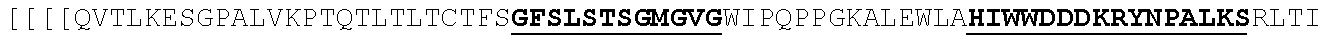

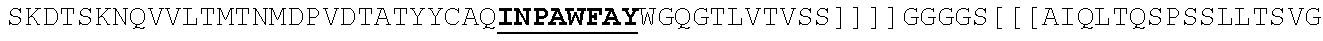

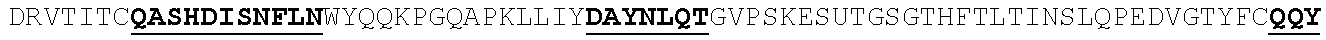

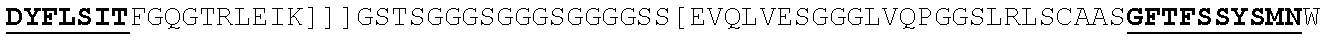

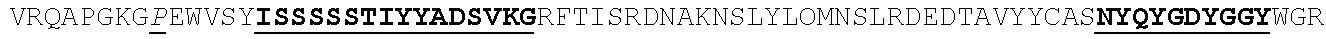

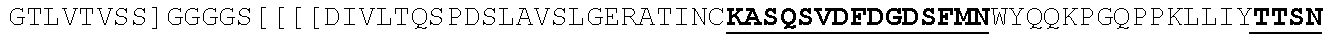

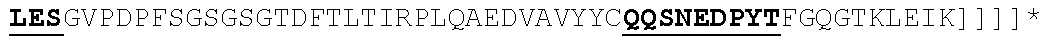

Tandem scFv's (TaFv's) of Ab100

Recombinant antibody fragments in which 2 sets of V_{H} and V_{L} domains (yielding functional scFv fragments) were recombinantly coded/expressed as a continuous single polypeptide chain according to the scheme: e.g. V_{H}A-V_{L}A-V_{H}B-V_{L}B and are referred to as Tandem scFv's (TaFv's). Since these recombinant antibody fragments were volitionally assembled by employing only variable domains, all the resulting construct combinations have no constant domains and, therefore all these constructs e.g. do not possess an Fc region.

Ab34: Full peptide sequence combined variable domains of AB1 and anti-CD3
(V_{H}B-V_{L}A-V_{H}A-V_{L}B):
(SEQ ID NO: 135)

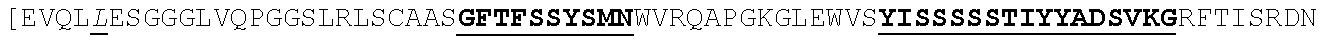

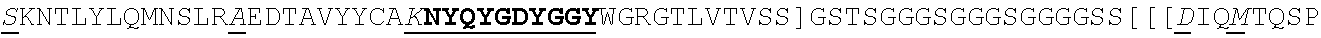

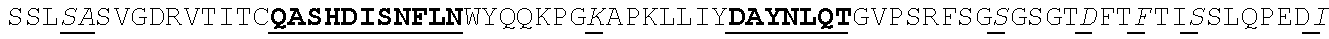

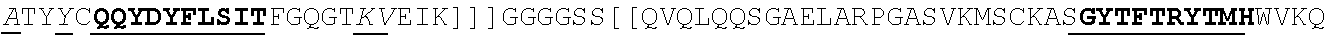

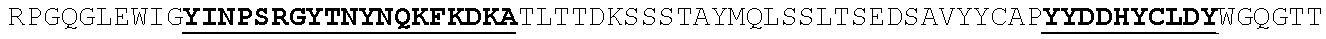

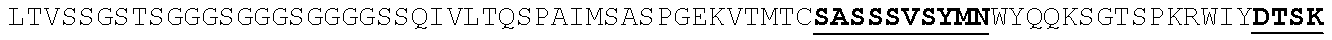

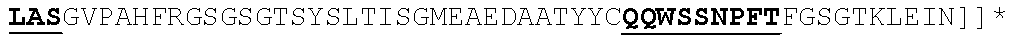

Ab35: Full peptide sequence combined variable domains of AB1 and anti-CD3
(V_{H}B-V_{L}A-V_{H}A-V_{L}B):
(SEQ ID NO: 136)

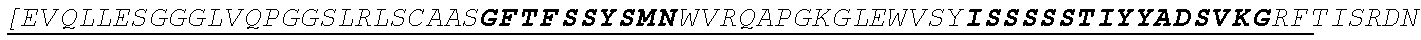

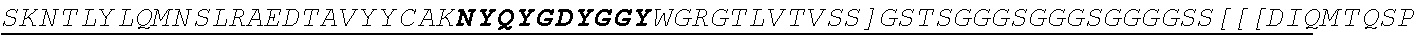

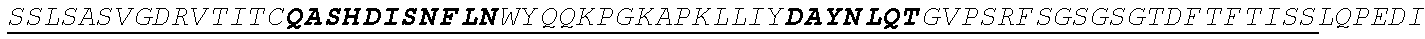

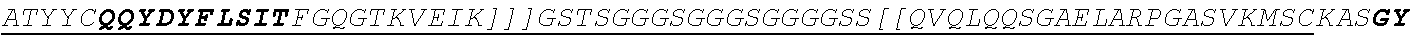

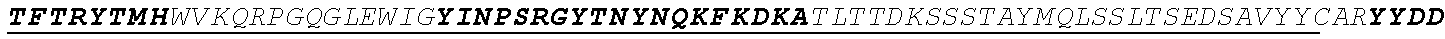

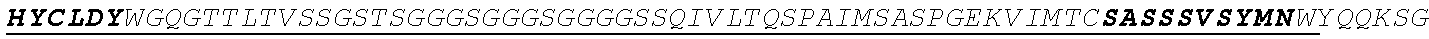

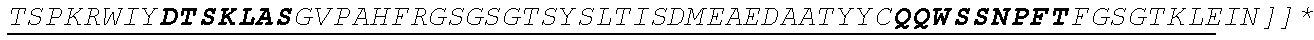

Ab36: Full peptide sequence combined variable domains of H01 and anti-CD3
(V_{H}B-V_{L}A-V_{H}A-V_{L}B):
(SEQ ID NO: 137)

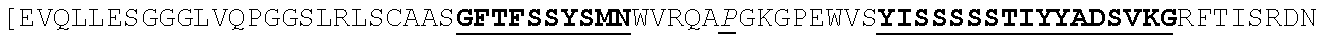

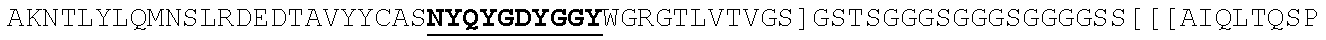

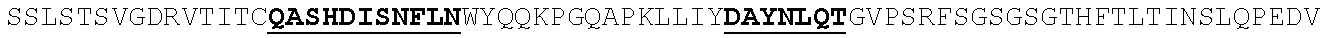

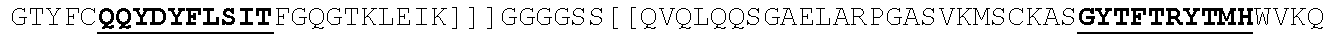

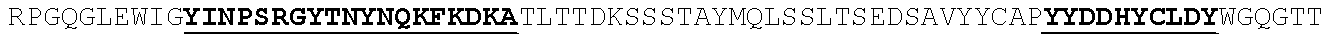

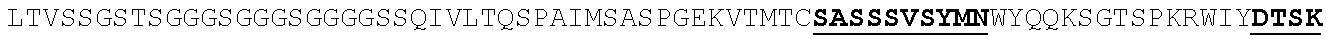

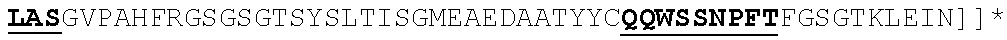

Ab37: Full peptide sequence combined variable domains of H01 and anti-CD3
(V_{H}B-V_{L}A-V_{H}A-V_{L}B):
(SEQ ID NO: 138)

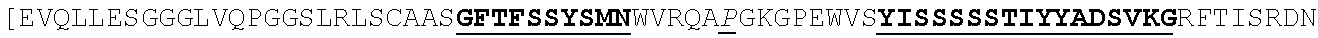

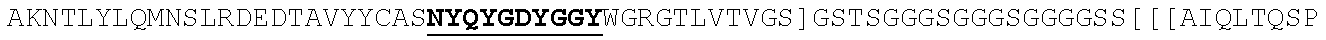

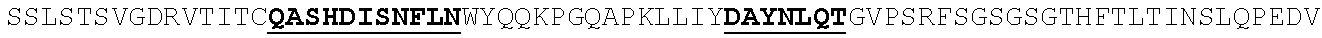

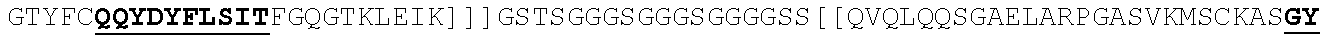

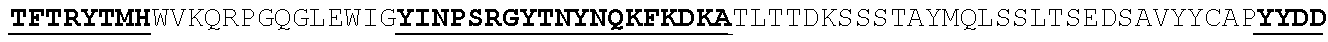

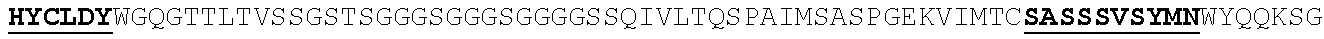

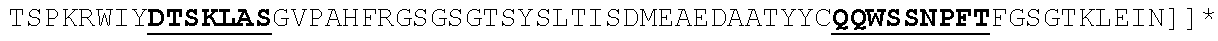

Ab38: Full peptide sequence combined variable domains of AB1 and anti-Cdl6
(V_{H}B-V_{L}A-V_{H}A-V_{L}B):
(SEQ ID NO: 139)

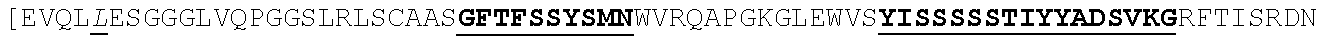

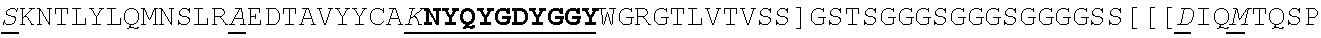

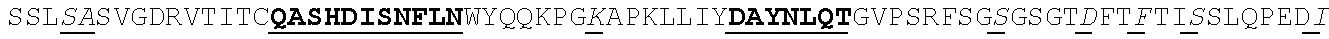

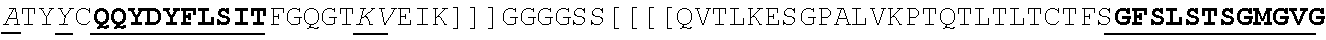

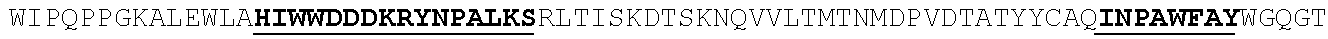

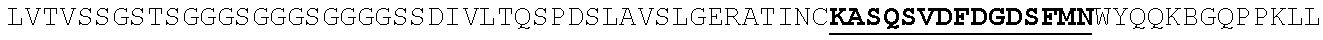

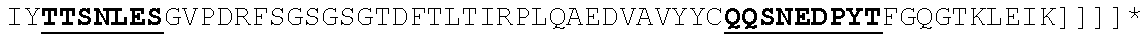

Ab39: Full peptide sequence combined variable domains of AB1 and anti-Cdl6
(V_{H}B-V_{L}A-V_{H}A-V_{L}B):
(SEQ ID NO: 140)

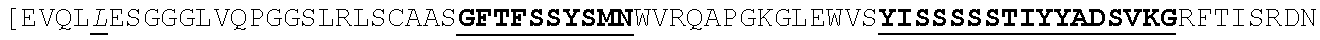

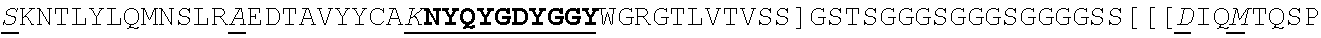

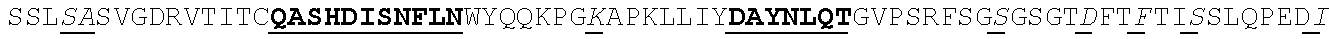

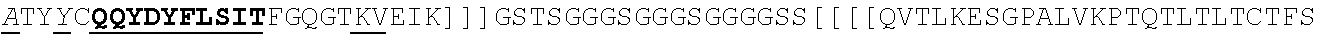

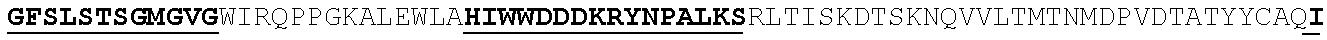

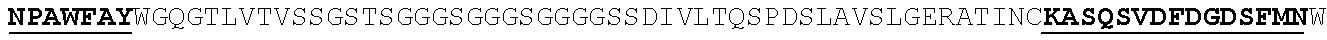

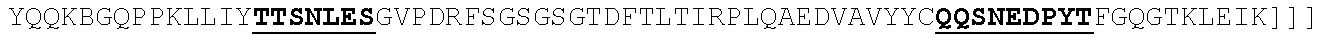

]*
Ab40: Full peptide sequence combined variable domains of H01 and anti-Cdl6
(V_{H}B-V_{L}A-V_{H}A-V_{L}B):
(SEQ ID NO: 141)

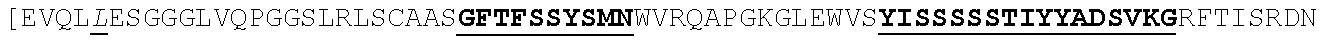

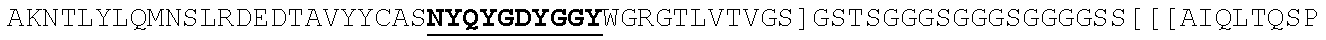

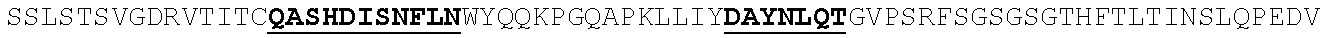

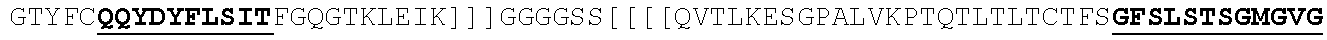

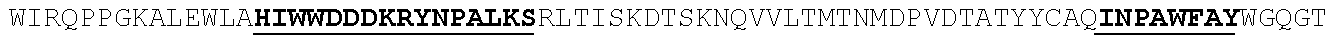

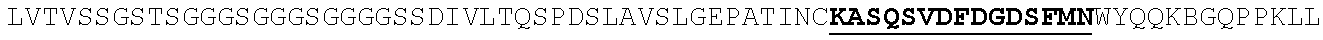

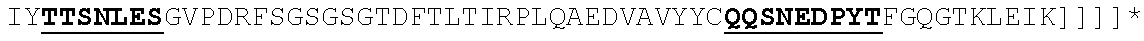

Ab41: Full peptide sequence combined variable domains of H01 and anti-Cdl6
(V_{H}B-V_{L}A-V_{H}A-V_{L}B):
(SEQ ID NO: 142)

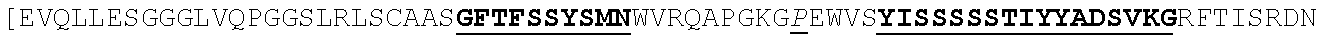

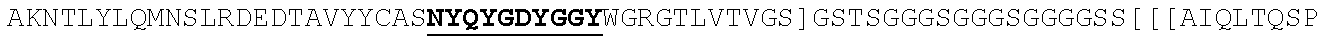

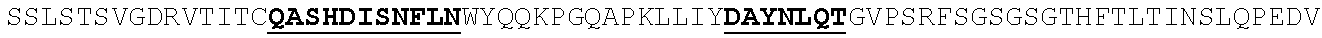

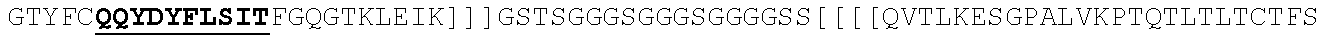

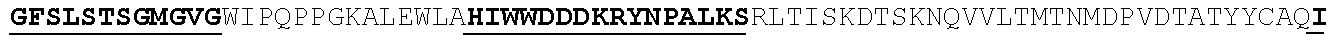

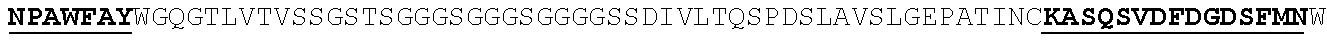

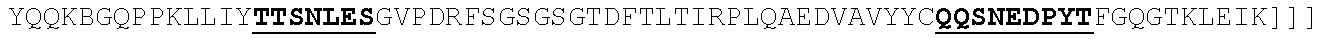

]*

Bispecific variants of Ab101 are disclosed below:

Ab46: Heavy chain (hc) full peptide sequence AB101 with C-terminal fused
anti-CD3 scFv:
(SEQ ID NO: 143)

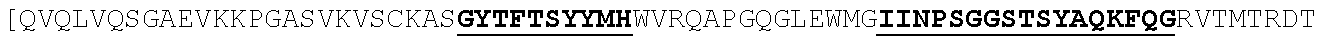

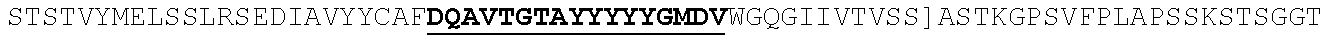

AALGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYTQK

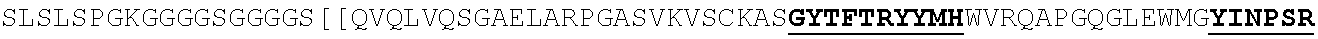

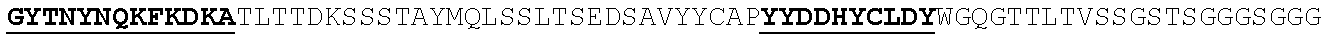

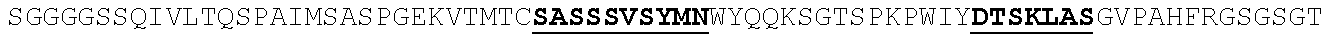

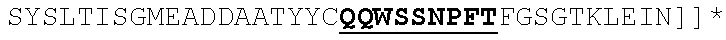

[symbol]

=anti-CD3 scFv (V_{H}-V_{L} or V_{L}-V_{H})

Light chain (lc) variable domain (V_{L}) peptide sequence AB101 (not
adressed):
(SEQ ID NO: 28)
EIVLTQSPGTLSLSPGERATLSCRASQSVNSNYLAWYQQKPGQAPRLLIYGTSSRATGIPDSFSGSGSGTDFTLT
ISRLEPEDFAVYYCQQFGSSPLTFGGGTKVEIK
Ab47: Light chain (lc) full peptide sequence AB101 with C-terminal fused
anti-CD 3 ScFv:
(SEQ ID NO: 144)

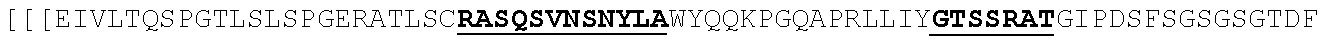

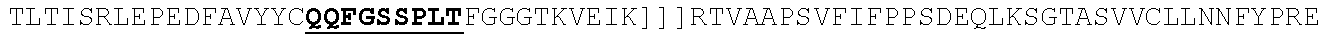

AKVQWKVDNALQSGNSQESVTEQDSKDSTYSLSSTLTLSKADYEKHKVYACEVTHQGLSSPVTKSFNRGECGGGG

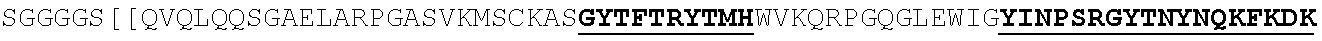

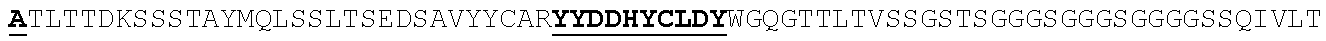

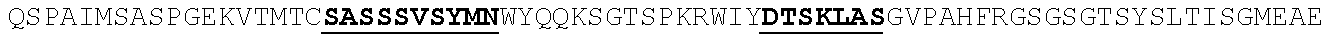

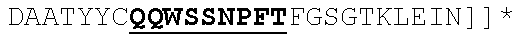

Heavy chain (hc) variable domain (V_{H}) peptide sequence AB101 (not
adressed):
(SEQ ID NO: 17)
QVQLVQSGAEVKKPGASVKVSCKASGYTFTSYYMHWVRQAPGQGLEWMGIINPSGGSTSYAQKFQGRVTMTRDTS
TSTVYMELSSLRSEDTAVYYCARDQAVTGTAYYYYYGMDVWGQGTTVTVSS
Ab48: Heavy chain (hc) full peptide sequence AB101 with C-terminal fused
anti-Cdl6 scFv:
(SEQ ID NO: 145)

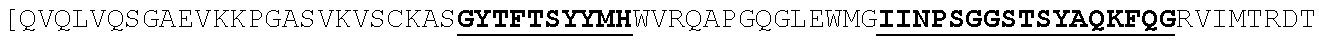

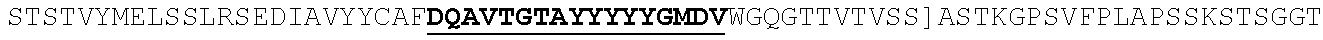

AALGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYTQK

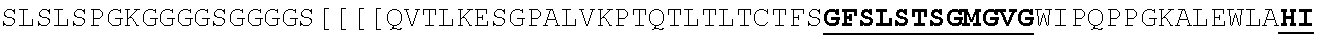

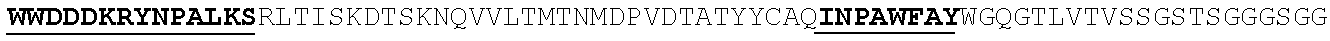

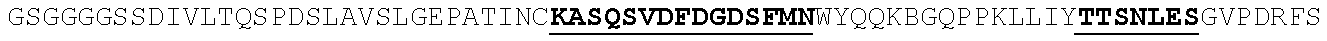

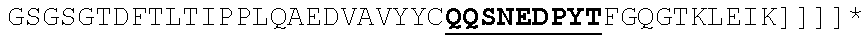

[symbol]

=anti-Cd16 scFv (V_{H}-V_{L} or V_{L}-V_{H})

Light chain (1c) variable domain (V_{L}) peptide sequence AB101:
(SEQ ID NO: 28)
EIVLTQSPGTLSLSPGERATLSCRASQSVNSNYLAWYQQKPGQAPRLLIYGTSSRATGIPDSFSGSGSGTDFTLT
ISRLEPEDFAVYYCQQFGSSPLTFGGGTKVEIK
Ab49: Light chain (1c) full peptide sequence AB101 with C-terminal fused
anti-Cdl6 scFv:
(SEQ ID NO: 146)

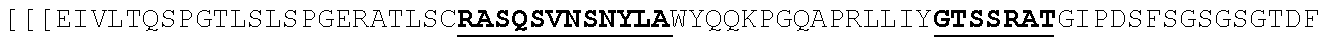

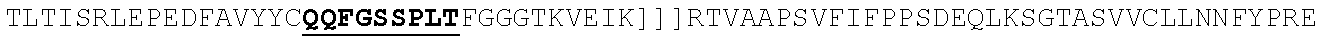

AKVQWKVDNALQSGNSQESVTEQDSKDSTYSLSSTLTLSKADYEKHKVYACEVTHQGLSSPVTKSFNRGECGGGG

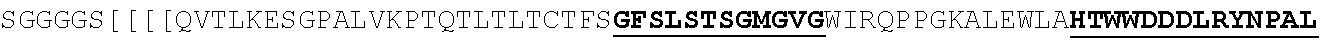

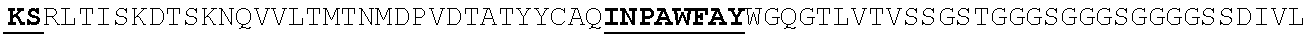

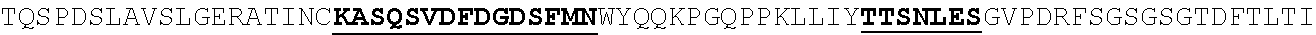

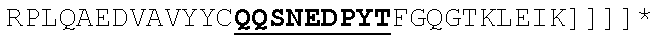

Heavy chain (hc) variable domain (V_{H}) peptide sequence AB101 (not
adressed):
(SEQ ID NO: 17)
QVQLVQSGAEVKKPGASVKVSCKASGYTFTSYYMHWVRQAPGQGLEWMGIINPSGGSTSYAQKFQGRVTMTRDTS
TSTVYMELSSLRSEDTAVYYCARDQAVTGTAYYYYYGMDVWGQGTTVTVSS
Single-chain diabodies (scDb’s) of AblOl:
Ab50: Full peptide sequence combined variable domains of AB101 and anti-
CD3
(V_{H}A-V_{L}B-V_{H}B-V_{L}A) :
(SEQ ID NO: 147)

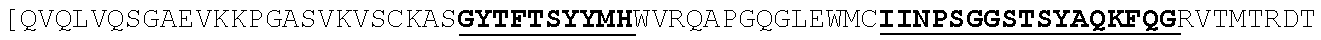

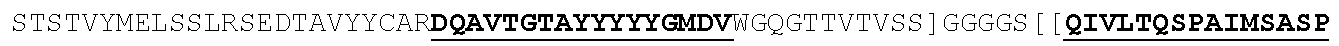

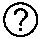

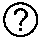

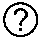

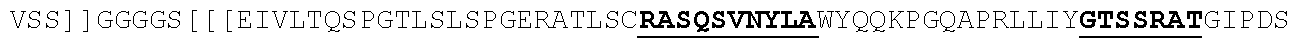

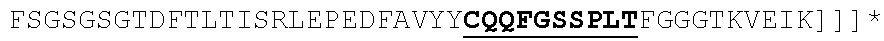

Ab51: Full peptide sequence combined variable domains of AB101 and anti-
CD3
(V_{H}B-V_{L}A-V_{H}A-V_{L}B):
(SEQ ID NO: 148)

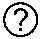

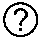

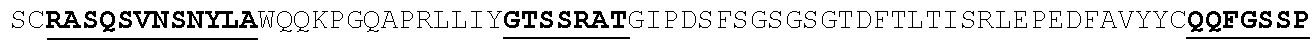

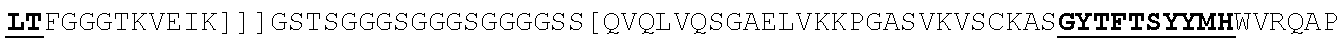

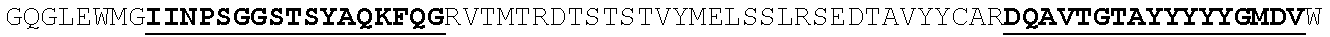

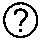

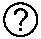

Ab52: Full peptide sequence combined variable domains of AB101 and anti-
Cdl6
(V_{H}B-V_{L}A-V_{H}A-V_{L}B):
(SEQ ID NO: 149)

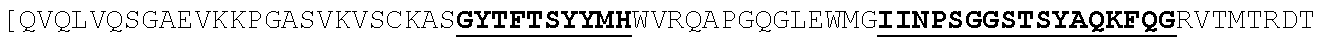

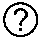

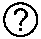

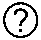

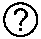

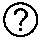

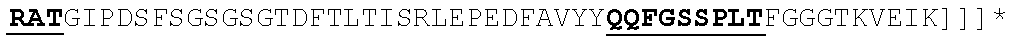

Ab53: Full peptide sequence combined variable domains of AB101 and anti-
Cdl6
(V_{H}B-V_{L}A-V_{H}A-V_{L}B):
(SEQ ID NO: 150)

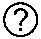

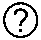

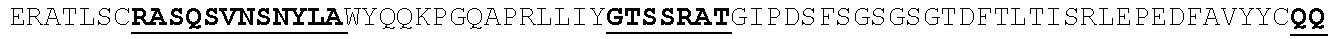

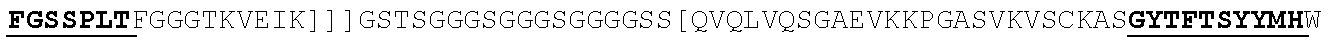

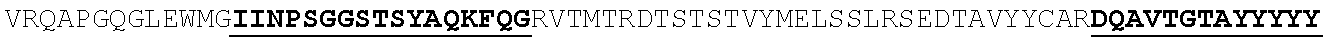

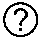

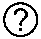

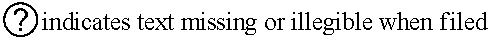

Tandem ScFv's (TaFv's) of Ab101:

Ab54: Full peptide sequence combined variable domains of (h)AB101 and
anti-CD3
(V_{H}B-V_{L}A-V_{H}A-V_{L}B):
(SEQ ID NO: 151)

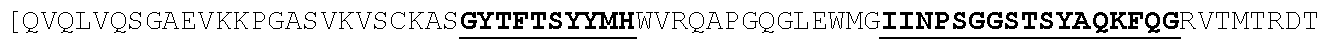

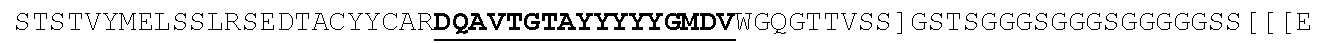

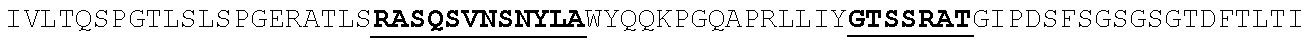

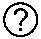

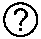

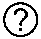

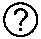

Ab55: Full peptide sequence combined variable domains of (h)AB101 and
anti-CD3
(V_{H}B-V_{L}A-V_{H}A-V_{L}B):
(SEQ ID NO: 152)

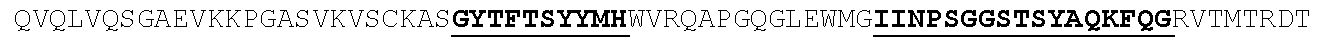

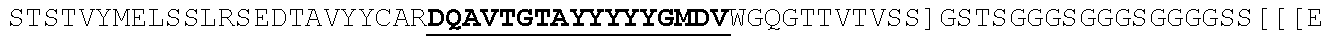

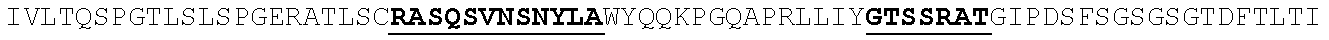

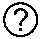

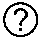

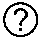

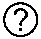

*
Ab56: Full peptide sequence combined variable domains of AB101 and anti-
Cdl6
(V_{H}B-V_{L}A-V_{H}A-V_{L}B):
(SEQ ID NO: 153)

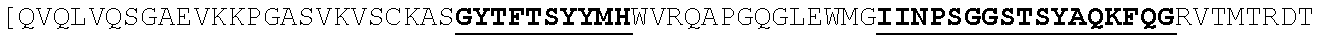

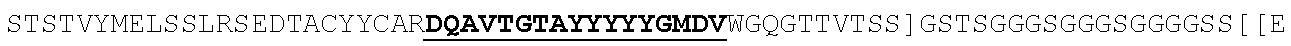

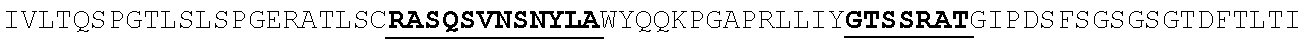

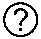

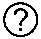

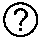

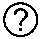

Ab57: Full peptide sequence combined variable domains of AB101 and anti-
Cdl6
(V_{H}B-V_{L}A-V_{H}A-V_{L}B):
(SEQ ID NO: 154)

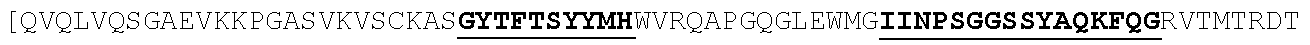

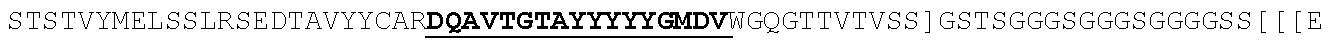

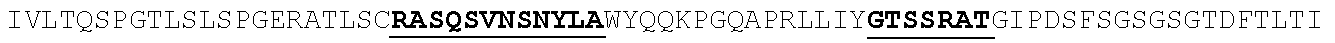

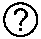

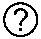

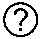

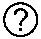

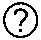

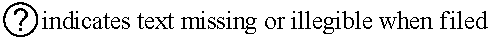

Tandem scFv's (TaFv's) of Ab44:

Ab58: Heavy Chain (Hc) Full Peptide Sequence AB44 with C-Terminal Fused Anti-CD3 scFv:

(SEQ ID NO: 155)

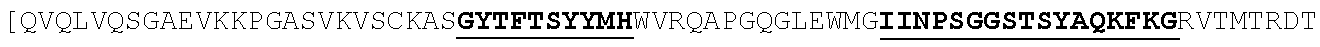

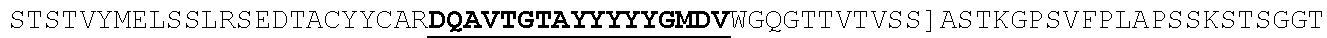

AALGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYTQK

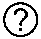

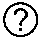

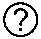

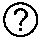

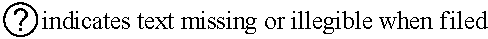

[symbol]

=anti-CD3 scFv (V_{H}-V_{L} or V_{L}-V_{H})

Light chain (1c) variable domain (V_{L}) peptide sequence Ab44 (not adressed):
(SEQ ID NO: 28)
EIVLTOSPGTLSLSPGERATLSCRASQSVNSNYLAWYOOKPGOAPRLLIYGTSSRATGIPDSFSGSGSGTDFTLT
ISRLEPEDFAVYYCQQFGSSPLTFGGGTKVEIK
Ab59: Light chain (1c) full peptide sequence A44 with C-terminal fused
anti-CD3 scFv:
(SEQ ID NO: 144)

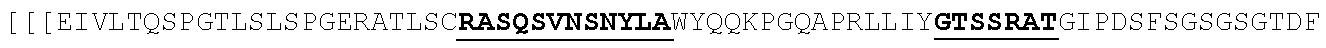

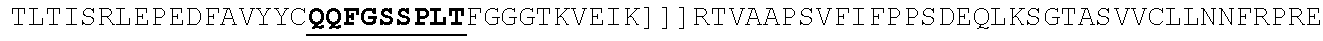

AKVQWKVDNALQSGNSQESVTEQDSKDSTYSLSSTLTLSKADYEKHKVYACEVTHQGLSSPVTKSFNRGECGGGG

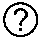

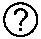

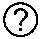

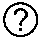

Heavy chain (hc) variable domain (V_{H}) peptide sequence Ab44:
(SEQ ID NO: 15)
QVQLVQSGAEVKKPGASVKVSCKASGYTFTSYYMHWVRQAPGQGLEWMGIINPSGGSTSYAQKFKGRVTMTRDTS
TSTVYMELSSLRSEDTAVYYCARDQAVTGTAYYYYYGMDVWGQGTTVTVSS
Ab60: Heavy chain (hc) full peptide sequence Ab44 with C-terminal fused
anti-Cdl6 scFv:
(SEQ ID NO: 156)

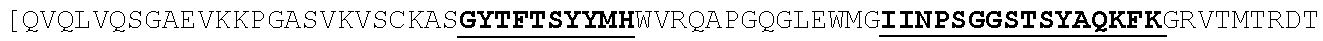

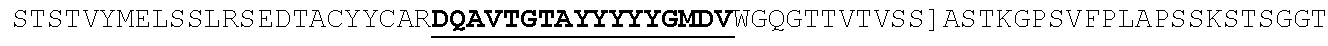

AALGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYTQK

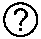

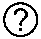

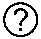

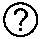

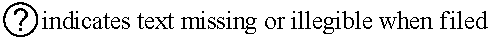

[symbol]

=anti-Cd16 scFv (V_{H}-V_{L} or V_{L}-V_{H})

Light chain (1c) variable domain (VL) peptide sequence Abl0l (not
addressed):
(SEQ ID NO: 28)
EIVLTQSPGTLSLSPGERATLSCRASQSVNSNYLAWYQQKPGQAPRLLIYGTSSRATGIPDSFSGSGSGTDFTLT
ISRLEPEDFAVYYCQQFGSSPLTFGGGTKVEIK
Ab61: Light chain (1c) full peptide sequence A44 with C-terminal fused
anti-Cdl6 scFv:
(SEQ ID NO: 146)

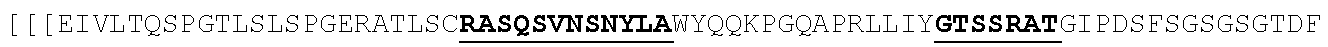

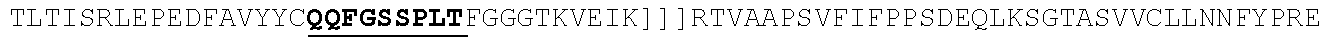

AKVQWKVDNALQSGNSQESVTEQDSKDSTYSLSSTLTLSKADYEKHKVYACEVTHQGLSSPVTKSFNRGECGGGG

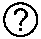

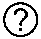

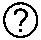

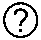

Heavy chain (hc) variable domain (V_{H}) peptide sequence Abl0l (not
adressed):
(SEQ ID NO: 15)
QVQLVQSGAEVKKPGASVKVSCKASGYTFTSYYMHWVRQAPGQGLEWMGIINPSGGSTSYAQKFKGRVTMTRDTS
TSTVYMELSSLRSEDTAVYYCARDQAVTGTAYYYYYGMDVWGQGTTVTVSS
Ab62: Full peptide sequence combined variable domains of Abl0l and anti-
CD3
(V_{H}B-V_{L}A-V_{H}A-V_{L}B):
(SEQ ID NO: 157)

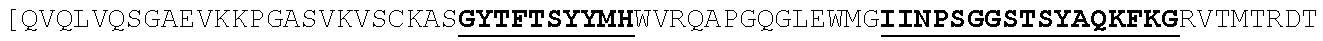

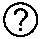

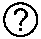

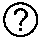

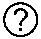

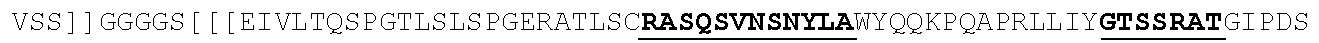

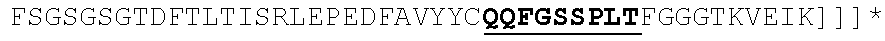

Ab63: Full peptide sequence combined variable domains of A44 and anti-CD3
(V_{H}B-V_{L}A-V_{H}A-V_{L}B):
(SEQ ID NO: 158)

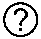

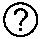

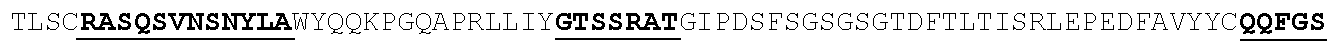

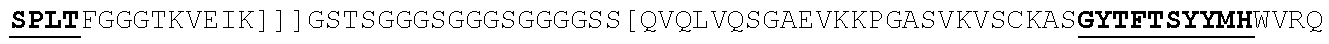

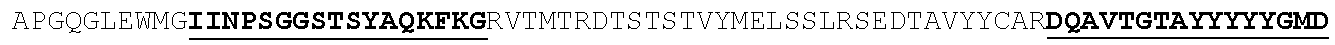

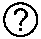

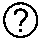

Ab64: Full peptide sequence combined variable domains of Ab44 and anti-
Cdl6
(V_{H}B-V_{L}A-V_{H}A-V_{L}B):
(SEQ ID NO: 159)

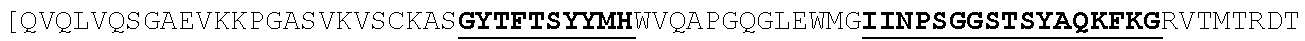

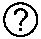

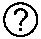

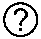

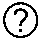

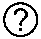

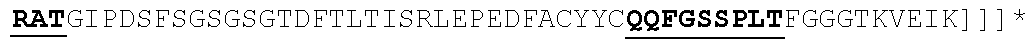

Ab65: Full peptide sequence combined variable domains of Ab44 and anti-
Cdl6
(V_{H}B-V_{L}A-V_{H}A-V_{L}B):
(SEQ ID NO: 160)

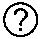

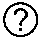

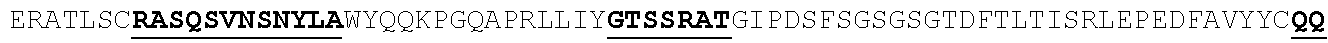

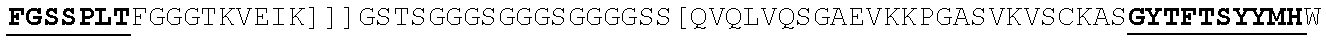

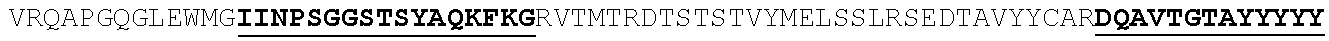

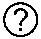

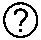

Ab66: Full peptide sequence combined variable domains of Ab44 and anti-CD3
(V_{H}B-V_{L}A-V_{H}A-V_{L}B):
(SEQ ID NO: 161)

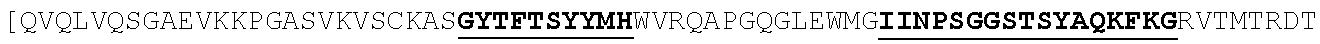

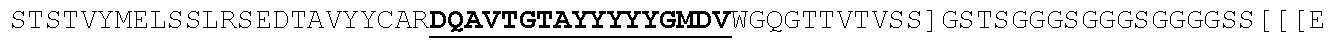

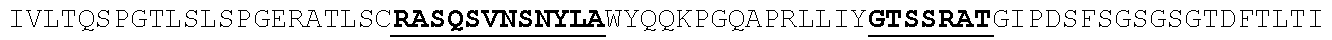

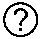

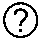

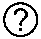

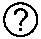

Ab67: Full peptide sequence combined variable domains of A44 and anti-CD3
(V_{H}B-V_{L}A-V_{H}A-V_{L}B):
(SEQ ID NO: 162)

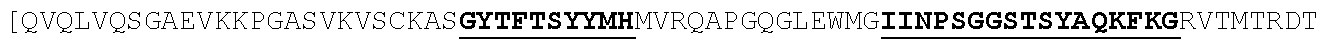

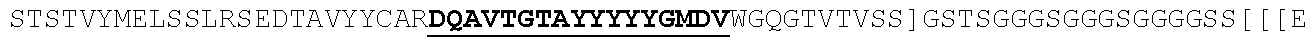

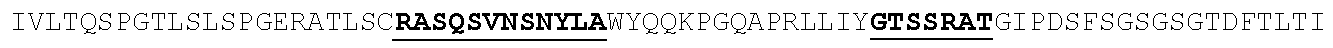

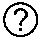

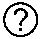

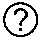

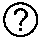

*
Ab68: Full peptide sequence combined variable domains of A44 and anti-
Cd16
(V_{H}B-V_{L}A-V_{H}A-V_{L}B):
(SEQ ID NO: 163)

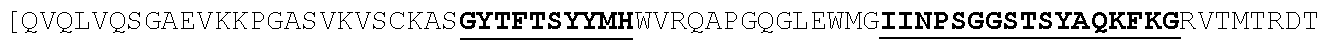

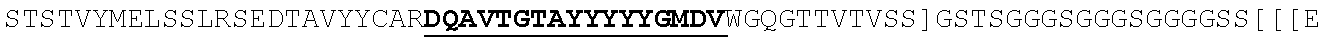

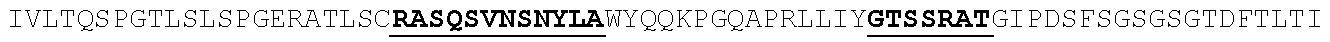

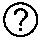

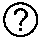

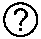

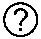

Ab69: Full peptide sequence combined variable domains of Ab44 and anti-
Cd16
(V_{H}B-V_{L}A-V_{H}A-V_{L}B):
(SEQ ID NO: 164)

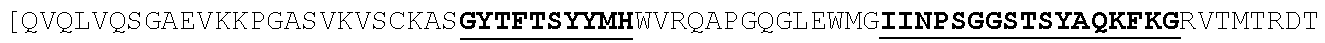

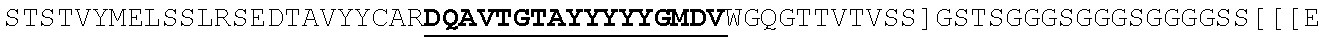

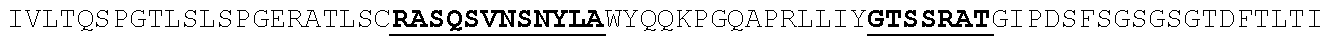

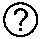

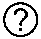

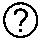

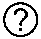

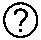

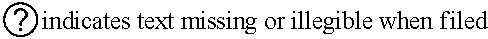

As described in more detail below the invention also encompasses antibody drug conjugates (ADCs) in which an anti-ABCB5 antibody is combined with a linker and payload (cytotoxic agent) for targeted drug delivery. Any of the antibodies or portions thereof described herein conjugated via linker with any cytotoxic payload with any feasible technology/chemistry at any feasible site of antibody molecule is an ADC of the invention.

The constructs described herein may include a linker. The linker may be, for instance, a peptide linker. Peptide linkers include for instance, long peptide linkers such as GSTSGGGSGGGSGGGGSS (SEQ ID NO. 84) and short peptide linkers such as GGGGSS (SEQ ID NO. 86). Numerous other linkers are described herein.

Exemplary recombinant antibody fragments applied for ADC/targeted drug-delivery purposes, with small molecule or peptide-based cytotoxic agents include the following constructs:

Ab100 derived anti-AB CB5 scFv fused with ribosome-inactivating protein (RIP; e.g. Gelonin)

Full peptide sequence combined scFv and Gelonin
(SEQ ID NO: 165)

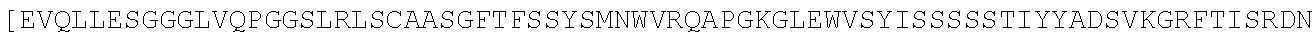

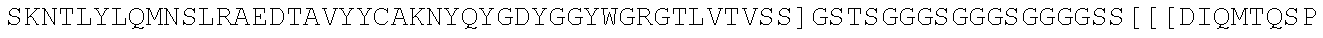

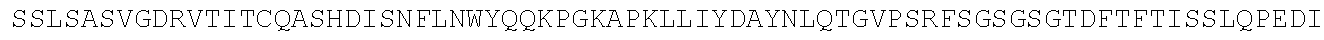

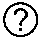

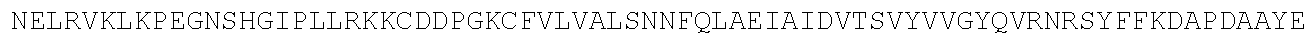

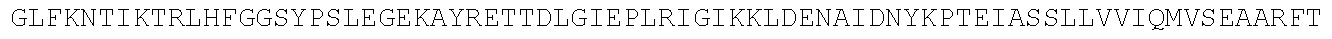

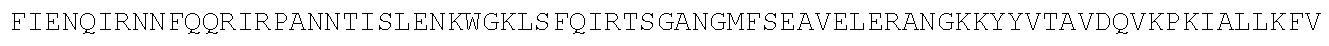

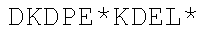

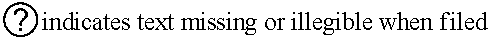

[symbol]

=Myc epitope tag (epitope tags are used for research—not in therapeutic version) [symbol]
=Gelonin
*_*=ER (endoplasmatic reticulum) localization/retention signal peptide; only present in forms from bacterial expression
The tag and ER are optional and may be removed. Thus SEQ ID NO 165 may optionally exclude the tag and ER.

Full peptide sequence combined scFv and Gelonin without epitope tag
(SEQ ID NO: 166)

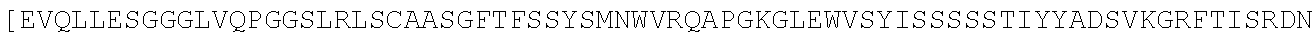

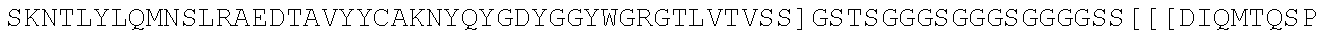

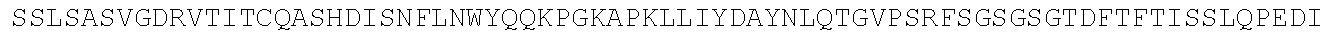

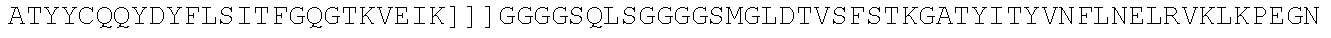

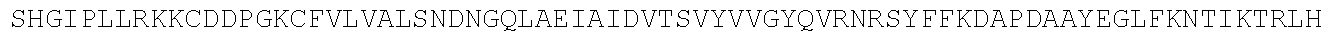

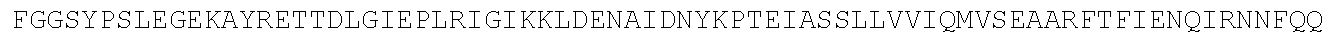

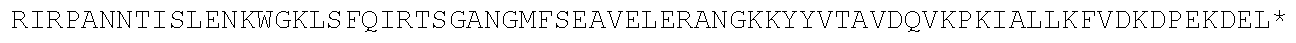

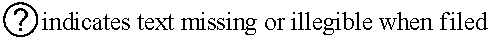

Ab101 derived anti-AB CB 5 scFv fused with ribosome-inactivating protein (RIP) (e.g. Gelonin from Gelonium multiflorum)

Full peptide sequence combined Ab 101 scFv and Gelonin
(SEQ ID NO: 167)

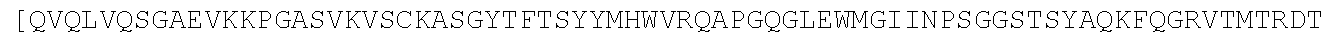

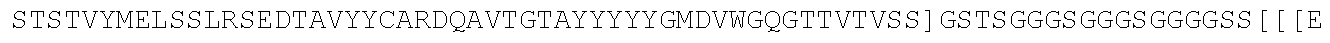

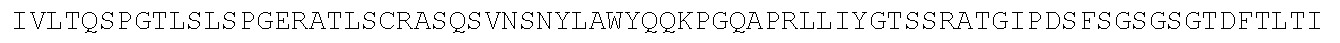

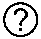

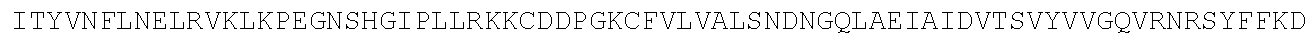

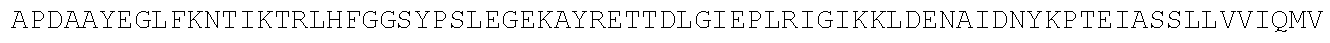

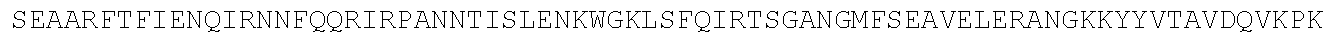

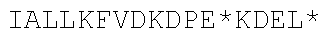

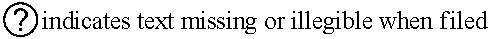

[symbol]

=Myc epitope tag [symbol]
=Gelonin
*_*=ER (endoplasmatic reticulum) localization/retention signal peptide; only present in forms from bacterial expression
The tag and ER are optional and may be removed. Thus SEQ ID NO 167 may optionally exclude the tag and ER.
Other forms included comprise e.g. anti-ABCB5 scFv/TaFv or similar with ribosome-inactivating proteins b) Exotoxin A (ETA; from Pseudomonas aeruginosa), c) Saporin (from Saponaria officinalis), d) Luffin P1 (from Luffa cylindrica) and e) Diphteria toxin (DT; from Corynebacterium diphtheriae)
Anti-ABCB5 scFv's and tandem scFv's (TaFv's) fused with RIPs

### With Gelonin (N- or C-terminal):

Full peptide sequence combined Ab 101 scFv and Gelonin without epitope tag
(SEQ ID NO: 168)

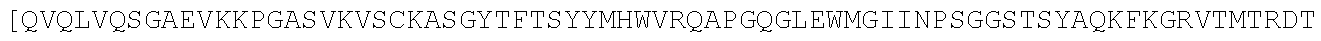

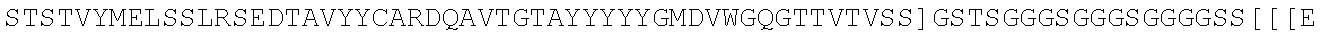

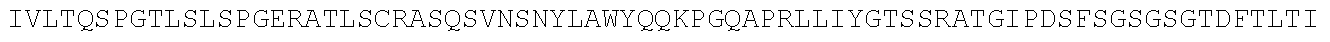

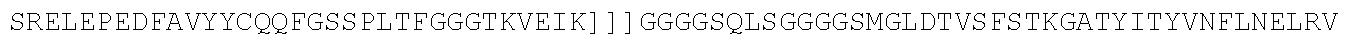

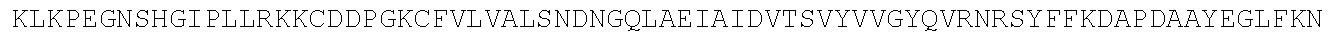

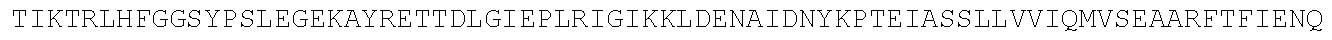

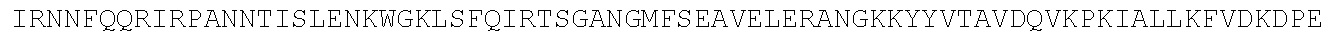

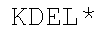

Additional Examples of ABCB5 antibody-drug-conjugates are provided herein. For instance, presented below are exemplary anti-ABCB5 single-chain fragments (Ab8, Ab101, and Ab44), either as strict mono-valent and mono-specific scFv's (VH-VL) or as mono-specific, but bivalent tandem Fv's (VH-VL-[long middle linker=peptide spacer]-VH-VL, combined with an additional peptide linker/spacer and polypeptide-based immunotoxins or payload [RIP=ribosomal inhibitor protein or ribosome-inactivating protein], i.e. a) gelonin (from Gelonium multiflorum), b) Exotoxin A (ETA; from Pseudomonas aeruginosa), c) Saporin (from Saponaria officinalis), d) Luffin P1 (from Luffa cylindrica) and e) Diphteria toxin (DT; from Corynebacterium diphtheriae).

[V_{H}] and [[[V_{L}]]]:

Ab70: Full peptide sequence combined variable domains to scFv of AB1xH01

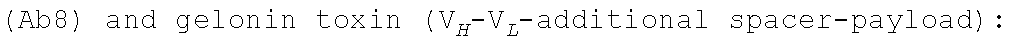

(SEQ ID NO: 169)

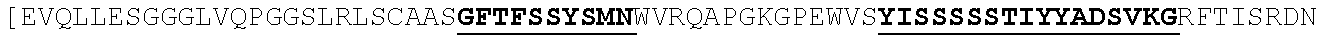

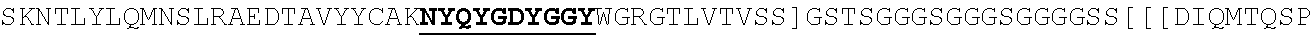

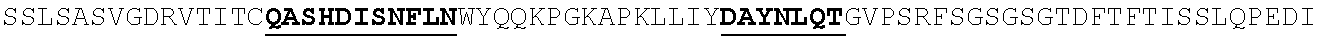

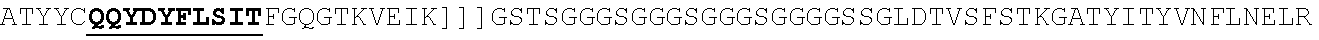

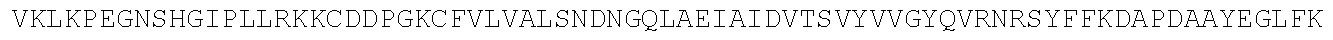

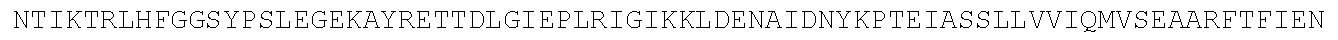

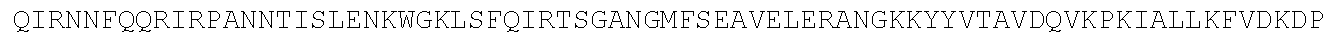

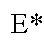

Ab71: Full peptide sequence combined variable domains to TaFv of AB1xH01

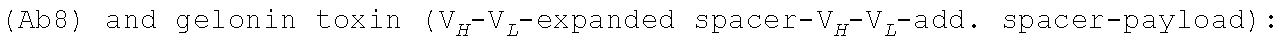

(SEQ ID NO: 170)

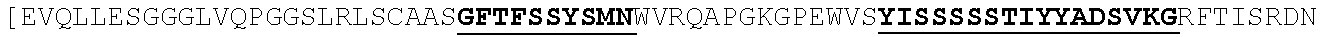

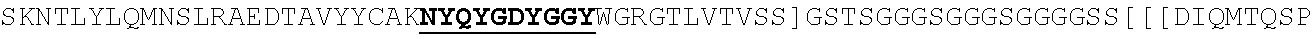

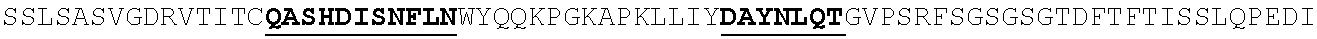

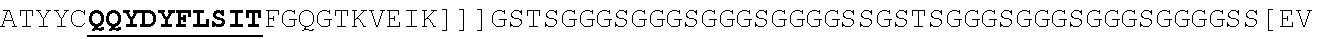

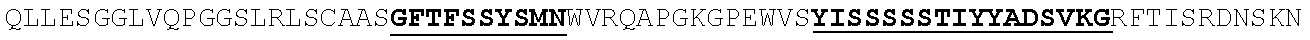

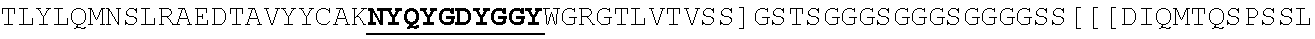

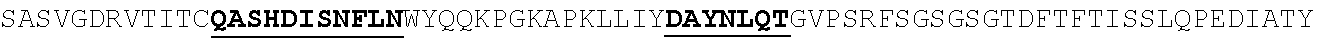

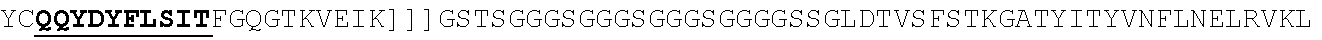

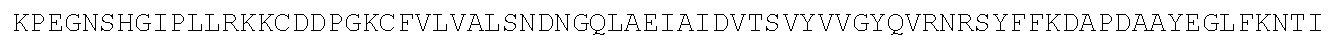

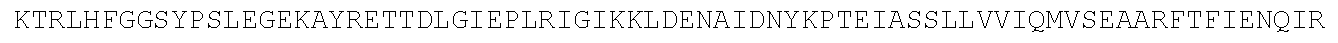

Ab72: Full peptide sequence combined variable domains to scFv of Abl0l and

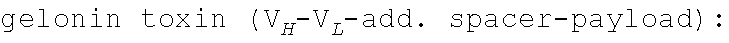

(SEQ ID NO: 171)

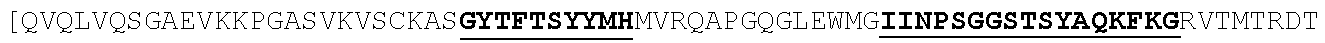

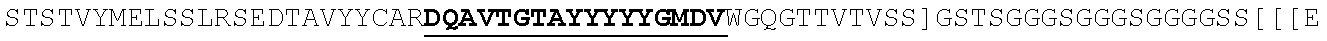

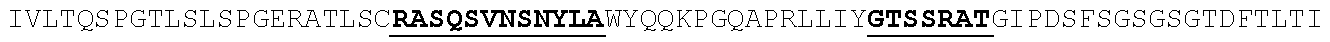

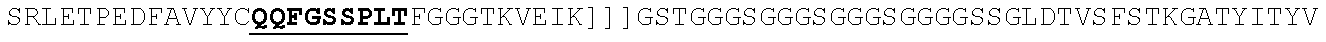

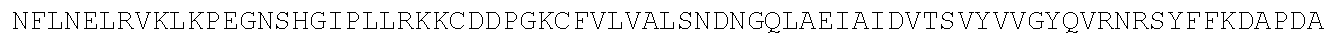

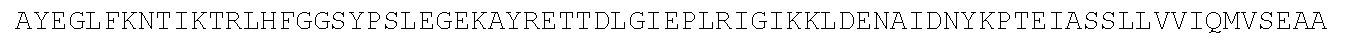

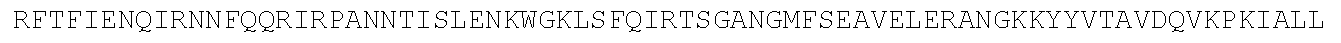

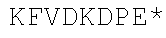

Ab73: Full peptide sequence combined variable domains to TaFv of Ab101 and

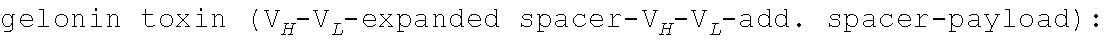

(SEQ ID NO: 172)

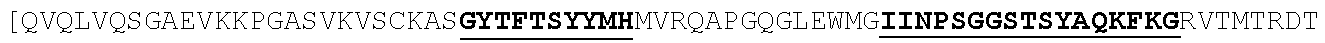

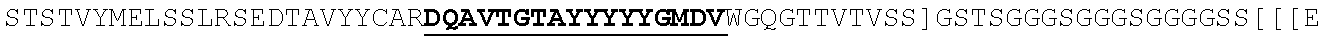

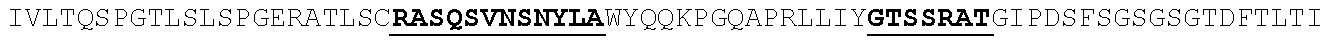

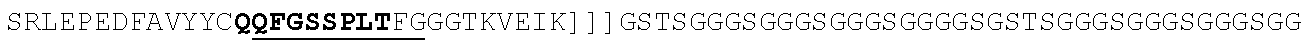

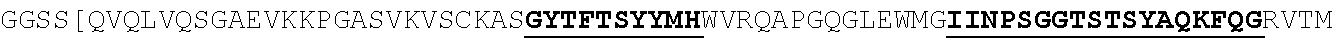

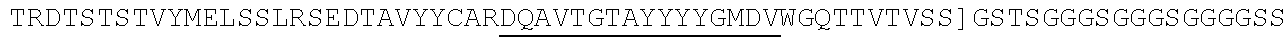

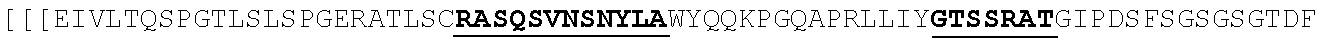

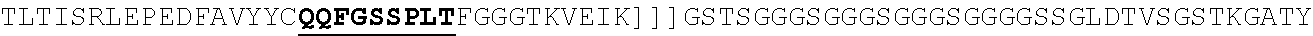

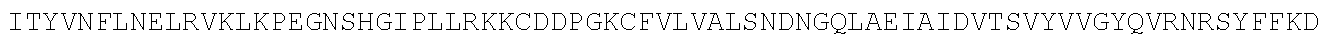

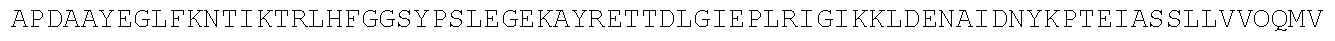

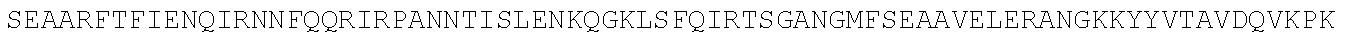

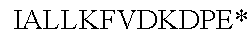

Ab74: Full peptide sequence combined variable domains to scFv of F01

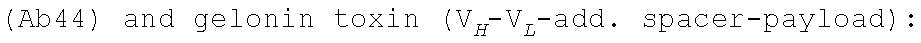

(SEQ ID NO: 19)

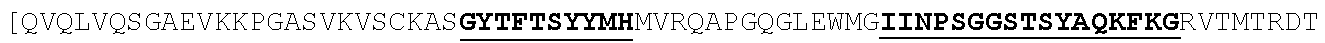

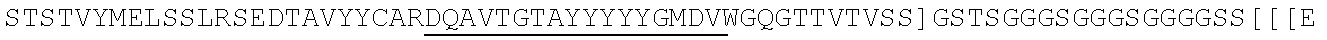

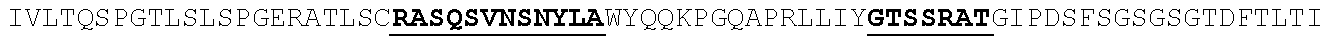

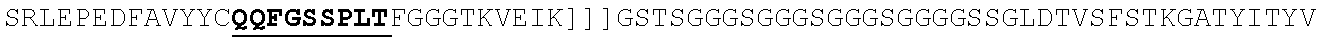

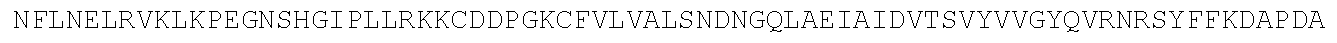

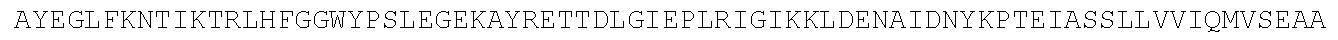

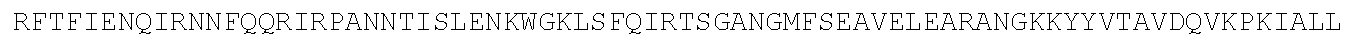

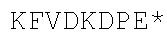

Ab75: Full peptide sequence combined variable domains to TaFv of F01 (Ab44)

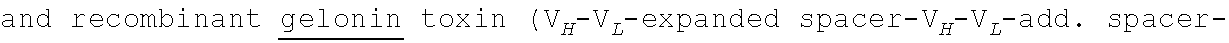

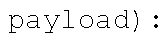

(SEQ ID NO: 19)

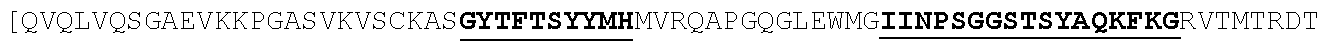

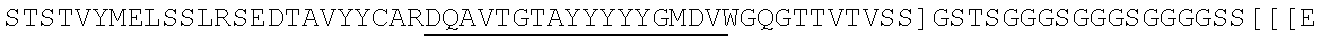

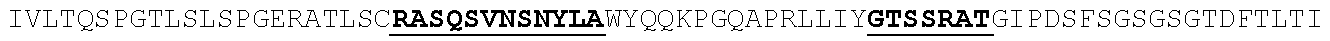

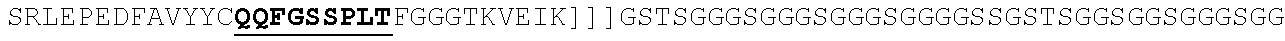

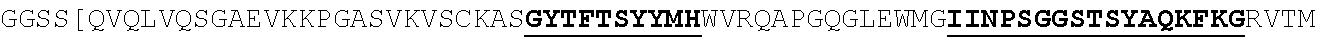

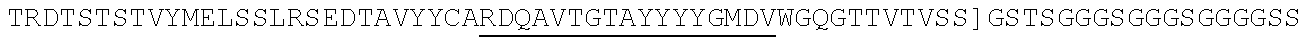

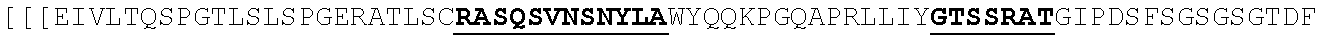

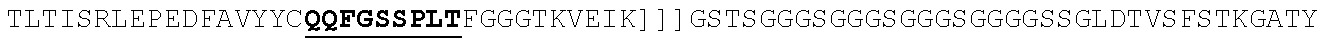

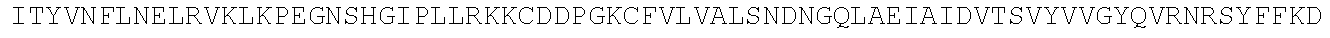

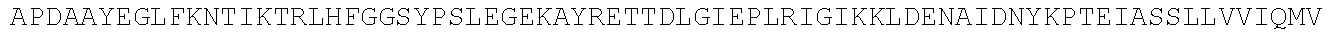

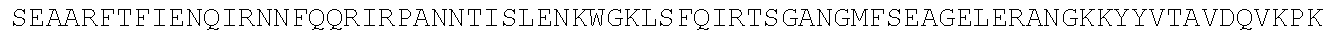

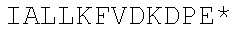

With Exotoxin A (N- or C-terminal):

Ab76: Full peptide sequence combined variable domains to scFv of Ab70 plus

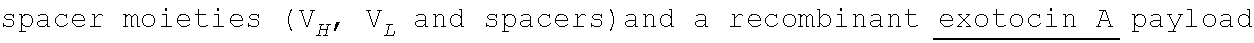

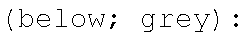

(SEQ ID NO: 99)

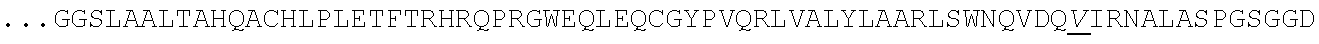

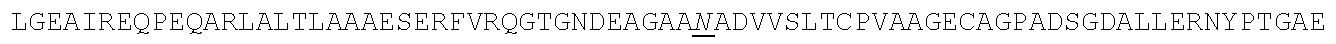

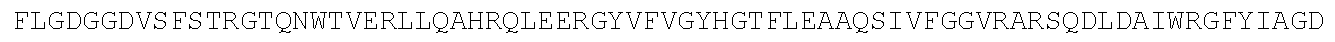

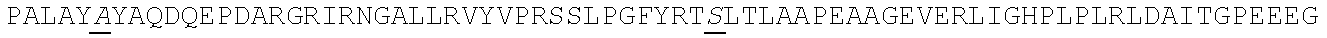

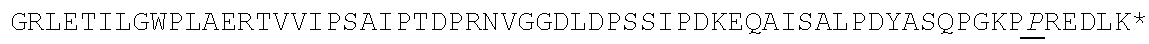

Ab77: Full peptide sequence combined variable domains to TaFv of Ab71 plus

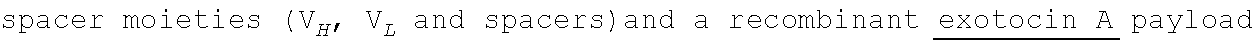

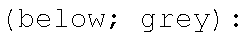

(SEQ ID NO: 99)

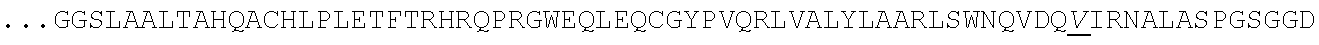

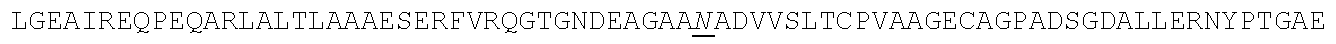

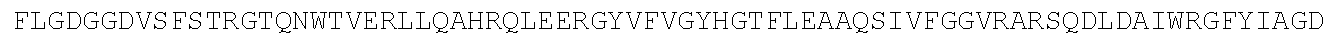

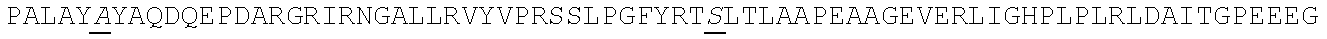

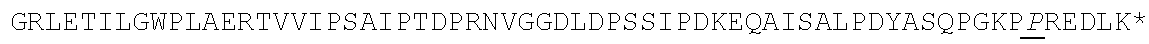

Ab78: Full peptide sequence combined variable domains to scFv of Ab72 plus

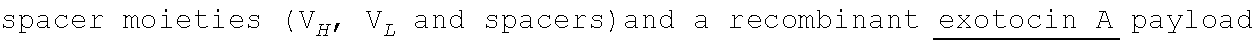

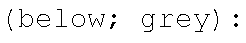

(SEQ ID NO: 99)

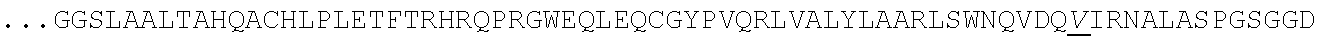

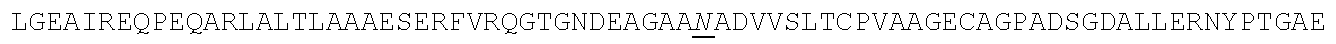

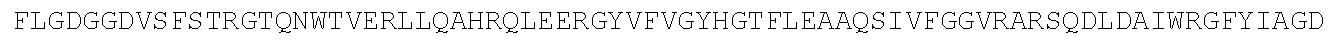

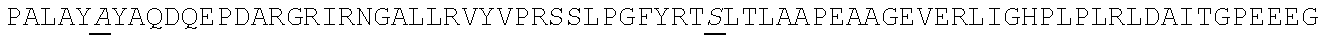

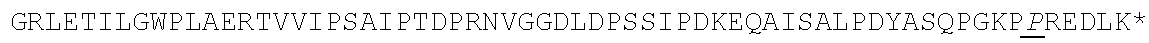

Ab79: Full peptide sequence combined variable domains to TaFv of Ab73 plus

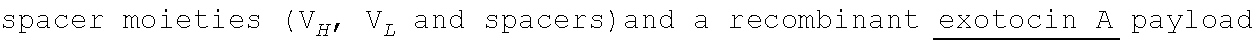

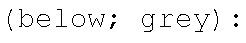

(SEQ ID NO: 99)

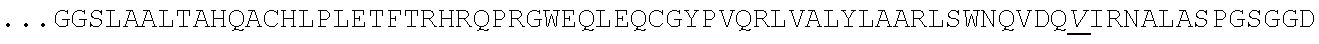

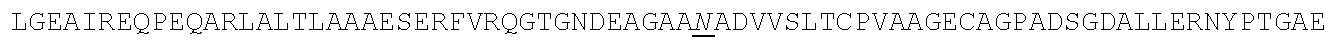

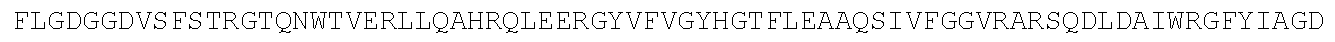

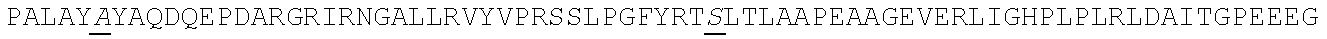

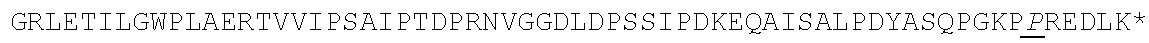

Ab80: Full peptide sequence combined variable domains to scFv of Ab74 plus

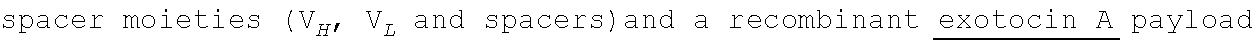

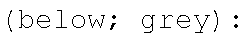

(SEQ ID NO: 99)

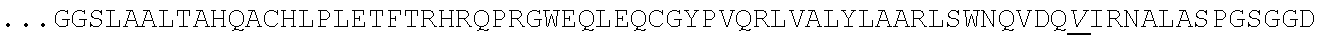

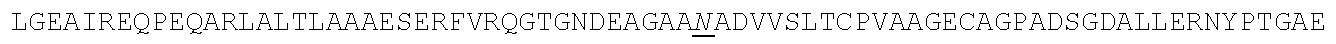

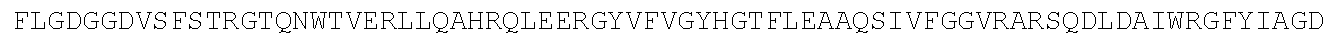

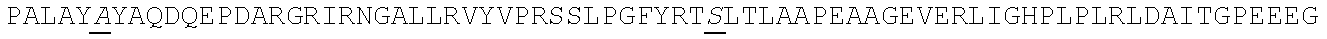

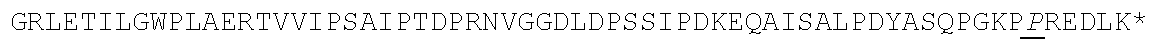

Ab81: Full peptide sequence combined variable domains to TaFv of Ab75 plus

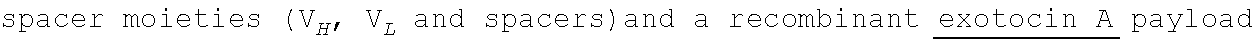

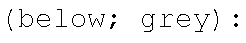

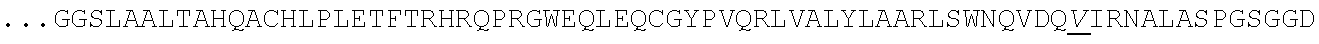

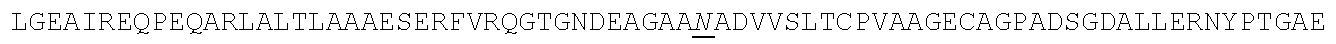

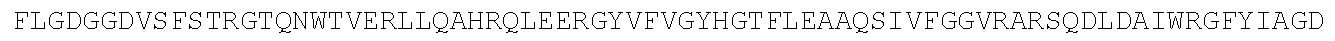

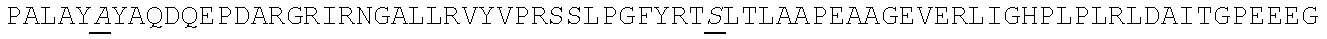

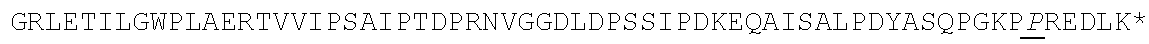

With Saporin (N- or C-terminal):

Ab82: Full peptide sequence combined variable domains to scFv of Ab70 plus

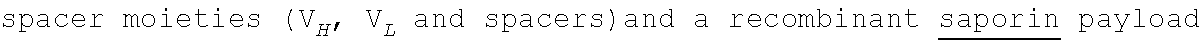

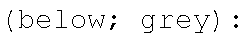

(SEQ ID NO: 100)

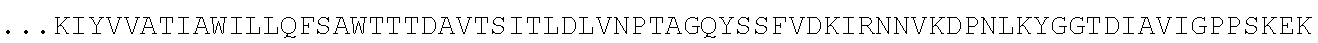

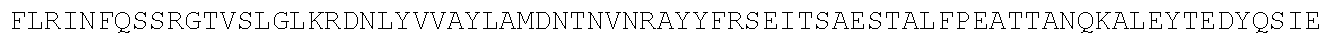

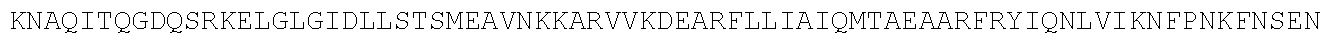

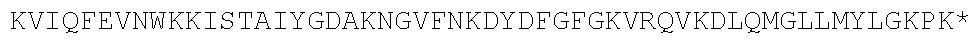

Ab83: Full peptide sequence combined variable domains to TaFv of Ab71 plus

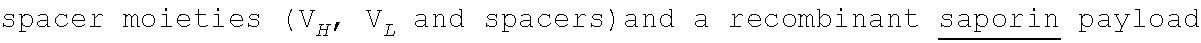

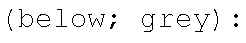

(SEQ ID NO: 100)

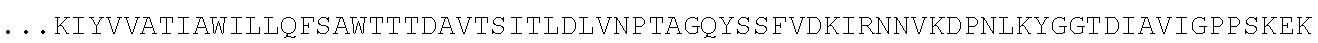

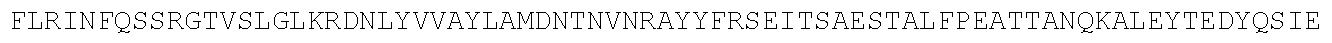

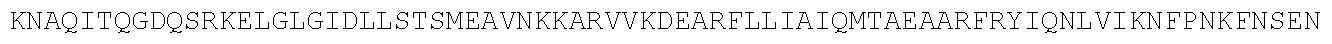

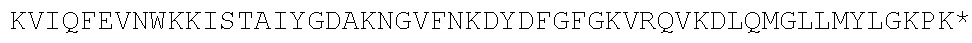

Ab84: Full peptide sequence combined variable domains to scFv of Ab72 plus

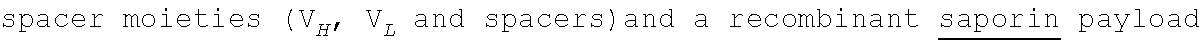

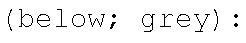

(SEQ ID NO: 100)

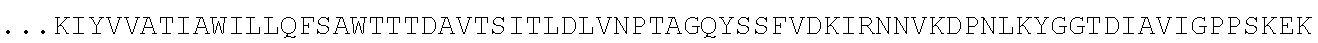

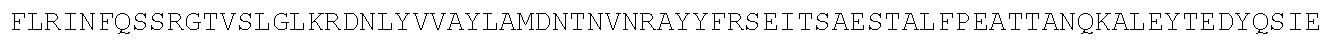

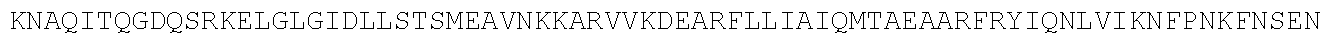

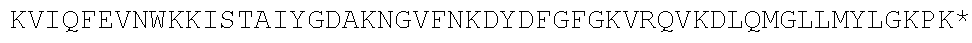

Ab85: Full peptide sequence combined variable domains to TaFv of Ab73 plus

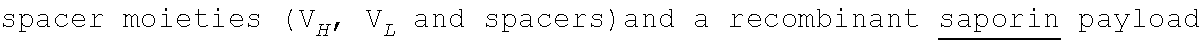

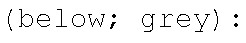

(SEQ ID NO: 100)

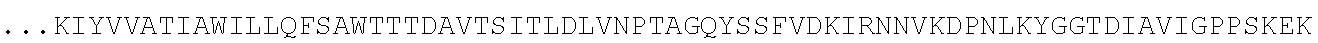

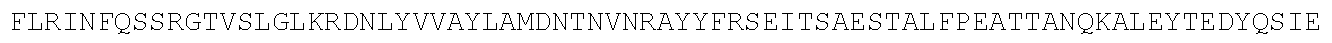

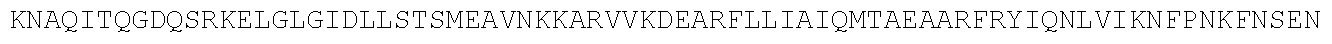

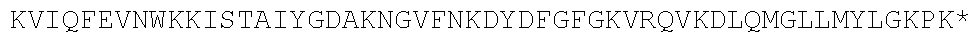

Ab86: Full peptide sequence combined variable domains to scFv of Ab74 plus

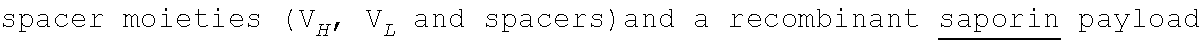

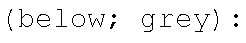

(SEQ ID NO: 100)

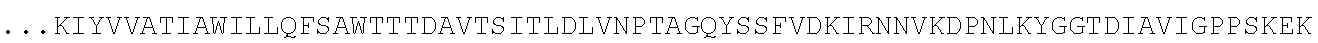

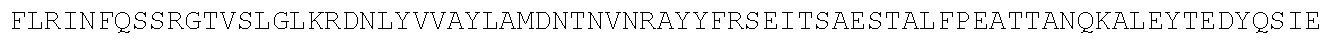

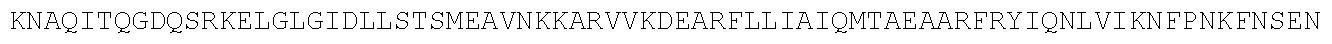

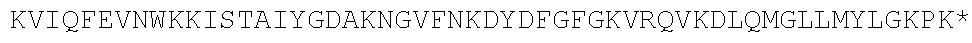

Ab87: Full peptide sequence combined variable domains to TaFv of Ab75 plus

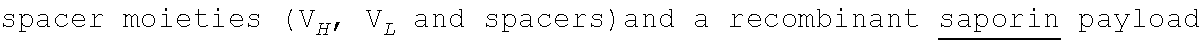

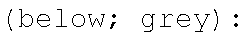

(SEQ ID NO: 100)

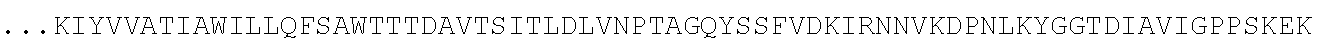

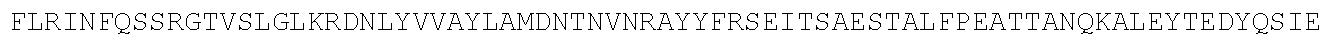

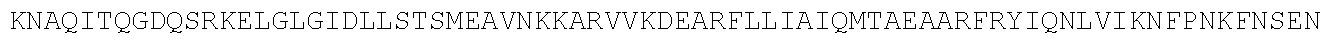

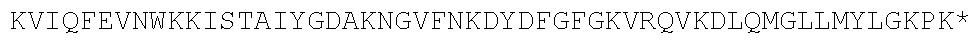

With Luffin P1 (N- or C-terminal):

Ab88: Full peptide sequence combined variable domains to scFv of Ab70 plus

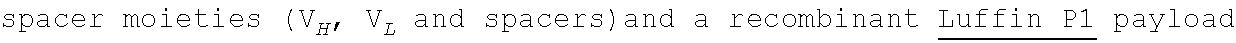

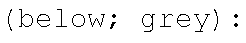

(SEQ ID NO: 101)

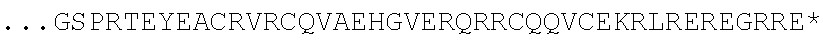

Ab89: Full peptide sequence combined variable domains to TaFv of Ab71 plus

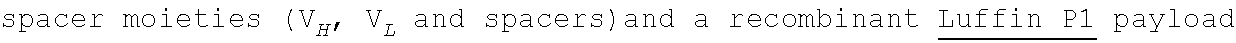

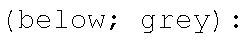

(SEQ ID NO: 101)

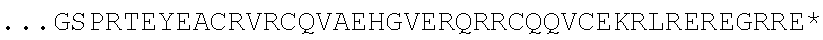

Ab90: Full peptide sequence combined variable domains to scFv of Ab72 plus

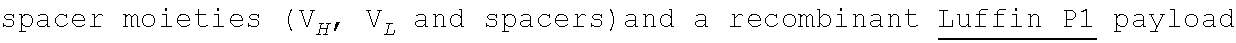

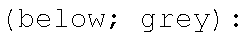

(SEQ ID NO: 101)

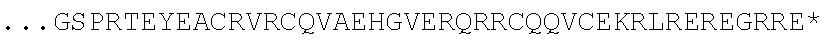

Ab91: Full peptide sequence combined variable domains to TaFv of Ab73 plus

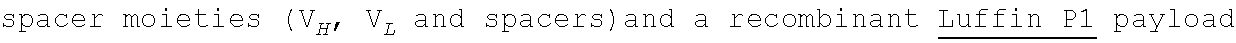

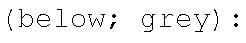

(SEQ ID NO: 101)

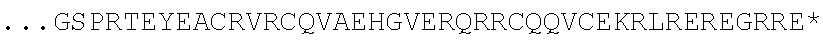

Ab92: Full peptide sequence combined variable domains to scFv of Ab74 plus

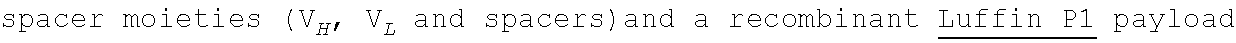

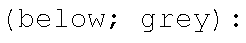

(SEQ ID NO: 101)

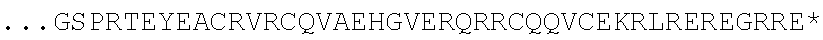

Ab93: Full peptide sequence combined variable domains to TaFv of Ab75 plus

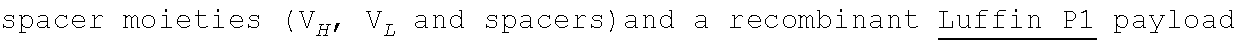

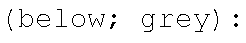

(SEQ ID NO: 101)

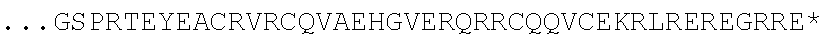

With Diphtheria toxin (N- or C-terminal):

Ab94: Full peptide sequence combined variable domains to scFv of Ab70 plus

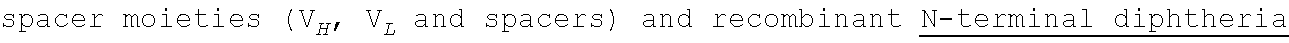

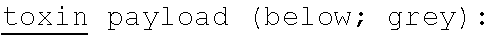

(SEQ ID NO: 30)

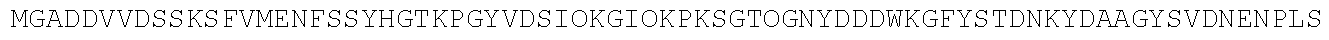

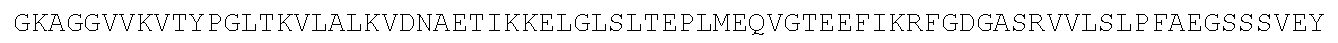

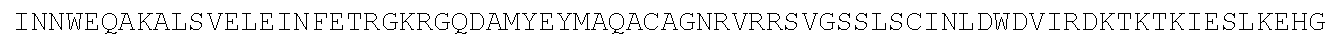

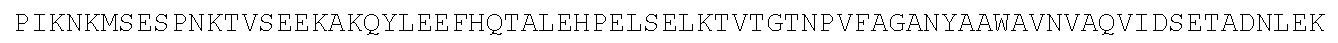

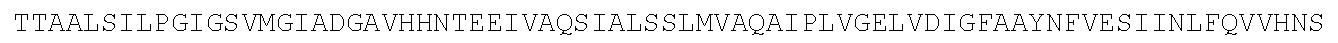

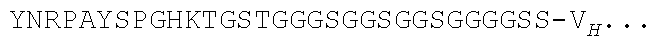

Ab95: Full peptide sequence combined variable domains to TaFv of Ab71 plus

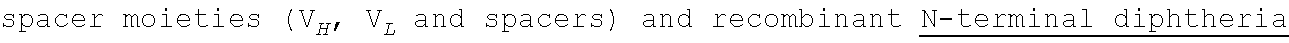

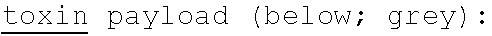

(SEQ ID NO: 30)

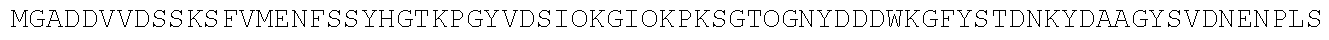

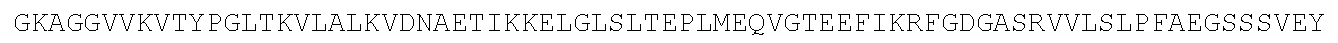

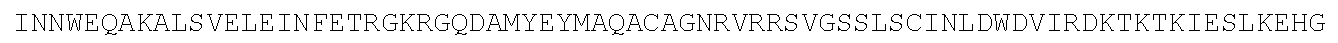

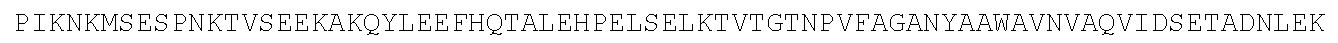

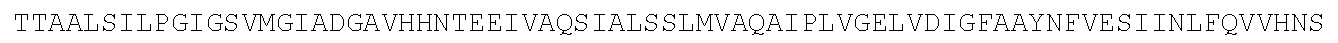

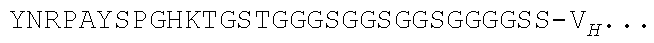

Ab96: Full peptide sequence combined variable domains to scFv of Ab72 plus

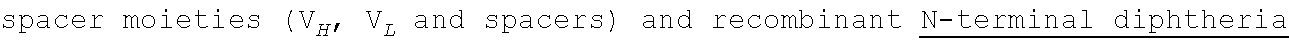

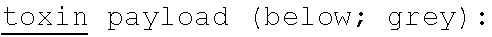

(SEQ ID NO: 30)

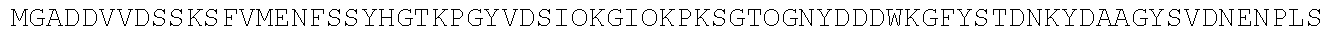

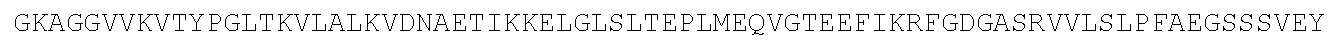

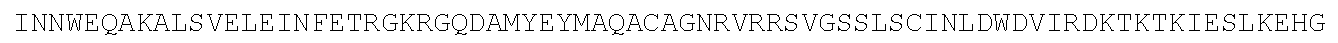

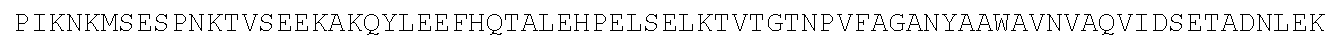

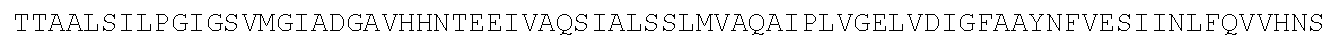

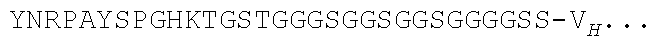

Ab97: Full peptide sequence combined variable domains to TaFv of Ab73 plus

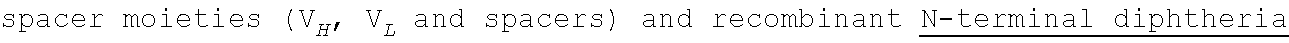

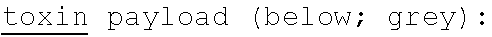

(SEQ ID NO: 30)

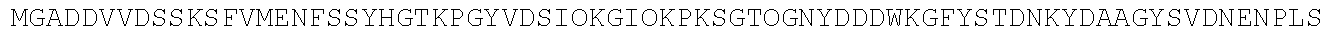

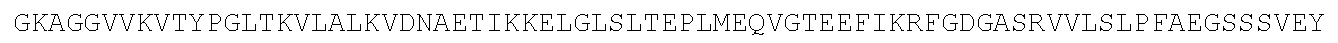

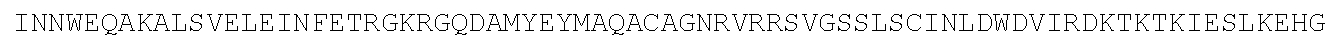

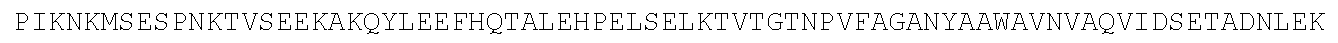

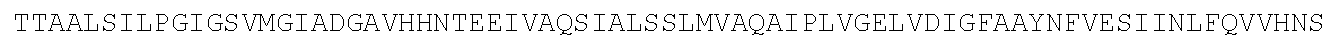

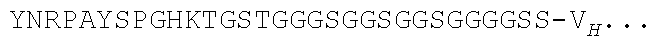

Ab98: Full peptide sequence combined variable domains to scFv of Ab74 plus

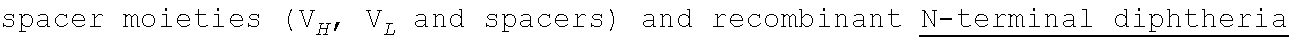

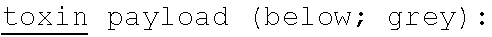

(SEQ ID NO: 30)

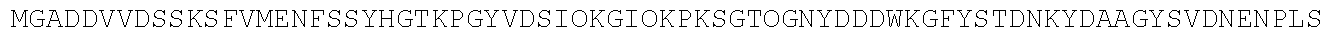

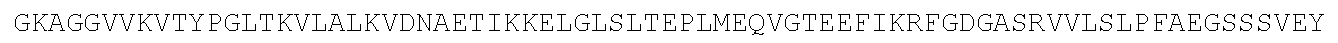

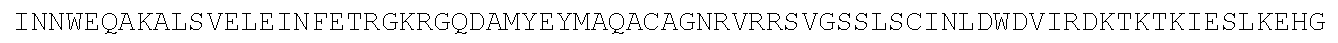

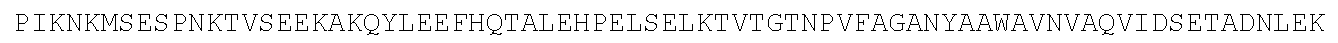

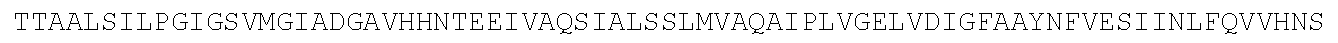

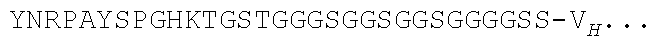

Ab99: Full peptide sequence combined variable domains to TaFv of Ab75 plus

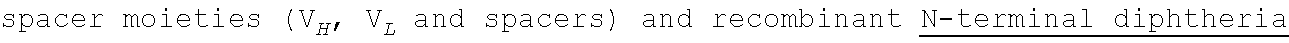

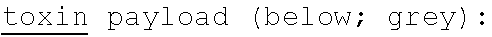

(SEQ ID NO: 30)

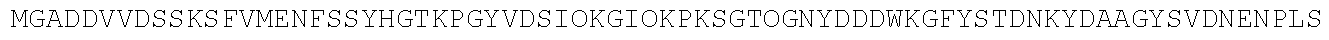

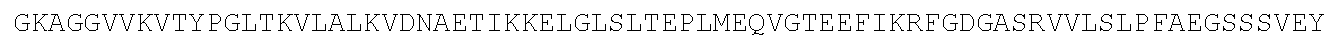

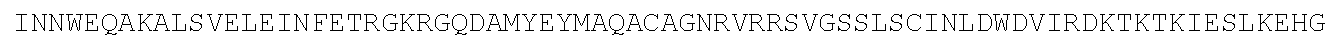

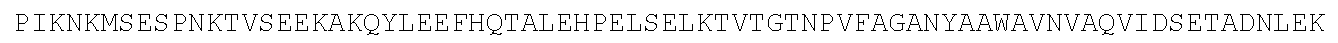

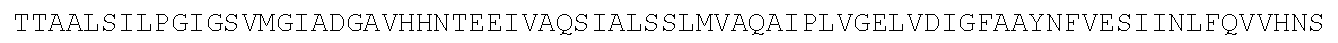

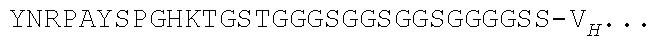

In some examples, the antibody binds the same epitope as an antibody comprising any of the VH chains known in the art and/or exemplified herein and/or competes against such an antibody from binding to the antigen. Such an antibody may comprise the same heavy chain CDRs as those exemplified herein. An antibody having the same CDR (e.g., CDR3) as a reference antibody means that the two antibodies have the same amino acid sequence in that CDR region as determined by the same methodology (e.g., the Kabat definition, the Chothia definition, the AbM definition, or the contact definition).

In some embodiments, the anti-ABCB5 antibody comprises a heavy chain variable region that comprises a heavy chain CDR1 (HC CDR1), a heavy chain CDR2 (HC CDR2), and a heavy chain CDR3 (HC CDR3). For example, following the Kabat definition, the HC CDR1 may comprise the amino acid sequence of GFTFSSYX₁MN (SEQ ID NO: 109) or GYTFTX₂YYMH (SEQ ID NO: 110), in which X₁ is S or D or T and X₂ is S or N; the HC CDR2 may comprise the amino acid sequence of YISSSX₃X₄TIYYADSVKG (SEQ ID NO: 111) or IINPSGGSTSYAQKFX₅G (SEQ ID NO: 112), in which X₃ is S or G, and X₄ is S or N; and X₅ is K or Q and/or the HC CDR3 may comprise the amino acid sequence of NYQYGDYGGY (SEQ ID NO: 66) or DX₆AVTGTAYYYYYGMDV (SEQ ID NO: 113), in which X₆ is Q or L.

In one embodiment, following the Kabat definition, the HC CDR1 may comprise the amino acid sequence of X₇ASHDISNFLN (SEQ ID NO: 114) or RASX₈SVNSX₉YLA (SEQ ID NO: 115), in which X₇ is Q or H; X₈ is L or Q; and X₉ is N or K; the HC CDR2 may comprise the amino acid sequence of DAYNLQT (SEQ ID NO: 77) or GTSSRAT (SEQ ID NO: 78); and/or the HC CDR3 may comprise the amino acid sequence of QQYDYFLSIT (SEQ ID NO: 79) or QQFGSSPLT (SEQ ID NO: 81).

Provided below are several exemplary anti-ABCB5 antibodies, ABCB5-Ab1-Ab101, including their heavy chain and light chain CDR sequences (by Kabat definition) and heavy chain and light chain full and variable region sequences.

TABLE 1
Heavy chain variable sequences of exemplary anti-ABCB5 antibodies

| Exemplary | | SEQ |
|---|---|---|
| Antibody | HC Sequence | ID NO. |
| ABCB5-Ab1 | EVQLLESGGGLVQPGGSLRLSCAASGFTFSSYSMNWVRQAPGKGLEWVSYISSSSSTIYYADSVKGR | 1 |
| | FTISRDNSKNTLYLQMNSLRAEDTAVYYCAKNYQYGDYGGYWGRGTLVTVSS | |
| ABCB5-Ab2 | EVQLVESGGGLVQPGGSLRLSCAASGFTFSSYDMNWVRQAPGKGLEWVSYISSSSNTIYYADSVKGR | 2 |
| | FTISRDNAKNSLYLQMNSLRDEDTAVYYCASNYQYGDYGGYWGRGTLVTVSS | |
| ABCB5-Ab3 | EVQLVESGGGLVQPGGSLRLSCAASGFTFSSYSMNWVRQAPGKGPEWVSYISSSGSTIYYADSVKGR | 3 |
| | FTISRDNAKNSLYLQMNSLRDEDTAVYYCASNYQYGDYGGYWGRGTLVTVSS | |
| ABCB5-Ab4 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab5 | EVQLVESGGGLVQPGGSLRLSCAASGFTFSSYSMNWVRQAPGKGLEWVSYISSSSSTIYYADSVKGR | 5 |
| | FTISRDNAKNSLYLQMNSLRDEDTAVYYCASNYQYGDYGGYWGRGTLVTVSS | |
| ABCB5-Ab6 | EVQLLESGGGLVQPGGSLRLSCAASGFTFSSYDMNWVRQAPGKGLEWVSYISSSSNTIYYADSVKGR | 6 |
| | FTISRDNSKNTLYLQMNSLRAEDTAVYYCAKNYQYGDYGGYWGRGTLVTVSS | |
| ABCB5-Ab7 | EVQLLESGGGLVQPGGSLRLSCAASGFTFSSYSMNWVRQAPGKGPEWVSYISSSGSTIYYADSVKGR | 7 |
| | FTISRDNSKNTLYLQMNSLRAEDTAVYYCAKNYQYGDYGGYWGRGTLVTVSS | |
| ABCB5-Ab8 | EVQLLESGGGLVQPGGSLRLSCAASGFTFSSYSMNWVRQAPGKGPEWVSYISSSSSTIYYADSVKGRF | 8 |
| | TISRDNSKNTLYLQMNSLRAEDTAVYYCAKNYQYGDYGGYWGRGTLVTVSS | |
| ABCB5-Ab9 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab10 | EVQLVESGGGLVQPGGSLRLSCAASGFTFSSYDMNWVRQAPGKGLEWVSYISSSSNTIYYADSVKGR | 2 |
| | FTISRDNAKNSLYLQMNSLRDEDTAVYYCASNYQYGDYGGYWGRGTLVTVSS | |
| ABCB5-Ab11 | EVQLVESGGGLVQPGGSLRLSCAASGFTFSSYSMNWVRQAPGKGPEWVSYISSSGSTIYYADSVKGR | 3 |
| | FTISRDNAKNSLYLQMNSLRDEDTAVYYCASNYQYGDYGGYWGRGTLVTVSS | |
| ABCB5-Ab12 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab13 | EVQLLESGGGLVQPGGSLRLSCAASGFTFSSYDMNWVRQAPGKGLEWVSYISSSSNTIYYADSVKGR | 6 |
| | FTISRDNSKNTLYLQMNSLRAEDTAVYYCAKNYQYGDYGGYWGRGTLVTVSS | |
| ABCB5-Ab14 | EVQLLESGGGLVQPGGSLRLSCAASGFTFSSYSMNWVRQAPGKGPEWVSYISSSGSTIYYADSVKGR | 7 |
| | FTISRDNSKNTLYLQMNSLRAEDTAVYYCAKNYQYGDYGGYWGRGTLVTVSS | |
| ABCB5-Ab15 | EVQLLESGGGLVQPGGSLRLSCAASGFTFSSYSMNWVRQAPGKGPEWVSYISSSSSTIYYADSVKGRF | 8 |
| | TISRDNSKNTLYLQMNSLRAEDTAVYYCAKNYQYGDYGGYWGRGTLVTVSS | |
| ABCB5-Ab16 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab17 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab18 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab19 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab20 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab21 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab22 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab23 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab24 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab25 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab26 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab27 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab28 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab29 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab30 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab31 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab32 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab33 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab34 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab35 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab36 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab37 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab38 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab39 | EVQLLESGGGLVQPGGSLRLSCAASGFTFSSYSMNWVRQAPGKGLEWVSYISSSSSTIYYADSVKGRF | 1 |
| | TISRDNSKNTLYLQMNSLRAEDTAVYYCAKNYQYGDYGGYWGRGTLVTVSS | |
| ABCB5-Ab40 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab41 | EVQLVESGGGLVQPGGSLRLSCAASGFTFSSYSMNWVRQAPGKGPEWVSYISSSSSTIYYADSVKGR | 4 |
| | FTISRDNAKNSLYLQMNSLRDEDTAVYYCASNYQYGDYGGYWGRGTLVTVSS | |
| ABCB5-Ab42 | QVQLVQSGAEVKKPGASVKVSCKTSGYTFTSYYMHWVRQSPGQGLEWMGIINPSGGSTSYAQKFQG | 13 |
| | RVTMTRDTSTSTVYMELSSLRSEDTAVYYCARDQAVTGTAYYYYYGMDVWGQGTTVTVSS | |
| ABCB5-Ab43 | QVQLVQSGAEVKKPGASVKVSCKASGYTFTSYYMHWVRQSPGQGLEWMGIINPSGGSTSYAQKFQ | 14 |
| | GRVTMTRDTSTSTVYMELSSLRSEDTAVYYCARDQAVTGTAYYYYYGMDVWGQGTTVTVSS | |
| ABCB5-Ab44 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab45 | QVQLVQSGAEVKKPGASVKVSCKASGYTFTNYYMHWVRQAPGQGPEWMGIINPSGGSTSYAQKFQ | 16 |
| | GRVTMTRDTSTSTVYMELSSLRSEDTAVYYCARDLAVTGTAYYYYYGMDVWGQGTTVTVSS | |
| ABCB5-Ab46 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab47 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab48 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab49 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab50 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab51 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab52 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab53 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab54 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab55 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab56 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab57 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab58 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab59 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab60 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab61 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab62 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab63 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab64 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab65 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab66 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab67 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab68 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab69 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab70 | EVQLLESGGGLVQPGGSLRLSCAASGFTFSSYSMNWVRQAPGKGPEWVSYISSSSSTIYYADSVKGR | 8 |
| | FTISRDNSKNTLYLQMNSLRAEDTAVYYCAKNYQYGDYGGYWGRGTLVTVSS | |
| ABCB5-Ab71 | EVQLLESGGGLVQPGGSLRLSCAASGFTFSSYSMNWVRQAPGKGPEWVSYISSSSSTIYYADSVKGR | 8 |
| | FTISRDNSKNTLYLQMNSLRAEDTAVYYCAKNYQYGDYGGYWGRGTLVTVSS | |
| | EVQLLESGGGLVQPGGSLRLSCAASGFTFSSYSMNWVRQAPGKGPEWVSYISSSSSTIYYADSVKGR | 8 |
| | FTISRDNSKNTLYLQMNSLRAEDTAVYYCAKNYQYGDYGGYWGRGTLVTVSS | |
| ABCB5-Ab72 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab73 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |
| ABCB5-Ab74 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab75 | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| | QVQLVQSGAEVKKPGASVKVSCKASGYTFTSYYMHWVRQAPGQGLEWMGIINPSGGSTSYAQKFK | 15 |
| | GRVTMTRDTSTSTVYMELSSLRSEDTAVYYCARDQAVTGTAYYYYYGMDVWGQGTTVTVSS | |
| ABCB5-Ab100 | EVQLVESGGGLVQPGGSLRLSCAASGFTFSSYSMNWVRQAPGKGLEWVSYISSSSSTIYYADSVKGR | 5 |
| | FTISRDNAKNSLYLQMNSLRDEDTAVYYCASNYQYGDYGGYWGRGTLVTVSS | |
| ABCB5-Ab101 | QVQLVQSGAEVKKPGASVKVSCKASGYTFTSYYMHWVRQAPGQGLEWMGIINPSGGSTSYAQKFQ | 17 |
| | GRVTMTRDTSTSTVYMELSSLRSEDTAVYYCARDQAVTGTAYYYYYGMDVWGQGTTVTVSS | |

TABLE 2
Light chain variable sequences of exemplary anti-ABCB5 antibodies

| Exemplary | | SEQ |
|---|---|---|
| Antibody | LC Sequence | ID NO. |
| ABCB5-Ab1 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab2 | AIQLTQSPSSLTGSVGDRVTITCHASHDISNFLNWYQQKPGQAPKLLIYDAYNLQTGVPSRFSGTGSG | 21 |
| | THFTLTINSLQPEDVGTYFCQQYDYFLSITFGQGTRLEIK | |
| ABCB5-Ab3 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab4 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab6 | DIQMTQSPSSLSASVGDRVTITCHASHDISNFLNWYQQKPGKAPKLLIYDAYNLQTGVPSRFSGSGSG | 23 |
| | TDFTFTISSLQPEDIATYYCQQYDYFLSITFGQGTKVEIK | |
| ABCB5-Ab7 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab8 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab9 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab10 | AIQLTQSPSSLTGSVGDRVTITCHASHDISNFLNWYQQKPGQAPKLLIYDAYNLQTGVPSRFSGTGSG | 21 |
| | THFTLTINSLQPEDVGTYFCQQYDYFLSITFGQGTRLEIK | |
| ABCB5-Ab11 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab12 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab13 | DIQMTQSPSSLSASVGDRVTITCHASHDISNFLNWYQQKPGKAPKLLIYDAYNLQTGVPSRFSGSGSG | 23 |
| | TDFTFTISSLQPEDIATYYCQQYDYFLSITFGQGTKVEIK | |
| ABCB5-Ab14 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab15 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab16 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab17 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab18 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab19 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab20 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab21 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab22 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab23 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab24 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab25 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab26 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab27 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab28 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab29 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab30 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab31 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab32 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab33 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab34 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab35 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab36 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab37 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab38 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab39 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab40 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab41 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab42 | EIVLTQSPGTLSLSPGERATLSCRASLSVNSNYLAWYQQKPGQAPRLLIYGTSSRATGIPDSFSGSGSG | 26 |
| | TDFTLTISRLEPEDFAVYYCQQFGSSPLTFGGGTKVEIK | |
| ABCB5-Ab43 | EIVLTQSPGTLSLSPGERATLSCRASQSVNSKYLAWYQQKPGQAPRLLIYGTSSRATGIPDSFSGSGSG | 27 |
| | TDFTLTISRLEPEDFAVYYCQQFGSSPLTFGGGTKVEIK | |
| ABCB5-Ab44 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab45 | EFVLTQSPGTLSLSPGERATLSCRASQSVNSNYLAWYQQKPGQAPRLLIYGTSSRATGIPDSFSGSGS | 29 |
| | GTDFTLTISRLEPEDFAVYYCQQFGSSPLTFGGGTKVEIK | |
| ABCB5-Ab46 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab47 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab48 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab49 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab50 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab51 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab52 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab53 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab54 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab55 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab56 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab57 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab58 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab59 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab60 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab61 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab62 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab63 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab64 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab65 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab66 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab67 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab68 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab69 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab70 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab71 | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| | DIQMTQSPSSLSASVGDRVTITCQASHDISNFLNWYQQKPGKAPKLLIYDAYNLQTGVPSRFSGSGSG | 20 |
| | TDFTFTISSLQPEDIATYYCQQYDYFLSITFGQGTKVEIK | |
| ABCB5-Ab72 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab73 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab74 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab75 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |
| ABCB5-Ab100 | AIQLTQSPSSLTGSVGDRVTITCQASHDISNFLNWYQQKPGQAPKLLIYDAYNLQTGVPSRFSGTGSG | 22 |
| | THFTLTINSLQPEDVGTYFCQQYDYFLSITFGQGTRLEIK | |
| ABCB5-Ab101 | EIVLTQSPGTLSLSPGERATLSCRASQSVNSNYLAWYQQKPGQAPRLLIYGTSSRATGIPDSFSGSGSG | 28 |
| | TDFTLTISRLEPEDFAVYYCQQFGSSPLTFGGGTKVEIK | |

TABLE 3
Heavy chain sequences of exemplary anti-ABCB5 antibodies

| Exemplary | | SEQ |
|---|---|---|
| Antibody | HC Sequence | ID NO. |
| ABCB5-Ab5 | EVQLVESGGGLVQPGGSLRLSCAASGFTFSSYSMNWVRQAPGKGLEWVSYISSSSSTIYYADSVKGRF | 31 |
| | TISRDNAKNSLYLQMNSLRDEDTAVYYCASNYQYGDYGGYWGRGTLVTVSSASTKGPSVFPLAPSSK | |
| | STSGGTAALGCLVKDYFPEPVTVSWNSGALTSGVHTFPAVLQSSGLYSLSSVVTVPSSSLGTQTYICNV | |
| | NHKPSNTKVDKKVEPKSCDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKTTPPVLDSDGSFF | |
| | LYSKLTVDKSRWQQGNVFSCSVMHEALKFHYTQKSLSLSPGK | |
| ABCB5-Ab9 | EVQLLESGGGLVQPGGSLRLSCAASGFTFSSYSMNWVRQAPGKGLEWVSYISSSSSTIYYADSVKGRF | 32 |
| | TISRDNSKNTLYLQMNSLRAEDTAVYYCAKNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALKFHYTQKSLSLSPGK | |
| ABCB5-Ab10 | EVQLVESGGGLVQPGGSLRLSCAASGFTFSSYDMNWVRQAPGKGLEWVSYISSSSNTIYYADSVKGR | 33 |
| | FTISRDNAKNSLYLQMNSLRDEDTAVYYCASNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALKFHYTQKSLSLSPGK | |
| ABCB5-Ab11 | EVQLVESGGGLVQPGGSLRLSCAASGFTFSSYSMNWVRQAPGKGPEWVSYISSSGSTIYYADSVKGRF | 34 |
| | TISRDNAKNSLYLQMNSLRDEDTAVYYCASNYQYGDYGGYWGRGTLVTVSSASTKGPSVFPLAPSSK | |
| | STSGGTAALGCLVKDYFPEPVTVSWNSGALTSGVHTFPAVLQSSGLYSLSSVVTVPSSSLGTQTYICNV | |
| | NHKPSNTKVDKKVEPKSCDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKTTPPVLDSDGSFF | |
| | LYSKLTVDKSRWQQGNVFSCSVMHEALKFHYTQKSLSLSPGK | |
| ABCB5-Ab12 | EVQLVESGGGLVQPGGSLRLSCAASGFTFSSYSMNWVRQAPGKGPEWVSYISSSSSTIYYADSVKGRF | 35 |
| | TISRDNAKNSLYLQMNSLRDEDTAVYYCASNYQYGDYGGYWGRGTLVTVSSASTKGPSVFPLAPSSK | |
| | STSGGTAALGCLVKDYFPEPVTVSWNSGALTSGVHTFPAVLQSSGLYSLSSVVTVPSSSLGTQTYICNV | |
| | NHKPSNTKVDKKVEPKSCDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKTTPPVLDSDGSFF | |
| | LYSKLTVDKSRWQQGNVFSCSVMHEALKFHYTQKSLSLSPGK | |
| ABCB5-Ab13 | EVQLLESGGGLVQPGGSLRLSCAASGFTFSSYDMNWVRQAPGKGLEWVSYISSSSNTIYYADSVKGR | 36 |
| | FTISRDNSKNTLYLQMNSLRAEDTAVYYCAKNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALKFHYTQKSLSLSPGK | |
| ABCB5-Ab14 | EVQLLESGGGLVQPGGSLRLSCAASGFTFSSYSMNWVRQAPGKGPEWVSYISSSGSTIYYADSVKGRF | 37 |
| | TISRDNSKNTLYLQMNSLRAEDTAVYYCAKNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALKFHYTQKSLSLSPGK | |
| ABCB5-Ab15 | EVQLLESGGGLVQPGGSLRLSCAASGFTFSSYSMNWVRQAPGKGPEWVSYISSSSSTIYYADSVKGRF | 37 |
| | TISRDNSKNTLYLQMNSLRAEDTAVYYCAKNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALKFHYTQKSLSLSPGK | |
| ABCB5-Ab16 | EVQLLESGGGLVQPGGSLRLSCAASGFTFSSYSMNWVRQAPGKGLEWVSYISSSSSTIYYADSVKGRF | 38 |
| | TISRDNSKNTLYLQMNSLRAEDTAVYYCAKNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVEDVSHE | |
| | DPEVKFNWYVDGVEVHNAKTKPREEQYNGAYRVVSVLTVLHQDWLNGKEYKCKVSNIYGPAPIEK | |
| | TISKAKGQPREPQVYALPPSREEMTKNQVSLTCLVKGFYPSDIAVEWVSNGQPENDYKTTPPVLDSDG | |
| | SFFLYSKLTVDKSRWQQGNVFSCSVLHEALHNHYTQKSLSLSPGK | |
| ABCB5-Ab17 | EVQLVESGGGLVQPGGSLRLSCAASGFTFSSYSMNWVRQAPGKGPEWVSYISSSSSTIYYADSVKGRF | 39 |
| | TISRDNAKNSLYLQMNSLRDEDTAVYYCASNYQYGDYGGYWGRGTLVTVSSASTKGPSVFPLAPSSK | |
| | STSGGTAALGCLVKDYFPEPVTVSWNSGALTSGVHTFPAVLQSSGLYSLSSVVTVPSSSLGTQTYICNV | |
| | NHKPSNTKVDKKVEPKSCDKTHTCPPCPAPELLGGPSVFLFPPKPKDTLMISRTPEVTCVVEDVSHEDP | |
| | EVKFNWYVDGVEVHNAKTKPREEQYNGAYRVVSVLTVLHQDWLNGKEYKCKVSNIYGPAPIEKTIS | |
| | KAKGQPREPQVYALPPSREEMTKNQVSLTCLVKGFYPSDIAVEWVSNGQPENDYKTTPPVLDSDGSF | |
| | FLYSKLTVDKSRWQQGNVFSCSVLHEALHNHYTQKSLSLSPGK | |
| ABCB5-Ab18 | EVQLLESGGGLVQPGGSLRLSCAASGFTFSSYSMNWVRQAPGKGLEWVSYISSSSSTIYYADSVKGRF | 40 |
| | TISRDNSKNTLYLQMNSLRAEDTAVYYCAKNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| ABCB5-Ab19 | EVQLVESGGGLVQPGGSLRLSCAASGFTFSSYSMNWVRQAPGKGPEWVSYISSSSSTIYYADSVKGRF | 41 |
| | TISRDNAKNSLYLQMNSLRDEDTAVYYCASNYQYGDYGGYWGRGTLVTVSSASTKGPSVFPLAPSSK | |
| | STSGGTAALGCLVKDYFPEPVTVSWNSGALTSGVHTFPAVLQSSGLYSLSSVVTVPSSSLGTQTYICNV | |
| | NHKPSNTKVDKKVEPKSCDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| ABCB5-Ab22 | EVQLLESGGGLVQPGGSLRLSCAASGFTFSSYSMNWVRQAPGKGLEWVSYISSSSSTIYYADSVKGRF | 40 |
| | TISRDNSKNTLYLQMNSLRAEDTAVYYCAKNYQYGDYGGYWGRGTLVTVSSASTKGPSVFPLAPSS | |
| | KSTSGGTAALGCLVKDYFPEPVTVSWNSGALTSGVHTFPAVLQSSGLYSLSSVVTVPSSSLGTQTYICN | |
| | VNHKPSNTKVDKKVEPKSCDKTHTCPPCPAPELLGGPSVFLFPPKPKDTLMISRTPEVTCVVVDVSHE | |
| | DPEVKFNWYVDGVEVHNAKTKPREEQYNSTYRVVSVLTVLHQDWLNGKEYKCKVSNKALPAPIEK | |
| | TISKAKGQPREPQVYTLPPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| ABCB5-Ab23 | EVQLVESGGGLVQPGGSLRLSCAASGFTFSSYSMNWVRQAPGKGPEWVSYISSSSSTIYYADSVKGRF | 41 |
| | TISRDNAKNSLYLQMNSLRDEDTAVYYCASNYQYGDYGGYWGRGTLVTVSSASTKGPSVFPLAPSSK | |
| | STSGGTAALGCLVKDYFPEPVTVSWNSGALTSGVHTFPAVLQSSGLYSLSSVVTVPSSSLGTQTYICNV | |
| | NHKPSNTKVDKKVEPKSCDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| ABCB5-Ab46 | QVQLVQSGAEVKKPGASVKVSCKASGYTFTSYYMHWVRQAPGQGLEWMGIINPSGGSTSYAQKFQG | 42 |
| | RVTMTRDTSTSTVYMELSSLRSEDTAVYYCARDQAVTGTAYYYYYGMDVWGQGTTVTVSSASTKG | |
| | PSVFPLAPSSKSTSGGTAALGCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALPAPIEKTISKAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| ABCB5-Ab48 | QVQLVQSGAEVKKPGASVKVSCKASGYTFTSYYMHWVRQAPGQGLEWMGIINPSGGSTSYAQKFQG | 42 |
| | RVTMTRDTSTSTVYMELSSLRSEDTAVYYCARDQAVTGTAYYYYYGMDVWGQGTTVTVSSASTKG | |
| | PSVFPLAPSSKSTSGGTAALGCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALPAPIEKTISKAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| ABCB5-Ab58 | QVQLVQSGAEVKKPGASVKVSCKASGYTFTSYYMHWVRQAPGQGLEWMGIINPSGGSTSYAQKFKG | 43 |
| | RVTMTRDTSTSTVYMELSSLRSEDTAVYYCARDQAVTGTAYYYYYGMDVWGQGTTVTVSSASTKG | |
| | PSVFPLAPSSKSTSGGTAALGCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALPAPIEKTISKAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| ABCB5-Ab60 | QVQLVQSGAEVKKPGASVKVSCKASGYTFTSYYMHWVRQAPGQGLEWMGIINPSGGSTSYAQKFKG | 43 |
| | RVTMTRDTSTSTVYMELSSLRSEDTAVYYCARDQAVTGTAYYYYYGMDVWGQGTTVTVSSASTKG | |
| | PSVFPLAPSSKSTSGGTAALGCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALPAPIEKTISKAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |
| ABCB5-Ab100 | EVQLVESGGGLVQPGGSLRLSCAASGFTFSSYSMNWVRQAPGKGLEWVSYISSSSSTIYYADSVKGRF | 44 |
| | TISRDNAKNSLYLQMNSLRDEDTAVYYCASNYQYGDYGGYWGRGTLVTVSSASTKGPSVFPLAPSSK | |
| | STSGGTAALGCLVKDYFPEPVTVSWNSGALTSGVHTFPAVLQSSGLYSLSSVVTVPSSSLGTQTYICNV | |
| | NHKPSNTKVDKKVEPKSCDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKCKVSNKALPAPIEKTIS | |
| | KAKGQPREPQVYTLPPSREEMTKNQVSLTCLVKGFYPSDIAVEWESNGQPENNYKTTPPVLDSDGSFF | |
| | LYSKLTVDKSRWQQGNVFSCSVMHEALHNHYTQKSLSLSPGK | |
| ABCB5-Ab101 | QVQLVQSGAEVKKPGASVKVSCKASGYTFTSYYMHWVRQAPGQGLEWMGIINPSGGSTSYAQKFQG | 42 |
| | RVTMTRDTSTSTVYMELSSLRSEDTAVYYCARDQAVTGTAYYYYYGMDVWGQGTTVTVSSASTKG | |
| | PSVFPLAPSSKSTSGGTAALGCLVKDYFPEPVTVSWNSGALTSGVHTFPAVLQSSGLYSLSSVVTVPSS | |
| | SLGTQTYICNVNHKPSNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQDWLNGKEYKCKVS | |
| | NKALPAPIEKTISKAKGQPREPQVYTLPPSREEMTKNQVSLTCLVKGFYPSDIAVEWESNGQPENNYK | |
| | TTPPVLDSDGSFFLYSKLTVDKSRWQQGNVFSCSVMHEALHNHYTQKSLSLSPGK | |

TABLE 4
Light chain sequences of exemplary anti-ABCB5 antibodies

| Exemplary | | SEQ |
|---|---|---|
| Antibody | LC Sequence | ID NO. |
| ABCB5-Ab20 | DIQMTQSPSSLSASVGDRVTITCQASHDISNFLNWYQQKPGKAPKLLIYDAYNLQTGVPSRFSGSGSGT | 45 |
| | DFTFTISSLQPEDIATYYCQQYDYFLSITFGQGTKVEIKRTVAAPSVFIFPPSDEQLKSGTASVVCLLNNF | |
| | YPREAKVQWKVDNALQSGNSQESVTEQDSKDSTYSLSSTLTLSKADYEKHKVYACEVTHQGLSSPVT | |
| | KSFNRGEC | |
| ABCB5-Ab21 | AIQLTQSPSSLTGSVGDRVTITCQASHDISNFLNWYQQKPGQAPKLLIYDAYNLQTGVPSRFSGTGSGT | 46 |
| | HFTLTINSLQPEDVGTYFCQQYDYFLSITFGQGTRLEIKRTVAAPSVFIFPPSDEQLKSGTASVVCLLNN | |
| | FYPREAKVQWKVDNALQSGNSQESVTEQDSKDSTYSLSSTLTLSKADYEKHKVYACEVTHQGLSSPV | |
| | TKSFNRGEC | |
| ABCB5-Ab24 | DIQMTQSPSSLSASVGDRVTITCQASHDISNFLNWYQQKPGKAPKLLIYDAYNLQTGVPSRFSGSGSGT | 45 |
| | DFTFTISSLQPEDIATYYCQQYDYFLSITFGQGTKVEIKRTVAAPSVFIFPPSDEQLKSGTASVVCLLNNF | |
| | YPREAKVQWKVDNALQSGNSQESVTEQDSKDSTYSLSSTLTLSKADYEKHKVYACEVTHQGLSSPVT | |
| | KSFNRGEC | |
| ABCB5-Ab25 | AIQLTQSPSSLTGSVGDRVTITCQASHDISNFLNWYQQKPGQAPKLLIYDAYNLQTGVPSRFSGTGSGT | 46 |
| | HFTLTINSLQPEDVGTYFCQQYDYFLSITFGQGTRLEIKRTVAAPSVFIFPPSDEQLKSGTASVVCLLNN | |
| | FYPREAKVQWKVDNALQSGNSQESVTEQDSKDSTYSLSSTLTLSKADYEKHKVYACEVTHQGLSSPV | |
| | TKSFNRGEC | |
| ABCB5-Ab47 | EIVLTQSPGTLSLSPGERATLSCRASQSVNSNYLAWYQQKPGQAPRLLIYGTSSRATGIPDSFSGSGSGT | 47 |
| | DFTLTISRLEPEDFAVYYCQQFGSSPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKADYEKHKVYACEVTHQGLSSPVT | |
| | KSFNRGEC | |
| ABCB5-Ab49 | EIVLTQSPGTLSLSPGERATLSCRASQSVNSNYLAWYQQKPGQAPRLLIYGTSSRATGIPDSFSGSGSGT | 47 |
| | DFTLTISRLEPEDFAVYYCQQFGSSPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKADYEKHKVYACEVTHQGLSSPVT | |
| | KSFNRGEC | |
| ABCB5-Ab59 | EIVLTQSPGTLSLSPGERATLSCRASQSVNSNYLAWYQQKPGQAPRLLIYGTSSRATGIPDSFSGSGSGT | 47 |
| | DFTLTISRLEPEDFAVYYCQQFGSSPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKADYEKHKVYACEVTHQGLSSPVT | |
| | KSFNRGEC | |
| ABCB5-Ab61 | EIVLTQSPGTLSLSPGERATLSCRASQSVNSNYLAWYQQKPGQAPRLLIYGTSSRATGIPDSFSGSGSGT | 47 |
| | DFTLTISRLEPEDFAVYYCQQFGSSPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKADYEKHKVYACEVTHQGLSSPVT | |
| | KSFNRGEC | |
| ABCB5-Ab100 | AIQLTQSPSSLTGSVGDRVTITCQASHDISNFLNWYQQKPGQAPKLLIYDAYNLQTGVPSRFSGTGSGT | 46 |
| | HFTLTINSLQPEDVGTYFCQQYDYFLSITFGQGTRLEIKRTVAAPSVFIFPPSDEQLKSGTASVVCLLNN | |
| | FYPREAKVQWKVDNALQSGNSQESVTEQDSKDSTYSLSSTLTLSKADYEKHKVYACEVTHQGLSSPV | |
| | TKSFNRGEC | |
| ABCB5-Ab101 | EIVLTQSPGTLSLSPGERATLSCRASQSVNSNYLAWYQQKPGQAPRLLIYGTSSRATGIPDSFSGSGSGT | 47 |
| | DFTLTISRLEPEDFAVYYCQQFGSSPLTFGGGTKVEIKRTVAAPSVFIFPPSDEQLKSGTASVVCLLNNF | |
| | YPREAKVQWKVDNALQSGNSQESVTEQDSKDSTYSLSSTLTLSKADYEKHKVYACEVTHQGLSSPVT | |
| | KSFNRGEC | |

TABLE 5
Heavy chain CDR sequences of exemplary anti-ABCB5 antibodies

| Exemplary | CDR1/ | CDR2/ | CDR3/ |
|---|---|---|---|
| Antibody | SEQ ID NO. | SEQ ID NO. | SEQ ID NO. |
| ABCB5-Ab1 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab2 | GFTFSSYDMN | YISSSSNTIYYADSVKG | NYQYGDYGGY |
| | (SEQ ID NO. 50) | (SEQ ID NO. 58) | (SEQ ID NO. 66) |
| ABCB5-Ab3 | GFTFSSYSMN | YISSSGSTIYYADSVKG | NYQYGDYGGY |
| | (SEQ ID NO. 49) | (SEQ ID NO. 59) | (SEQ ID NO. 66) |
| ABCB5-Ab4 | GFTFSSYSMN | YISSSGSTIYYADSVKG | NYQYGDYGGY |
| | (SEQ ID NO. 49) | (SEQ ID NO. 57) | (SEQ ID NO. 66) |
| ABCB5-Ab5 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab6 | GFTFSSYDMN | YISSSSNTIYYADSVKG | NYQYGDYGGY |
| | (SEQ ID NO. 50) | (SEQ ID NO. 58) | (SEQ ID NO. 66) |
| ABCB5-Ab7 | GFTFSSYSMN | YISSSGSTIYYADSVKG | NYQYGDYGGY |
| | (SEQ ID NO. 49) | (SEQ ID NO. 59) | (SEQ ID NO. 66) |
| ABCB5-Ab8 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab9 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Abl0 | GFTFSSYDMN | YISSSSNTIYYADSVKG | NYQYGDYGGY |
| | (SEQ ID NO. 50) | (SEQ ID NO. 58) | (SEQ ID NO. 66) |
| ABCB5-Ab11 | GFTFSSYSMN | YISSSGSTIYYADSVKG | NYQYGDYGGY |
| | (SEQ ID NO. 49) | (SEQ ID NO. 59) | (SEQ ID NO. 66) |
| ABCB5-Ab12 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab13 | GFTFSSYDMN | YISSSSNTIYYADSVKG | NYQYGDYGGY |
| | (SEQ ID NO. 50) | (SEQ ID NO. 58) | (SEQ ID NO. 66) |
| ABCB5-Abl4 | GFTFSSYSMN | YISSSGSTIYYADSVKG | NYQYGDYGGY |
| | (SEQ ID NO. 49) | (SEQ ID NO. 59) | (SEQ ID NO. 66) |
| ABCB5-Ab15 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Abl6 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Abl7 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab18 | GFTFSSYSMN | YISSSSSTIYYADSVKG | NYQYGDYGGY |
| | (SEQ ID NO. 49) | (SEQ ID NO. 57) | (SEQ ID NO. 66) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Abl9 | GFTFSSYSMN | YISSSSSTIYYADSVKG | NYQYGDYGGY |
| | (SEQ ID NO. 49) | (SEQ ID NO. 57) | (SEQ ID NO. 66) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Ab20 | GFTFSSYSMN | YISSSSSTIYYADSVKG | NYQYGDYGGY |
| | (SEQ ID NO. 49) | (SEQ ID NO. 57) | (SEQ ID NO. 66) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Ab21 | [symbol] | [symbol] | [symbol] |
| | (SEQ ID NO. 49) | (SEQ ID NO. 57) | (SEQ ID NO. 66) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Ab22 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab23 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab24 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab25 | [symbol] | YISSSSSTIYYADSVKG | NYQYODYGGY |
| | (SEQ ID NO. 49) | (SEQ ID NO. 57) | (SEQ ID NO. 66) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab26 | GFTFSSYSMN | YISSSSSTIYYADSVKG | NYQYGDYGGY |
| | (SEQ ID NO. 49) | (SEQ ID NO. 57) | (SEQ ID NO. 66) |
| | 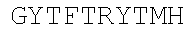 | 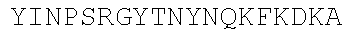 | 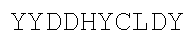 |
| | (SEQ ID NO. 51) | (SEQ ID NO. 60) | (SEQ ID NO. 67) |
| ABCB5-Ab27 | GFTFSSYSMN | YISSSSSTIYYADSVKG | NYQYGDYGGY |
| | (SEQ ID NO. 49) | (SEQ ID NO. 57) | (SEQ ID NO. 66) |
| | 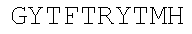 | 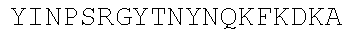 | 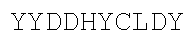 |
| | (SEQ ID NO. 51) | (SEQ ID NO. 60) | (SEQ ID NO. 67) |
| ABCB5-Ab28 | GFTFSSYSMN | YISSSSSTIYYADSVKG | NYQYGDYGGY |
| | (SEQ ID NO. 49) | (SEQ ID NO. 57) | (SEQ ID NO. 66) |
| | 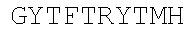 | 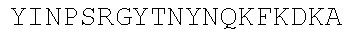 | 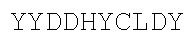 |
| | (SEQ ID NO. 51) | (SEQ ID NO. 60) | (SEQ ID NO. 67) |
| ABCB5-Ab29 | GFTFSSYSMN | YISSSSSTIYYADSVKG | NYQYGDYGGY |
| | (SEQ ID NO. 49) | (SEQ ID NO. 57) | (SEQ ID NO. 66) |
| | 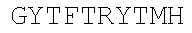 | 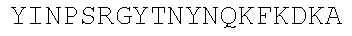 | 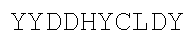 |
| | (SEQ ID NO. 51) | (SEQ ID NO. 60) | (SEQ ID NO. 67) |
| ABCB5-Ab30 | GFTFSSYSMN | YISSSSSTIYYADSVKG | NYQYGDYGGY |
| | (SEQ ID NO. 49) | (SEQ ID NO. 57) | (SEQ ID NO. 66) |
| | 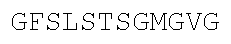 | 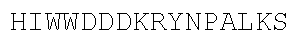 | 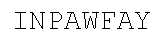 |
| | (SEQ ID NO. 53) | (SEQ ID NO. 62) | (SEQ ID NO. 69) |
| ABCB5-Ab31 | GFTFSSYSMN | YISSSSSTIYYADSVKG | NYQYGDYGGY |
| | (SEQ ID NO. 49) | (SEQ ID NO. 57) | (SEQ ID NO. 66) |
| | 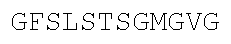 | 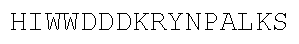 | 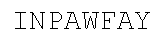 |
| | (SEQ ID NO. 53) | (SEQ ID NO. 62) | (SEQ ID NO. 69) |
| ABCB5-Ab32 | GFTFSSYSMN | YISSSSSTIYYADSVKG | NYQYGDYGGY |
| | (SEQ ID NO. 49) | (SEQ ID NO. 57) | (SEQ ID NO. 66) |
| | 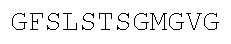 | 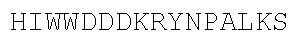 | 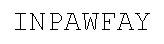 |
| | (SEQ ID NO. 53) | (SEQ ID NO. 62) | (SEQ ID NO. 69) |
| ABCB5-Ab33 | GFTFSSYSMN | YISSSSSTIYYADSVKG | NYQYGDYGGY |
| | (SEQ ID NO. 49) | (SEQ ID NO. 57) | (SEQ ID NO. 66) |
| | 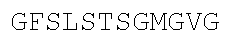 | 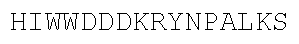 | 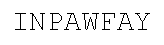 |
| | (SEQ ID NO. 53) | (SEQ ID NO. 62) | (SEQ ID NO. 69) |
| ABCB5-Ab34 | GFTFSSYSMN | YISSSSSTIYYADSVKG | NYQYGDYGGY |
| | (SEQ ID NO. 49) | (SEQ ID NO. 57) | (SEQ ID NO. 66) |
| | 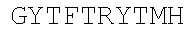 | 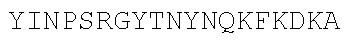 | 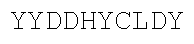 |
| | (SEQ ID NO. 51) | (SEQ ID NO. 60) | (SEQ ID NO. 67) |
| ABCB5-Ab35 | GFTFSSYSMN | YISSSSSTIYYADSVKG | NYQYGDYGGY |
| | (SEQ ID NO. 49) | (SEQ ID NO. 57) | (SEQ ID NO. 66) |
| | 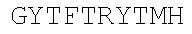 | 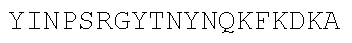 | 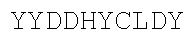 |
| | (SEQ ID NO. 51) | (SEQ ID NO. 60) | (SEQ ID NO. 67) |
| ABCB5-Ab36 | GFTFSSYSMN | YISSSSSTIYYADSVKG | NYQYGDYGGY |
| | (SEQ ID NO. 49) | (SEQ ID NO. 57) | (SEQ ID NO. 66) |
| | 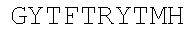 | 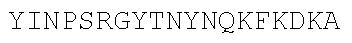 | 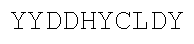 |
| | (SEQ ID NO. 51) | (SEQ ID NO. 60) | (SEQ ID NO. 67) |
| ABCB5-Ab37 | GFTFSSYSMN | YISSSSSTIYYADSVKG | NYQYGDYGGY |
| | (SEQ ID NO. 49) | (SEQ ID NO. 57) | (SEQ ID NO. 66) |
| | 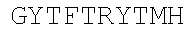 | 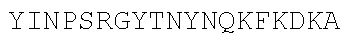 | 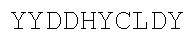 |
| | (SEQ ID NO. 51) | (SEQ ID NO. 60) | (SEQ ID NO. 67) |
| ABCB5-Ab38 | GFTFSSYSMN | YISSSSSTIYYADSVKG | NYQYGDYGGY |
| | (SEQ ID NO. 49) | (SEQ ID NO. 57) | (SEQ ID NO. 66) |
| | 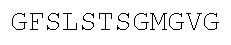 | 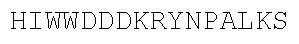 | 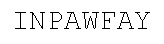 |
| | (SEQ ID NO. 53) | (SEQ ID NO. 62) | (SEQ ID NO. 69) |
| ABCB5-Ab39 | GFTFSSYSMN | YISSSSSTIYYADSVKG | NYQYGDYGGY |
| | (SEQ ID NO. 49) | (SEQ ID NO. 57) | (SEQ ID NO. 66) |
| | 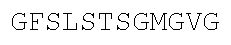 | 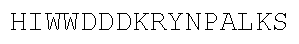 | 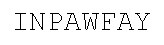 |
| | (SEQ ID NO. 53) | (SEQ ID NO. 62) | (SEQ ID NO. 69) |
| ABCB5-Ab40 | GFTFSSYSMN | YISSSSSTIYYADSVKG | NYQYGDYGGY |
| | (SEQ ID NO. 49) | (SEQ ID NO. 57) | (SEQ ID NO. 66) |
| | 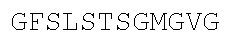 | 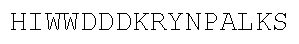 | 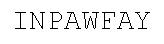 |
| | (SEQ ID NO. 53) | (SEQ ID NO. 62) | (SEQ ID NO. 69) |
| ABCB5-Ab41 | GFTFSSYSMN | YISSSSSTIYYADSVKG | NYQYGDYGGY |
| | (SEQ ID NO. 49) | (SEQ ID NO. 57) | (SEQ ID NO. 66) |
| | 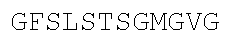 | 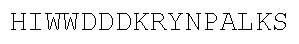 | 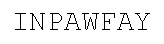 |
| | (SEQ ID NO. 53) | (SEQ ID NO. 62) | (SEQ ID NO. 69) |
| ABCB5-Ab42 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab43 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab44 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab45 | GYTFTNYYMH | IINPSGGSTSYAQKFQG | DLAVTGTAYYYYYGMDV |
| | (SEQ ID NO. 56) | (SEQ ID NO. 64) | (SEQ ID NO. 72) |
| ABCB5-Ab46 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Ab47 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Ab48 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab49 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab50 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 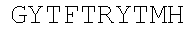 | 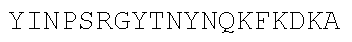 | 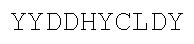 |
| | (SEQ ID NO. 51) | (SEQ ID NO. 60) | (SEQ ID NO. 67) |
| ABCB5-Ab51 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 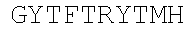 | 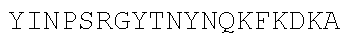 | 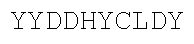 |
| | (SEQ ID NO. 51) | (SEQ ID NO. 60) | (SEQ ID NO. 67) |
| ABCB5-Ab52 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 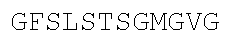 | 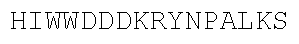 | 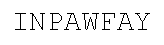 |
| | (SEQ ID NO. 53) | (SEQ ID NO. 62) | (SEQ ID NO. 69) |
| ABCB5-Ab53 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 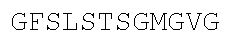 | 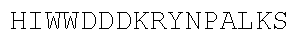 | 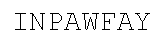 |
| | (SEQ ID NO. 53) | (SEQ ID NO. 62) | (SEQ ID NO. 69) |
| ABCB5-Ab54 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 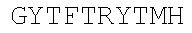 | 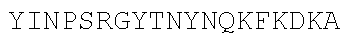 | 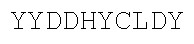 |
| | (SEQ ID NO. 51) | (SEQ ID NO. 60) | (SEQ ID NO. 67) |
| ABCB5-Ab55 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 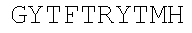 | 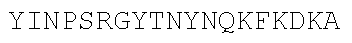 | 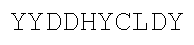 |
| | (SEQ ID NO. 51) | (SEQ ID NO. 60) | (SEQ ID NO. 67) |
| ABCB5-Ab56 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 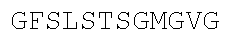 | 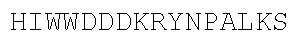 | 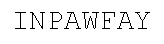 |
| | (SEQ ID NO. 53) | (SEQ ID NO. 62) | (SEQ ID NO. 69) |
| ABCB5-Ab57 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | 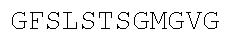 | 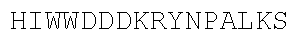 | 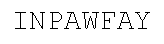 |
| | (SEQ ID NO. 53) | (SEQ ID NO. 62) | (SEQ ID NO. 69) |
| ABCB5-Ab58 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Ab59 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GYTFTRYTMH | YINPSRGYTNYNQKFKDKA | YYDDHYCLDY |
| | (SEQ ID NO. 51) | (SEQ ID NO. 60) | (SEQ ID NO. 67) |
| ABCB5-Ab60 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab61 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GFSLSTSGMGVG | HIWWDDDKRYNPALKS | INPAWFAY |
| | (SEQ ID NO. 53) | (SEQ ID NO. 62) | (SEQ ID NO. 69) |
| ABCB5-Ab62 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 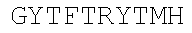 | 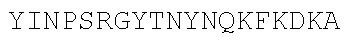 | 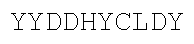 |
| | (SEQ ID NO. 51) | (SEQ ID NO. 60) | (SEQ ID NO. 67) |
| ABCB5-Ab63 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 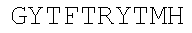 | 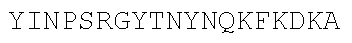 | 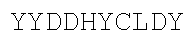 |
| | (SEQ ID NO. 51) | (SEQ ID NO. 60) | (SEQ ID NO. 67) |
| ABCB5-Ab64 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 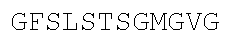 | 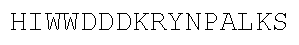 | 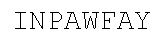 |
| | (SEQ ID NO. 53) | (SEQ ID NO. 62) | (SEQ ID NO. 69) |
| ABCB5-Ab65 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 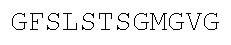 | 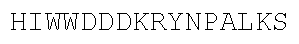 | 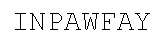 |
| | (SEQ ID NO. 53) | (SEQ ID NO. 62) | (SEQ ID NO. 69) |
| ABCB5-Ab66 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 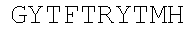 | 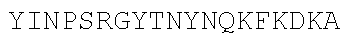 | 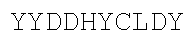 |
| | (SEQ ID NO. 51) | (SEQ ID NO. 60) | (SEQ ID NO. 67) |
| ABCB5-Ab67 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 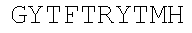 | 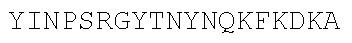 | 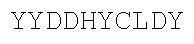 |
| | (SEQ ID NO. 51) | (SEQ ID NO. 60) | (SEQ ID NO. 67) |
| ABCB5-Ab68 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 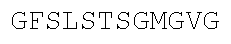 | 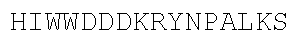 | 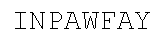 |
| | (SEQ ID NO. 53) | (SEQ ID NO. 62) | (SEQ ID NO. 69) |
| ABCB5-Ab69 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | 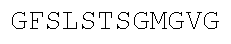 | 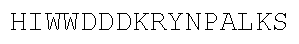 | 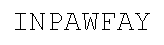 |
| | (SEQ ID NO. 53) | (SEQ ID NO. 62) | (SEQ ID NO. 69) |
| ABCB5-Ab70 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab71 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab72 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab73 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab74 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab75 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab76 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab77 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab78 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab79 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab80 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab81 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab82 | GFTFSSYSMN | YISSSSSTIYYADSVKG ) | NYQYGDYGGY |
| | (SEQ ID NO. 49) | (SEQ ID NO. 57 | (SEQ ID NO. 66) |
| ABCB5-Ab83 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab84 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab85 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab86 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab87 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab88 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab89 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab90 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab91 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab92 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab93 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab94 | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab95 | GFTFSSYSMN | YISSSSSTIYYADSVKG | NYQYGDYGGY |
| | (SEQ ID NO. 49) | (SEQ ID NO. 57) | (SEQ ID NO. 66) |
| | GFTFSSYSMN | YISSSSSTIYYADSVKG | NYQYGDYGGY |
| | (SEQ ID NO. 49) | (SEQ ID NO. 57) | (SEQ ID NO. 66) |
| ABCB5-Ab96 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab97 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |
| ABCB5-Ab98 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| ABCB5-Ab99 | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |
| | GYTFTSYYMH | IINPSGGSTSYAQKFKG | DQAVTGTAYYYYYGMDV |
| | (SEQ ID NO. 55) | (SEQ ID NO. 65) | (SEQ ID NO. 71) |

| | | | |
|---|---|---|---|
| ABCB5-Abl00 | GFTFSSYSMN | YISSSSSTIYYADSVKG | NYQYGDYGGY |
| | (SEQ ID NO. 49) | (SEQ ID NO. 57) | (SEQ ID NO. 66) |

| | | | |
|---|---|---|---|
| ABCB5-Abl01 | GYTFTSYYMH | IINPSGGSTSYAQKFQG | DQAVTGTAYYYYYGMDV |
| | (SEQ ID NO. 55) | (SEQ ID NO. 64) | (SEQ ID NO. 71) |

[symbol]

indicates data missing or illegible when filed

TABLE 6
Light chain CDR sequences of exemplary anti-ABCB5 antibodies

| Exemplary | CDR1/ | CDR2/ | CDR3/ |
|---|---|---|---|
| Antibody | SEQ ID NO. | SEQ ID NO. | SEQ ID NO. |
| ABCB5-Ab1 | QASHDISNFLN (SEQ ID NO. 73) | DAYNLQT (SEQ ID NO. 77) | QQYDYFLSIT (SEQ ID NO. 79) |
| ABCB5-Ab2 | HASHDISNFLN (SEQ ID NO. 74) | DAYNLQT (SEQ ID NO. 77) | QQYDYFLSIT (SEQ ID NO. 79) |
| ABCB5-Ab3 | QASHDISNFLN (SEQ ID NO. 73) | DAYNLQT (SEQ ID NO. 77) | QQYDYFLSIT (SEQ ID NO. 79) |
| ABCB5-Ab4 | QASHDISNFLN (SEQ ID NO. 73) | DAYNLQT (SEQ ID NO. 77) | QQYDYFLSIT (SEQ ID NO. 79) |
| ABCB5-Ab6 | HASHDISNFLN (SEQ ID NO. 74) | DAYNLQT (SEQ ID NO. 77) | QQYDYFLSIT (SEQ ID NO. 79) |
| ABCB5-Ab7 | QASHDISNFLN (SEQ ID NO. 73) | DAYNLQT (SEQ ID NO. 77) | QQYDYFLSIT (SEQ ID NO. 79) |
| ABCB5-Ab8 | QASHDISNFLN (SEQ ID NO. 73) | DAYNLQT (SEQ ID NO. 77) | QQYDYFLSIT (SEQ ID NO. 79) |
| ABCB5-Ab9 | QASHDISNFLN (SEQ ID NO. 73) | DAYNLQT (SEQ ID NO. 77) | QQYDYFLSIT (SEQ ID NO. 79) |
| ABCB5-Ab10 | HASHDISNFLN (SEQ ID NO. 74) | DAYNLQT (SEQ ID NO. 77) | QQYDYFLSIT (SEQ ID NO. 79) |
| ABCB5-Ab11 | QASHDISNFLN (SEQ ID NO. 73) | DAYNLQT (SEQ ID NO. 77) | QQYDYFLSIT (SEQ ID NO. 79) |
| ABCB5-Ab12 | QASHDISNFLN (SEQ ID NO. 73) | DAYNLQT (SEQ ID NO. 77) | QQYDYFLSIT (SEQ ID NO. 79) |
| ABCB5-Ab13 | HASHDISNFLN (SEQ ID NO. 74) | DAYNLQT (SEQ ID NO. 77) | QQYDYFLSIT (SEQ ID NO. 79) |
| ABCB5-Ab14 | QASHDISNFLN (SEQ ID NO. 73) | DAYNLQT (SEQ ID NO. 77) | QQYDYFLSIT (SEQ ID NO. 79) |
| ABCB5-Ab15 | QASHDISNFLN (SEQ ID NO. 73) | DAYNLQT (SEQ ID NO. 77) | QQYDYFLSIT (SEQ ID NO. 79) |
| ABCB5-Ab16 | QASHDISNFLN (SEQ ID NO. 73) | DAYNLQT (SEQ ID NO. 77) | QQYDYFLSIT (SEQ ID NO. 79) |
| ABCB5-Ab17 | QASHDISNFLN (SEQ ID NO. 73) | DAYNLQT (SEQ ID NO. 77) | QQYDYFLSIT (SEQ ID NO. 79) |
| ABCB5-Ab18 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: | DTSKLAS (SEQ ID NO. 61) | QQWSSNPFT (SEQ ID NO. 103) |
| | 52) | | |
| ABCB5-Ab19 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: | DTSKLAS (SEQ ID NO. 61) | QQWSSNPFT (SEQ ID NO. 103) |
| | 52) | | |
| ABCB5-Ab20 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO. 52) | DTSKLAS (SEQ ID NO. 61) | QQWSSNPFT (SEQ ID NO. 103) |
| ABCB5-Ab21 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO. 52) | DTSKLAS (SEQ ID NO. 61) | QQWSSNPFT (SEQ ID NO. 103) |
| ABCB5-Ab22 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO: 70) |
| | 54) | 63) | |
| ABCB5-Ab23 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO: 70) |
| | 54) | 63) | |
| ABCB5-Ab24 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID | TTSNLES (SEQ ID NO. 63) | QQSNEDPYT (SEQ ID NO. 70) |
| | NO. 54) | | |
| ABCB5-Ab25 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID | TTSNLES (SEQ ID NO. 63) | QQSNEDPYT (SEQ ID NO. 70) |
| | NO. 54) | | |
| ABCB5-Ab26 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab27 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab28 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab29 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab30 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab31 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab32 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab33 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab34 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab35 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab36 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab37 | QASHDISNFLN (SEQ ID NO. 73) | DAYNLQT (SEQ ID NO. 77) | QQYDYFLSIT (SEQ ID NO. 79) |
| | SASSSVSYMN (SEQ ID NO: 52) | DTSKLAS (SEQ ID NO. 61) | QQWSSNPFT (SEQ ID NO: 103) |
| ABCB5-Ab38 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab39 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab40 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab41 | QASHDISNFLN (SEQ ID NO. 73) | DAYNLQT (SEQ ID NO. 77) | QQYDYFLSIT (SEQ ID NO. 79) |
| | KASQSVDFDGDSFMN (SEQ ID NO. | TTSNLES (SEQ ID NO: | QQSNEDPYT (SEQ ID NO. 70) |
| | 54) | 63) | |
| ABCB5-Ab42 | RASLSVNSNYLA (SEQ ID NO. 75) | GTSSRAT (SEQ ID NO. 78) | QQFGSSPLT (SEQ ID NO. 81) |
| ABCB5-Ab43 | RASQSVNSKYLA (SEQ ID NO. 76) | GTSSRAT (SEQ ID NO. 78) | QQFGSSPLT (SEQ ID NO. 81) |
| ABCB5-Ab44 | RASQSVNSNYLA (SEQ ID NO. 80) | GTSSRAT (SEQ ID NO. 78) | QQFGSSPLT (SEQ ID NO. 81) |
| ABCB5-Ab45 | RASQSVNSNYLA (SEQ ID NO. 80) | GTSSRAT (SEQ ID NO. 78) | QQFGSSPLT (SEQ ID NO. 81) |
| ABCB5-Ab46 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO: 52) | DTSKLAS (SEQ ID NO. 61) | QQWSSNPFT (SEQ ID NO: 103) |
| ABCB5-Ab47 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab48 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab49 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID | TTSNLES (SEQ ID NO. 63) | QQSNEDPYT (SEQ ID NO. 70) |
| | NO. 54) | | |
| ABCB5-Ab50 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab51 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab52 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab53 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab54 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab55 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab56 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab57 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab58 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab59 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab60 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab61 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID | TTSNLES (SEQ ID NO. 63) | QQSNEDPYT (SEQ ID NO. 70) |
| | NO. 54) | | |
| ABCB5-Ab62 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab63 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab64 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab65 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab66 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab67 | RASQSVNSNYLA (SEQ ID NO. 80) | GTSSRAT (SEQ ID NO. 78) | QQFGSSPLT (SEQ ID NO. 81) |
| | SASSSVSYMN (SEQ ID NO. 52) | DTSKLAS (SEQ ID NO. 61) | QQWSSNPFT (SEQ ID NO. 103) |
| ABCB5-Ab68 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab69 | RASQSVNSNYLA (SEQ ID NO. 80) | GTSSRAT (SEQ ID NO. 78) | QQFGSSPLT (SEQ ID NO. 81) |
| | KASQSVDFDGDSFMN (SEQ ID NO. | TTSNLES (SEQ ID NO. 63) | QQSNEDPYT (SEQ ID NO. 70) |
| | 54) | | |
| ABCB5-Ab70 | QASHDISNFLN (SEQ ID NO. 73) | DAYNLQT (SEQ ID NO. 77) | QQYDYFLSIT (SEQ ID NO. 79) |
| ABCB5-Ab71 | QASHDISNFLN (SEQ ID NO. 73) | DAYNLQT (SEQ ID NO. 77) | QQYDYFLSIT (SEQ ID NO. 79) |
| | QASHDISNFLN (SEQ ID NO. 73) | DAYNLQT (SEQ ID NO. 77) | QQYDYFLSIT (SEQ ID NO. 79) |
| ABCB5-Ab72 | RASQSVNSNYLA (SEQ ID NO. 80) | GTSSRAT (SEQ ID NO. 78) | QQFGSSPLT (SEQ ID NO. 81) |
| ABCB5-Ab73 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab74 | RASQSVNSNYLA (SEQ ID NO. 80) | GTSSRAT (SEQ ID NO. 78) | QQFGSSPLT (SEQ ID NO. 81) |
| ABCB5-Ab75 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab76 | QASHDISNFLN (SEQ ID NO. 73) | DAYNLQT (SEQ ID NO. 77) | QQYDYFLSIT (SEQ ID NO. 79) |
| ABCB5-Ab77 | QASHDISNFLN (SEQ ID NO. 73) | DAYNLQT (SEQ ID NO. 77) | QQYDYFLSIT (SEQ ID NO. 79) |
| | QASHDISNFLN (SEQ ID NO. 73) | DAYNLQT (SEQ ID NO. 77) | QQYDYFLSIT (SEQ ID NO. 79) |
| ABCB5-Ab78 | RASQSVNSNYLA (SEQ ID NO. 80) | GTSSRAT (SEQ ID NO. 78) | QQFGSSPLT (SEQ ID NO. 81) |
| ABCB5-Ab79 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab80 | RASQSVNSNYLA (SEQ ID NO. 80) | GTSSRAT (SEQ ID NO. 78) | QQFGSSPLT (SEQ ID NO. 81) |
| ABCB5-Ab81 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab82 | QASHDISNFLN (SEQ ID NO. 73) | DAYNLQT (SEQ ID NO. 77) | QQYDYFLSIT (SEQ ID NO. 79) |
| ABCB5-Ab83 | QASHDISNFLN (SEQ ID NO. 73) | DAYNLQT (SEQ ID NO. 77) | QQYDYFLSIT (SEQ ID NO. 79) |
| | QASHDISNFLN (SEQ ID NO. 73) | DAYNLQT (SEQ ID NO. 77) | QQYDYFLSIT (SEQ ID NO. 79) |
| ABCB5-Ab84 | RASQSVNSNYLA (SEQ ID NO. 80) | GTSSRAT (SEQ ID NO. 78) | QQFGSSPLT (SEQ ID NO. 81) |
| ABCB5-Ab85 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab86 | RASQSVNSNYLA (SEQ ID NO. 80) | GTSSRAT (SEQ ID NO. 78) | QQFGSSPLT (SEQ ID NO. 81) |
| ABCB5-Ab87 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab88 | QASHDISNFLN (SEQ ID NO. 73) | DAYNLQT (SEQ ID NO. 77) | QQYDYFLSIT (SEQ ID NO. 79) |
| ABCB5-Ab89 | QASHDISNFLN (SEQ ID NO. 73) | DAYNLQT (SEQ ID NO. 77) | QQYDYFLSIT (SEQ ID NO. 79) |
| | QASHDISNFLN (SEQ ID NO. 73) | DAYNLQT (SEQ ID NO. 77) | QQYDYFLSIT (SEQ ID NO. 79) |
| ABCB5-Ab90 | RASQSVNSNYLA (SEQ ID NO. 80) | GTSSRAT (SEQ ID NO. 78) | QQFGSSPLT (SEQ ID NO. 81) |
| ABCB5-Ab91 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab92 | RASQSVNSNYLA (SEQ ID NO. 80) | GTSSRAT (SEQ ID NO. 78) | QQFGSSPLT (SEQ ID NO. 81) |
| ABCB5-Ab93 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab94 | QASHDISNFLN (SEQ ID NO. 73) | DAYNLQT (SEQ ID NO. 77) | QQYDYFLSIT (SEQ ID NO. 79) |
| ABCB5-Ab95 | QASHDISNFLN (SEQ ID NO. 73) | DAYNLQT (SEQ ID NO. 77) | QQYDYFLSIT (SEQ ID NO. 79) |
| | QASHDISNFLN (SEQ ID NO. 73) | DAYNLQT (SEQ ID NO. 77) | QQYDYFLSIT (SEQ ID NO. 79) |
| ABCB5-Ab96 | RASQSVNSNYLA (SEQ ID NO. 80) | GTSSRAT (SEQ ID NO. 78) | QQFGSSPLT (SEQ ID NO. 81) |
| ABCB5-Ab97 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab98 | RASQSVNSNYLA (SEQ ID NO. 80) | GTSSRAT (SEQ ID NO. 78) | QQFGSSPLT (SEQ ID NO. 81) |
| ABCB5-Ab99 | RASQSVNSNYLA (SEQ ID NO. 80) | GTSSRAT (SEQ ID NO. 78) | QQFGSSPLT (SEQ ID NO. 81) |
| | RASQSVNSNYLA (SEQ ID NO. 80) | GTSSRAT (SEQ ID NO. 78) | QQFGSSPLT (SEQ ID NO. 81) |
| ABCB5-Ab100 | QASHDISNFLN (SEQ ID NO. 73) | DAYNLQT (SEQ ID NO. 77) | QQYDYFLSIT (SEQ ID NO. 79) |
| ABCB5-Ab101 | RASQSVNSNYLA (SEQ ID NO. 80) | GTSSRAT (SEQ ID NO. 78) | QQFGSSPLT (SEQ ID NO. 81) |

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

In further embodiments, the anti-ABCB5 antibodies may include modifications to improve properties of the antibody, for example, stability, oxidation, isomerization and deamidation. In some embodiments, the anti-ABCB5 antibody disclosed herein may comprise heavy chain CDRs that collectively are at least 80% (e.g., 85%, 90%, 95%, or 98%) identical to the heavy chain CDRs of a reference antibody such as Ab100 or Ab101. Alternatively or in addition, the antibody may comprise light chain CDRs that collectively are at least 80% (e.g., 85%, 90%, 95%, or 98%) identical to the light chain CDRs of the reference antibody. In some embodiments, the anti-ABCB5 antibody may comprise a heavy chain variable region that is at least 80% (e.g., 85%, 90%, 95%, or 98%) identical to the heavy chain variable region of a reference antibody such as Ab100 or Ab101 and/or a light chain variable region that is at least 80% (e.g., 85%, 90%, 95%, or 98%) identical to the light chain variable region of the reference antibody.

The “percent identity” of two amino acid sequences may be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-ABCB5 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4.

The light chain of any of the anti-ABCB5 antibodies described herein may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (imgt.org) or at vbase2.org/vbstat.php., both of which are incorporated by reference herein.

When needed, the anti-ABCB5 antibody as described herein may comprise a modified constant region. For example, it may comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Provided below are several exemplary anti-ABCB5 antibodies including other features, fusion constructs, etc.

TABLE 7
Other Features of exemplary anti-ABCB5 antibodies

| Exemplary | | |
|---|---|---|
| Antibody | Linker | Region of 2^{nd} Ag specificity |
| ABCB5-Ab18 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab19 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab20 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab21 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab22 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab23 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab24 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab25 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab26 | GGGGS | QIVLTQSPAIMSASPGEKVTMTCSASSSVSYMNWYQQKSGTSPKRWIYDTSKLASGVPAHFR |
| | (SEQ ID NO. 83) | GSGSGTSYSLTISGMEAEDAATYYCQQWSSNPFTFGSGTKLEINGSTSGGGSGGGSGGGGSSQ |
| | | VQLQQSGAELARPGASVKMSCKASGYTFTRYTMHWVKQRPGQGLEWIGYINPSRGYTNYN |
| | | QKFKDKATLTTDKSSSTAYMQLSSLTSEDSAVYYCARYYDDHYCLDYWGQGTTLTVSS |
| | | (SEQ ID NO. 92) |
| ABCB5-Ab27 | GGGGS | QVQLQQSGAELARPGASVKMSCKASGYTFTRYTMHWVKQRPGQGLEWIGYINPSRGYTNY |
| | (SEQ ID NO. 83) | NQKFKDKATLTTDKSSSTAYMQLSSLTSEDSAVYYCARYYDDHYCLDYWGQGTTLTVSS |
| | | (SEQ ID NO. 93) |
| | | QIVLTQSPAIMSASPGEKVTMTCSASSSVSYMNWYQQKSGTSPKRWIYDTSKLASGVPAHFR |
| | | GSGSGTSYSLTISGMEAEDAATYYCQQWSSNPFTFGSGTKLEIN |
| | | (SEQ ID NO. 94) |
| | GSTSGGGSGGG | Linker between Light-Heavy chains |
| | SGGGGSS | |
| | (SEQ ID NO. 84) | |
| ABCB5-Ab28 | SGGGGS (before) | QIVLTQSPAIMSASPGEKVTMTCSASSSVSYMNWYQQKSGTSPKRWIYDTSKLASGVPAHFR |
| | (SEQ ID NO. 85) | GSGSGTSYSLTISGMEAEDAATYYCQQWSSNPFTFGSGTKLEINGSTSGGGSGGGSGGGGSSQ |
| | GGGGS (after) | VQLQQSGAELARPGASVKMSCKASGYTFTRYTMHWVKQRPGQGLEWIGYINPSRGYTNYN |
| | (SEQ ID NO. 83) | QKFKDKATLTTDKSSSTAYMQLSSLTSEDSAVYYCARYYDDHYCLDYWGQGTTLTVSS |
| | | (SEQ ID NO. 92) |
| ABCB5-Ab29 | GGGGS | QVQLQQSGAELARPGASVKMSCKASGYTFTRYTMHWVKQRPGQGLEWIGYINPSRGYTNY |
| | (SEQ ID NO. 83) | NQKFKDKATLTTDKSSSTAYMQLSSLTSEDSAVYYCARYYDDHYCLDYWGQGTTLTVSS |
| | | (SEQ ID NO. 93) |
| | | QIVLTQSPAIMSASPGEKVTMTCSASSSVSYMNWYQQKSGTSPKRWIYDTSKLASGVPAHFR |
| | | GSGSGTSYSLTISGMEAEDAATYYCQQWSSNPFTFGSGTKLEIN |
| | | (SEQ ID NO. 94) |
| | GSTSGGGSGGG | Linker between Light-Heavy chains |
| | SGGGGSS | |
| | (SEQ ID NO. 84) | |
| ABCB5-Ab30 | GGGGS | DIVLTQSPDSLAVSLGERATINCKASQSVDFDGDSFMNWYQQKPGQPPKLLIYTTSNLESGVP |
| | (SEQ ID NO. 83) | DRFSGSGSGTDFTLTIRPLQAEDVAVYYCQQSNEDPYTFGQGTKLEIKGSTSGGGSGGGSGGG |
| | | GSSQVTLKESGPALVKPTQTLTLTCTFSGFSLSTSGMGVGWIRQPPGKALEWLAHIWWDDDK |
| | | RYNPALKSRLTISKDTSKNQVVLTMTNMDPVDTATYYCAQINPAWFAYWGQGTLVTVSS |
| | | (SEQ ID NO. 95) |
| ABCB5-Ab31 | GGGGS | QVTLKESGPALVKPTQTLTLTCTFSGFSLSTSGMGVGWIRQPPGKALEWLAHIWWDDDKRY |
| | (SEQ ID NO. 83) | NPALKSRLTISKDTSKNQVVLTMTNMDPVDTATYYCAQINPAWFAYWGQGTLVTVSS |
| | | (SEQ ID NO. 96) |
| | | DIVLTQSPDSLAVSLGERATINCKASQSVDFDGDSFMNWYQQKPGQPPKLLIYTTSNLESGVP |
| | | DRFSGSGSGTDFTLTIRPLQAEDVAVYYCQQSNEDPYTFGQGTKLEIK |
| | | (SEQ ID NO. 97) |
| | GSTSGGGSGGG | Linker between Light-Heavy chains |
| | SGGGGSS | |
| | (SEQ ID NO. 84) | |
| ABCB5-Ab32 | SGGGGS (before) | DIVLTQSPDSLAVSLGERATINCKASQSVDFDGDSFMNWYQQKPGQPPKLLIYTTSNLESGVP |
| | (SEQ ID NO. 85) | DRFSGSGSGTDFTLTIRPLQAEDVAVYYCQQSNEDPYTFGQGTKLEIKGSTSGGGSGGGSGGG |
| | GGGGS (after) | GSSQVTLKESGPALVKPTQTLTLTCTFSGFSLSTSGMGVGWIRQPPGKALEWLAHIWWDDDK |
| | (SEQ ID NO. 83) | RYNPALKSRLTISKDTSKNQVVLTMTNMDPVDTATYYCAQINPAWFAYWGQGTLVTVSS |
| | | (SEQ ID NO. 95) |
| ABCB5-Ab33 | GGGGS | QVTLKESGPALVKPTQTLTLTCTFSGFSLSTSGMGVGWIRQPPGKALEWLAHIWWDDDKRY |
| | (SEQ ID NO. 83) | NPALKSRLTISKDTSKNQVVLTMTNMDPVDTATYYCAQINPAWFAYWGQGTLVTVSS |
| | | (SEQ ID NO. 96) |
| | | DIVLTQSPDSLAVSLGERATINCKASQSVDFDGDSFMNWYQQKPGQPPKLLIYTTSNLESGVP |
| | | DRFSGSGSGTDFTLTIRPLQAEDVAVYYCQQSNEDPYTFGQGTKLEIK |
| | | (SEQ ID NO. 97) |
| | GSTSGGGSGGG | Linker between Light-Heavy chains |
| | SGGGGSS | |
| | (SEQ ID NO. 84) | |
| ABCB5-Ab34 | GGGGSS | QVQLQQSGAELARPGASVKMSCKASGYTFTRYTMHWVKQRPGQGLEWIGYINPSRGYTNY |
| | (SEQ ID NO. 86) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | GSTSGGGSGGG | Linker between Heavy-Light chains |
| | SGGGGSS | |
| | (SEQ ID NO. 84) | |
| ABCB5-Ab35 | GSTSGGGSGGG | QVQLQQSGAELARPGASVKMSCKASGYTFTRYTMHWVKQRPGQGLEWIGYINPSRGYTNY |
| | SGGGGSS | NQKFKDKATLTTDKSSSTAYMQLSSLTSEDSAVYYCARYYDDHYCLDYWGQGTTLTVSSGS |
| | (SEQ ID NO. 84) | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | | Linker between Heavy-Light chains |
| ABCB5-Ab36 | GGGGSS | QVQLQQSGAELARPGASVKMSCKASGYTFTRYTMHWVKQRPGQGLEWIGYINPSRGYTNY |
| | (SEQ ID NO. 86) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | GSTSGGGSGGG | Linker between Heavy-Light chains |
| | SGGGGSS | |
| | (SEQ ID NO. 84) | |
| ABCB5-Ab37 | GSTSGGGSGGG | QVQLQQSGAELARPGASVKMSCKASGYTFTRYTMHWVKQRPGQGLEWIGYINPSRGYTNY |
| | SGGGGSS | NQKFKDKATLTTDKSSSTAYMQLSSLTSEDSAVYYCARYYDDHYCLDYWGQGTTLTVSSGS |
| | (SEQ ID NO. 84) | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | | Linker between Heavy-Light chains |
| ABCB5-Ab38 | GGGGSS | QVTLKESGPALVKPTQTLTLTCTFSGFSLSTSGMGVGWIRQPPGKALEWLAHIWWDDDKRY |
| | (SEQ ID NO. 86) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | GSTSGGGSGGG | Linker between Heavy-Light chains |
| | SGGGGSS | |
| | (SEQ ID NO. 84) | |
| ABCB5-Ab39 | GSTSGGGSGGG | QVTLKESGPALVKPTQTLTLTCTFSGFSLSTSGMGVGWIRQPPGKALEWLAHIWWDDDKRY |
| | SGGGGSS | NPALKSRLTISKDTSKNQVVLTMTNMDPVDTATYYCAQINPAWFAYWGQGTLVTVSSGSTS |
| | (SEQ ID NO. 84) | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | | Linker between Heavy-Light chains |
| ABCB5-Ab40 | GGGGSS | QVTLKESGPALVKPTQTLTLTCTFSGFSLSTSGMGVGWIRQPPGKALEWLAHIWWDDDKRY |
| | (SEQ ID NO. 86) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | GSTSGGGSGGG | Linker between Heavy-Light chains |
| | SGGGGSS | |
| | (SEQ ID NO. 84) | |
| ABCB5-Ab41 | GSTSGGGSGGG | QVTLKESGPALVKPTQTLTLTCTFSGFSLSTSGMGVGWIRQPPGKALEWLAHIWWDDDKRY |
| | SGGGGSS | NPALKSRLTISKDTSKNQVVLTMTNMDPVDTATYYCAQINPAWFAYWGQGTLVTVSSGSTS |
| | (SEQ ID NO. 84) | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | | Linker between Heavy-Light chains |
| ABCB5-Ab46 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab47 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab48 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab49 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab50 | GGGGS | QIVLTQSPAIMSASPGEKVTMTCSASSSVSYMNWYQQKSGTSPKRWIYDTSKLASGVPAHFR |
| | (SEQ ID NO. 83) | GSGSGTSYSLTISGMEAEDAATYYCQQWSSNPFTFGSGTKLEINGSTSGGGSGGGSGGGGSSQ |
| | | VQLQQSGAELARPGASVKMSCKASGYTFTRYTMHWVKQRPGQGLEWIGYINPSRGYTNYN |
| | | QKFKDKATLTTDKSSSTAYMQLSSLTSEDSAVYYCARYYDDHYCLDYWGQGTTLTVSS |
| | | (SEQ ID NO. 92) |
| ABCB5-Ab51 | GGGGS | QVQLQQSGAELARPGASVKMSCKASGYTFTRYTMHWVKQRPGQGLEWIGYINPSRGYTNY |
| | (SEQ ID NO. 83) | NQKFKDKATLTTDKSSSTAYMQLSSLTSEDSAVYYCARYYDDHYCLDYWGQGTTLTVSS |
| | | (SEQ ID NO. 93) |
| | | QIVLTQSPAIMSASPGEKVTMTCSASSSVSYMNWYQQKSGTSPKRWIYDTSKLASGVPAHFR |
| | | GSGSGTSYSLTISGMEAEDAATYYCQQWSSNPFTFGSGTKLEIN |
| | | (SEQ ID NO. 94) |
| | GSTSGGGSGGG | Linker between Light-Heavy chains |
| | SGGGGSS | |
| | (SEQ ID NO. 84) | |
| ABCB5-Ab52 | GGGGS | DIVLTQSPDSLAVSLGERATINCKASQSVDFDGDSFMNWYQQKPGQPPKLLIYTTSNLESGVP |
| | (SEQ ID NO. 83) | DRFSGSGSGTDFTLTIRPLQAEDVAVYYCQQSNEDPYTFGQGTKLEIKGSTSGGGSGGGSGGG |
| | | GSSQVTLKESGPALVKPTQTLTLTCTFSGFSLSTSGMGVGWIRQPPGKALEWLAHIWWDDDK |
| | | RYNPALKSRLTISKDTSKNQVVLTMTNMDPVDTATYYCAQINPAWFAYWGQGTLVTVSS |
| | | (SEQ ID NO. 95) |
| ABCB5-Ab53 | GGGGS | QVTLKESGPALVKPTQTLTLTCTFSGFSLSTSGMGVGWIRQPPGKALEWLAHIWWDDDKRY |
| | (SEQ ID NO. 83) | NPALKSRLTISKDTSKNQVVLTMTNMDPVDTATYYCAQINPAWFAYWGQGTLVTVSS |
| | | (SEQ ID NO. 96) |
| | | DIVLTQSPDSLAVSLGERATINCKASQSVDFDGDSFMNWYQQKPGQPPKLLIYTTSNLESGVP |
| | | DRFSGSGSGTDFTLTIRPLQAEDVAVYYCQQSNEDPYTFGQGTKLEIK |
| | | (SEQ ID NO. 97) |
| | GSTSGGGSGGG | Linker between Light-Heavy chains |
| | SGGGGSS | |
| | (SEQ ID NO. 84) | |
| ABCB5-Ab54 | GGGGSS | QVQLQQSGAELARPGASVKMSCKASGYTFTRYTMHWVKQRPGQGLEWIGYINPSRGYTNY |
| | (SEQ ID NO. 86) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | GSTSGGGSGGG | Linker between Heavy-Light chains |
| | SGGGGSS | |
| | (SEQ ID NO. 84) | |
| ABCB5-Ab55 | GSTSGGGSGGG | QVQLQQSGAELARPGASVKMSCKASGYTFTRYTMHWVKQRPGQGLEWIGYINPSRGYTNY |
| | SGGGGSS | NQKFKDKATLTTDKSSSTAYMQLSSLTSEDSAVYYCARYYDDHYCLDYWGQGTTLTVSSGS |
| | (SEQ ID NO. 84) | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | | Linker between Heavy-Light chains |
| ABCB5-Ab56 | GGGGSS | QVTLKESGPALVKPTQTLTLTCTFSGFSLSTSGMGVGWIRQPPGKALEWLAHIWWDDDKRY |
| | (SEQ ID NO. 86) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | GSTSGGGSGGG | Linker between Heavy-Light chains |
| | SGGGGSS | |
| | (SEQ ID NO. 84) | |
| ABCB5-Ab57 | GSTSGGGSGGG | QVTLKESGPALVKPTQTLTLTCTFSGFSLSTSGMGVGWIRQPPGKALEWLAHIWWDDDKRY |
| | SGGGGSS | NPALKSRLTISKDTSKNQVVLTMTNMDPVDTATYYCAQINPAWFAYWGQGTLVTVSSGSTS |
| | (SEQ ID NO. 84) | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | | Linker between Heavy-Light chains |
| ABCB5-Ab58 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab59 | GGGGSGGGGS | QVQLQQSGAELARPGASVKMSCKASGYTFTRYTMHWVKQRPGQGLEWIGYINPSRGYTNY |
| | (SEQ ID NO. 82) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| ABCB5-Ab60 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab61 | GGGGSGGGGS | QVTLKESGPALVKPTQTLTLTCTFSGFSLSTSGMGVGWIRQPPGKALEWLAHIWWDDDKRY |
| | (SEQ ID NO. 82) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| ABCB5-Ab62 | GGGGS (SEQ ID | QIVLTQSPAIMSASPGEKVTMTCSASSSVSYMNWYQQKSGTSPKRWIYDTSKLASGVPAHFR |
| | NO. 83) | GSGSGTSYSLTISGMEAEDAATYYCQQWSSNPFTFGSGTKLEINGSTSGGGSGGGSGGGGSSQ |
| | | VQLQQSGAELARPGASVKMSCKASGYTFTRYTMHWVKQRPGQGLEWIGYINPSRGYTNYN |
| | | QKFKDKATLTTDKSSSTAYMQLSSLTSEDSAVYYCARYYDDHYCLDYWGQGTTLTVSS |
| | | (SEQ ID NO. 92) |
| ABCB5-Ab63 | GGGGS (SEQ ID | QVQLQQSGAELARPGASVKMSCKASGYTFTRYTMHWVKQRPGQGLEWIGYINPSRGYTNY |
| | NO. 83) | NQKFKDKATLTTDKSSSTAYMQLSSLTSEDSAVYYCARYYDDHYCLDYWGQGTTLTVSS |
| | | (SEQ ID NO. 93) |
| | | QIVLTQSPAIMSASPGEKVTMTCSASSSVSYMNWYQQKSGTSPKRWIYDTSKLASGVPAHFR |
| | | GSGSGTSYSLTISGMEAEDAATYYCQQWSSNPFTFGSGTKLEIN |
| | | (SEQ ID NO. 94) |
| | GSTSGGGSGGG | Linker between Light-Heavy chains |
| | SGGGGSS | |
| | (SEQ ID NO. 84) | |
| ABCB5-Ab64 | GGGGS (SEQ ID | DIVLTQSPDSLAVSLGERATINCKASQSVDFDGDSFMNWYQQKPGQPPKLLIYTTSNLESGVP |
| | NO. 83) | DRFSGSGSGTDFTLTIRPLQAEDVAVYYCQQSNEDPYTFGQGTKLEIKGSTSGGGSGGGSGGG |
| | | GSSQVTLKESGPALVKPTQTLTLTCTFSGFSLSTSGMGVGWIRQPPGKALEWLAHIWWDDDK |
| | | RYNPALKSRLTISKDTSKNQVVLTMTNMDPVDTATYYCAQINPAWFAYWGQGTLVTVSS |
| | | (SEQ ID NO. 95) |
| ABCB5-Ab65 | GGGGS (SEQ ID | QVTLKESGPALVKPTQTLTLTCTFSGFSLSTSGMGVGWIRQPPGKALEWLAHIWWDDDKRY |
| | NO. 83) | NPALKSRLTISKDTSKNQVVLTMTNMDPVDTATYYCAQINPAWFAYWGQGTLVTVSS |
| | | (SEQ ID NO. 96) |
| | | DIVLTQSPDSLAVSLGERATINCKASQSVDFDGDSFMNWYQQKPGQPPKLLIYTTSNLESGVP |
| | | DRFSGSGSGTDFTLTIRPLQAEDVAVYYCQQSNEDPYTFGQGTKLEIK |
| | | (SEQ ID NO. 97) |
| | GSTSGGGSGGG | Linker between Light-Heavy chains |
| | SGGGGSS | |
| | (SEQ ID NO. 84) | |
| ABCB5-Ab66 | GGGGSS | QVQLQQSGAELARPGASVKMSCKASGYTFTRYTMHWVKQRPGQGLEWIGYINPSRGYTNY |
| | (SEQ ID NO. 86) | NQKFKDKATLTTDKSSSTAYMQLSSLTSEDSAVYYCARYYDDHYCLDYWGQGTTLTVSSGS |
| | | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | GSTSGGGSGGG | Linker between Heavy-Light chains |
| | SGGGGSS | |
| | (SEQ ID NO. 84) | |
| ABCB5-Ab67 | GSTSGGGSGGG | QVQLQQSGAELARPGASVKMSCKASGYTFTRYTMHWVKQRPGQGLEWIGYINPSRGYTNY |
| | SGGGGSS | NQKFKDKATLTTDKSSSTAYMQLSSLTSEDSAVYYCARYYDDHYCLDYWGQGTTLTVSSGS |
| | (SEQ ID NO. 84) | TSGGGSGGGSGGGGSSQIVLTQSPAIMSASPGEKVTMTCSASSSVSYMNWYQQKSGTSPKRW |
| | | IYDTSKLASGVPAHFRGSGSGTSYSLTISGMEAEDAATYYCQQWSSNPFTFGSGTKLEIN |
| | | (SEQ ID NO. 90) |
| | | Linker between Heavy-Light chains |
| ABCB5-Ab68 | GGGGSS | QVTLKESGPALVKPTQTLTLTCTFSGFSLSTSGMGVGWIRQPPGKALEWLAHIWWDDDKRY |
| | (SEQ ID NO. 86) | NPALKSRLTISKDTSKNQVVLTMTNMDPVDTATYYCAQINPAWFAYWGQGTLVTVSSGSTS |
| | | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | GSTSGGGSGGG | Linker between Heavy-Light chains |
| | SGGGGSS | |
| | (SEQ ID NO. 84) | |
| ABCB5-Ab69 | GSTSGGGSGGG | QVTLKESGPALVKPTQTLTLTCTFSGFSLSTSGMGVGWIRQPPGKALEWLAHIWWDDDKRY |
| | SGGGGSS | NPALKSRLTISKDTSKNQVVLTMTNMDPVDTATYYCAQINPAWFAYWGQGTLVTVSSGSTS |
| | (SEQ ID NO. 84) | GGGSGGGSGGGGSSDIVLTQSPDSLAVSLGERATINCKASQSVDFDGDSFMNWYQQKPGQPP |
| | | KLLIYTTSNLESGVPDRFSGSGSGTDFTLTIRPLQAEDVAVYYCQQSNEDPYTFGQGTKLEIK |
| | | (SEQ ID NO. 91) |
| | | Linker between Heavy-Light chains |
| ABCB5-Ab70 | GSTSGGGSGGG | GLDTVSFSTKGATYITYVNFLNELRVKLKPEGNSHGIPLLRKKCDDPGKCFVLVALSNDNGQL |
| | SGGGSGGGGSS | AEIAIDVTSVYVVGYQVRNRSYFFKDAPDAAYEGLFKNTIKTRLHFGGSYPSLEGEKAYRETT |
| | (SEQ ID NO. 87) | DLGIEPLRIGIKKLDENAIDNYKPTEIASSLLVVIQMVSEAARFTFIENQIRNNFQQRIRPANNTI |
| | | SLENKWGKLSFQIRTSGANGMFSEAVELERANGKKYYVTAVDQVKPKIALLKFVDKDPE |
| | | (SEQ ID NO. 98) |
| | GSTSGGGSGGG | Linker between Heavy-Light chains |
| | SGGGGSS | |
| | (SEQ ID NO. 84) | |
| ABCB5-Ab71 | GSTSGGGSGGG | GLDTVSFSTKGATYITYVNFLNELRVKLKPEGNSHGIPLLRKKCDDPGKCFVLVALSNDNGQL |
| | SGGGSGGGGSS | AEIAIDVTSVYVVGYQVRNRSYFFKDAPDAAYEGLFKNTIKTRLHFGGSYPSLEGEKAYRETT |
| | (SEQ ID NO. 87) | DLGIEPLRIGIKKLDENAIDNYKPTEIASSLLVVIQMVSEAARFTFIENQIRNNFQQRIRPANNTI |
| | | SLENKWGKLSFQIRTSGANGMFSEAVELERANGKKYYVTAVDQVKPKIALLKFVDKDPE |
| | | (SEQ ID NO. 98) |
| | GSTSGGGSGGG | Linker between Heavy-Light chains |
| | SGGGGSS | |
| | (SEQ ID NO. 84) | |
| | GSTSGGGSGGG | Linker between Light-Heavy chains |
| | SGGGSGGGGSS | |
| | GSTSGGGSGGG | |
| | SGGGSGGGGSS | |
| | (SEQ ID NO. 88) | |
| ABCB5-Ab72 | GSTSGGGSGGG | GLDTVSFSTKGATYITYVNFLNELRVKLKPEGNSHGIPLLRKKCDDPGKCFVLVALSNDNGQL |
| | SGGGSGGGGSS | AEIAIDVTSVYVVGYQVRNRSYFFKDAPDAAYEGLFKNTIKTRLHFGGSYPSLEGEKAYRETT |
| | (SEQ ID NO. 87) | DLGIEPLRIGIKKLDENAIDNYKPTEIASSLLVVIQMVSEAARFTFIENQIRNNFQQRIRPANNTI |
| | | SLENKWGKLSFQIRTSGANGMFSEAVELERANGKKYYVTAVDQVKPKIALLKFVDKDPE |
| | | (SEQ ID NO. 98) |
| | GSTSGGGSGGG | Linker between Heavy-Light chains |
| | SGGGGSS | |
| | (SEQ ID NO. 84) | |
| ABCB5-Ab73 | GSTSGGGSGGG | GLDTVSFSTKGATYITYVNFLNELRVKLKPEGNSHGIPLLRKKCDDPGKCFVLVALSNDNGQL |
| | SGGGSGGGGSS | AEIAIDVTSVYVVGYQVRNRSYFFKDAPDAAYEGLFKNTIKTRLHFGGSYPSLEGEKAYRETT |
| | (SEQ ID NO. 87) | DLGIEPLRIGIKKLDENAIDNYKPTEIASSLLVVIQMVSEAARFTFIENQIRNNFQQRIRPANNTI |
| | | SLENKWGKLSFQIRTSGANGMFSEAVELERANGKKYYVTAVDQVKPKIALLKFVDKDPE |
| | | (SEQ ID NO. 98) |
| | GSTSGGGSGGG | Linker between Heavy-Light chains |
| | SGGGGSS | |
| | (SEQ ID NO. 84) | |
| | GSTSGGGSGGG | Linker between Light-Heavy chainss |
| | SGGGSGGGGSS | |
| | GSTSGGGSGGG | |
| | SGGGSGGGGSS | |
| | (SEQ ID NO. 88) | |
| ABCB5-Ab74 | GSTSGGGSGGG | GLDTVSFSTKGATYITYVNFLNELRVKLKPEGNSHGIPLLRKKCDDPGKCFVLVALSNDNGQL |
| | SGGGSGGGGSS | AEIAIDVTSVYVVGYQVRNRSYFFKDAPDAAYEGLFKNTIKTRLHFGGSYPSLEGEKAYRETT |
| | (SEQ ID NO. 87) | DLGIEPLRIGIKKLDENAIDNYKPTEIASSLLVVIQMVSEAARFTFIENQIRNNFQQRIRPANNTI |
| | | SLENKWGKLSFQIRTSGANGMFSEAVELERANGKKYYVTAVDQVKPKIALLKFVDKDPE |
| | | (SEQ ID NO. 98) |
| | GSTSGGGSGGG | Linker between Heavy-Light chains |
| | SGGGGSS | |
| | (SEQ ID NO. 84) | |
| ABCB5-Ab75 | GSTSGGGSGGG | GLDTVSFSTKGATYITYVNFLNELRVKLKPEGNSHGIPLLRKKCDDPGKCFVLVALSNDNGQL |
| | SGGGSGGGGSS | AEIAIDVTSVYVVGYQVRNRSYFFKDAPDAAYEGLFKNTIKTRLHFGGSYPSLEGEKAYRETT |
| | (SEQ ID NO. 87) | DLGIEPLRIGIKKLDENAIDNYKPTEIASSLLVVIQMVSEAARFTFIENQIRNNFQQRIRPANNTI |
| | | SLENKWGKLSFQIRTSGANGMFSEAVELERANGKKYYVTAVDQVKPKIALLKFVDKDPE |
| | | (SEQ ID NO. 98) |
| | GSTSGGGSGGG | Linker between Heavy-Light chains |
| | SGGGGSS | |
| | (SEQ ID NO. 84) | |
| | GSTSGGGSGGG | Linker between Light-Heavy chains |
| | SGGGSGGGGSS | |
| | GSTSGGGSGGG | |
| | SGGGSGGGGSS | |
| | (SEQ ID NO. 88) | |
| ABCB5- | GSTSGGGSGGG | GGSLAALTAHQACHLPLETFTRHRQPRGWEQLEQCGYPVQRLVALYLAARLSWNQVDQVIR |
| Ab76 | SGGGSGGGGSS | NALASPGSGGDLGEAIREQPEQARLALTLAAAESERFVRQGTGNDEAGAANADVVSLTCPVA |
| Ab77 | (SEQ ID NO. 87) | AGECAGPADSGDALLERNYPTGAEFLGDGGDVSFSTRGTQNWTVERLLQAHRQLEERGYVF |
| Ab78 | | VGYHGTFLEAAQSIVFGGVRARSQDLDAIWRGFYIAGDPALAYAYAQDQEPDARGRIRNGAL |
| Ab79 | | LRVYVPRSSLPGFYRTSLTLAAPEAAGEVERLIGHPLPLRLDAITGPEEEGGRLETILGWPLAER |
| Ab80 | | TVVIPSAIPTDPRNVGGDLDPSSIPDKEQAISALPDYASQPGKPPREDLK |
| Ab81 | | (SEQ ID NO. 99) |
| ABCB5- | GSTSGGGSGGG | KIYVVATIAWILLQFSAWTTTDAVTSITLDLVNPTAGQYSSFVDKIRNNVKDPNLKYGGTDIA |
| Ab82 | SGGGSGGGGSS | VIGPPSKEKFLRINFQSSRGTVSLGLKRDNLYVVAYLAMDNTNVNRAYYFRSEITSAESTALFP |
| Ab83 | (SEQ ID NO. 87) | EATTANQKALEYTEDYQSIEKNAQITQGDQSRKELGLGIDLLSTSMEAVNKKARVVKDEARF |
| Ab84 | | LLIAIQMTAEAARFRYIQNLVIKNFPNKFNSENKVIQFEVNWKKISTAIYGDAKNGVFNKDYD |
| Ab85 | | FGFGKVRQVKDLQMGLLMYLGKPK |
| Ab86 | | (SEQ ID NO. 100) |
| Ab87 | | |
| ABCB5- | GSTSGGGSGGG | GSPRTEYEACRVRCQVAEHGVERQRRCQQVCEKRLREREGRRE |
| Ab88 | SGGGSGGGGSS | (SEQ ID NO. 101) |
| Ab89 | (SEQ ID NO. 87) | |
| Ab90 | | |
| Ab91 | | |
| Ab92 | | |
| Ab93 | | |
| ABCB5- | GSTGGGSGGGS | MGADDWDSSKSFVMENFSSYHGTKPGYVDSIQKGIQKPKSGTQGNYDDDWKGFYSTDNKY |
| Ab94 | GGGSGGGGSS | DAAGYSVDNENPLSGKAGGVVKVTYPGLTKVLALKVDNAETIKKELGLSLTEPLMEQVGTE |
| Ab95 | (SEQ ID NO. 89) | EFIKRFGDGASRVVLSLPFAEGSSSVEYINNWEQAKALSVELEINFETRGKRGQDAMYEYMA |
| Ab96 | | QACAGNRVRRSVGSSLSCINLDWDVIRDKTKTKIESLKEHGPIKNKMSESPNKTVSEEKAKQY |
| Ab97 | | LEEFHQTALEHPELSELKTVTGTNPVFAGANYAAWAVNVAQVIDSETADNLEKTTAALSILP |
| Ab98 | | GIGSVMGIADGAVHHNTEEIVAQSIALSSLMVAQAIPLVGELVDIGFAAYNFVESIINLFQVVH |
| Ab99 | | NSYNRPAYSPGHKT |
| | | (SEQ ID NO. 102) |

TABLE 8
Bispecific Formats

| Exemplary | |
|---|---|
| Antibody | Structure |
| ABCB5-Ab18 | IgG-scFv - bispecific ABCB5-Ab58 format - fused to anti-CD3 scFv |
| ABCB5-Ab19 | IgG-scFv - bispecific ABCB5-Ab58 format - fused to anti-CD3 scFv |
| ABCB5-Ab20 | IgG-scFv - bispecific ABCB5-Ab58 format - fused to anti-CD3 scFv |
| ABCB5-Ab21 | IgG-scFv - bispecific ABCB5-Ab58 format - fused to anti-CD3 scFv |
| ABCB5-Ab22 | IgG-scFv - bispecific ABCB5-Ab58 format - fused to anti-Cd16 scFv |
| ABCB5-Ab23 | IgG-scFv - bispecific ABCB5-Ab58 format - fused to anti-Cd16 scFv |
| ABCB5-Ab24 | IgG-scFv - bispecific ABCB5-Ab58 format - fused to anti-Cd16 scFv |
| ABCB5-Ab25 | IgG-scFv - bispecific ABCB5-Ab58 format - fused to anti-Cd16 scFv |
| ABCB5-Ab26 | Single chain diabodies (scDb) - bispecific ABCB5-Ab58 format - fused to anti-CD3 scFv |
| ABCB5-Ab27 | Single chain diabodies (scDb) - bispecific ABCB5-Ab58 format - fused to anti-CD3 scFv |
| ABCB5-Ab28 | Single chain diabodies (scDb) - bispecific ABCB5-Ab58 format - fused to anti-CD3 scFv |
| ABCB5-Ab29 | Single chain diabodies (scDb) - bispecific ABCB5-Ab58 format - fused to anti-CD3 scFv |
| ABCB5-Ab30 | Single chain diabodies (scDb) - bispecific ABCB5-Ab58 format - fused to anti-Cd16 scFv |
| ABCB5-Ab31 | Single chain diabodies (scDb) - bispecific ABCB5-Ab58 format - fused to anti-Cd16 scFv |
| ABCB5-Ab32 | Single chain diabodies (scDb) - bispecific ABCB5-Ab58 format - fused to anti-Cd16 scFv |
| ABCB5-Ab33 | Single chain diabodies (scDb) - bispecific ABCB5-Ab58 format - fused to anti-Cd16 scFv |
| ABCB5-Ab34 | Tandem scFv's (taFv) - bispecific ABCB5-Ab58 format - fused to anti-CD3 scFv |
| ABCB5-Ab35 | Tandem scFv's (taFv) - bispecific ABCB5-Ab58 format - fused to anti-CD3 scFv |
| ABCB5-Ab36 | Tandem scFv's (taFv) - bispecific ABCB5-Ab58 format - fused to anti-CD3 scFv |
| ABCB5-Ab37 | Tandem scFv's (taFv) - bispecific ABCB5-Ab58 format - fused to anti-CD3 scFv |
| ABCB5-Ab38 | Tandem scFv's (taFv) - bispecific ABCB5-Ab58 format - fused to anti-Cd16 scFv |
| ABCB5-Ab39 | Tandem scFv's (taFv) - bispecific ABCB5-Ab58 format - fused to anti-Cd16 scFv |
| ABCB5-Ab40 | Tandem scFv's (taFv) - bispecific ABCB5-Ab58 format - fused to anti-Cd16 scFv |
| ABCB5-Ab41 | Tandem scFv's (taFv) - bispecific ABCB5-Ab58 format - fused to anti-Cd16 scFv |
| ABCB5-Ab46 | IgG-scFv - bispecific ABCB5-Ab59 format - fused to anti-CD3 scFv |
| ABCB5-Ab47 | IgG-scFv - bispecific ABCB5-Ab59 format - fused to anti-CD3 scFv |
| ABCB5-Ab48 | IgG-scFv - bispecific ABCB5-Ab59 format - fused to anti-Cd16 scFv |
| ABCB5-Ab49 | IgG-scFv - bispecific ABCB5-Ab59 format - fused to anti-Cd16 scFv |
| ABCB5-Ab50 | Single chain diabodies (scDb) - bispecific ABCB5-Ab59 format - fused to anti-CD3 scFv |
| ABCB5-Ab51 | Single chain diabodies (scDb) - bispecific ABCB5-Ab59 format - fused to anti-CD3 scFv |
| ABCB5-Ab52 | Single chain diabodies (scDb) - bispecific ABCB5-Ab59 format - fused to anti-Cd16 scFv |
| ABCB5-Ab53 | Single chain diabodies (scDb) - bispecific ABCB5-Ab59 format - fused to anti-Cd16 scFv |
| ABCB5-Ab54 | Tandem scFv's (taFv) - bispecific ABCB5-Ab59 format - fused to anti-CD3 scFv |
| ABCB5-Ab55 | Tandem scFv's (taFv) - bispecific ABCB5-Ab59 format - fused to anti-CD3 scFv |
| ABCB5-Ab56 | Tandem scFv's (taFv) - bispecific ABCB5-Ab59 format - fused to anti-Cd16 scFv |
| ABCB5-Ab57 | Tandem scFv's (taFv) - bispecific ABCB5-Ab59 format - fused to anti-Cd16 scFv |

In some embodiments, the anti-ABCB5 antibody described herein binds the same epitope in an ABCB5 antigen as a reference antibody disclosed herein (e.g., ABCB5-Ab100 or ABCB5-Ab101) or competes against the reference antibody from binding to the ABCB5 antigen. A “reference antibody” is an antibody that binds to ABCB5 and provides a comparator, functionally or structurally, to an ABCB5 antibody of the invention. In some embodiments the reference antibody is a commercially available antibody. In other embodiments the reference antibody is a parent antibody. A “parent antibody” as used herein refers to an antibody template which is modified to create child antibodies which maintain some or all of the parent antibodies desirable characteristics such as binding specificity, binding affinity or the functionality of parental antibody i.e., as assessed in biological assays. In some embodiments Ab100 and Ab101 are exemplary parent antibodies.

An “epitope” refers to the site on a target compound that is bound by an antibody such as a Fab or full length antibody. An epitope can be linear, which is typically 6-15 amino acid in length. Alternatively, the epitope can be conformational. An antibody that binds the same epitope as a reference antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the reference antibody. Whether two antibodies compete against each other from binding to the cognate antigen can be determined by a competition assay, which is well known in the art. Such antibodies can be identified as known to those skilled in the art, e.g., those having substantially similar structural features (e.g., complementary determining regions), and/or those identified by assays known in the art. For example, competition assays can be performed using one of the reference antibodies to determine whether a candidate antibody binds to the same epitope as the reference antibody or competes against its binding to the ABCB5 antigen.

In some embodiments the epitope is an extracellular loop. For instance, the antibodies of the invention may bind with high specificity and/or affinity to the 1^{st}, 2^{nd} or 3^{rd} extracellular loop (EC loop) of human ABC transporter protein ABCB5 transcript variant 2 or beta isoform [NCBI accession NM_178559.5]. Exemplary epitopes include for instance any of the following sequences or portions thereof: RFGAYLIQAGRMTPEG (SEQ ID NO. 104), TMFGNNDKTTLKHDAE (SEQ ID NO. 105), VTGMIETAAMTGFANKDKQELKHAGKIATEALENIRTIVSLTREKAFEQMYEEMLQT QHRNTSKKAQI (SEQ ID NO. 106), or QDIKKADEQMESMTYSTERKTNSLPLHSVKSIKSDFIDKAEESTQSKEISLPEVSLLK (SEQ ID NO. 107). Alternatively the antibodies may bind with high specificity and/or affinity to an intracellular loop (nucleotide-binding domain; NBD1 or 2) or an intracellular domain/region (other than NDB).

An exemplary soluble partial recombinant ABCB5 standard protein has the following sequence:

NBD1-spacer-EC1-spacer-ICD2-spacer-EC3-spacer-
NBD2:
(SEQ ID NO. 108)
MVDENDIRALNVRHYRDHIGVVSQEPVLFGTTISNNIKYGRDDVTDE
EMERAAREANAYDFIMEFPNKFNTLVGEKGAQ [symbol] IAIARAL
VRNPKILILDEATSALDSESKSAVQAALEKASKGRTTIVVAHRLSTI
RSADLIVTLKDGMLAEKGAHAELMAKRGLYYSLVMSQDIKKADEQME
SMTYSTERKTNSLPLHSVKSIKSDFIDKAEESTQSKEISLPEVSLLK
ILKLNKPEWPFVGSTSGGGSGGGSGGGSGGGGSS [C] TMFGNNDK
TTLKHDAE [C] GSTSGGGSGGGSGGGSGGGGSSVTGMIETAAMTG
FANKDKQELKHAGKIATEALENIRTIVSLTREKAFEQMYEEMLQTQH
RNTSKKAQIGSTSGGGSGGGSGGGSGGGGSS [C] RFGAYLIQAGR
MTPEG [C] GSTSGGGSGGGSGGGSGGGGSSEYSKAKSGAAHLFAL
LEKKPNIDSRSQEGKKPDTCEGNLEFREVSFFYPCRPDVFILRGLSL
SIERGKTVAFVGSSGCGKSTSVQLLQRLYDPVQGQVLFDGVDAKELN
VQWLRSQIAIVPQEPVLFNCSIAENIAYGDNSRVVPLDEIKEAANAA
NIHSFIEGLPEKYNTQVGLKGAQ [symbol] LAIARALLQKPKILLL
DEATSALDNDSEKVVQHALDKARTGRTCLVVTHRLSAIQNADLIVVL
HNGKIKEQGTHQELLRNRDIYFKLVNAQSVQ.
NBD1:
(SEQ ID NO: 48)
MVDENDIRALNVRHYRDHIGVVSQEPVLFGTTISNNIKYGRDDVTDE
EMERAAREANAYDFIMEFPNKFNTLVGEKGAQMSGGQKQRIAIARAL
VRNPKILILDEATSALDSESKSAVQAALEKASKGRTTIVVAHRLSTI
RSADLIVTLKDGMLAEKGAHAELMAKRGLYYSLVMSQDIKKADEQME
SMTYSTERKTNSLPLHSVKSIKSDFIDKAEESTQSKEISLPEVSLLK
ILKLNKPEWPFV
Spacer 1:
(SEQ ID NO: 87)
GSTSGGGSGGGSGGGSGGGGSS[C]
EC1:
(SEQ ID NO: 105)
TMFGNNDKTTLKHDAE
Spacer 2:
(SEQ ID NO: 87)
[C]GSTSGGGSGGGSGGGSGGGGSS
ICD2:
(SEQ ID NO: 106)
VTGMIETAAMTGFANKDKQELKHAGKIATEALENIRTIVSLTREKAF
EQMYEEMLQTQHRNTSKKAQI
Spacer 3:
(SEQ ID NO: 87)
GSTSGGGSGGGSGGGSGGGGSS[C]
EC3:
(SEQ ID NO: 104)
RFGAYLIQAGRMTPEG
Spacer 4:
(SEQ ID NO: 87)
[C]GSTSGGGSGGGSGGGSGGGGSS
NBD2:
(SEQ ID NO: 68)
EYSKAKSGAAHLFALLEKKPNIDSRSQEGKKPDTCEGNLEFREVSFFY
PCRPDVFILRGLSLSIERGKTVAFVGSSGCGKSTSVQLLQRLYDPVQG
QVLFDGVDAKELNVQWLRSQIAIVPQEPVLFNCSIAENIAYGDNSRVV
PLDEIKEAANAANIHSFIEGLPEKYNTQVGLKGAQLSGGQKQRLAIAR
ALLQKPKILLLDEATSALDNDSEKVVQHALDKARTGRTCLVVTHRLSA
IQNADLIVVLHNGKIKEQGTHQELLRNRDIYFKLVNAQSVQ

Additional Cysteine residues [C] can be introduced at the beginning/end of the particular spacers in order to emulate the native extracellular loop (EC) structure of the corresponding sequence stretches as actually present in native ABCB5 protein.

In some embodiments, an anti-ABCB5 antibody disclosed herein may comprise the same regions/residues responsible for antigen-binding as a reference antibody (e.g., Ab100 or Ab101), such as the same specificity-determining residues in the CDRs or the whole CDRs. The regions/residues that are responsible for antigen-binding can be identified from amino acid sequences of the heavy chain/light chain sequences of the reference antibody (shown above) by methods known in the art. See, e.g., bioinf.org.uk/abs; Almagro, J. Mol. Recognit. 17:132-143 (2004); Chothia et al., J. Mol. Biol. 227:799-817 (1987), as well as others known in the art or disclosed herein. Determination of CDR regions in an antibody is well within the skill of the art, for example, the methods disclosed herein, e.g., the Kabat method (Kabat et al. Sequences of Proteins of Immunological Interest, (5th ed., 1991, National Institutes of Health, Bethesda Md.)) or the Chothia method (Chothia et al., 1989, Nature 342:877; Al-Iazikani et al (1997) J. Molec. Biol. 273:927-948)). As used herein, a CDR may refer to the CDR defined by any method known in the art. Two antibodies having the same CDR means that the two antibodies have the same amino acid sequence of that CDR as determined by the same method. In specific examples, the anti-ABCB5 antibodies disclosed herein have the same V_{H} and/or V_{L} as a reference antibody, such as Ab100 or Ab101.

Also within the scope of the present disclosure are functional variants of any of the exemplary anti-ABCB5 antibodies as disclosed herein. A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, anti-tumor activity, or a combination thereof) as the reference antibody.

In some examples, the anti-ABCB5 antibody disclosed herein comprises a HC CDR1, a HC CDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as Ab100 or Ab101. “Collectively” means that the total number of amino acid variations in all of the three HC CDRs is within the defined range. Alternatively or in addition, the anti-ABCB5 antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody.

In some examples, the anti-ABCB5 antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody such as Ab100 or Ab101. In specific examples, the antibody comprises a HC CDR3, which contains no more than 5 amino acid variations (e.g., no more than 4, 3, 2, or 1 amino acid variation) as the HC CDR3 of a reference antibody such as Ab100 or Ab101. Alternatively or in addition, an anti-ABCB5 antibody may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody. In specific examples, the antibody comprises a LC CDR3, which contains no more than 5 amino acid variations (e.g., no more than 4, 3, 2, or 1 amino acid variation) as the LC CDR3 of the reference antibody.

The present disclosure provides several types of compositions that are polynucleotide or polypeptide based, including variants and derivatives. These include, for example, substitutional, insertional, deletion and covalent variants and derivatives. The term “derivative” is used synonymously with the term “variant” but generally refers to a molecule that has been modified and/or changed in any way relative to a reference molecule or starting molecule.

As such, polynucleotides encoding peptides or polypeptides containing substitutions, insertions and/or additions, deletions and covalent modifications with respect to reference sequences, in particular the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. Substitutions may be single, where only one amino acid in the molecule has been substituted, or they may be multiple, where two or more amino acids have been substituted in the same molecule.

As used herein, the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Features” when referring to polypeptide or polynucleotide are defined as distinct amino acid sequence-based or nucleotide-based components of a molecule respectively. Features of the polypeptides include surface manifestations, local conformational shape, folds, loops, half-loops, domains, half-domains, sites, termini, or any combination thereof.

As used herein, when referring to polypeptides, the term “domain” refers to a motif of a polypeptide having one or more identifiable structural or functional characteristics or properties (e.g., binding capacity, serving as a site for protein-protein interactions).

As used herein, when referring to polypeptides, the terms “site” as it pertains to amino acid based embodiments is used synonymously with “amino acid residue” and “amino acid side chain.” As used herein, when referring to polynucleotides, the terms “site” as it pertains to nucleotide based embodiments is used synonymously with “nucleotide.” A site represents a position within a peptide or polypeptide or polynucleotide that may be modified, manipulated, altered, derivatized or varied within the polypeptide or polynucleotide based molecules.

As used herein, the terms “termini” or “terminus” when referring to polypeptides or polynucleotides refers to an extremity of a polypeptide or polynucleotide, respectively. Such extremity is not limited only to the first or final site of the polypeptide or polynucleotide, but may include additional amino acids or nucleotides in the terminal regions. Polypeptide-based molecules may be characterized as having both an N-terminus (terminated by an amino acid with a free amino group (NH₂)) and a C-terminus (terminated by an amino acid with a free carboxyl group (COOH)). Proteins are in some cases made up of multiple polypeptide chains brought together by disulfide bonds or by non-covalent forces (e.g., multimers, oligomers). These proteins have multiple N- and C-termini. Alternatively, the termini of the polypeptides may be modified such that they begin or end, as the case may be, with a non-polypeptide based moiety such as an organic conjugate.

As recognized by those skilled in the art, protein fragments, functional protein domains, and homologous proteins are also considered to be within the scope of polypeptides of interest. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of 20, 30, 40, 50, or 100 amino acids which are 40%, 50%, 60%, 70%, 80%, 90%, 95%, or 100% identical to any of the sequences described herein can be utilized in accordance with the disclosure. In some embodiments, a polypeptide includes 2, 3, 4, 5, 6, 7, 8, 9, 10, or more mutations as shown in any of the sequences provided or referenced herein. In another example, any protein that includes a stretch of 20, 30, 40, 50, or 100 amino acids that are greater than 80%, 90%, 95%, or 100% identical to any of the sequences described herein, wherein the protein has a stretch of 5, 10, 15, 20, 25, or 30 amino acids that are less than 80%, 75%, 70%, 65% or 60% identical to any of the sequences described herein can be utilized in accordance with the disclosure.

Polypeptide or polynucleotide molecules of the present disclosure may share a certain degree of sequence similarity or identity with the reference molecules (e.g., reference polypeptides or reference polynucleotides), for example, with Ab100 or Ab101 (e.g., engineered or designed molecules or wild-type molecules).

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

As used herein, the term “homology” refers to the overall relatedness between polymeric molecules, e.g. between nucleic acid molecules (e.g., DNA molecules and/or RNA molecules) and/or between polypeptide molecules. Polymeric molecules (e.g., nucleic acid molecules (e.g., DNA molecules and/or RNA molecules) and/or polypeptide molecules) that share a threshold level of similarity or identity determined by alignment of matching residues are termed homologous. Homology is a qualitative term that describes a relationship between molecules and can be based upon the quantitative similarity or identity. Similarity or identity is a quantitative term that defines the degree of sequence match between two compared sequences. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). Two polynucleotide sequences are considered homologous if the polypeptides they encode are at least 50%, 60%, 70%, 80%, 90%, 95%, or even 99% for at least one stretch of at least 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. Two protein sequences are considered homologous if the proteins are at least 50%, 60%, 70%, 80%, or 90% identical for at least one stretch of at least 20 amino acids.

Homology implies that the compared sequences diverged in evolution from a common origin. The term “homolog” refers to a first amino acid sequence or nucleic acid sequence (e.g., gene (DNA or RNA) or protein sequence) that is related to a second amino acid sequence or nucleic acid sequence by descent from a common ancestral sequence. The term “homolog” may apply to the relationship between genes and/or proteins separated by the event of speciation or to the relationship between genes and/or proteins separated by the event of genetic duplication. “Orthologs” are genes (or proteins) in different species that evolved from a common ancestral gene (or protein) by speciation. Typically, orthologs retain the same function in the course of evolution. “Paralogs” are genes (or proteins) related by duplication within a genome. Orthologs retain the same function in the course of evolution, whereas paralogs evolve new functions, even if these are related to the original one.

In some embodiments, the heavy chain constant region used in the anti-ABCB5 antibodies described herein may comprise mutations (e.g., amino acid residue substitutions) to modulate (e.g., enhance or reduce) the ADCC activity. In some examples, the heavy chain constant region may comprise an amino acid residue mutation at one or more of positions E233, L234, L235, G236, A327, A330, K322, E318, K320, and P331 (numbering according to the EU index) to reduce ADCC activity and/or CDC activity. The mutations may comprise E233P, L234V, L235A, deltaG236, A327G, A330S, P331S, K322A, E318A, K320A, K322A, or a combination thereof. In other examples, the heavy chain constant region may comprise an amino acid residue mutation at one or more of positions S298, S239, K334, M252, S254, A330, 1332, K326, E335, and T256 (numbering according to the EU index) to enhance the ADCC activity and/or CDC activity. The amino acid residue mutations may comprise S298A, K334A, M252Y, S254T, S239D, A330L, 1332E, K326W, E335S, and T256E, or a combination thereof. In some instances, the heavy chain constant region of an anti-ABCB5 antibody described herein may be from human IgG1 and comprises a mutation at position K214 (EU index numbering), for example, the K214R substitution.

In some embodiments, the heavy chain constant region used in the anti-ABCB5 antibodies described herein may comprise mutations (e.g., amino acid residue substitutions) to enhance a desired characteristic of the antibody, for example, increasing the binding activity to the neonatal Fc receptor (FcRn) and thus the serum half-life of the antibodies. It was known that binding to FcRn is critical for maintaining antibody homeostasis and regulating the serum half-life of antibodies. One or more (e.g., 1, 2, 3, 4, 5, or more) mutations (e.g., amino acid residue substitutions) may be introduced into the constant region at suitable positions (e.g., in CH2 region) to enhance FcRn binding and enhance the half-life of the antibody. The amino acid residue mutations may comprise, for instance, T250Q, M428L, M252Y, S254T, T256E, H433K, N434F, or a combination thereof.

The present disclosure also provides germlined variants of any of the exemplary anti-ABCB5 antibodies disclosed herein. A germlined variant contains one or more mutations in the framework regions as relative to its parent antibody towards the corresponding germline sequence. To make a germline variant, the heavy or light chain variable region sequence of the parent antibody or a portion thereof (e.g., a framework sequence) can be used as a query against an antibody germline sequence database (e.g., bioinfo.org.uk/abs/, vbase2.org, or imgt.org) to identify the corresponding germline sequence used by the parent antibody and amino acid residue variations in one or more of the framework regions between the germline sequence and the parent antibody. One or more amino acid substitutions can then be introduced into the parent antibody based on the germline sequence to produce a germlined variant. As described herein, the anti-ABCB5 antibody can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies.

In another aspect, the invention relates to a conjugate comprising an antibody of the invention coupled to a moiety such as a pharmaceutically active moiety. The pharmaceutically active moiety may be, for instance, a compound that stabilizes the antibody, a label, or a therapeutic agent.

As used herein the term “coupled” refers to the joining or connection of two or more objects together. When referring to chemical or biological compounds, coupled can refer to a covalent connection between the two or more chemical or biological compounds. By way of a non-limiting example, an antibody of the invention can be coupled with a compound such as a peptide to form an antibody coupled compound/peptide. An antibody coupled peptide can be formed through specific chemical reactions designed to conjugate the antibody to the peptide.

In certain embodiments the pharmaceutically active moiety can be linked directly to the antibody or in other embodiments it may be covalently coupled with the antibody through a linker. The linker can be modified chemically to allow for the conjugation of the antibody to the pharmaceutically active moiety. The linker can, for example, include, but is not limited to, a peptide linker (such as the linkers described above), a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain. The PEG linkers can, for example, comprise 2-24 PEG units. Sulfamide linkers have been found to improve the solubility of a linker-conjugate, which in turn significantly improves the efficiency of the conjugation and reduces both in process and product aggregation. Other linkers known in the art, include a linker which contains hydrophilic regions represented by PEG and an extension lacking chiral centers that is coupled to a targeting agent (WO 2008/070291); and a linker system having a novel hydrophilic spacer group (WO 01/88535). The design of the linker is important because it may impact both the efficacy and safety of the ADCs. The linker should provide sufficient stability during systemic circulation but allow for the rapid and efficient intracellular release of the drug in an active form.

The ADC may also include a spacer unit. A spacer links the antibody to the drug, with an optional linker and stretcher. Spacer units typically are of two general types: self-immolative and non self-immolative. A non self-immolative spacer unit is one in which part or all of the spacer unit remains bound to the drug after enzymatic cleavage of the antibody-drug conjugate. Examples of a non self-immolative spacer unit include, but are not limited to a (glycine-glycine) spacer unit and a glycine spacer unit. To release the drug, an independent hydrolysis reaction may take place within the target cell to cleave the glycine-drug unit bond. In some embodiments, a non self-immolative the spacer is Gly. Alternatively, an ADC contains a self-immolative spacer that can release the drug without the need for a separate hydrolysis step. In these embodiments, the spacer may be substituted and unsubstituted 4-aminobutyric acid amides, appropriately substituted bicyclo[2.2.1] and bicyclo[2.2.2] ring systems and 2-aminophenylpropionic acid amides.

As used herein, the term “conjugate” refers to an antibody, including antibody fragments thereof covalently coupled to a pharmaceutically active moiety. The term “conjugated to” refers to an antibody or a fragment thereof of invention covalently linked to or covalently connected to a pharmaceutically active moiety directly or indirectly via a linker. The pharmaceutically active moiety may be a peptide or a non-peptide organic moiety (i.e., “small molecule”).

Thus, the ADC has as its most basic structure an Ab-Drug. Optionally the structure is Ab-linker-Drug; Ab-linker-spacer-Drug; or Ab-linker-spacer-stretcher-Drug. Numerous methods for producing these constructs have been described in the art and the constructs of the invention are not limited to any one particular method. Exemplary bioconjugation methods and components thereof are described for instance in US20190262466; WO2002088172, U.S. Pat. Nos. 6,884,869, 7,098,308, 7,256,257, WO2004010957, WO2005001038, U.S. Pat. Nos. 7,659,241, 8,906,376, 7,659,241, WO2005081711, each of which is incorporated by reference.

In some embodiments the pharmaceutically active moiety is a therapeutic agent. An antibody coupled to a therapeutic agent is referred to herein as an antibody drug conjugate (ADC). An ADC, as used herein, refers to an anti-ABCB5 antibody (full length or any of variant, fragment or other form of an antibody) combined with a therapeutic payload (cytotoxic agent) optionally through a linker and/or spacer. For example, Ab101, and several of its affinity maturated variants has successfully been combined chemically through an enzymatically trimmed glycostructure at N297 residue to drugs including a MMAE payload. The scope of the invention, is however, not limited to the particular nature/origin of the anti-ABCB5 antibody or antibody fragment, the valency (one scFv for monovalent binding or multivalent anti-ABCB5 species like diabodies, [Fab]2 fragments etc.), the nature, length and composition of linker and to the nature of active agent.

An exemplary therapeutic agent for use in the ADCs of the invention is an antitumor compound. The antitumor compound has an antitumor effect and has a substituent or a partial structure that can be connected to directly to the antibody or indirectly to the antibody through a linker. Upon cleavage of a part or the whole of the linker in tumor cells, the antitumor compound is released so that the antitumor compound exhibits an antitumor effect. Examples of useful antitumor compounds include, doxorubicin, calicheamicin, dolastatin 10, auristatins such as monomethyl auristatin E (MMAE) and monomethyl auristatin F (MMAF), maytansinoids such as DM1 and DM4, a pyrrolobenzodiazepine dimer SG2000 (SJG-136), a camptothecin derivative SN-38, duocarmycins such as CC 1065, amanitin, daunorubicin, mitomycin C, bleomycin, cyclocytidine, vincristine, vinblastine, methotrexate, platinum-based antitumor agents (cisplatin and derivatives thereof), Taxol and derivatives thereof, and exatecan (a camptothecin derivative ((1S,9S)-1-amino-9-ethyl-5-fluoro-2,3-dihydro 9-hydroxy-4-methyl-1H,12H benzo[de]pyrano[3′,4′:6,7]indolizino[1,2-b]quinoline 10,13(9H,15H)-dione).

The auristatin peptides, auristatin E (AE) and monomethylauristatin E (MMAE), synthetic analogs of dolastatin, have been conjugated as drug moieties to various antibodies and are useful in the ADC of the invention.

The ADC of the invention may also be cysteine-engineered antibodies that are FAB antibody fragments (thioFab) and full-length, IgG (thioMab) antibodies (US 2007/0092940). ThioFab and ThioMab antibodies are conjugated through linkers at the newly introduced cysteine thiols with thiol-reactive linker reagents and drug-linker reagents to prepare ADC.

Dolastatins and auristatins have been shown to interfere with microtubule dynamics, GTP hydrolysis, and nuclear and cellular division and have anticancer activity. Various forms of a dolastatin or auristatin drug moiety may be covalently attached to an antibody through the N (amino) terminus or the C (carboxyl) terminus of the peptidic drug moiety. Exemplary auristatin embodiments include the N-terminus linked monomethylauristatin drug moieties DE and DF, disclosed in: WO 2005/081711, such as MMAE, and MMAF. The N-terminus of the MMAE or MMAF drug moiety may be covalently attached via a linker to an engineered cysteine of the antibody.

Other exemplary auristatin drug moieties include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Other therapeutic agents that are useful in combination with the antibodies of the invention include Pseudomonas aeruginosa exotoxin A (aka monatox), diphteria toxin, saporin, luffin P1 etc. or their recombinant forms.

In some embodiments the ADC have a minimal therapeutic index. The term “therapeutic index” (TI) as used herein refers to the ratio of the dose of drug that is toxic (i.e. causes adverse effects at an incidence or severity not compatible with the targeted indication) for 50% of the population (TD₅₀) divided by the dose that leads to the desired pharmacological effect in 50% of the population (effective dose or ED₅₀). Hence, TI=TD₅₀/ED₅₀. The therapeutic index may be determined by clinical trials or for example by plasma exposure tests. See also Muller, et al. Nature Reviews Drug Discovery 2012, 11, 751-761. At an early development stage, the clinical TI of a drug candidate is often not yet known. However, understanding the preliminary TI of a drug candidate is of utmost importance as early as possible, since TI is an important indicator of the probability of the successful development of a drug. In animal models TI is typically defined as the quantitative ratio between efficacy (minimal effective dose in a mouse xenograft) and safety (maximum tolerated dose in mouse or rat).

The term “therapeutic efficacy” refers to the capacity of a substance to achieve a certain therapeutic effect, e.g. reduction in tumor volume. Therapeutic effects can be measured determining the extent in which a substance can achieve the desired effect, typically in comparison with another substance under the same circumstances. A suitable measure for the therapeutic efficacy is the ED₅₀ value, which may for example be determined during clinical trials or by plasma exposure tests. In case of preclinical therapeutic efficacy determination, the therapeutic effect of an ADC, can be validated by patient-derived tumor xenografts in mice in which case the efficacy refers to the ability of the ADC to provide a beneficial effect. Alternatively the tolerability of said ADC in a rodent safety study can also be a measure of the therapeutic effect.

In the antibody-drug conjugate, the number of conjugated drug molecules per antibody molecule may be an important factor having an influence on the efficacy and safety thereof. The production of the antibody-drug conjugate is carried out by specifying reaction conditions such as the amount of starting materials and reagents used for reaction, so as to attain a constant number of conjugated drug molecules. Unlike the chemical reaction of a low molecular-weight compound, a mixture containing different numbers of conjugated drug molecules is usually obtained. The number of conjugated drug molecules per antibody molecule is defined and indicated as an average value, i.e., the average number of conjugated drug molecules. Unless otherwise specified, i.e., except in the case of representing an ADC having a specific number of conjugated drug molecules that is included in an ADC mixture having different numbers of conjugated drug molecules, the number of conjugated drug molecules according to the present invention typically means an average value. The number of therapeutic molecules conjugated to an antibody molecule is controlled, and as an average number of conjugated drug molecules per antibody, approximately 1 to 10 therapeutic molecules can be conjugated.

In some embodiments, the pharmaceutically active moiety may be a compound that stabilizes the antibody, such that the antibody coupled moiety has an extended/increased half-life compared to the antibody alone. A moiety for extending the half-life of the antibody, for example via covalent linkage may be albumin, albumin variants, albumin-binding proteins and/or domains, transferrin and fragments and analogues thereof. Additional half-life extending moieties that can be incorporated into the conjugates of the invention include, for example, polyethylene glycol (PEG) molecules, such as PEG5000 or PEG20,000, fatty acids and fatty acid esters of different chain lengths, for example laurate, myristate, stearate, arachidate, behenate, oleate, arachidonate, octanedioic acid, tetradecanedioic acid, octadecanedioic acid, docosanedioic acid, and the like, polylysine, octane, carbohydrates (dextran, cellulose, oligo- or polysaccharides) for desired properties. These moieties can be direct fusions with the protein scaffold coding sequences and can be generated by standard cloning and expression techniques. Alternatively, well known chemical coupling methods can be used to attach the moieties to recombinantly and chemically produced conjugates of the invention.

Methods of conjugating antibodies of the invention with the pharmaceutically active moieties of the invention are known in the art. Briefly, the antibodies of the invention can be reduced with a reducing agent (e.g., TCEP (tris(2-carboxyethyl) phosphine), purified (e.g., by protein A adsorption or gel filtration), and conjugated with the pharmaceutically active moiety (e.g., by providing a lyophilized peptide to the reduced antibody under conditions that allow for conjugation). After the conjugation reaction, the conjugate can be purified by ion exchange chromatography or hydrophobic interaction chromatography (HIC) with a final purification step of protein A adsorption. In certain embodiments, the antibodies of the invention can be purified prior to being reduced utilizing HIC methods.

A pegyl moiety can, for example, be added to the peptide molecules of the invention by incorporating a cysteine residue to the C-terminus of the molecule and attaching a pegyl group to the cysteine using well known methods.

Peptide molecules of the invention incorporating additional moieties can be compared for functionality by several well-known assays. For example, the biological or pharmacokinetic activities of a therapeutic peptide of interest, alone or in a conjugate according to the invention, can be assayed using known in vitro or in vivo assays and compared.

In some embodiments, the antibodies of the present disclosure are covalently linked to a carrier or targeting group, or including two encoding regions that together produce a fusion protein (e.g., bearing a targeting group and therapeutic protein or peptide) as a peptide conjugate. The peptide conjugates include a naturally occurring substance, such as a protein (e.g., human serum albumin (HSA), low-density lipoprotein (LDL), high-density lipoprotein (HDL), or globulin); an carbohydrate (e.g., a dextran, pullulan, chitin, chitosan, inulin, cyclodextrin or hyaluronic acid); or a lipid. In some embodiments, the conjugate can comprise a cationic polymer such as, but not limited to, polyamine, polylysine, polyalkylenimine, and polyethylenimine that can be grafted to with poly(ethylene glycol).

In some embodiments, the conjugate can be a biomolecule-polymer conjugate, which comprises a long-acting continuous-release system to provide a greater therapeutic efficacy. The synergistic biomolecule-polymer conjugate can be those described in U.S. Pub. No. US2013/0195799. In some embodiments, the conjugate can be an aptamer conjugate as described in Intl. Pat. Pub. No. WO2012/040524. In some embodiments, the conjugate can be an amine containing polymer conjugate as described in U.S. Pat. No. 8,507,653. Each of the references is herein incorporated by reference in its entirety. In some embodiments, the polynucleotides can be conjugated to SMARTT POLYMER TECHNOLOGY® (PHASERX®, Inc. Seattle, Wash.).

The present disclosure also features chimeric antigen receptors targeting ABCB5 and cells expressing ABCB5. Chimeric antigen receptors (CARs) as disclosed herein are artificial cell-surface receptors that, when built into effector cells such as T cells or NK cells, redirect binding specificity of ABCB5⁺ expressing cells, thereby eliminating the target cells via, e.g., the effector activity of the effector immune cells. A CAR construct often comprises an extracellular antigen binding domain fused to at least an intracellular signaling domain. Cartellieri et al., J Biomed Biotechnol 2010:956304, 2010. The extracellular antigen binding domain, which can be a single-chain antibody fragment (scFv), is specific to an ABCB5 antigen and the intracellular signaling domain can mediate cell signaling that leads to activation of immune cells. As such, immune cells expressing a CAR construct specific to ABCB5 can bind to target cells expressing ABCB5, leading to activation of the immune cells and elimination of the target cells. Such immune cells may be referred to as CAR-T or CAR-NK cells.

A CAR-T cell, as used herein, refers to T cells into which a chimeric receptor has been introduced to redirect their specificity towards an antigen of choice, ABCB5. Such receptors comprise an ectodomain that recognizes antigen independent of MHC restriction, in combination with cytoplasmic signaling domains. Many different peptides can be introduced into the T cells as the ectodomain of the chimeric antigen receptors. Examples include a nanobody, a monoclonal antibody, a humanized antibody, a chimeric antibody, a human antibody, or an antibody fragment, including any of the antibody construct described herein.

Natural killer cells (NK Cells) are peripheral blood lymphocytes that play a role in innate immune function. NK cells express a variety of activating and inhibitory receptors that are responsible for discriminating between healthy cells, and virally infected cells or cancerous cells. Unlike T cells, NK cells exert their cytotoxic effect on target cells in an antigen independent manner. As a result, NK cells do not require antigen priming and can display robust cytotoxicity in the absence of specific antigen. CARs can introduce a certain antigen specificity to an immune effector cell, such as NK cells. Thus, the compositions of the invention include pharmaceutical compositions comprising NK cells, both primary cells and cell lines that have been engineered with at least one antigen binding domain (ABCB5) and are referred to as CAR-NK cells.

Any of the anti-ABCB5 antibodies described herein can be used to produce the CAR constructs also described herein. For example, the V_{H} and V_{L} domains of an anti-ABCB5 antibody can be fused to the intracellular signaling domain(s) to produce a CAR construct using the conventional recombinant technology. In some examples, the V_{H} and V_{L} domains of an anti-ABCB5 are connected via a peptide linker to form a scFv fragment.

The CAR construct disclosed herein may comprise one or more intracellular signaling domains. In some examples, CAR comprises an intracellular signaling domain that includes an immunoreceptor tyrosine-based activation motif (ITAM). Such an intracellular signaling domain may be from CD3, a CD137 (4-1BB) signaling domain, a CD28 signaling domain, a CD3 Zeta signal domain, and any combination thereof.

The CAR construct disclosed herein may further comprise a transmembrane-hinge domain, which can be obtained from a suitable cell-surface receptor, for example, CD28 or CD8. Alternatively, a transmembrane domain composed of an artificial polypeptide may be used.

The intracellular signal domain transmits the signals necessary for exertion of the effector function of the T or NK cell. More specifically, when the extracellular domain binds with the target ABCB5, an intracellular signal domain transmits the signals necessary for activation of the cells. The intracellular signal domain includes the domain for transmitting the signals through for instance the TCR complex, and the domain for transmitting the costimulatory signals. Examples of the costimulatory molecule include CD28, 4-1BB (CD137), CD2, CD4, CD5, CD134, OX-40, CD40, and ICOS.

A leader sequence or signal peptide may also be used to promote CAR secretion. For example, the leader sequence of the GM-CSF receptor may be used. In addition, the structure is preferably composed of an extracellular domain and a transmembrane domain linked together through a spacer domain. More specifically, the CAR according to a preferred embodiment contains a spacer domain between the extracellular domain and transmembrane domain. The spacer domain is used for promoting linking between the CAR and target ABCB5.

The engineered T or NK cells may be bispecific, that is, express bispecific CARs or multiple different CARs, wherein their affinity is for two distinct epitopes or antigens. Bispecific CAR-T or NKs can be used either for increasing the number of potential binding sites on cancer cells or in cancer ECM or, alternatively, for localizing cancer cells to other immune effector cells which express ligands specific to the T or NK-CAR. For use in cancer therapy, a bispecific CAR may bind to a target tumor cell or tumor ABCB5 and to an effector cell, e.g. a T cell, NK cell or macrophage. The engineered T or NK cells of the current disclosure may comprise a bispecific CAR or multiple CARs expressed by the same T or NK cell. This allows the T or NK cells to target two different epitopes of ABCB5 or antigens simultaneously.

Also provided are isolated nucleic acid molecules and vectors encoding any of the anti-ABCB5 CARs as disclosed herein, and host cells, such as host immune cells (e.g., T cells and natural killer cells), comprising the nucleic acid molecules or vectors. Immune cells expressing anti-ABCB5 CARs, which comprises a ABCB5-specific antibody binding fragment, can be used for the treatment of diseases mediated by ABCB5⁺ cells.

Antibodies capable of binding ABCB5 as described herein can be made by any method known in the art. If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the activity of ABCB5. It will be apparent to one of skill in the art that one or more polynucleotide changes can be made to the antibody and still maintain its binding specificity to the target antigen.

The antibodies are referred to as synthetic or isolated. The phrase “isolated antibody or antibody fragment” refers to an antibody or antibody fragment that is substantially free of other antibodies having different antigenic specificities (e.g., an isolated antibody specifically binding a target antigen is substantially free of antibodies that specifically do not bind the target antigen). Moreover, an isolated antibody or antibody fragment can be substantially free of other cellular material and/or chemicals. Isolated antibodies according to embodiment of the invention can be synthetic. A synthetic antibody is an antibody that is not naturally occurring. The antibodies, while derived from human immunoglobulin sequences, can be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

In other embodiments, antibodies specific to a target antigen (e.g., ABCB5) can be made by the conventional hybridoma technology. The full-length target antigen or a fragment thereof or epitope, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein. The techniques may be performed for instance in a ABCB5 knock out animal such as a mouse. This may result in a more diverse population of ABCB5 antibodies. Knock out animals made me made for instance using CRISPR cas techniques. The antibodies may also be isolated from humans that have enhanced immunity to ABCB5.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-ABCB5 monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of interfering with the ABCB5 activity. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R₁N═C═NR, where R and R₁ are different alkyl groups, can yield a population of antibodies (e.g., monoclonal antibodies).

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

“Full length antibody” as used herein refers to an antibody having two full length antibody heavy chains and two full length antibody light chains. A full length antibody heavy chain (HC) consists of a heavy chain variable region (VH) and constant domains (CH1, CH2, and CH3). A full length antibody light chain (LC) consists of a light chain variable region (VL) and constant domain (CL). The full length antibody can be lacking the C-terminal lysine (K) in either one or both heavy chains. The full length antibody may be prepared using any of the above methods or other methods known in the art.

Full length bispecific antibodies can be generated for example using Fab half molecule exchange between two monospecific bivalent antibodies by introducing substitutions at the heavy chain CH3 interface in each half molecule to favor heterodimer formation of two antibody half molecules having distinct specificity either in vitro in cell-free environment or using co-expression. The exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the arms may be engineered to favor heterodimerization over homodimerization. The resulting product is a bispecific antibody having two arms or half molecules which each can bind a distinct epitope.

“Homodimerization” as used herein, with respect to the antibodies, refers to an interaction of two heavy chains having identical CH3 amino acid sequences. “Homodimer” as used herein, with respect to the antibodies, refers to an antibody having two heavy chains with identical CH3 amino acid sequences.

“Heterodimerization” as used herein, with respect to the antibodies, refers to an interaction of two heavy chains having non-identical CH3 amino acid sequences. “Heterodimer” as used herein, with respect to the antibodies, refers to an antibody having two heavy chains with non-identical CH3 amino acid sequences.

The “knob-in-hole” strategy (see, e.g., PCT Intl. Publ. No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532.

In addition to methods described above, bispecific antibodies can be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies can be adapted to produce a phage or yeast scFv library and scFv clones specific to ABCB5 can be identified from the library following routine procedures. Positive clones can be subjected to further screening to identify those that inhibit ABCB5 activity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined.

The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the ABCB5 polypeptide have been replaced (swapped) with sequences from a closely related, but antigenically distinct protein. By assessing binding of the antibody to the mutant ABCB5, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art. In some examples, an anti-ABCB5 antibody is prepared by recombinant technology as exemplified below.

Nucleic acids encoding the heavy and light chain of an anti-ABCB5 antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters (Brown, M. et al., Cell, 49:603-612 (1987)), those using the tetracycline repressor (tetR)(Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987)); Gossen and Bujard (1992); (M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-ABCB5 antibody, as also described herein. The recombinant expression vector can be introduced into a suitable host cell by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-ABCB5 antibody and the other encoding the light chain of the anti-ABCB5 antibody. Both of the two recombinant expression vectors can be introduced into a suitable host cell by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-ABCB5 antibody as described herein, vectors (e.g., expression vectors) containing such; and host cells comprising the vectors are within the scope of the present disclosure.

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art.

The pharmaceutical compositions to be used in the present methods can comprise pharmaceutically acceptable carriers, excipients, or stabilizers in the form of lyophilized formulations or aqueous solutions. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules.

In other examples, the pharmaceutical composition described herein can be formulated in a sustained release format, which affects binding selectively to tissue or tumors by implementing certain protease biology technology, for example, by peptide masking of the antibody's antigen binding site to allow selective protease cleavability by one or multiple proteases in the tumor microenvironment, such as Probody™ or Conditionally Active Biologics™. An activation may be formulated to be reversible in a normal microenvironment.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation. For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

Any of the antibodies, as well as the encoding nucleic acids or nucleic acid sets, vectors comprising such, or host cells comprising the vectors, described herein are useful for treating cancer or other malignancies and any other ABCB5 mediated disorder.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having cancer. A subject having a target disease or disorder can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors for that disease/disorder.

The methods and compositions described herein may be used to treat cancer.

Examples of cancers that may be treated with the methods and compositions described herein include, but are not limited to: lung cancer, melanoma, renal cancer, liver cancer, myeloma, prostate cancer, breast cancer, colorectal cancer, gastric cancer, pancreatic cancer, thyroid cancer, hematological cancer, lymphoma, leukemia, skin cancer, ovarian cancer, bladder cancer, urothelial carcinoma, head and neck cancer, metastatic lesion(s) of the cancer, and all types of cancer which are diagnosed for high mutational burden. In a particular embodiment, the cancer has a high mutation burden. Subjects having or at risk for various cancers can be identified by routine medical procedures.

In some examples, the human patient has microsatellite instability-high (MSI-H) or mismatch repair deficient (dMMR), found in soft tissue cancer, glioblastoma, esophageal and EGJ carcinoma, breast carcinoma, non-small cell lung cancer, ovarian surface epithelial carcinomas, cancer of unknown primary, small cell lung cancer, non-epithelial ovarian cancer, pancreatic adenocarcinoma, other female genital tract malignancies, uveal melanoma, retroperitoneal or peritoneal sarcoma, thyroid carcinoma, uterine sarcoma, cholangiocarcinoma, prostate adenocarcinoma, hepatocellular carcinoma, neuroendocrine tumors, cervical cancer, colorectal adenocarcinoma, small intestinal malignancies, gastric adenocarcinoma and endometrial cancer.

As used herein, “an effective amount” refers to the amount of each active agent required to confer therapeutic effect on the subject, either alone or in combination with one or more other active agents. In some embodiments, the therapeutic effect is reduced ABCB5 activity and/or enhanced tumor killing. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the antibody. To assess efficacy of the antibody, an indicator of the disease/disorder can be followed.

Generally, for administration of any of the antibodies described herein, an initial candidate dosage can be about 2 mg/kg. For the purpose of the present disclosure, a typical daily, weekly, every two weeks, or every three weeks dosage might range from about any of 0.1 mg/kg to 3 mg/kg to 30 mg/kg to 100 mg/kg to 300 mg/kg to 0.6 mg/kg, 1 mg/kg, 3 mg/kg, to 10 mg/kg, to 30 mg/kg to 100 mg/kg or more, depending on the factors mentioned above. For repeated administrations over several days, weeks, months, or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 3 mg/kg every 3 weeks, followed by a maintenance dose of about 1 mg/kg of the antibody once in 6 weeks, or followed by a maintenance dose of about 1 mg/kg every 3 weeks. However, other dosage regimens may be useful, depending on the pattern of pharmacokinetic decay that the practitioner wishes to achieve. For example, dosing of 1 mg/kg once in every 3 weeks in combination treatment with at least one additional anti-cancer agent is contemplated. In some embodiments, dosing ranging from about 3 μg/mg to about 3 mg/kg (such as about 3 μg/mg, about 10 μg/mg, about 30 μg/mg, about 100 μg/mg, about 300 μg/mg, about 1 mg/kg, and about 3 mg/kg) may be used. In some embodiments, dosing frequency is once every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.1 to 5.0 mg/kg may be administered. In some examples, the dosage of the anti-ABCB5 antibody described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the antibody, and the discretion of the attending physician. Typically the clinician will administer an antibody, until a dosage is reached that achieves the desired result. In some embodiments, the desired result is a reduction of the size of the tumor, increased progression-free survival period and/or overall survival. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying the development or progression of the disease, or reducing disease severity.

Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the antibodies described herein are administered to a subject in need of the treatment at an amount sufficient to inhibit the activity of the target by at least 20% (e.g., 30%, 40%, 50%, 60%, 70%, 80%, 90% or greater) in vivo. In other embodiments, the antibody is administered in an amount effective in reducing the activity level of a target by at least 20% (e.g., 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered parenterally, topically, orally, by inhalation spray, rectally, nasally, buccally, vaginally or via an implanted reservoir. The term “parenteral” as used herein includes subcutaneous, intracutaneous, intravenous, intraperitoneal, intratumor, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application.

Therapeutic compositions containing a polynucleotide (e.g., those encoding the antibodies described herein) may be administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

When co-administered with an additional therapeutic agent, suitable therapeutically effective dosages for each agent may be lowered due to the additive action or synergy.

The efficacy of the methods described herein may be assessed by any method known in the art and would be evident to a skilled medical professional. For example, the efficacy of the antibody-based immunotherapy may be assessed by survival of the subject or cancer burden in the subject or tissue or sample thereof. In some embodiments, the antibody based therapy is assessed based on the safety or toxicity of the therapy in the subject, for example by the overall health of the subject and/or the presence of adverse events or severe adverse events.

Any of the anti-ABCB5 antibodies disclosed herein can also be used for detecting the presence of ABCB5 (e.g., ABCB5+ cells) in vitro or in vivo. Results obtained from such detection methods can be used for diagnostic purposes (e.g., diagnosing diseases associated with ABCB5⁺ cells) or for scientific research purposes (e.g., studying bioactivity and/or regulation of ABCB5⁺ cells).

For assay uses such as diagnostic uses, an anti-ABCB5 antibody as described herein may be conjugated with a detectable label (e.g., an imaging agent such as a contrast agent) for detecting presence of ABCB5 (e.g., ABCB5⁺ cells), either in vivo or in vitro. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

Furthermore, the conjugates of the present invention may have one or more polymorph or amorphous crystalline forms and as such are intended to be included in the scope of the invention. In addition, the conjugates may form solvates, for example with water (i.e., hydrates) or common organic solvents. As used herein, the term “solvate” means a physical association of the conjugates of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope polymorphs and solvates of the conjugates of the present invention. Thus, in the methods of treatment of the present invention, the term “administering” shall encompass the means for treating, ameliorating or preventing a syndrome, disorder or disease described herein with the conjugates of the present invention or a polymorph or solvate thereof, which would obviously be included within the scope of the invention albeit not specifically disclosed.

In another embodiment, the invention relates to the conjugates of the invention for use as a medicament.

In one example, an anti-ABCB5 antibody as described herein can be attached to a detectable label, which is a compound that is capable of releasing a detectable signal, either directly or indirectly, such that the aptamer can be detected, measured, and/or qualified, in vitro or in vivo. Examples of such “detectable labels” are intended to include, but are not limited to, fluorescent labels, chemiluminescent labels, colorimetric labels, enzymatic markers, radioactive isotopes, and affinity tags such as biotin. Such labels can be conjugated to the aptamer, directly or indirectly, by conventional methods.

In some embodiments, the detectable label is an agent suitable for imaging ABCB5⁺ cells in vivo, which can be a radioactive molecule, a radiopharmaceutical, or an iron oxide particle. Radioactive molecules suitable for in vivo imaging include, but are not limited to, ¹²²I, ¹²³I, ¹²⁴I, ¹²⁵I, ¹³¹I, ¹⁸F, ⁷⁵Br, ⁷⁶Br, ⁷⁶Br, ⁷⁷Br, ²¹¹At, ²²⁵Ac, ¹⁷⁷Lu, ¹⁵³Sm, ¹⁸⁶Re, ¹⁸⁸Re, ⁶⁷Cu, ²¹³Bi, ²¹²Bi, ²¹²Pb, and ⁶⁷Ga. Exemplary radiopharmaceuticals suitable for in vivo imaging include ¹¹¹In Oxyquinoline, ¹³¹I Sodium iodide, ^{99m}Tc Mebrofenin, and ^{99m}Tc Red Blood Cells, ¹²³I Sodium iodide, ^{99m}Tc Exametazime, ^{99m}Tc Macroaggregate Albumin, ^{99m}Tc Medronate, ^{99m}Tc Mertiatide, ^{99m}Tc Oxidronate, ^{99m}Tc Pentetate, ^{99m}Tc Pertechnetate, ^{99m}Tc Sestamibi, ^{99m}Tc Sulfur Colloid, ^{99m}Tc Tetrofosmin, Thallium-201, and Xenon-133. The reporting agent can also be a dye, e.g., a fluorophore, which is useful in detecting a disease mediated by ABCB5⁺ cells in tissue samples.

To perform a diagnostic assay in vitro, an anti-ABCB5 antibody can be brought in contact with a sample suspected of containing ABCB5, e.g., ABCB5⁺ cells. The antibody and the sample may be incubated under suitable conditions for a suitable period to allow for binding of the antibody to the ABCB5 antigen. Such an interaction can then be detected via routine methods, e.g., ELISA histological staining or FACS.

To perform a diagnostic assay in vivo, a suitable amount of anti-ABCB5 antibodies, conjugated with a label (e.g., an imaging agent or a contrast agent), can be administered to a subject in need of the examination. Presence of the labeled antibody can be detected based on the signal released from the label by routine methods.

The present disclosure also provides kits for the therapeutic or diagnostic applications as disclosed herein. Such kits can include one or more containers comprising an anti-ABCB5 antibody, e.g., any of those described herein.

In some embodiments, the kit can comprise instructions for use in accordance with any of the methods described herein. The included instructions can comprise a description of administration of the anti-ABCB5 antibody to treat, delay the onset, or alleviate a target disease as those described herein. The kit may further comprise a description of selecting an individual suitable for treatment based on identifying whether that individual has the target disease. In still other embodiments, the instructions comprise a description of administering an antibody to an individual at risk of the target disease.

The instructions relating to the use of an anti-ABCB5 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder such as cancer. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-ABCB5 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

Also provided herein are kits for use in detecting ABCB5⁺ cells in a sample and/or isolation of ABCB5⁺ cells. Such a kit may comprise any of the anti-ABCB5 antibodies described herein. In some instances, the anti-ABCB5 antibody can be conjugated with a detectable label as those described herein. Alternatively or in addition, the kit may comprise a secondary antibody capable of binding to anti-ABCB5 antibody. The kit may further comprise instructions for using the anti-ABCB5 antibody for detecting ABCB5⁺ cells.

### EXAMPLES

### Example 1—Anti-ABCB5 Ab100 Original/First Generation Antibodies

### Initial SDS-PAGE Characterization of Ab100 (Bio-Rad Protean™ Stain-Free Gels)

Methods: Process development included stable cell line generation/expression (CHOvolution™ CHO K1 SEFEX), fed-batch (GlycanTune™ C+ feed), bioreactor 10 liter, run 01/02, and purification via Mabselect (Prot. A affinity chromatography)/Sephadex (gel filtration to PBS) column. The resultant antibody is referred to as AB100.

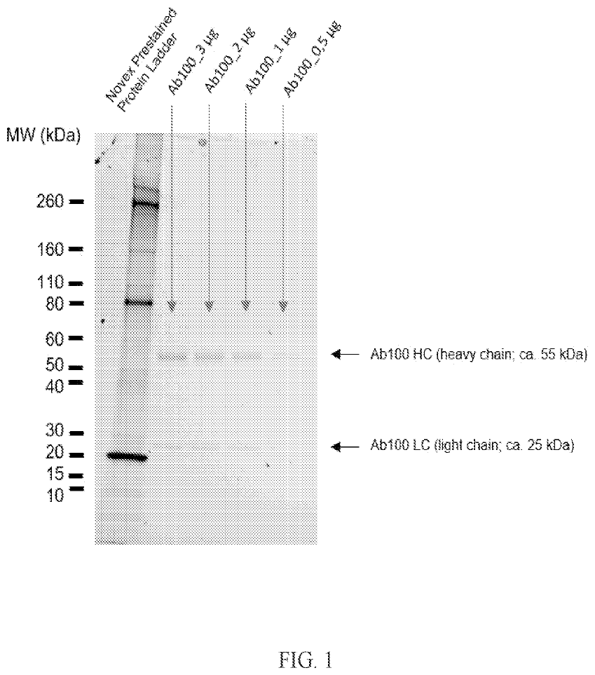

Results: Ab100 (the purified antibody) was characterized to determine structure. A basic assessment of antibody batch purity, stability, degradation etc. was conducted by SDS-PAGE under denaturating (96° C., 5 min) and reducing (100 mM DTT) conditions (see FIG. 1). Thus, detection of the antibody (purified antibody Ab100) using gel electrophoresis for quality control showed an intact antibody.

### ELISA Binding Studies

Linear peptide ELISA (20 μM peptides)

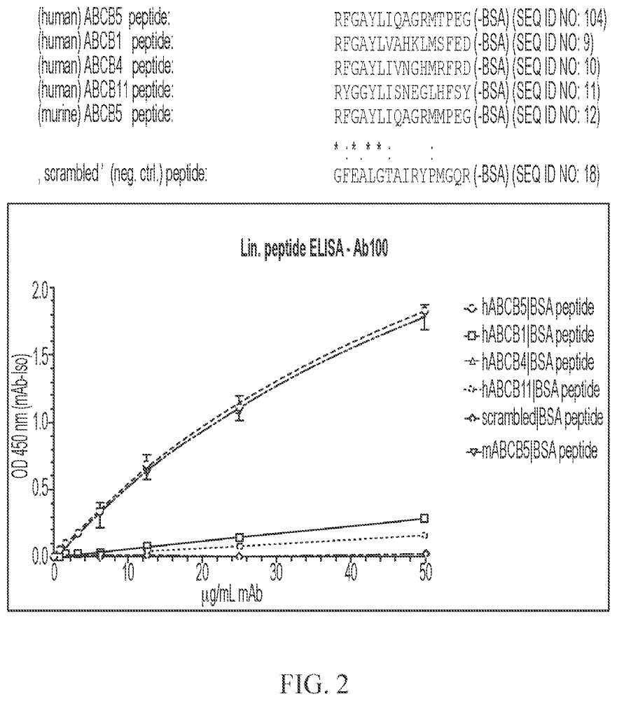

Specificity for ABCB5 of Ab100. ELISAs run against ABCB5 peptide used to make the antibody was examined. The linear peptide ELISA binding study was a comparative assessment of quantitative binding to ABCB5 linear epitope peptide (3rd extracellular loop) and corresponding peptide segments of homologous (ABCB 1, 4, 11) and orthologous (murine ABCB5) proteins—to assess specificity and/or cross-reactivity (see FIG. 2). Three human homologous proteins that vary in sequence slightly were used to demonstrate specificity. Ab100 did not bind to any appreciable extent to any of the three controls. The antibody does demonstrate binding to murine ABCB5 (one amino acid substitution relative to human), which is a desirable property for Toxicity studies. A primate sequence was not tested as the epitope sequence is identical to the human epitope. A scrambled peptide was used as a negative control. All of the tested peptides are all linear peptides, synthetically created to resemble extracellular loop. The X-axis is the Ab concentration. The Y-axis is the optical density of ELISA (signal representing binding). A non-specific signal with isotype antibody has been subtracted, which is why a non-specific isotype antibody is not shown.

### ABCB5 Recombinant Protein ELISA

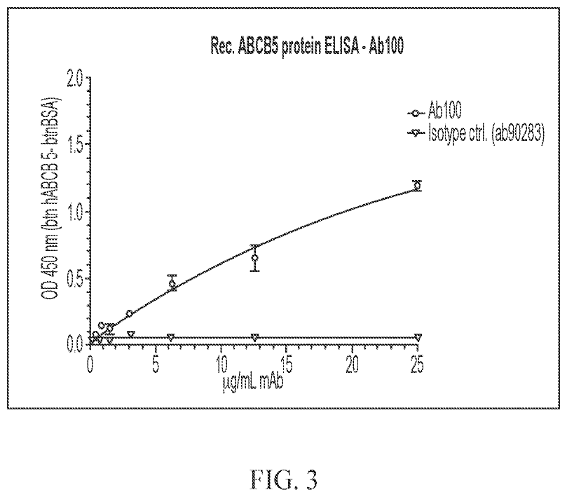

The ABCB5 recombinant human protein ELISA binding study was an evaluation of specific binding to recombinant ABCB5 protein (see FIG. 3). Shows binds recombinant intact protein, shows not just good biding to peptide but also intact protein. Isotype included here as control.

### Cyclic (Biotinylated) Peptide ELISA (200 nM Peptides)

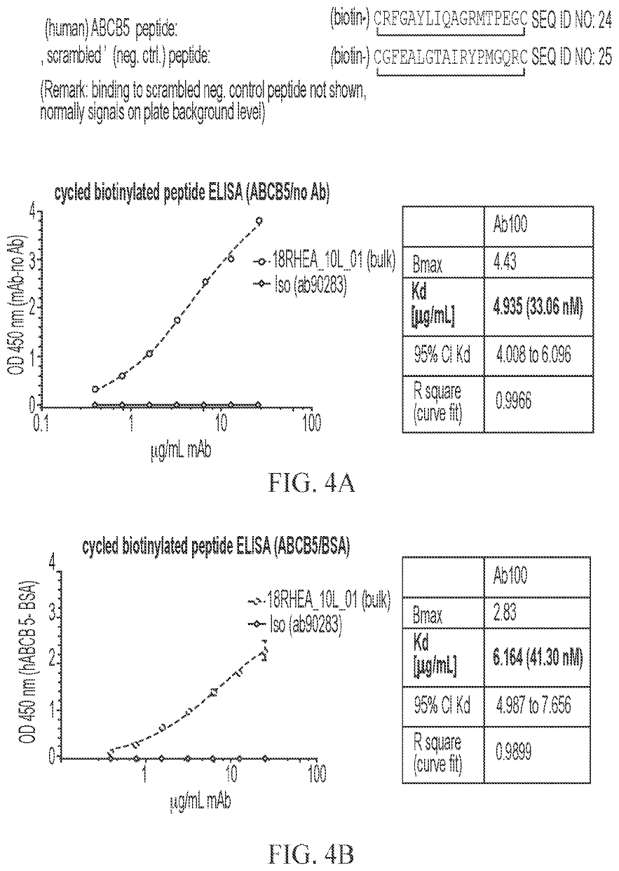

A cyclic version of the peptide epitope was generated from the linear peptide to create a three dimensional structure. The cyclic version may resemble the loop better. Thus, the cyclic (biotinylated) peptide ELISA binding study evaluated the specific binding and affinity of the native cyclic ABCB5 peptide (cyclization by disulfide bond between additional cysteines at both ends) with N-terminal biotin modification for immobilization (on streptavidin-precoated plates) (see FIGS. 4A-4B). The data is also shown numerically in the Tables below:

4B (BSA Control subtracted)

| | Ab100 |
|---|---|
| Bmax | 2.83 |
| Kd (ug/mL) | 6.164 (41.3 nM) |
| 95% Cl Kd | 4.987 to 7.656 |
| R square | 0.9899 |
| (curve fit) | |

4A (no Ab Control subtracted)

| | Ab100 |
|---|---|
| Bmax | 4.43 |
| Kd (ug/mL) | 4.935 (33.06 nM) |
| 95% Cl Kd | 4.008 to 6.096 |
| R square | 0.9966 |
| (curve fit) | |

Flow Cytometry (FACS) Binding Studies—Human Tumor Cell Lines with ABCB5+ Subpopulation

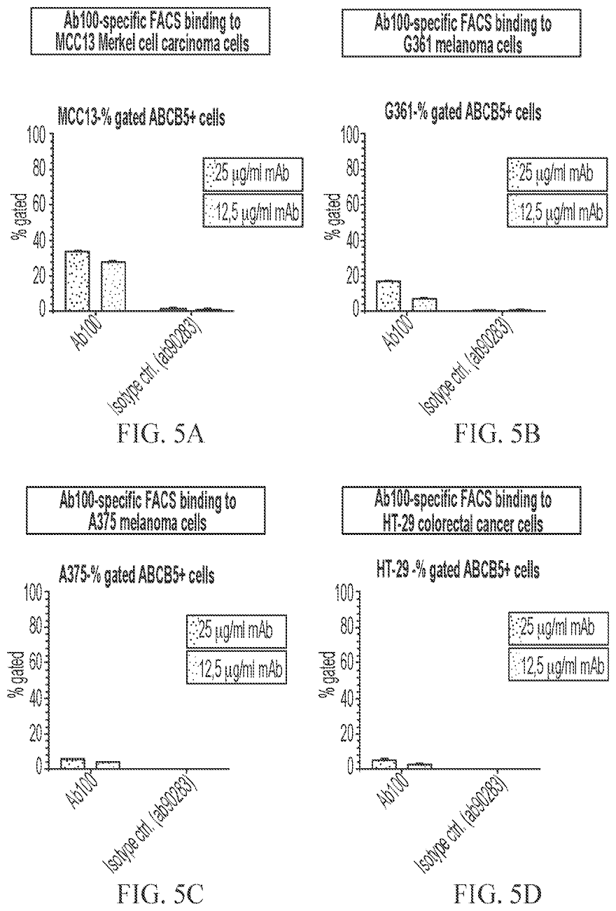

An evaluation of specific binding to cellular ABCB5 at human tumor cell lines was conducted using FACS analysis. Detection occurred with APC-labeled 2nd anti-human antibody (see FIGS. 5A-5D). FIGS. 5A-5D show the percentage of ABCB5+ cells within a population of cancer cells at two different concentrations of antibody (dark bar is 25 ug/mL; lighter bar is 12.5 ug/mL). FIG. 5A shows specific ABCB5+ stained cells in a population of Merkel cell carcinoma cells. FIG. 5B shows specific ABCB5+ stained cells in a population of G361 melanoma cells. FIG. 5C shows specific ABCB5+ stained cells in a population of A375 melanoma cells. FIG. 5D shows specific ABCB5+ stained cells in a population of HT-29 colorectal cancer cells. The different levels in % gated populations of the different cell lines reflects the different number of ABCB5+ cells within the population. The data demonstrate that the antibodies are capable of specifically recognizing cell surface ABCB5 in different populations of cells.

Immunofluorescence (IF) Staining—Human Tumor Cell Lines w/ABCB5+ Subpopulation

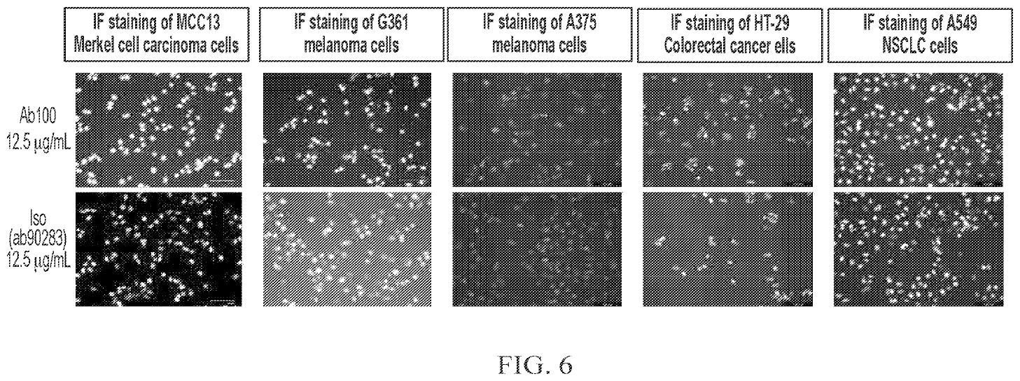

An evaluation and visualization of specific binding to cellular ABCB5 at human tumor cell lines was conducted. Binding of the anti ABCB5 antibody to 5 tumor cell lines was examined and the data is shown in FIG. 6. FIGS. 6A-6E show the staining of ABCB5+ cells within a population of cancer cells (re indicating ABCB5). FIG. 6A shows specific ABCB5+ stained cells in a population of Merkel cell carcinoma cells. FIG. 6B shows specific ABCB5+ stained cells in a population of G361 melanoma cells. FIG. 6C shows specific ABCB5+ stained cells in a population of A375 melanoma cells. FIG. 6D shows specific ABCB5+ stained cells in a population of HT-29 colorectal cancer cells. FIG. 6E shows specific ABCB5+ stained cells in a population of A549 NSCLC cells.

### Antibody-Mediated Cytotoxic (Anti-Tumor) Effector Functions

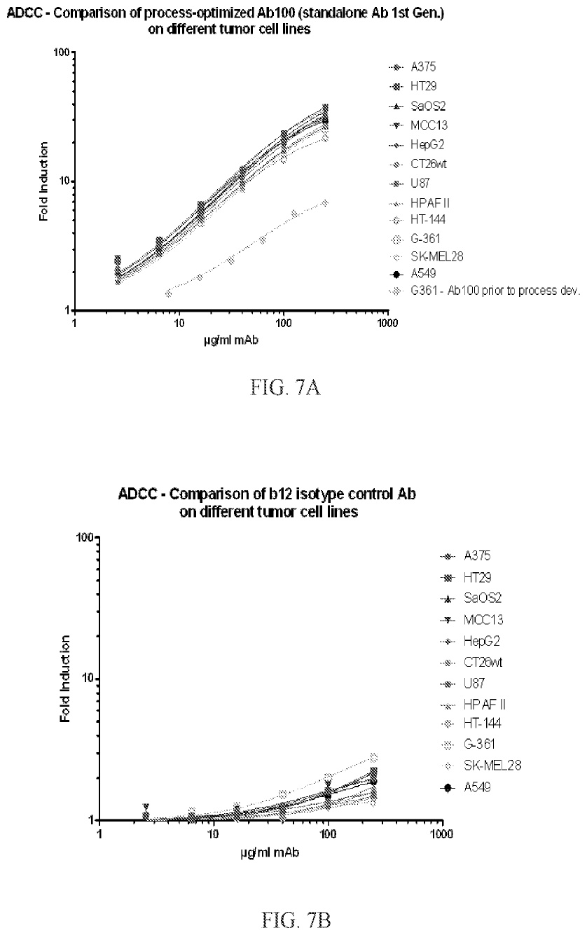

An antibody-dependent cellular cytotoxicity (ADCC) reporter bioassay was conducted to determine average potency/efficacy on tumor cell lines (see FIG. 7A-7B). The fold induction of ADCC was measured in a panel of cells in response to treatment with Ab100 (FIG. 7A) or isotype control (FIG. 7B).

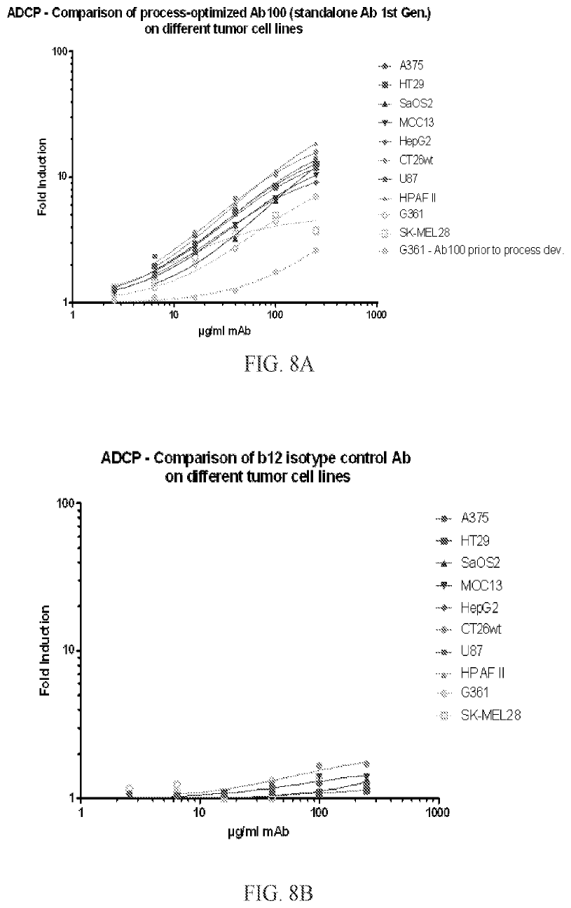

An antibody-dependent cellular phagocytosis (ADCP) reporter bioassay was conducted to determine average potency/efficacy on tumor cell lines (see FIG. 8A-8B). The fold induction of ADCP was measured in a panel of cells in response to treatment with Ab100 (FIG. 8A) or isotype control (FIG. 8B).

(Cell-Based) pAkt/Akt ELISA—Human Tumor Cell Lines w/ABCB5+ Subpopulation

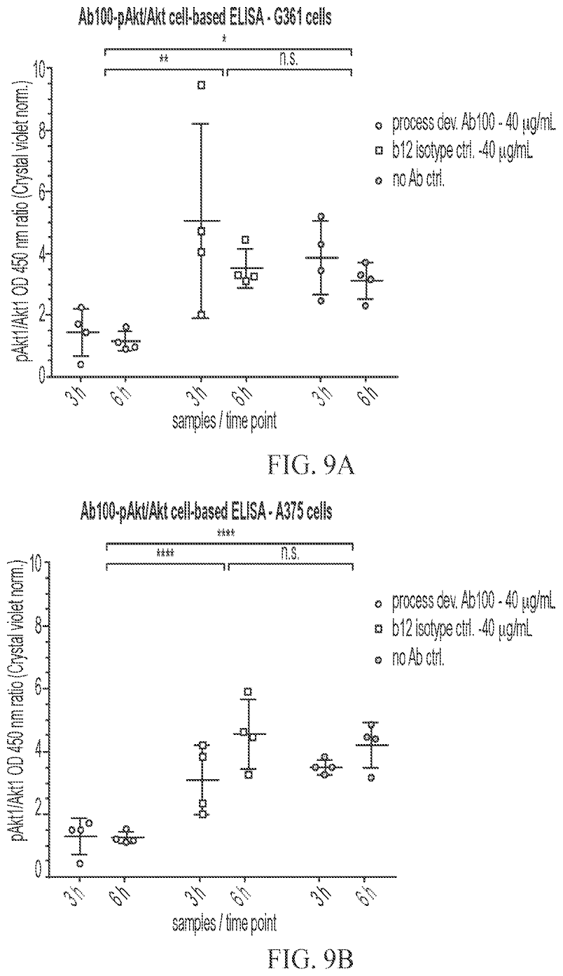

In order to assess intracellular signaling capability of Ab100, a ratio of pAkt1 to Akt1 in cells (G361-FIG. 9A or A375-FIG. 9B) treated with AB100 or isotype control was measured. The results are shown in FIGS. 9A-9B. The change in phosphorylation observed reflects the ability of ABCB5 antibody to affect intracellular signaling. Blocking ABCB5 using a specific antibody inhibits the downstream reaction involved in phosphorylation. Thus the ratio is diminished in response to Ab100 treatment. The Ab significantly lowered the ratio relative to controls.

### Example 2—Anti-ABCB5 In Vivo Anti-Tumor Efficacy Testing (Mouse Models)

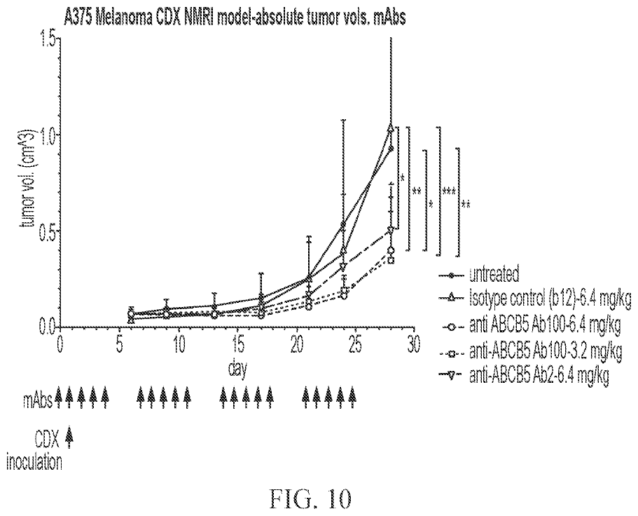

### Experiment 1—see FIG. 10

The tumor model used in the study was an A375 human cell derived tumor xenograft (CDX) melanoma (tumor prevention model). It is referred to as a tumor prevention model because the antibody is delivered before an established tumor develops. 10⁷ cells were administered s.c. The mouse strain was NMRI (immunocompromised nude mouse model) nu/nu (female). Test items included mAbs: Ab100, Ab2, and Isotype control. as well as antibody-drug-conjugates (ADCs). The following dosing scheme was followed for mAbs: 6.4 & 3.2 mg/kg—days 1-5/week, 4 weeks—i.p. (n=8) for Ab100, 6.4 mg/kg—days 1-5/week, 4 weeks—i.p. (n=8) for Ab2, and 6.4 mg/kg—days 1-5/week, 4 weeks—i.p. (n=8) for isotype control.). The following dosing scheme was followed for the antibody-drug conjugates: 2.5 mg/kg—1×/week, 4 weeks—i.v. (n=8) and 2.5 mg/kg—1×/week, 4 weeks—i.v. (n=8) for isotype control.

DAR measures amount of drug on the Antibody and can be a useful assessment for the amount of drug to be delivered. Black arrows refer to the days that Ab was administered and the red arrow indicates tumor inoculation day. The data presented in FIG. 10 demonstrates that Ab100 and Ab2 (not conjugated to a drug) significantly reduced tumor volume in the mice relative to untreated and isotype control. The observed reduction in tumor volume for the ADC was the largest. The Ab-drug conjugates significantly reduced tumor volume in the mice relative to untreated and isotype control.

### Experiment 2

A CT26.wt (CRL-2638) murine syngeneic colorectal cancer (tumor prevention model) was used in this study. 10⁶ cells were administered s.c. The mouse strain was Balb/c (female). Test items included antibody-drug-conjugates and isotype control. The following dosing scheme was followed for the test items: 4 mg/kg—days 1/3/7/10/13/15/17 (n=7) and 2 mg/kg—days 1/3/7/10/13/15/17 (n=7) for Ab101—drug conjugate and 4 mg/kg—days 1/3/7/10/13/15/17 (n=7) and 2 mg/kg—days 1/3/7/10/13/15/17 (n=7) for isotype control, all i.v. The data demonstrated that Ab101—drug conjugate significantly reduced tumor volume in an in vivo mouse model of colorectal cancer. A significant dose difference was observed between 2 and 4 mg/kg of antibody.

### Experiment 3

A A375 (CRL-1619) human CDX melanoma (overall survival analysis) was used to study antibodies and antibody-drug-conjugates. 10⁷ cells s.c. were administered. The mouse strain was NMRI nu/nu (female). Test items included multiple antibody-drug-conjugates. The data demonstrated that multiple antibody-drug-conjugates based on Ab101, Ab44 and Ab42 significantly increased survival time in the melanoma mice relative to untreated and isotype control.

### Experiment 4

A CT26.wt (CRL-2638) murine syngeneic colorectal cancer (tumor prevention model) with 10⁶ cells s.c. was used. The mouse strain was Balb/c (female). Test items included antibody-drug-conjugates with Ab101, Ab44, and isotype ctrl. The data demonstrated that Ab101 and Ab44 (Ab conjugated to a drug) significantly increased absolute tumor volume in the mice relative to untreated and isotype control.

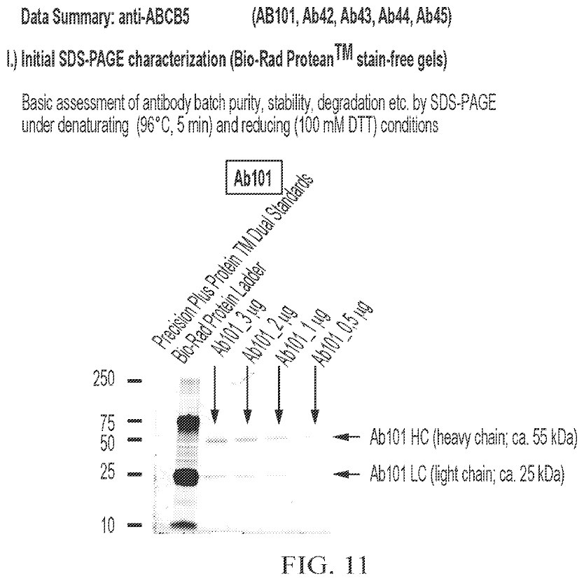

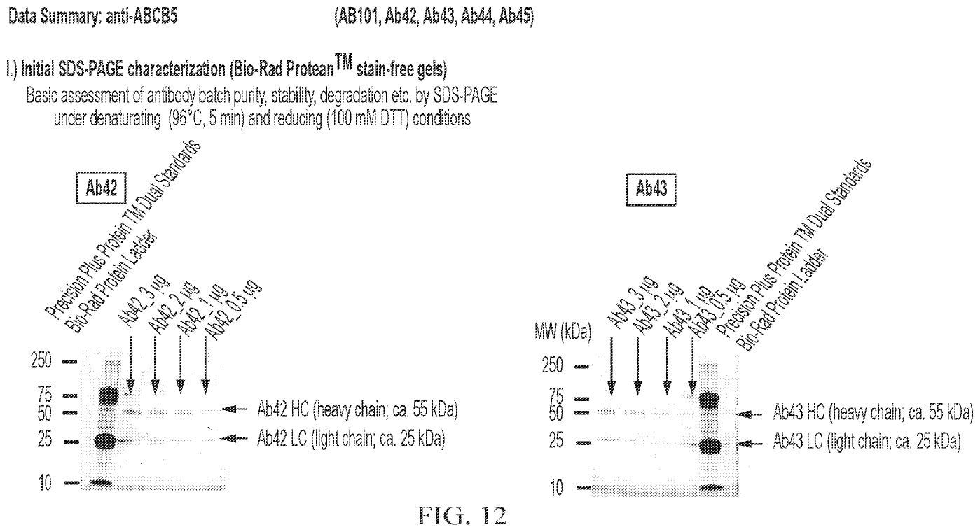

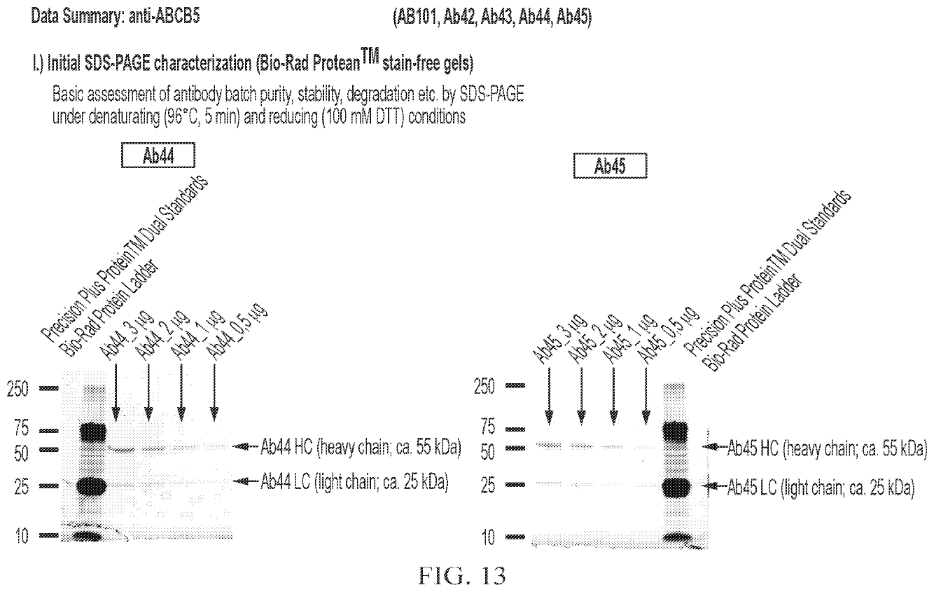

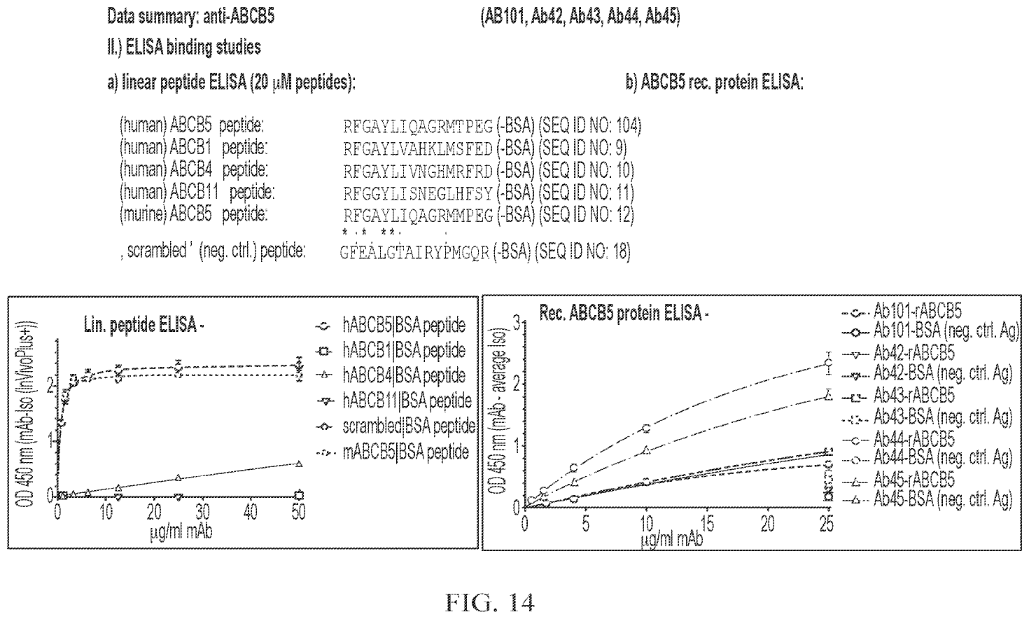

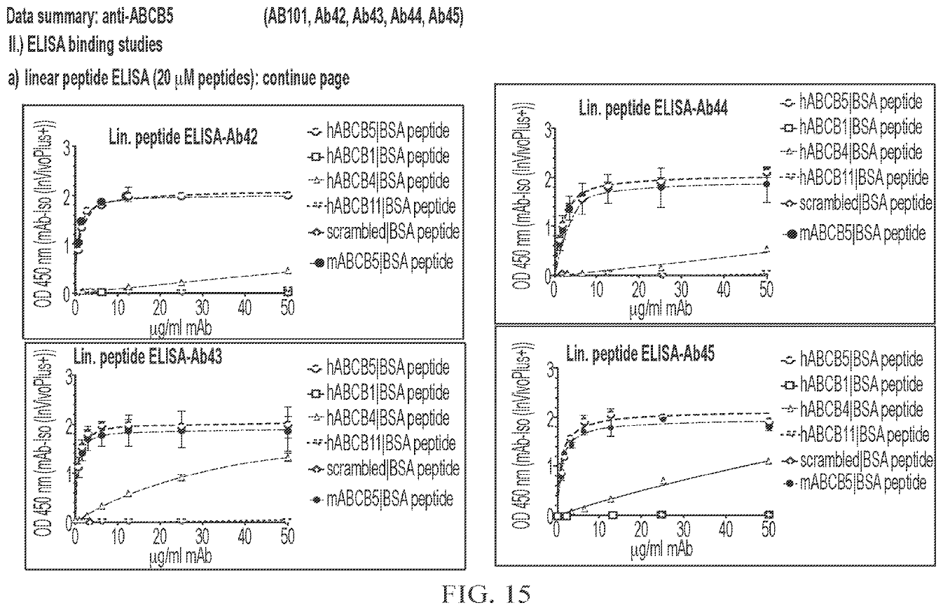

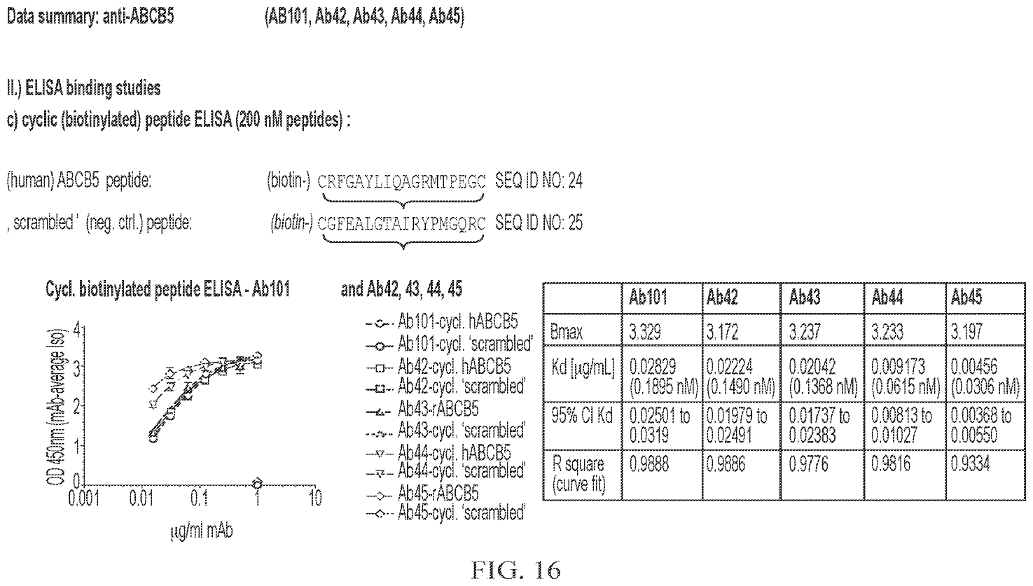

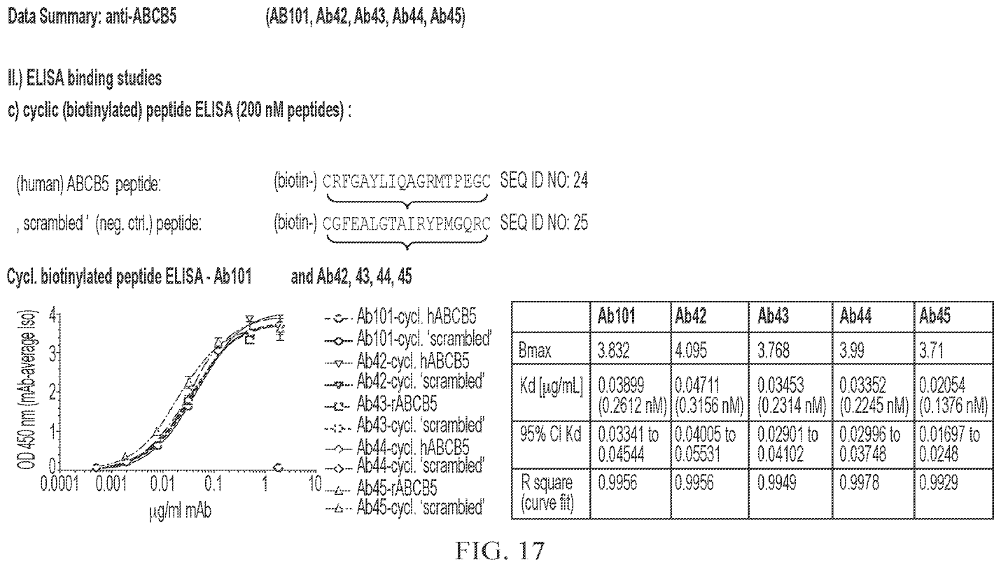

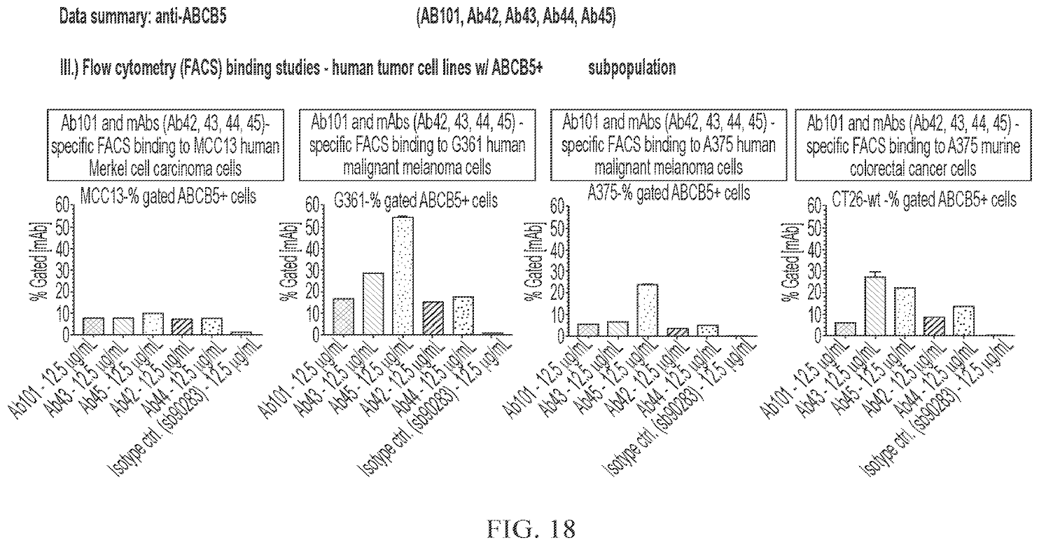

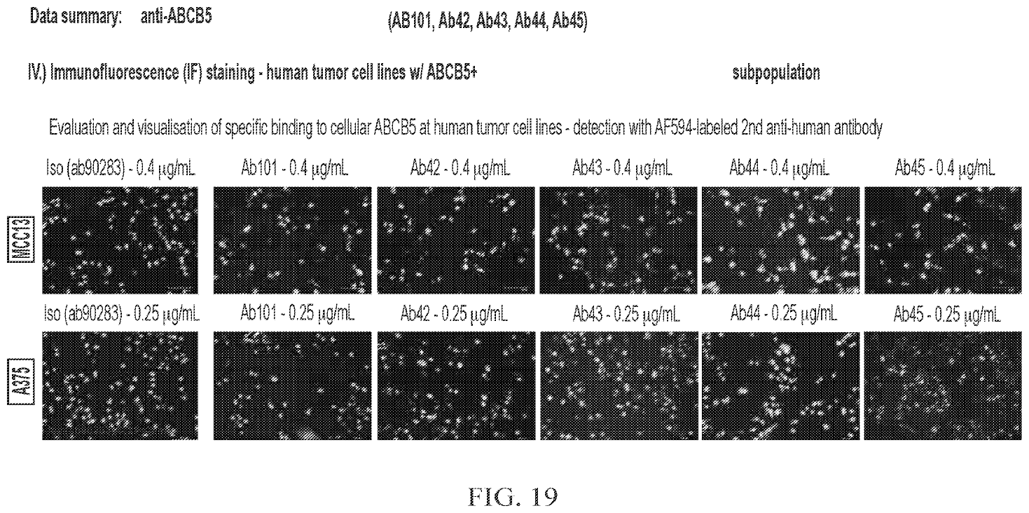

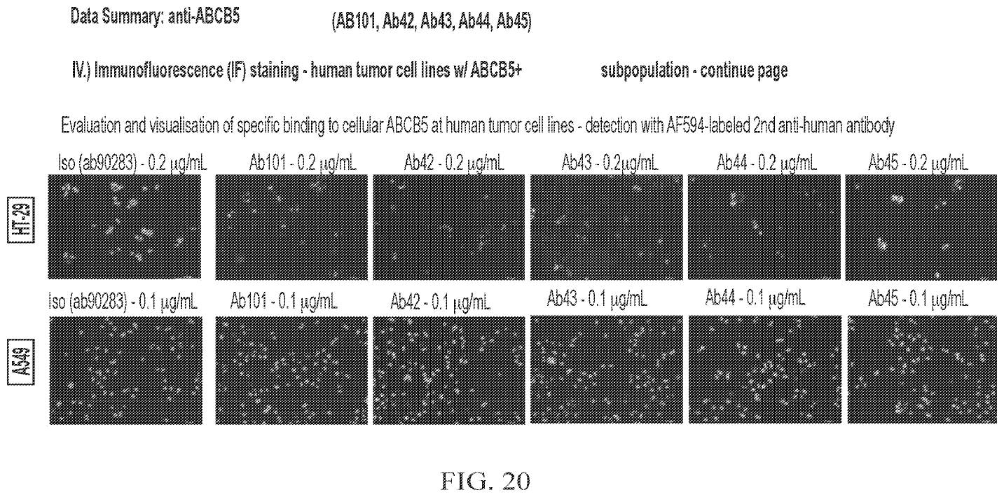

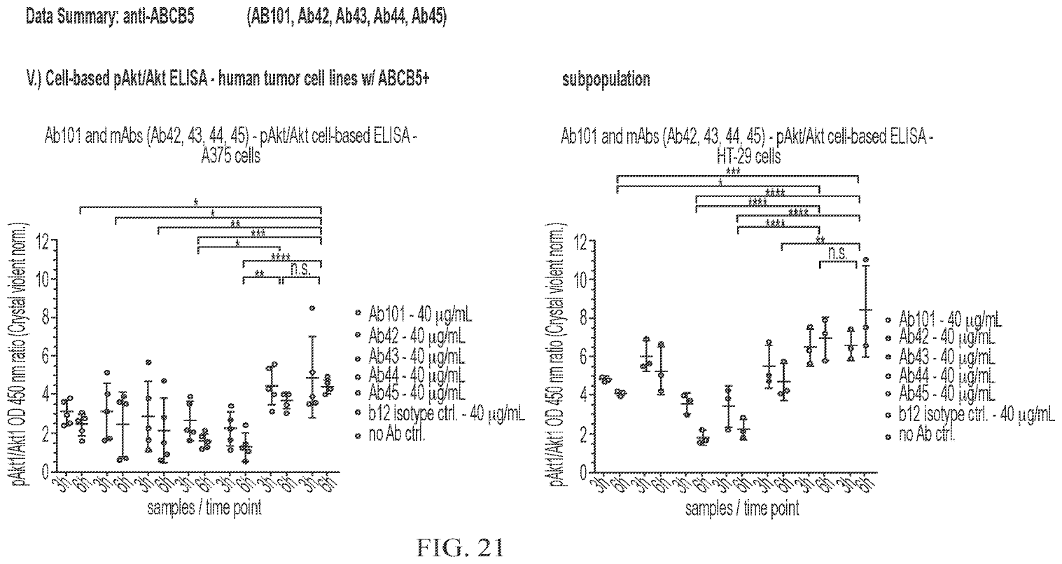

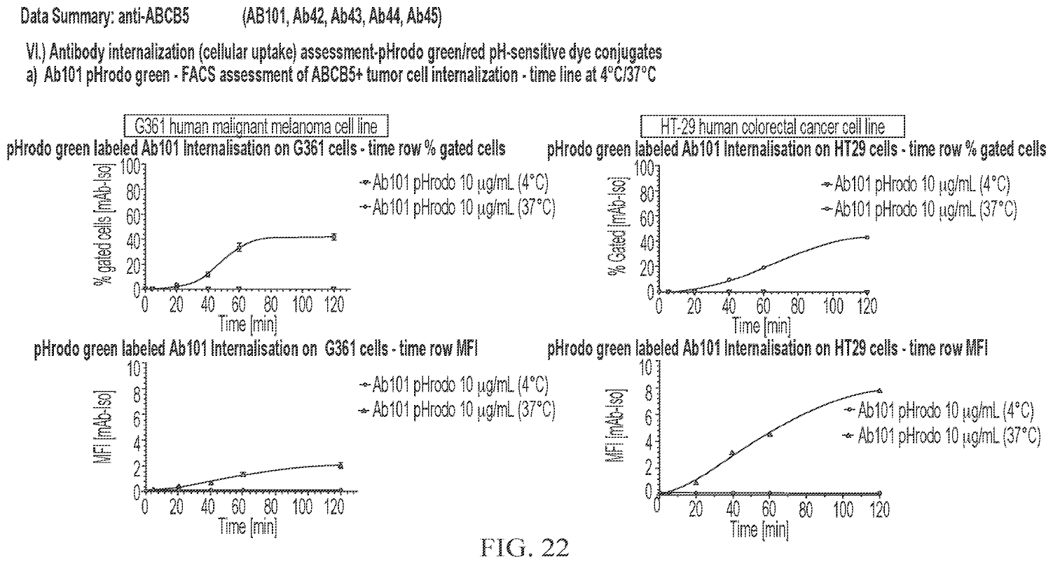

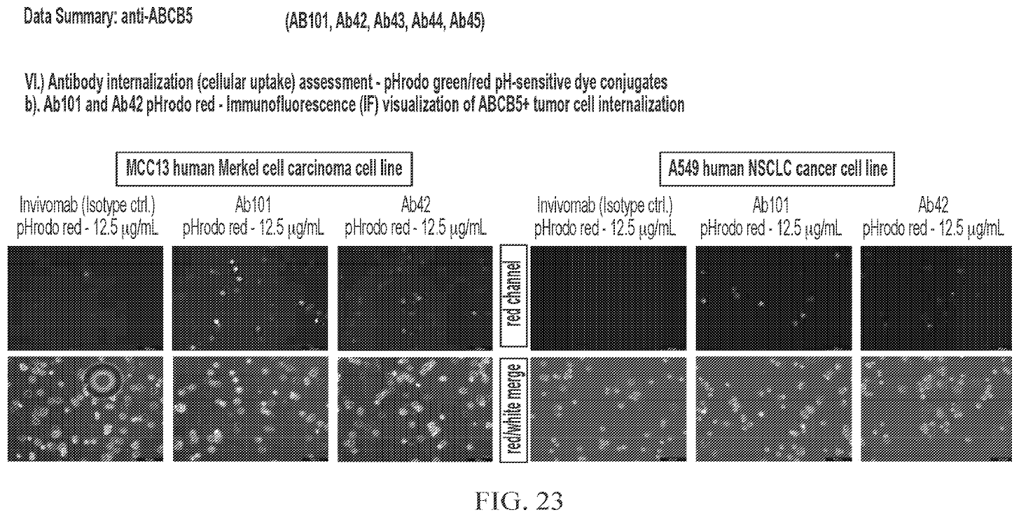

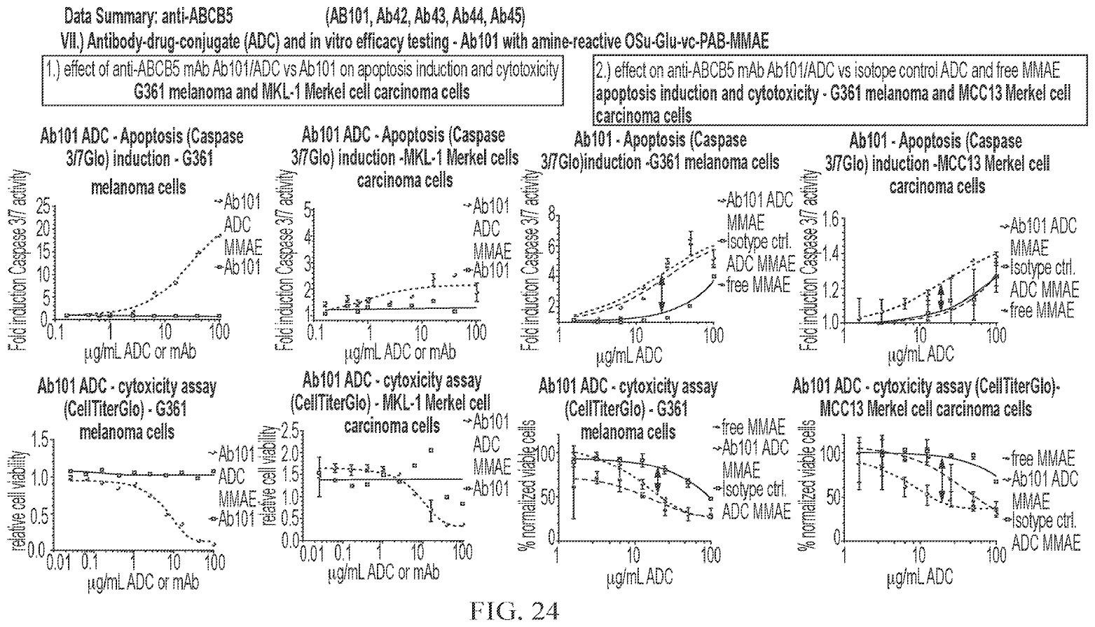

Additional experiments were carried out on numerous other antibodies disclosed herein. Some of the data is shown in FIGS. 11-24.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature. Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. An antibody binding to ATP-binding cassette transporter family member B5 (ABCB5),
wherein the antibody comprises a heavy chain variable domain (V_{H}), which comprises (i) a heavy chain complementary determining region 1 (HC CDR1) having at least 90% sequence identity to a sequence set forth as any one of SEQ ID NOs: 49-56, (ii) a heavy chain complementary determining region 2 (HC CDR2) having at least 90% sequence identity to a sequence set forth as any one of SEQ ID NOs: 57-65; and (iii) a heavy chain complementary determining region 3 (HC CDR3) having at least 90% sequence identity to a sequence set forth as any one of SEQ ID NOs: 66-72; and/or
wherein the antibody comprises a light chain variable domain (V_{L}), which comprises (i) a light chain complementary determining region 1 (LC CDR1) having at least 90% sequence identity to a sequence set forth as any one of SEQ ID NOs: 51-54, 73-76 and 80; (ii) a light chain complementary determining region 2 (LC CDR2) having at least 90% sequence identity to a sequence set forth as any one of SEQ ID NOs: 60-63 and 77-78; and (iii) a light chain complementary determining region 3 (LC CDR3) having at least 90% sequence identity to a sequence set forth as any one of SEQ ID NOs: 67, 69, 70, 79, 81, and 103, optionally wherein the antibody is not AB100 or Ab101.

2. An antibody binding to ATP-binding cassette transporter family member B5 (ABCB5),
wherein the antibody comprises a heavy chain variable domain (V_{H}), which comprises (i) a heavy chain complementary determining region 1 (HC CDR1) set forth as GFTFSSYX₁MN (SEQ ID NO: 109) or GYTFTX₂YYMH (SEQ ID NO: 110), in which X₁ is S or D or T and X₂ is S or N, (ii) a heavy chain complementary determining region 2 (HC CDR2) set forth as YISSSX₃X₄TIYYADSVKG (SEQ ID NO: 111) or IINPSGGSTSYAQKFX₅G (SEQ ID NO: 112), in which X₃ is S or G, and X₄ is S or N; and X₅ is K or Q and (iii) a heavy chain complementary determining region 3 (HC CDR3) set forth as NYQYGDYGGY (SEQ ID NO: 66) or DX₆AVTGTAYYYYYGMDV (SEQ ID NO: 113), in which X₆ is Q or L; and/or
wherein the antibody comprises a light chain variable domain (V_{L}), which comprises (i) a light chain complementary determining region 1 (LC CDR1) set forth as X₇ASHDISNFLN (SEQ ID NO: 114) or RASX₈SVNSX₉YLA (SEQ ID NO: 115), in which X₇ is Q or H; X₈ is L or Q; and X₉ is N or K (ii) a light chain complementary determining region 2 (LC CDR2) set forth as DAYNLQT (SEQ ID NO: 77) or GTSSRAT (SEQ ID NO: 78) and (iii) a light chain complementary determining region 3 (LC CDR3) set forth as QQYDYFLSIT (SEQ ID NO: 79) or QQFGSSPLT (SEQ ID NO: 81), optionally wherein the antibody is not AB100 or Ab101.

3. An antibody binding to ATP-binding cassette transporter family member B5 (ABCB5), wherein the antibody binds the same epitope as Ab100 or Ab101 or competes against Ab100 or Ab101 from binding to the ABCB5.

4. An antibody which recognizes an epitope of human ABCB5 comprising SEQ ID NO. 104 or having at least 80% sequence identity thereto.

5. The antibody of any one of claims 1 to 4, wherein the antibody specifically binds human ABCB5.

6. The antibody of any one of claims 1 to 4, wherein the antibody cross-reacts with human ABCB5 and a non-human ABCB5.

7. The antibody of any one of claims 1-6, wherein the antibody binds ABCB5 expressed on cell surface.

8. The antibody of claim 3 or 4, wherein the antibody comprises a HC CDR1, a HC CDR2, and a HC CDR3, which collectively contains no more than 10 amino acid variations as compared with the HC CDR1, HC CDR2, and HC CDR3 of Ab100 or Ab101; and/or a LC CDR1, a LC CDR2, and a LC CDR3, which collectively contains no more than 10 amino acid variations as compared with the LC CDR1, LC CDR2, and LC CDR3 of Ab100 or Ab101.

9. The antibody of claim 8, wherein the antibody comprises a HC CDR1, a HC CDR2, and a HC CDR3, which collectively contains no more than 8 amino acid variations as compared with the HC CDR1, HC CDR2, and HC CDR3 of Ab100 or Ab101.

10. The antibody of claim 9, wherein the antibody comprise a HC CDR1, a HC CDR2, and a HC CDR3, which collectively contains no more than 5 amino acid variations as compared with the HC CDR1, HC CDR2, and HC CDR3 of Ab100 or Ab101.

11. The antibody of any one of claims 8-10, wherein the antibody comprise a LC CDR1, a LC CDR2, and a LC CDR3, which collectively contains no more than 8 amino acid variations as compared with the LC CDR1, LC CDR2, and LC CDR3 of Ab100 or Ab101.

12. The antibody of claim 11, wherein the antibody comprise a LC CDR1, a LC CDR2, and a LC CDR3, which collectively contains no more than 5 amino acid variations as compared with the HC CDR1, HC CDR2, and HC CDR3 of Ab100 or Ab101.

13. The antibody of claim 2, wherein the antibody comprises a HC CDR1, a HC CDR2, and a HC CDR3, at least one of which contains no more than 5 amino acid variations as the counterpart HC CDR of Ab100 or Ab101; and/or a LC CDR1, a LC CDR2, and a LC CDR3, at least one of which contains no more than 5 amino acid variations as the counterpart LC CDR of Ab100 or Ab101.

14. The antibody of claim 13, wherein the antibody comprises a HC CDR1, a HC CDR2, and a HC CDR3, at least one of which contains no more than 2 amino acid variations as the counterpart HC CDR of Ab100 or Ab101.

15. The antibody of claim 13 or claim 14, wherein the at least one HC CDR is HC CDR3.

16. The antibody of claim 13 or claim 14, wherein the antibody comprises a LC CDR1, a LC CDR2, and a LC CDR3, at least one of which contains no more than 2 amino acid variations as the counterpart LC CDR of Ab100 or Ab101.

17. The antibody of claim 2, wherein the antibody comprises the same heavy chain complementary determining regions (HC CDRs) and/or the same light chain complementary determining regions (LC CDRs) as Ab100 or Ab101.

18. The antibody of claim 17, wherein the antibody comprises the same heavy chain variable domain as Ab100 or Ab101 and/or the same light chain variable domain as Ab100 or Ab101.

19. The antibody of any one of claims 1-17, wherein the antibody comprises a heavy chain variable domain that is at least 85% identical to the heavy chain variable domain of Ab100 or Ab101, and/or a light chain variable domain that is at least 85% identical to the light chain variable domain of Ab100 or Ab101.

20. The antibody of any one of claims 1-19 wherein the antibody is a human antibody or a humanized antibody.

21. The antibody of any one of claims 1-19, wherein the antibody is a full-length antibody.

22. The antibody of claim 21, wherein the full-length antibody is an IgG molecule.

23. The antibody of claim 21 or claim 22, wherein the antibody contains an altered Fc fragment relative to a naturally-occurring counterpart, or wherein the antibody contains an afucosylated Fc fragment, or wherein the antibody's antigen binding site is masked to allow protease mediated activation.

24. The antibody of claim 22, wherein the antibody contains an altered IgG1 Fc fragment, which comprises K214R.

25. The antibody of claim 23, wherein the antibody comprises a sequence having at least 90% sequence identity to a heavy chain variable sequence set forth as any of SEQ ID NO: 1-8 and 13-17 and a light chain variable sequence set forth as any of SEQ ID NO: 20-23 and 26-29.

26. The antibody of claim 23, wherein the antibody comprises a sequence having at least 90% sequence identity to a heavy chain sequence set forth as any of SEQ ID NO: 31-44 and a light chain sequence set forth as any of SEQ ID NO: 45-47.

27. The antibody of any one of claims 1-19, wherein the antibody a single-chain diabody (scDb), a bi-, tri-, tetra-, penta- or hexa-valent scFv tandem repeat (TaFv), is an antigen-binding fragment.

28. The antibody of claim 27, wherein the antigen-binding fragment is Fab, Fab′, F(ab′)2, or Fv.

29. The antibody of any one of claims 1-19, wherein the antibody is a single-chain antibody, a bispecific antibody or a nanobody.

30. The antibody of any one of claims 1-29, wherein the antibody is conjugated to a detectable label.

31. An antibody-drug conjugate (ADC), comprising an antibody of any one of claims 1-30 coupled to a therapeutic agent.

32. The ADC of claim 31, wherein the antibody is an scFv or a bi-, tri-, tetra-, penta- or hexa-valent scFv tandem repeat (TaFv) bivalent tandem scFv repeat (TaFv) thereof.

33. The ADC of claim 31 or 32, wherein the therapeutic agent is an auristatin peptide, auristatin E(AE), monomethylauristatin E(MMAE), or synthetic analog of dolastatin.

34. An antibody-drug conjugate (ADC), comprising an antibody binding specifically to ATP-binding cassette transporter family member B5 (ABCB5), coupled to a therapeutic agent through a linker.

35. The ADC of claim 34, wherein the antibody is an scFv or a bi-, tri-, tetra-, penta- or hexa-valent scFv tandem repeat (TaFv) bivalent tandem scFv repeat (TaFv) thereof.

36. The ADC of claim 34 or 35, wherein the therapeutic agent is an auristatin peptide, auristatin E(AE), monomethylauristatin E(MMAE), or synthetic analog of dolastatin.

37. The ADC of any of claims 34-36, wherein the linker is a flexible linker.

38. The ADC of any of claims 34-36, wherein the linker is selected from the group consisting of a peptide linker, a hydrocarbon linker, a polyethylene glycol (PEG) linker, a polypropylene glycol (PPG) linker, a polysaccharide linker, a polyester linker, a hybrid linker consisting of PEG and an embedded heterocycle, and a hydrocarbon chain.

39. The ADC of claim 36, wherein the linker is a PEG linker comprising 2-24 PEG units.

40. The ADC of claim 36, wherein the linker is a sulfamide linker.

41. The ADC of claim 36, wherein the linker is a peptide linker comprising GSTSGGGSGGGSGGGGSS (SEQ ID NO. 84) or GGGGSS (SEQ ID NO. 86).

42. A bispecific antibody, wherein the antibody has a region of antigen binding specificity for ATP-binding cassette transporter family member B5 (ABCB5) and a region of antigen binding specificity for an immune effector cell antigen.

43. The bispecific antibody of claim 42, wherein the immune effector cell antigen is Cd16 or CD3.

44. The bispecific antibody of claim 42, wherein region of antigen binding specificity for an immune effector cell antigen is an anti-Cd16 scFv or anti-CD3 scFv.

45. The bispecific antibody of claim 42, wherein region of antigen binding specificity for ABCB5 is an anti-ABCB5 scFv or an anti-ABCB5 monoclonal antibody.

46. The bispecific antibody of claim 42, wherein antibody comprises IgG-scFv fusion proteins [IgG-scFv's].

47. The bispecific antibody of claim 42, wherein antibody comprises Single-chain diabodies (scDb's).

48. The bispecific antibody of claim 42, wherein antibody comprises tandem scFv's (TaFv's).

49. A nucleic acid or a nucleic acid set, which collectively encode the antibody binding to ABCB5 set forth in any one of claim 1-29 or 41-47.

50. The nucleic acid or nucleic acid set of claim 49, which is a vector or a vector set.

51. The nucleic acid or nucleic acid set of claim 50, wherein the vector(s) is an expression vector(s).

52. A host cell, comprising the vector or vector set of claim 51.

53. The host cell of claim 52, which is selected from the group consisting of a bacterial cell, a yeast cell, an insect cell, a plant cell, and a mammalian cell.

54. A genetically engineered immune cell, which expresses a chimeric receptor comprising an extracellular domain and at least one cytoplasmic signaling domain, wherein the extracellular domain is a single chain antibody binding to ATP-binding cassette transporter family member B5 (ABCB5).

55. The genetically engineered immune cell of claim 55, wherein the single chain antibody comprises a heavy chain variable domain and/or a light chain variable domain set forth in any one of SEQ ID NOs 1-8, 13-17 and 20-23, 26-29 respectively.

56. A pharmaceutical composition, comprising (a) a antibody binding to ABCB5 set forth in any one of claims 1-29 and (b) a pharmaceutically acceptable carrier.

57. A method for treating cancer in a subject, the method comprising administering to a subject in need thereof an effective amount of the pharmaceutical composition of claim 56.

58. The method of claim 57 wherein the human patient has a metastatic cancer.

59. The method of any one of claims 57-58, wherein the subject has undergone or is undergoing an additional treatment of the disease.

60. The method of claim 59, wherein the disease is cancer and the treatment of cancer is surgery, a chemotherapy, an immune therapy, a radiotherapy, or a combination thereof.

61. A method for detecting presence of ABCB5, the method comprising contacting an anti-ABCB5 antibody of claims 1-22, alone or in combination with other anti-ABCB5 antibodies specifically binding to and/or capturing other ABCB5 epitopes, optionally selected from the group consisting of (RFGAYLIQAGRMTPEG (SEQ ID NO. 104), TMFGNNDKTTLKHDAE (SEQ ID NO. 105), VTGMIETAAMTGFANKDKQELKHAGKIATEALENIRTIVSLTREKAFEQMYEEMLQT QHRNTSKKAQI (SEQ ID NO. 106), and QDIKKADEQMESMTYSTERKTNSLPLHSVKSIKSDFIDKAEESTQSKEISLPEVSLLK (SEQ ID NO. 107), with a biological sample suspected of containing ABCB5, and measuring binding of the anti-ABCB5 antibody to ABCB5 in the sample.

62. The method of claim 60, wherein the treatment comprises administering to the subject an immune checkpoint antagonist.

63. A method for detecting presence of ABCB5, the method comprising contacting an anti-ABCB5 antibody of claims 1-29 with a biological sample suspected of containing ABCB5, and measuring binding of the anti-ABCB5 antibody to ABCB5 in the sample.

64. The method of claim 62 or claim 63, wherein the biological sample is in vivo and the contacting step is performed by administering the subject an effective of the anti-ABCB5 antibody.

65. A method for treating a tumor in a subject, the method comprising: obtaining immune leukocyte cells like T cells, NK cells monocytes and/or macrophages or combinations thereof from a subject having a tumor; transducing the T cells in vitro with a vector that contains a nucleic acid encoding a chimeric antigen receptor (CAR) including a scFv that specifically recognizes ABCB5, whereby the transduced T cells, NK cells, monocytes and/or macrophagesimmune cells express the CAR; expanding the transduced TCAR immune cells in vitro; and infusing the expanded transduced TCAR immune cells into the subject having a tumor, whereby an anti-tumor immuneT cell response is raised, wherein cells in the tumor express ABCB5.

66. The method of claim 63, wherein the antibody is conjugated to a detectable label.

67. The method of claim 63 or claim 66 wherein the biological sample is in vivo and the contacting step is performed by administering the subject an effective of the anti-ABCB5 antibody.

68. A method for treating a tumor in a subject, the method comprising: obtaining T cells from a subject having a tumor; transducing the T cells in vitro with a vector that contains a nucleic acid encoding a chimeric antigen receptor (CAR) including a scFv that specifically recognizes ABCB5, whereby the transduced T cells express the CAR; expanding the transduced T cells in vitro; and infusing the expanded transduced T cells into the subject having a tumor, whereby an anti-tumor T cell response is raised, wherein cells in the tumor express ABCB5.

69. An isolated chimeric antigen receptor (CAR) comprising an ABCB5 binding domain, a transmembrane domain and an intracellular signalling domain wherein the ABCB5 binding domain comprises a human variable heavy chain (V_{H}) domain.

70. The CAR of claim 69, wherein the ABCB5 binding domain comprises an antibody of any one of claims 1-29.

71. An isolated cell or cell population comprising one or more CAR as defined in any of claims 69 to 70.

72. A cell or cell population according to claim 71 wherein said cell is selected from the group consisting of a T cell, a Natural Killer (NK) cell, a cytotoxic T lymphocyte (CTL), and a regulatory T cell.

73. A pharmaceutical composition comprising a cell or cell population as defined in claim 71 or 72 and a pharmaceutical acceptable carrier, excipient or diluent.

74. A method for treating cancer comprising administering a cell or cell population according to claim 62 or pharmaceutical composition according to claim 63.

75. A cell according to claim 72 or a pharmaceutical composition according to claim 29 for use in therapy.

76. A cell according to claim 72 or a pharmaceutical composition according to claim 73 for use in the treatment of cancer.

77. The use of a cell according to claim 72 or a pharmaceutical composition according to claim 63 in the manufacture of a medicament for the treatment of cancer.


SEQUENCE LISTING (173 sequences)

SEQ ID NO: 1 - protein, 119 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSS

SEQ ID NO: 2 - protein, 119 aa, Artificial Sequence
  Synthetic
EVQLVESGGGLVQPGGSLRLSCAASGFTFSSYDMNWVRQAPGKGLEWVSYISSSSNTIYYADSVKGRFTISRDNAKNSLYLQMNSLRDEDTAVYYCASNYQYGDYGGYWGRGTLVTVSS

SEQ ID NO: 3 - protein, 119 aa, Artificial Sequence
  Synthetic
EVQLVESGGGLVQPGGSLRLSCAASGFTFSSYSMNWVRQAPGKGPEWVSYISSSGSTIYYADSVKGRFTISRDNAKNSLYLQMNSLRDEDTAVYYCASNYQYGDYGGYWGRGTLVTVSS

SEQ ID NO: 4 - protein, 119 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSS

SEQ ID NO: 5 - protein, 119 aa, Artificial Sequence
  Synthetic
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCASNYQYGDYGGYWGRGTLVTVSS

SEQ ID NO: 6 - protein, 119 aa, Artificial Sequence
  Synthetic
EVQLLESGGGLVQPGGSLRLSCAASGFTFSSYDMNWVRQAPGKGLEWVSYISSSSNTIYYADSVKGRFTISRDNSKNTLYLQMNSLRAEDTAVYYCAKNYQYGDYGGYWGRGTLVTVSS

SEQ ID NO: 7 - protein, 119 aa, Artificial Sequence
  Synthetic
EVQLLESGGGLVQPGGSLRLSCAASGFTFSSYSMNWVRQAPGKGPEWVSYISSSGSTIYYADSVKGRFTISRDNSKNTLYLQMNSLRAEDTAVYYCAKNYQYGDYGGYWGRGTLVTVSS

SEQ ID NO: 8 - protein, 119 aa, Artificial Sequence
  Synthetic
EVQLLESGGGLVQPGGSLRLSCAASGFTFSSYSMNWVRQAPGKGPEWVSYISSSSSTIYYADSVKGRFTISRDNSKNTLYLQMNSLRAEDTAVYYCAKNYQYGDYGGYWGRGTLVTVSS

SEQ ID NO: 9 - protein, 16 aa, Artificial Sequence
  Synthetic
RFGAYLVAHKLMSFED

SEQ ID NO: 10 - protein, 16 aa, Artificial Sequence
  Synthetic
RFGAYLIVNGHMRFRD

SEQ ID NO: 11 - protein, 16 aa, Artificial Sequence
  Synthetic
RYGGYLISNEGLHFSY

SEQ ID NO: 12 - protein, 16 aa, Artificial Sequence
  Synthetic
RFGAYLIQAGRMMPEG

SEQ ID NO: 13 - protein, 126 aa, Artificial Sequence
  Synthetic
QVQLVQSGAEVKKPGASVKVSCKTSGYTFTSYYMHWVRQSPGQGLEWMGIINPSGGSTSYAQKFQGRVTMTRDTSTSTVYMELSSLRSEDTAVYYCARDQAVTGTAYYYYYGMDVWGQGTTVTVSS

SEQ ID NO: 14 - protein, 126 aa, Artificial Sequence
  Synthetic
QVQLVQSGAEVKKPGASVKVSCKASGYTFTSYYMHWVRQSPGQGLEWMGIINPSGGSTSYAQKFQGRVTMTRDTSTSTVYMELSSLRSEDTAVYYCARDQAVTGTAYYYYYGMDVWGQGTTVTVSS

SEQ ID NO: 15 - protein, 126 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSS

SEQ ID NO: 16 - protein, 126 aa, Artificial Sequence
  Synthetic
QVQLVQSGAEVKKPGASVKVSCKASGYTFTNYYMHWVRQAPGQGPEWMGIINPSGGSTSYAQKFQGRVTMTRDTSTSTVYMELSSLRSEDTAVYYCARDLAVTGTAYYYYYGMDVWGQGTTVTVSS

SEQ ID NO: 17 - protein, 126 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSS

SEQ ID NO: 18 - protein, 16 aa, Artificial Sequence
  Synthetic
GFEALGTAIRYPMGQR

SEQ ID NO: 19 - protein, 821 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGSGGGGSSGSTSGGGSGGGSGGGSGGGGSSQVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGSGGGGSSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPE

SEQ ID NO: 20 - protein, 108 aa, Artificial Sequence
  Synthetic
DIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIK

SEQ ID NO: 21 - protein, 108 aa, Artificial Sequence
  Synthetic
AIQLTQSPSSLTGSVGDRVTITCHASHDISNFLNWYQQKPGQAPKLLIYDAYNLQTGVPSRFSGTGSGTHFTLTINSLQPEDVGTYFCQQYDYFLSITFGQGTRLEIK

SEQ ID NO: 22 - protein, 108 aa, Artificial Sequence
  Synthetic
AIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIK

SEQ ID NO: 23 - protein, 108 aa, Artificial Sequence
  Synthetic
DIQMTQSPSSLSASVGDRVTITCHASHDISNFLNWYQQKPGKAPKLLIYDAYNLQTGVPSRFSGSGSGTDFTFTISSLQPEDIATYYCQQYDYFLSITFGQGTKVEIK

SEQ ID NO: 24 - protein, 18 aa, Artificial Sequence
  Synthetic
CRFGAYLIQAGRMTPEGC

SEQ ID NO: 25 - protein, 18 aa, Artificial Sequence
  Synthetic
CGFEALGTAIRYPMGQRC

SEQ ID NO: 26 - protein, 108 aa, Artificial Sequence
  Synthetic
EIVLTQSPGTLSLSPGERATLSCRASLSVNSNYLAWYQQKPGQAPRLLIYGTSSRATGIPDSFSGSGSGTDFTLTISRLEPEDFAVYYCQQFGSSPLTFGGGTKVEIK

SEQ ID NO: 27 - protein, 108 aa, Artificial Sequence
  Synthetic
EIVLTQSPGTLSLSPGERATLSCRASQSVNSKYLAWYQQKPGQAPRLLIYGTSSRATGIPDSFSGSGSGTDFTLTISRLEPEDFAVYYCQQFGSSPLTFGGGTKVEIK

SEQ ID NO: 28 - protein, 108 aa, Artificial Sequence
  Synthetic
EIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIK

SEQ ID NO: 29 - protein, 108 aa, Artificial Sequence
  Synthetic
EFVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIK

SEQ ID NO: 30 - protein, 408 aa, Artificial Sequence
  Synthetic
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTGSTGGGSGGGSGGGSGGGGSS

SEQ ID NO: 31 - protein, 449 aa, Artificial Sequence
  Synthetic
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 32 - protein, 449 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 33 - protein, 449 aa, Artificial Sequence
  Synthetic
EVQLVESGGGLVQPGGSLRLSCAASGFTFSSYDMNWVRQAPGKGLEWVSYISSSSNTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 34 - protein, 449 aa, Artificial Sequence
  Synthetic
EVQLVESGGGLVQPGGSLRLSCAASGFTFSSYSMNWVRQAPGKGPEWVSYISSSGSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 35 - protein, 449 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 36 - protein, 449 aa, Artificial Sequence
  Synthetic
EVQLLESGGGLVQPGGSLRLSCAASGFTFSSYDMNWVRQAPGKGLEWVSYISSSSNTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 37 - protein, 449 aa, Artificial Sequence
  Synthetic
EVQLLESGGGLVQPGGSLRLSCAASGFTFSSYSMNWVRQAPGKGPEWVSYISSSGSTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 38 - protein, 449 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVEDVSHEDPEVKFNWYVDGVEVHNAKTKPREEQYNGAYRVVSVLTVLHQDWLNGKEYKCKVSNIYGPAPIEKTISKAKGQPREPQVYALPPSREEMTKNQVSLTCLVKGFYPSDIAVEWVSNGQPENDYKTTPPVLDSDGSFFLYSKLTVDKSRWQQGNVFSCSVLHEALHNHYTQKSLSLSPGK

SEQ ID NO: 39 - protein, 449 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVEDVSHEDPEVKFNWYVDGVEVHNAKTKPREEQYNGAYRVVSVLTVLHQDWLNGKEYKCKVSNIYGPAPIEKTISKAKGQPREPQVYALPPSREEMTKNQVSLTCLVKGFYPSDIAVEWVSNGQPENDYKTTPPVLDSDGSFFLYSKLTVDKSRWQQGNVFSCSVLHEALHNHYTQKSLSLSPGK

SEQ ID NO: 40 - protein, 449 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 449 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 456 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 456 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 449 aa, Artificial Sequence
  Synthetic
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 215 aa, Artificial Sequence
  Synthetic
DIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 215 aa, Artificial Sequence
  Synthetic
AIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 215 aa, Artificial Sequence
  Synthetic
EIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 247 aa, Artificial Sequence
  Synthetic
MVDENDIRALNVRHYRDHIGVVSQEPVLFGTTISNNIKYGRDDVTDEEMERAAREANAYDFIMEFPNKFNTLVGEKGAQMSGGQKQRIAIARALVRNPKILILDEATSALDSESKSAVQAALEKASKGRTTIVVAHRLSTIRSADLIVTLKDGMLAEKGAHAELMAKRGLYYSLVMSQDIKKADEQMESMTYSTERKTNSLPLHSVKSIKSDFIDKAEESTQSKEISLPEVSLLKILKLNKPEWPFV

SEQ ID NO: 49 - protein, 10 aa, Artificial Sequence
  Synthetic
GFTFSSYSMN

SEQ ID NO: 50 - protein, 10 aa, Artificial Sequence
  Synthetic
GFTFSSYDMN

SEQ ID NO: 51 - protein, 10 aa, Artificial Sequence
  Synthetic
GYTFTRYTMH

SEQ ID NO: 52 - protein, 10 aa, Artificial Sequence
  Synthetic
SASSSVSYMN

SEQ ID NO: 53 - protein, 12 aa, Artificial Sequence
  Synthetic
GFSLSTSGMGVG

SEQ ID NO: 54 - protein, 15 aa, Artificial Sequence
  Synthetic
KASQSVDFDGDSFMN

SEQ ID NO: 55 - protein, 10 aa, Artificial Sequence
  Synthetic
GYTFTSYYMH

SEQ ID NO: 56 - protein, 10 aa, Artificial Sequence
  Synthetic
GYTFTNYYMH

SEQ ID NO: 57 - protein, 17 aa, Artificial Sequence
  Synthetic
YISSSSSTIYYADSVKG

SEQ ID NO: 58 - protein, 17 aa, Artificial Sequence
  Synthetic
YISSSSNTIYYADSVKG

SEQ ID NO: 59 - protein, 17 aa, Artificial Sequence
  Synthetic
YISSSGSTIYYADSVKG

SEQ ID NO: 60 - protein, 19 aa, Artificial Sequence
  Synthetic
YINPSRGYTNYNQKFKDKA

SEQ ID NO: 61 - protein, 7 aa, Artificial Sequence
  Synthetic
DTSKLAS

SEQ ID NO: 62 - protein, 16 aa, Artificial Sequence
  Synthetic
HIWWDDDKRYNPALKS

SEQ ID NO: 63 - protein, 7 aa, Artificial Sequence
  Synthetic
TTSNLES

SEQ ID NO: 64 - protein, 17 aa, Artificial Sequence
  Synthetic
IINPSGGSTSYAQKFQG

SEQ ID NO: 65 - protein, 17 aa, Artificial Sequence
  Synthetic
IINPSGGSTSYAQKFKG

SEQ ID NO: 66 - protein, 10 aa, Artificial Sequence
  Synthetic
NYQYGDYGGY

SEQ ID NO: 67 - protein, 10 aa, Artificial Sequence
  Synthetic
YYDDHYCLDY

SEQ ID NO: 68 - protein, 281 aa, Artificial Sequence
  Synthetic
EYSKAKSGAAHLFALLEKKPNIDSRSQEGKKPDTCEGNLEFREVSFFYPCRPDVFILRGLSLSIERGKTVAFVGSSGCGKSTSVQLLQRLYDPVQGQVLFDGVDAKELNVQWLRSQIAIVPQEPVLFNCSIAENIAYGDNSRVVPLDEIKEAANAANIHSFIEGLPEKYNTQVGLKGAQLSGGQKQRLAIARALLQKPKILLLDEATSALDNDSEKVVQHALDKARTGRTCLVVTHRLSAIQNADLIVVLHNGKIKEQGTHQELLRNRDIYFKLVNAQSVQ

SEQ ID NO: 69 - protein, 8 aa, Artificial Sequence
  Synthetic
INPAWFAY

SEQ ID NO: 70 - protein, 9 aa, Artificial Sequence
  Synthetic
QQSNEDPYT

SEQ ID NO: 71 - protein, 17 aa, Artificial Sequence
  Synthetic
DQAVTGTAYYYYYGMDV

SEQ ID NO: 72 - protein, 17 aa, Artificial Sequence
  Synthetic
DLAVTGTAYYYYYGMDV

SEQ ID NO: 73 - protein, 11 aa, Artificial Sequence
  Synthetic
QASHDISNFLN

SEQ ID NO: 74 - protein, 11 aa, Artificial Sequence
  Synthetic
HASHDISNFLN

SEQ ID NO: 75 - protein, 12 aa, Artificial Sequence
  Synthetic
RASLSVNSNYLA

SEQ ID NO: 76 - protein, 12 aa, Artificial Sequence
  Synthetic
RASQSVNSKYLA

SEQ ID NO: 77 - protein, 7 aa, Artificial Sequence
  Synthetic
DAYNLQT

SEQ ID NO: 78 - protein, 7 aa, Artificial Sequence
  Synthetic
GTSSRAT

SEQ ID NO: 79 - protein, 10 aa, Artificial Sequence
  Synthetic
QQYDYFLSIT

SEQ ID NO: 80 - protein, 12 aa, Artificial Sequence
  Synthetic
RASQSVNSNYLA

SEQ ID NO: 81 - protein, 9 aa, Artificial Sequence
  Synthetic
QQFGSSPLT

SEQ ID NO: 82 - protein, 10 aa, Artificial Sequence
  Synthetic
GGGGSGGGGS

SEQ ID NO: 83 - protein, 5 aa, Artificial Sequence
  Synthetic
GGGGS

SEQ ID NO: 84 - protein, 18 aa, Artificial Sequence
  Synthetic
GSTSGGGSGGGSGGGGSS

SEQ ID NO: 85 - protein, 6 aa, Artificial Sequence
  Synthetic
SGGGGS

SEQ ID NO: 86 - protein, 6 aa, Artificial Sequence
  Synthetic
GGGGSS

SEQ ID NO: 87 - protein, 24 aa, Artificial Sequence
  Synthetic
  MISC_FEATURE (1)..(1): may be absent
  MISC_FEATURE (24)..(24): may be absent
CGSTSGGGSGGGSGGGSGGGGSSC

SEQ ID NO: 88 - protein, 44 aa, Artificial Sequence
  Synthetic
GSTSGGGSGGGSGGGSGGGGSSGSTSGGGSGGGSGGGSGGGGSS

SEQ ID NO: 89 - protein, 21 aa, Artificial Sequence
  Synthetic
GSTGGGSGGGSGGGSGGGGSS

SEQ ID NO: 90 - protein, 243 aa, Artificial Sequence
  Synthetic
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 91 - protein, 247 aa, Artificial Sequence
  Synthetic
QVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 92 - protein, 243 aa, Artificial Sequence
  Synthetic
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 93 - protein, 119 aa, Artificial Sequence [sequence omitted]

SEQ ID NO: 94 - protein, 106 aa, Artificial Sequence [sequence omitted]

SEQ ID NO: 95 - protein, 247 aa, Artificial Sequence
  Synthetic
DIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSS

SEQ ID NO: 96 - protein, 118 aa, Artificial Sequence
  Synthetic
QVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSS

SEQ ID NO: 97 - protein, 111 aa, Artificial Sequence
  Synthetic
DIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 98 - protein, 251 aa, Artificial Sequence
  Synthetic
GLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPE

SEQ ID NO: 99 - protein, 361 aa, Artificial Sequence
  Synthetic
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYAYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 100 - protein, 276 aa, Artificial Sequence
  Synthetic
KIYVVATIAWILLQFSAWTTTDAVTSITLDLVNPTAGQYSSFVDKIRNNVKDPNLKYGGTDIAVIGPPSKEKFLRINFQSSRGTVSLGLKRDNLYVVAYLAMDNTNVNRAYYFRSEITSAESTALFPEATTANQKALEYTEDYQSIEKNAQITQGDQSRKELGLGIDLLSTSMEAVNKKARVVKDEARFLLIAIQMTAEAARFRYIQNLVIKNFPNKFNSENKVIQFEVNWKKISTAIYGDAKNGVFNKDYDFGFGKVRQVKDLQMGLLMYLGKPK

SEQ ID NO: 101 - protein, 43 aa, Artificial Sequence
  Synthetic
GSPRTEYEACRVRCQVAEHGVERQRRCQQVCEKRLREREGRRE

SEQ ID NO: 102 - protein, 387 aa, Artificial Sequence
  Synthetic
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKT

SEQ ID NO: 103 - protein, 9 aa, Artificial Sequence
  Synthetic
QQWSSNPFT

SEQ ID NO: 104 - protein, 16 aa, Artificial Sequence
  Synthetic
RFGAYLIQAGRMTPEG

SEQ ID NO: 105 - protein, 16 aa, Artificial Sequence
  Synthetic
TMFGNNDKTTLKHDAE

SEQ ID NO: 106 - protein, 68 aa, Artificial Sequence
  Synthetic
VTGMIETAAMTGFANKDKQELKHAGKIATEALENIRTIVSLTREKAFEQMYEEMLQTQHRNTSKKAQI

SEQ ID NO: 107 - protein, 58 aa, Artificial Sequence
  Synthetic
QDIKKADEQMESMTYSTERKTNSLPLHSVKSIKSDFIDKAEESTQSKEISLPEVSLLK

SEQ ID NO: 108 - protein, 720 aa, Artificial Sequence
  Synthetic
  MISC_FEATURE (270)..(270): may be absent
  MISC_FEATURE (287)..(287): may be absent
  MISC_FEATURE (400)..(400): may be absent
  MISC_FEATURE (417)..(417): may be absent
MVDENDIRALNVRHYRDHIGVVSQEPVLFGTTISNNIKYGRDDVTDEEMERAAREANAYDFIMEFPNKFNTLVGEKGAQMSGGQKQRIAIARALVRNPKILILDEATSALDSESKSAVQAALEKASKGRTTIVVAHRLSTIRSADLIVTLKDGMLAEKGAHAELMAKRGLYYSLVMSQDIKKADEQMESMTYSTERKTNSLPLHSVKSIKSDFIDKAEESTQSKEISLPEVSLLKILKLNKPEWPFVGSTSGGGSGGGSGGGSGGGGSSCTMFGNNDKTTLKHDAECGSTSGGGSGGGSGGGSGGGGSSVTGMIETAAMTGFANKDKQELKHAGKIATEALENIRTIVSLTREKAFEQMYEEMLQTQHRNTSKKAQIGSTSGGGSGGGSGGGSGGGGSSCRFGAYLIQAGRMTPEGCGSTSGGGSGGGSGGGSGGGGSSEYSKAKSGAAHLFALLEKKPNIDSRSQEGKKPDTCEGNLEFREVSFFYPCRPDVFILRGLSLSIERGKTVAFVGSSGCGKSTSVQLLQRLYDPVQGQVLFDGVDAKELNVQWLRSQIAIVPQEPVLFNCSIAENIAYGDNSRVVPLDEIKEAANAANIHSFIEGLPEKYNTQVGLKGAQLSGGQKQRLAIARALLQKPKILLLDEATSALDNDSEKVVQHALDKARTGRTCLVVTHRLSAIQNADLIVVLHNGKIKEQGTHQELLRNRDIYFKLVNAQSVQ

SEQ ID NO: 109 - protein, 10 aa, Artificial Sequence
  Synthetic
  misc_feature (8)..(8): Xaa can be Ser or Asp
GFTFSSYXMN

SEQ ID NO: 110 - protein, 10 aa, Artificial Sequence
  Synthetic
  misc_feature (6)..(6): Xaa can be Ser or Asn
GYTFTXYYMH

SEQ ID NO: 111 - protein, 17 aa, Artificial Sequence
  Synthetic
  misc_feature (6)..(6): Xaa can be Ser or Gly
  misc_feature (7)..(7): Xaa can be Ser or Asn
YISSSXXTIYYADSVKG

SEQ ID NO: 112 - protein, 17 aa, Artificial Sequence
  Synthetic
  misc_feature (16)..(16): Xaa can be Lys or Gln
IINPSGGSTSYAQKFXG

SEQ ID NO: 113 - protein, 17 aa, Artificial Sequence
  Synthetic
  misc_feature (2)..(2): Xaa can be Gln or Leu
DXAVTGTAYYYYYGMDV

SEQ ID NO: 114 - protein, 11 aa, Artificial Sequence
  Synthetic
  misc_feature (1)..(1): Xaa can be Gln or His
XASHDISNFLN

SEQ ID NO: 115 - protein, 12 aa, Artificial Sequence
  Synthetic
  misc_feature (4)..(4): Xaa can be Leu or Gln
  misc_feature (9)..(9): Xaa can be Asn or Lys
RASXSVNSXYLA

SEQ ID NO: 116 - protein, 449 aa, Artificial Sequence; identical to SEQ ID NO: 44
  Synthetic

SEQ ID NO: 117 - protein, 456 aa, Artificial Sequence; identical to SEQ ID NO: 42
  Synthetic

SEQ ID NO: 118 - protein, 449 aa, Artificial Sequence
  Synthetic
EVQLLESGGGLVQPGGSLRLSCAASGFTFSSYSMNWVRQAPGKGPEWVSYISSSSSTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 119 - protein, 702 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 120 - protein, 702 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 121 - protein, 468 aa, Artificial Sequence
  Synthetic
DIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 122 - protein, 468 aa, Artificial Sequence
  Synthetic
AIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 123 - protein, 706 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 124 - protein, 706 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 125 - protein, 472 aa, Artificial Sequence
  Synthetic
DIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 126 - protein, 472 aa, Artificial Sequence
  Synthetic
AIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 127 - protein, 480 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIK

SEQ ID NO: 128 - protein, 480 aa, Artificial Sequence
  Synthetic
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGSTSGGGSGGGSGGGGSSEVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 129 - protein, 481 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIK

SEQ ID NO: 130 - protein, 480 aa, Artificial Sequence
  Synthetic
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKGSTSGGGSGGGSGGGGSSEVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 131 - protein, 484 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIK

SEQ ID NO: 132 - protein, 484 aa, Artificial Sequence
  Synthetic
QVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGSTSGGGSGGGSGGGGSSEVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 133 - protein, 485 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIK

SEQ ID NO: 134 - protein, 484 aa, Artificial Sequence
  Synthetic
QVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKGSTSGGGSGGGSGGGGSSEVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 135 - protein, 494 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 136 - protein, 506 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 137 - protein, 494 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSGSTSGGGSGGGSGGGGSSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 138 - protein, 506 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSGSTSGGGSGGGSGGGGSSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 139 - protein, 498 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 140 - protein, 510 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 141 - protein, 498 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSGSTSGGGSGGGSGGGGSSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 142 - protein, 510 aa, Artificial Sequence
  Synthetic
EVQLVESGGGLVQPGGSLRLSCAASGFTFSSYSMNWVRQAPGKGPEWVSYISSSSSTIYYADSVKGRFTISRDNAKNSLYLQMNSLRDEDTAVYYCASNYQYGDYGGYWGRGTLVTVSSGSTSGGGSGGGSGGGGSSAIQLTQSPSSLTGSVGDRVTITCQASHDISNFLNWYQQKPGQAPKLLIYDAYNLQTGVPSRFSGTGSGTHFTLTINSLQPEDVGTYFCQQYDYFLSITFGQGTRLEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 143 - protein, 709 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 144 - protein, 468 aa, Artificial Sequence
  Synthetic
EIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 145 - protein, 713 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 146 - protein, 472 aa, Artificial Sequence
  Synthetic
EIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 147 - protein, 487 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIK

SEQ ID NO: 148 - protein, 487 aa, Artificial Sequence
  Synthetic
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 149 - protein, 491 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIK

SEQ ID NO: 150 - protein, 491 aa, Artificial Sequence
  Synthetic
QVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 151 - protein, 501 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 152 - protein, 513 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 153 - protein, 505 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 154 - protein, 517 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 155 - protein, 709 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 156 - protein, 713 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 157 - protein, 487 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIK

SEQ ID NO: 158 - protein, 487 aa, Artificial Sequence
  Synthetic
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 159 - protein, 491 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIK

SEQ ID NO: 160 - protein, 491 aa, Artificial Sequence
  Synthetic
QVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGGGGSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGGGGSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 161 - protein, 501 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 162 - protein, 513 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGSTSGGGSGGGSGGGGSSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIN

SEQ ID NO: 163 - protein, 505 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 164 - protein, 517 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGGSSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSSGSTSGGGSGGGSGGGGSSDIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIK

SEQ ID NO: 165 - protein, 524 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGGGGSEQKLISEEDLQLSGGGGSMGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPEKDEL

SEQ ID NO: 166 - protein, 514 aa, Artificial Sequence
  Synthetic
EVQLLESGGGLVQPGGSLRLSCAASGFTFSSYSMNWVRQAPGKGLEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGGGGSQLSGGGGSMGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPEKDEL

SEQ ID NO: 167 - protein, 531 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGGGGSEQKLISEEDLQLSGGGGSMGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPEKDEL

SEQ ID NO: 168 - protein, 521 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGGGGSQLSGGGGSMGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPEKDEL

SEQ ID NO: 169 - protein, 518 aa, Artificial Sequence
  Synthetic
EVQLLESGGGLVQPGGSLRLSCAASGFTFSSYSMNWVRQAPGKGPEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGSTSGGGSGGGSGGGSGGGGSSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPE

SEQ ID NO: 170 - protein, 807 aa, Artificial Sequence
  Synthetic
EVQLLESGGGLVQPGGSLRLSCAASGFTFSSYSMNWVRQAPGKGPEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGSTSGGGSGGGSGGGSGGGGSSGSTSGGGSGGGSGGGSGGGGSSEVQLLESGGGLVQPGGSLRLSCAASGFTFSSYSMNWVRQAPGKGPEWVSYISSSSSTIYYADSVKGRFTISRDNSKNTLYLQMNSLRAEDTAVYYCAKNYQYGDYGGYWGRGTLVTVSSGSTSGGGSGGGSGGGGSSDIQMTQSPSSLSASVGDRVTITCQASHDISNFLNWYQQKPGKAPKLLIYDAYNLQTGVPSRFSGSGSGTDFTFTISSLQPEDIATYYCQQYDYFLSITFGQGTKVEIKGSTSGGGSGGGSGGGSGGGGSSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPE

SEQ ID NO: 171 - protein, 525 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGSGGGGSSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPE

SEQ ID NO: 172 - protein, 821 aa, Artificial Sequence
  Synthetic
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGSGGGGSSGSTSGGGSGGGSGGGSGGGGSSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGSGGGGSSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPE

SEQ ID NO: 173 - protein, 525 aa, Artificial Sequence
  Synthetic
QVQLVQSGAEVKKPGASVKVSCKASGYTFTSYYMHWVRQAPGQGLEWMGIINPSGGSTSYAQKFKGRVTMTRDTSTSTVYMELSSLRSEDTAVYYCARDQAVTGTAYYYYYGMDVWGQGTTVTVSSGSTSGGGSGGGSGGGGSSEIVLTQSPGTLSLSPGERATLSCRASQSVNSNYLAWYQQKPGQAPRLLIYGTSSRATGIPDSFSGSGSGTDFTLTISRLEPEDFAVYYCQQFGSSPLTFGGGTKVEIKGSTSGGGSGGGSGGGSGGGGSSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPE